SEQUENCE LISTING (160 sequences)

SEQ ID NO: 1 - protein, 70 aa, Homo sapiens
EAEEDGDLQCLCVKTTSQVRPRHITSLEVIKAGPHCPTAQLIATLKNGRKICLDLQAPLYKKIIKKLLES

SEQ ID NO: 2 - protein, 70 aa, Homo sapiens
EAEEDGDLQCLCVKTTSQVRPRHITSLEVIKAGPHCPTAQLIATLKNGRKICLDLQAPLYQEIIQELLES

SEQ ID NO: 3 - protein, 70 aa, Homo sapiens
EAEEDGDLQSLCVKTTSQVRPRHITSLEVIKAGPHSPTAQLIATLKNGRKICLDLQAPLYKKIIKKLLES

SEQ ID NO: 4 - protein, 70 aa, Homo sapiens
EAEEDGDLQSLCVKTTSQVRPRHITSLEVIKAGPHSPTAQLIATLKNGRKICLDLQAPLYQEIIQELLES

SEQ ID NO: 5 - protein, 70 aa, Homo sapiens
EAEEDGDLRCLCVKTTSQVRPRHITSLEVIKAGPHCPTAQLIATLKNGRKICLDLQAPLYKKIIKKLLES

SEQ ID NO: 6 - protein, 70 aa, Homo sapiens
EAEEDGDLRCLCVKTTSQVRPRHITSLEVIKAGPHCPTAQLIATLKNGRKICLDLQAPLYQEIIQELLES

SEQ ID NO: 7 - protein, 70 aa, Homo sapiens
EAEEDGALACLCVKTTSQVRPRHITSLEVIKAGPHCPTAQLIATLKNGRKICLDLQAPLYKKIIKKLLES

SEQ ID NO: 8 - protein, 70 aa, Homo sapiens
EAEEDGALACLCVKTTSQVRPRHITSLEVIKAGPHCPTAQLIATLKNGRKICLDLQAPLYQEIIQELLES

SEQ ID NO: 9 - protein, 70 aa, Homo sapiens
EAEEDGDLQCSCQKTASQVRPRHITSLEVIKAGPHCPTAQLIATLKNGRKICLDLQAPLYKKIIKKLLES

SEQ ID NO: 10 - protein, 70 aa, Homo sapiens
EAEEDGDLQCSCQKTASQVRPRHITSLEVIKAGPHCPTAQLIATLKNGRKICLDLQAPLYQEIIQELLES

SEQ ID NO: 11 - protein, 70 aa, Homo sapiens
EAEEDGDLQCLCVKTTSASRPRAITSLEVIKAGPHCPTAQLIATLKNGRKICLDLQAPLYKKIIKKLLES

SEQ ID NO: 12 - protein, 70 aa, Homo sapiens
EAEEDGDLQCLCVKTTSASRPRAITSLEVIKAGPHCPTAQLIATLKNGRKICLDLQAPLYQEIIQELLES

SEQ ID NO: 13 - protein, 73 aa, Homo sapiens
EAEEDGDLQCLCVKTTSQVRPRHITSLEVIKAGPHCPTAQLIATLKNGRKICLDLQAPLYQEIIQELLESYYY

SEQ ID NO: 14 - protein, 70 aa, Homo sapiens
EAEEDGDLQCSCVKTTSQVRPRHITSLEVIKAGPHCPTAQLIATLKNGRKICLDLQAPLYKKIIKKLLES

SEQ ID NO: 15 - protein, 70 aa, Homo sapiens
EAEEDGDLQCSCVKTTSQVRPRHITSLEVIKAGPHCPTAQLIATLKNGRKICLDLQAPLYQEIIQELLES

SEQ ID NO: 16 - protein, 70 aa, Homo sapiens
EAEEDGDLQCLCQKTTSQVRPRHITSLEVIKAGPHCPTAQLIATLKNGRKICLDLQAPLYKKIIKKLLES

SEQ ID NO: 17 - protein, 70 aa, Homo sapiens
EAEEDGDLQCLCQKTTSQVRPRHITSLEVIKAGPHCPTAQLIATLKNGRKICLDLQAPLYQEIIQELLES

SEQ ID NO: 18 - protein, 70 aa, Homo sapiens
EAEEDGDLQCLCVKTASQVRPRHITSLEVIKAGPHCPTAQLIATLKNGRKICLDLQAPLYKKIIKKLLES

SEQ ID NO: 19 - protein, 70 aa, Homo sapiens
EAEEDGDLQCLCVKTASQVRPRHITSLEVIKAGPHCPTAQLIATLKNGRKICLDLQAPLYQEIIQELLES

SEQ ID NO: 20 - protein, 70 aa, Homo sapiens
EAEEDGDLQCLCVKTTSAVRPRHITSLEVIKAGPHCPTAQLIATLKNGRKICLDLQAPLYKKIIKKLLES

SEQ ID NO: 21 - protein, 70 aa, Homo sapiens
EAEEDGDLQCLCVKTTSAVRPRHITSLEVIKAGPHCPTAQLIATLKNGRKICLDLQAPLYQEIIQELLES

SEQ ID NO: 22 - protein, 70 aa, Homo sapiens
EAEEDGDLQCLCVKTTSQVRPRAITSLEVIKAGPHCPTAQLIATLKNGRKICLDLQAPLYKKIIKKLLES

SEQ ID NO: 23 - protein, 70 aa, Homo sapiens
EAEEDGDLQCLCVKTTSQVRPRAITSLEVIKAGPHCPTAQLIATLKNGRKICLDLQAPLYQEIIQELLES

SEQ ID NO: 24 - protein, 70 aa, Homo sapiens
EAEEDGDLQCLCVKTTSQSRPRHITSLEVIKAGPHCPTAQLIATLKNGRKICLDLQAPLYKKIIKKLLES

SEQ ID NO: 25 - protein, 70 aa, Homo sapiens
EAEEDGDLQCLCVKTTSQSRPRHITSLEVIKAGPHCPTAQLIATLKNGRKICLDLQAPLYQEIIQELLES

SEQ ID NO: 26 - protein, 62 aa, Homo sapiens
ACLCVKTTSQVRPRHITSLEVIKAGPHCPTAQLIATLKNGRKICLDLQAPLYKKIIKKLLES

SEQ ID NO: 27 - protein, 62 aa, Homo sapiens
ACLCVKTTSQVRPRHITSLEVIKAGPHCPTAQLIATLKNGRKICLDLQAPLYQEIIQELLES

SEQ ID NO: 28 - protein, 62 aa, Homo sapiens
ACSCQKTASQVRPRHITSLEVIKAGPHCPTAQLIATLKNGRKICLDLQAPLYKKIIKKLLES

SEQ ID NO: 29 - protein, 62 aa, Homo sapiens
ACSCQKTASQVRPRHITSLEVIKAGPHCPTAQLIATLKNGRKICLDLQAPLYQEIIQELLES

SEQ ID NO: 30 - protein, 64 aa, Homo sapiens
DLQCLCVKTTSQVRPRHITSLEVIKAGPHCPTAQLIATLKNGRKICLDLQAPLYQEIIQELLES

SEQ ID NO: 31 - protein, 6 aa, Homo sapiens
EAEEDG

SEQ ID NO: 32 - protein, 101 aa, Homo sapiens
MSSAAGFCASRPGLLFLGLLLLPLVVAFASAEAEEDGDLQCLCVKTTSQVRPRHITSLEVIKAGPHCPTAQLIATLKNGRKICLDLQAPLYKKIIKKLLES

SEQ ID NO: 33 - protein, 104 aa, Homo sapiens
MSSAARSRLTRATRQEMLFLALLLLPVVVAFARAEAEEDGDLQCLCVKTTSQVRPRHITSLEVIKAGPHCPTAQLIATLKNGRKICLDLQALLYKKIIKEHLES

SEQ ID NO: 34 - protein, 25 aa, Artificial Sequence
  Description of Artificial Sequence Synthetic peptide
PHSPTAQLIATLKNGQKISLDLQAP

SEQ ID NO: 35 - protein, 25 aa, Artificial Sequence
  Description of Artificial Sequence Synthetic peptide
PHSPTVQLIATLKNGQKISLDLQAP

SEQ ID NO: 36 - protein, 25 aa, Artificial Sequence
  Description of Artificial Sequence Synthetic peptide
PYSPTAQLIATLKNGQKISLDLQEP

SEQ ID NO: 37 - protein, 25 aa, Artificial Sequence
  Description of Artificial Sequence Synthetic peptide
PHSPQTELIVKLKNGQKISLDLQAP

SEQ ID NO: 38 - protein, 25 aa, Artificial Sequence
  Description of Artificial Sequence Synthetic peptide
PHSPTAQLIATLKNGQKISVDLQAP

SEQ ID NO: 39 - protein, 25 aa, Artificial Sequence
  Description of Artificial Sequence Synthetic peptide
AHSPTAQLIATLKNGQKISLDLQAP

SEQ ID NO: 40 - protein, 25 aa, Artificial Sequence
  Description of Artificial Sequence Synthetic peptide
AHSPTVQLIATLKNGQQISLDLQAP

SEQ ID NO: 41 - protein, 25 aa, Artificial Sequence
  Description of Artificial Sequence Synthetic peptide
AYSPTAQLIATLKNGQKISLDLQEP

SEQ ID NO: 42 - protein, 25 aa, Artificial Sequence
  Description of Artificial Sequence Synthetic peptide
AHSPQTELIVKLKNGQKISLDLQAP

SEQ ID NO: 43 - protein, 25 aa, Artificial Sequence
  Description of Artificial Sequence Synthetic peptide
AHSPTAQLIATLKNGQKISVDLQAP

SEQ ID NO: 44 - protein, 25 aa, Artificial Sequence
  Description of Artificial Sequence Synthetic peptide
PHSATAQLIATLKNGQKISLDLQAP

SEQ ID NO: 45 - protein, 25 aa, Artificial Sequence
  Description of Artificial Sequence Synthetic peptide
PHSATVQLIATLKNGQKISLDLQAP

SEQ ID NO: 46 - protein, 25 aa, Artificial Sequence
  Description of Artificial Sequence Synthetic peptide
PYSATAQLIATLKNGQKISLDLQEP

SEQ ID NO: 47 - protein, 25 aa, Artificial Sequence
  Description of Artificial Sequence Synthetic peptide
PHSAQTELIVKLKNGQKISLDLQAP

SEQ ID NO: 48 - protein, 25 aa, Artificial Sequence
  Description of Artificial Sequence Synthetic peptide
PHSATAQLIATLKNGQKISVDLQAP

SEQ ID NO: 49 - protein, 25 aa, Artificial Sequence
  Description of Artificial Sequence Synthetic peptide
AHSATAQLIATLKNGQKISLDLQAP

SEQ ID NO: 50 - protein, 25 aa, Artificial Sequence
  Description of Artificial Sequence Synthetic peptide
AHSATVQLIATLKNGQQISLDLQAP

SEQ ID NO: 51 - protein, 25 aa, Artificial Sequence
  Description of Artificial Sequence Synthetic peptide
AYSATAQLIATLKNGQKISLDLQEP

SEQ ID NO: 52 - protein, 25 aa, Artificial Sequence
  Description of Artificial Sequence Synthetic peptide
AHSAQTELIVKLKNGQKISLDLQAP

SEQ ID NO: 53 - protein, 25 aa, Artificial Sequence
  Description of Artificial Sequence Synthetic peptide
AHSATAQLIATLKNGQKISVDLQAP

SEQ ID NO: 54 - protein, 27 aa, Artificial Sequence
  Description of Artificial Sequence Synthetic peptide
PHSPTAQLIATLKNGQKISLDLQAPLY

SEQ ID NO: 55 - protein, 27 aa, Artificial Sequence
  Description of Artificial Sequence Synthetic peptide
PHSPTVQLIATLKNGQKISLDLQAPLY

SEQ ID NO: 56 - protein, 27 aa, Artificial Sequence
  Description of Artificial Sequence Synthetic peptide
AHSATAQLIATLKNGQKISLDLQAPLY

SEQ ID NO: 57 - protein, 27 aa, Artificial Sequence
  Description of Artificial Sequence Synthetic peptide
PHSPQTELIVKLKNGQKISLDLQAPRY

SEQ ID NO: 58 - protein, 27 aa, Artificial Sequence
  Description of Artificial Sequence Synthetic peptide
PHSPTAQLIATLKNGQKISLDLQAPRY

SEQ ID NO: 59 - protein, 27 aa, Artificial Sequence
  Description of Artificial Sequence Synthetic peptide
PHSTAAQLIATLKNGQKISLDLQAPLY

SEQ ID NO: 60 - protein, 25 aa, Artificial Sequence
  Description of Artificial Sequence Synthetic peptide
PHCPTAQLIATLKNGRKICLDLQAP

SEQ ID NO: 61 - protein, 25 aa, Artificial Sequence
  Description of Artificial Sequence Synthetic peptide
PHSPTPQLIATLKNGQKISLDLQAP

SEQ ID NO: 62 - protein, 27 aa, Artificial Sequence
  Description of Artificial Sequence Synthetic peptide
PHSTAPQLIATLKNGQKISLDLQAPLY

SEQ ID NO: 63 - protein, 20 aa, Artificial Sequence
  Description of Artificial Sequence Synthetic peptide
PHSPTAQLIATLKNGQKISL

SEQ ID NO: 64 - protein, 20 aa, Artificial Sequence
  Description of Artificial Sequence Synthetic peptide
PHSPTVQLIATLKNGQKISL

SEQ ID NO: 65 - protein, 20 aa, Artificial Sequence
  Description of Artificial Sequence Synthetic peptide
PYSPTAQLIATLKNGQKISL

SEQ ID NO: 66 - protein, 20 aa, Artificial Sequence
  Description of Artificial Sequence Synthetic peptide
PHSPQTELIVKLKNGQKISL

SEQ ID NO: 67 - protein, 20 aa, Artificial Sequence
  Description of Artificial Sequence Synthetic peptide
PHSPTAQLIATLKNGQKISV

SEQ ID NO: 68 - protein, 20 aa, Artificial Sequence
  Description of Artificial Sequence Synthetic peptide
AHSPTAQLIATLKNGQKISL

SEQ ID NO: 69 - protein, 20 aa, Artificial Sequence
  Description of Artificial Sequence Synthetic peptide
AHSPTVQLIATLKNGQQISL

SEQ ID NO: 70 - protein, 20 aa, Artificial Sequence
  Description of Artificial Sequence Synthetic peptide
AYSPTAQLIATLKNGQKISL

SEQ ID NO: 71 - protein, 20 aa, Artificial Sequence
  Description of Artificial Sequence Synthetic peptide
AHSPQTELIVKLKNGQKISL

SEQ ID NO: 72 - protein, 20 aa, Artificial Sequence
  Description of Artificial Sequence Synthetic peptide
AHSPTAQLIATLKNGQKISV

SEQ ID NO: 73 - protein, 20 aa, Artificial Sequence
  Description of Artificial Sequence Synthetic peptide
PHSATAQLIATLKNGQKISL

SEQ ID NO: 74 - protein, 20 aa, Artificial Sequence
  Description of Artificial Sequence Synthetic peptide
PHSATVQLIATLKNGQKISL

SEQ ID NO: 75 - protein, 20 aa, Artificial Sequence
  Description of Artificial Sequence Synthetic peptide
PYSATAQLIATLKNGQKISL

SEQ ID NO: 76 - protein, 20 aa, Artificial Sequence
  Description of Artificial Sequence Synthetic peptide
PHSAQTELIVKLKNGQKISL

SEQ ID NO: 77 - protein, 20 aa, Artificial Sequence
  Description of Artificial Sequence Synthetic peptide
PHSATAQLIATLKNGQKISV

SEQ ID NO: 78 - protein, 20 aa, Artificial Sequence
  Description of Artificial Sequence Synthetic peptide
AHSATAQLIATLKNGQKISL

SEQ ID NO: 79 - protein, 20 aa, Artificial Sequence
  Description of Artificial Sequence Synthetic peptide
AHSATVQLIATLKNGQQISL

SEQ ID NO: 80 - protein, 20 aa, Artificial Sequence
  Description of Artificial Sequence Synthetic peptide
AYSATAQLIATLKNGQKISL

SEQ ID NO: 81 - protein, 20 aa, Artificial Sequence
  Description of Artificial Sequence Synthetic peptide
AHSAQTELIVKLKNGQKISL

SEQ ID NO: 82 - protein, 20 aa, Artificial Sequence
  Description of Artificial Sequence Synthetic peptide
AHSATAQLIATLKNGQKISV

SEQ ID NO: 83 - protein, 20 aa, Artificial Sequence
  Description of Artificial Sequence Synthetic peptide
PHSPTAQLIATLKNGRKISL

SEQ ID NO: 84 - protein, 20 aa, Artificial Sequence
  Description of Artificial Sequence Synthetic peptide
PHSPTVQLIATLKNGRKISL

SEQ ID NO: 85 - protein, 20 aa, Artificial Sequence
  Description of Artificial Sequence Synthetic peptide
PYSPTAQLIATLKNGRKISL

SEQ ID NO: 86 - protein, 20 aa, Artificial Sequence
  Description of Artificial Sequence Synthetic peptide
PHSPQTELIVKLKNGRKISL

SEQ ID NO: 87 - protein, 20 aa, Artificial Sequence
  Description of Artificial Sequence Synthetic peptide
PHSPTAQLIATLKNGRKISV

SEQ ID NO: 88 - protein, 20 aa, Artificial Sequence
  Description of Artificial Sequence Synthetic peptide
AHSPTAQLIATLKNGRKISL

SEQ ID NO: 89 - protein, 20 aa, Artificial Sequence
  Description of Artificial Sequence Synthetic peptide
AHSPTVQLIATLKNGRQISL

SEQ ID NO: 90 - protein, 20 aa, Artificial Sequence
  Description of Artificial Sequence Synthetic peptide
AYSPTAQLIATLKNGRKISL

SEQ ID NO: 91 - protein, 20 aa, Artificial Sequence
  Description of Artificial Sequence Synthetic peptide
AHSPQTELIVKLKNGRKISL

SEQ ID NO: 92 - protein, 20 aa, Artificial Sequence
  Description of Artificial Sequence Synthetic peptide
AHSPTAQLIATLKNGRKISV

SEQ ID NO: 93 - protein, 20 aa, Artificial Sequence
  Description of Artificial Sequence Synthetic peptide
PHSATAQLIATLKNGRKISL

SEQ ID NO: 94 - protein, 20 aa, Artificial Sequence
  Description of Artificial Sequence Synthetic peptide
PHSATVQLIATLKNGRKISL

SEQ ID NO: 95 - protein, 20 aa, Artificial Sequence
  Description of Artificial Sequence Synthetic peptide
PYSATAQLIATLKNGRKISL

SEQ ID NO: 96 - protein, 20 aa, Artificial Sequence
  Description of Artificial Sequence Synthetic peptide
PHSAQTELIVKLKNGRKISL

SEQ ID NO: 97 - protein, 20 aa, Artificial Sequence
  Description of Artificial Sequence Synthetic peptide
PHSATAQLIATLKNGRKISV

SEQ ID NO: 98 - protein, 20 aa, Artificial Sequence
  Description of Artificial Sequence Synthetic peptide
AHSATAQLIATLKNGRKISL

SEQ ID NO: 99 - protein, 20 aa, Artificial Sequence
  Description of Artificial Sequence Synthetic peptide
AHSATVQLIATLKNGRQISL

SEQ ID NO: 100 - protein, 20 aa, Artificial Sequence
  Description of Artificial Sequence Synthetic peptide
AYSATAQLIATLKNGRKISL

SEQ ID NO: 101 - protein, 20 aa, Artificial Sequence
  Description of Artificial Sequence Synthetic peptide
AHSAQTELIVKLKNGRKISL

SEQ ID NO: 102 - protein, 20 aa, Artificial Sequence
  Description of Artificial Sequence Synthetic peptide
AHSATAQLIATLKNGRKISV

SEQ ID NO: 103 - protein, 23 aa, Artificial Sequence
  Description of Artificial Sequence Synthetic peptide
PHSPTAQLIATLKNGQKISLELR

SEQ ID NO: 104 - protein, 23 aa, Artificial Sequence
  Description of Artificial Sequence Synthetic peptide
PHSPTVQLIATLKNGQKISLELR

SEQ ID NO: 105 - protein, 23 aa, Artificial Sequence
  Description of Artificial Sequence Synthetic peptide
PYSPTAQLIATLKNGQKISLELR

SEQ ID NO: 106 - protein, 23 aa, Artificial Sequence
  Description of Artificial Sequence Synthetic peptide
PHSPQTELIVKLKNGQKISLELR

SEQ ID NO: 107 - protein, 23 aa, Artificial Sequence
  Description of Artificial Sequence Synthetic peptide
PHSPTAQLIATLKNGQKISVELR

SEQ ID NO: 108 - protein, 23 aa, Artificial Sequence
  Description of Artificial Sequence Synthetic peptide
AHSPTAQLIATLKNGQKISLELR

SEQ ID NO: 109 - protein, 23 aa, Artificial Sequence
  Description of Artificial Sequence Synthetic peptide
AHSPTVQLIATLKNGQQISLELR

SEQ ID NO: 110 - protein, 23 aa, Artificial Sequence
  Description of Artificial Sequence Synthetic peptide
AYSPTAQLIATLKNGQKISLELR

SEQ ID NO: 111 - protein, 23 aa, Artificial Sequence
  Description of Artificial Sequence Synthetic peptide
AHSPQTELIVKLKNGQKISLELR

SEQ ID NO: 112 - protein, 23 aa, Artificial Sequence
  Description of Artificial Sequence Synthetic peptide
AHSPTAQLIATLKNGQKISVELR

SEQ ID NO: 113 - protein, 23 aa, Artificial Sequence
  Description of Artificial Sequence Synthetic peptide
PHSATAQLIATLKNGQKISLELR

SEQ ID NO: 114 - protein, 23 aa, Artificial Sequence
  Description of Artificial Sequence Synthetic peptide
PHSATVQLIATLKNGQKISLELR

SEQ ID NO: 115 - protein, 23 aa, Artificial Sequence
  Description of Artificial Sequence Synthetic peptide
PYSATAQLIATLKNGQKISLELR

SEQ ID NO: 116 - protein, 23 aa, Artificial Sequence
  Description of Artificial Sequence Synthetic peptide
PHSAQTELIVKLKNGQKISLELR

SEQ ID NO: 117 - protein, 23 aa, Artificial Sequence
  Description of Artificial Sequence Synthetic peptide
PHSATAQLIATLKNGQKISVELR

SEQ ID NO: 118 - protein, 23 aa, Artificial Sequence
  Description of Artificial Sequence Synthetic peptide
AHSATAQLIATLKNGQKISLELR

SEQ ID NO: 119 - protein, 23 aa, Artificial Sequence
  Description of Artificial Sequence Synthetic peptide
AHSATVQLIATLKNGQQISLELR

SEQ ID NO: 120 - protein, 23 aa, Artificial Sequence
  Description of Artificial Sequence Synthetic peptide
AYSATAQLIATLKNGQKISLELR

SEQ ID NO: 121 - protein, 23 aa, Artificial Sequence
  Description of Artificial Sequence Synthetic peptide
AHSAQTELIVKLKNGQKISLELR

SEQ ID NO: 122 - protein, 23 aa, Artificial Sequence
  Description of Artificial Sequence Synthetic peptide
AHSATAQLIATLKNGQKISVELR

SEQ ID NO: 123 - protein, 23 aa, Artificial Sequence
  Description of Artificial Sequence Synthetic peptide
PHSPTAQLIATLKNGRKISLELR

SEQ ID NO: 124 - protein, 23 aa, Artificial Sequence
  Description of Artificial Sequence Synthetic peptide
PHSPTVQLIATLKNGRKISLELR

SEQ ID NO: 125 - protein, 23 aa, Artificial Sequence
  Description of Artificial Sequence Synthetic peptide
DYSPTAQLIATLKNGRKISLELR

SEQ ID NO: 126 - protein, 23 aa, Artificial Sequence
  Description of Artificial Sequence Synthetic peptide
PHSPQTELIVKLKNGRKISLELR

SEQ ID NO: 127 - protein, 23 aa, Artificial Sequence
  Description of Artificial Sequence Synthetic peptide
PHSPTAQLIATLKNGRKISVELR

SEQ ID NO: 128 - protein, 23 aa, Artificial Sequence
  Description of Artificial Sequence Synthetic peptide
AHSPTAQLIATLKNGRKISLELR

SEQ ID NO: 129 - protein, 23 aa, Artificial Sequence
  Description of Artificial Sequence Synthetic peptide
AHSPTVQLIATLKNGRQISLELR

SEQ ID NO: 130 - protein, 23 aa, Artificial Sequence
  Description of Artificial Sequence Synthetic peptide
AYSPTAQLIATLKNGRKISLELR

SEQ ID NO: 131 - protein, 23 aa, Artificial Sequence
  Description of Artificial Sequence Synthetic peptide
AHSPQTELIVKLKNGRKISLELR

SEQ ID NO: 132 - protein, 23 aa, Artificial Sequence
  Description of Artificial Sequence Synthetic peptide
AHSPTAQLIATLKNGRKISVELR

SEQ ID NO: 133 - protein, 23 aa, Artificial Sequence
  Description of Artificial Sequence Synthetic peptide
PHSATAQLIATLKNGRKISLELR

SEQ ID NO: 134 - protein, 23 aa, Artificial Sequence
  Description of Artificial Sequence Synthetic peptide
PHSATVQLIATLKNGRKISLELR

SEQ ID NO: 135 - protein, 23 aa, Artificial Sequence
  Description of Artificial Sequence Synthetic peptide
PYSATAQLIATLKNGRKISLELR

SEQ ID NO: 136 - protein, 23 aa, Artificial Sequence
  Description of Artificial Sequence Synthetic peptide
PHSAQTELIVKLKNGRKISLELR

SEQ ID NO: 137 - protein, 23 aa, Artificial Sequence
  Description of Artificial Sequence Synthetic peptide
PHSATAQLIATLKNGRKISVELR

SEQ ID NO: 138 - protein, 23 aa, Artificial Sequence
  Description of Artificial Sequence Synthetic peptide
AHSATAQLIATLKNGRKISLELR

SEQ ID NO: 139 - protein, 23 aa, Artificial Sequence
  Description of Artificial Sequence Synthetic peptide
AHSATVQLIATLKNGRQISLELR

SEQ ID NO: 140 - protein, 23 aa, Artificial Sequence
  Description of Artificial Sequence Synthetic peptide
AYSATAQLIATLKNGRKISLELR

SEQ ID NO: 141 - protein, 23 aa, Artificial Sequence
  Description of Artificial Sequence Synthetic peptide
AHSAQTELIVKLKNGRKISLELR

SEQ ID NO: 142 - protein, 23 aa, Artificial Sequence
  Description of Artificial Sequence Synthetic peptide
AHSATAQLIATLKNGRKISVELR

SEQ ID NO: 143 - protein, 27 aa, Artificial Sequence
  Description of Artificial Sequence Synthetic peptide
PHSPTAQLIATLKNGQKISLELRAPLY

SEQ ID NO: 144 - protein, 27 aa, Artificial Sequence
  Description of Artificial Sequence Synthetic peptide
PHSPTVQLIATLKNGQKISLELRAPLY

SEQ ID NO: 145 - protein, 27 aa, Artificial Sequence
  Description of Artificial Sequence Synthetic peptide
AHSATAQLIATLKNGQKISLELRAPLY

SEQ ID NO: 146 - protein, 27 aa, Artificial Sequence
  Description of Artificial Sequence Synthetic peptide
PHSPQTELIVKLKNGQKISLELRAPRY

SEQ ID NO: 147 - protein, 27 aa, Artificial Sequence
  Description of Artificial Sequence Synthetic peptide
PHSPTAQLIATLKNGQKISLELRAPRY

SEQ ID NO: 148 - protein, 27 aa, Artificial Sequence
  Description of Artificial Sequence Synthetic peptide
PHSATAQLIATLKNGQKISLELRAPLY

SEQ ID NO: 149 - protein, 27 aa, Artificial Sequence
  Description of Artificial Sequence Synthetic peptide
PHSPTAQLIATLKNGRKISLELRAPLY

SEQ ID NO: 150 - protein, 27 aa, Artificial Sequence
  Description of Artificial Sequence Synthetic peptide
PHSPTVQLIATLKNGRKISLELRAPLY

SEQ ID NO: 151 - protein, 27 aa, Artificial Sequence
  Description of Artificial Sequence Synthetic peptide
AHSATAQLIATLKNGRKISLELRAPLY

SEQ ID NO: 152 - protein, 27 aa, Artificial Sequence
  Description of Artificial Sequence Synthetic peptide
PHSPQTELIVKLKNGRKISLELRAPRY

SEQ ID NO: 153 - protein, 25 aa, Artificial Sequence
  Description of Artificial Sequence Synthetic peptide
PHCPTAQLIATLKNGRKICLDLQAP

SEQ ID NO: 154 - protein, 25 aa, Artificial Sequence
  Description of Artificial Sequence Synthetic peptide
PHSPTPQLIATLKNGQKISLDLQAP

SEQ ID NO: 155 - protein, 27 aa, Artificial Sequence
  Description of Artificial Sequence Synthetic peptide
PHSTAPQLIATLKNGQKISLELRAPLY

SEQ ID NO: 156 - protein, 25 aa, Artificial Sequence
  Description of Artificial Sequence Synthetic peptide
PHSPTAQLIATLKNGQKISLDLQAP

SEQ ID NO: 157 - protein, 23 aa, Artificial Sequence
  Description of Artificial Sequence Synthetic peptide
PHSPTAQLIATLKNGRKISLDLQ

SEQ ID NO: 158 - protein, 20 aa, Artificial Sequence
  Description of Artificial Sequence Synthetic peptide
PTAQLIATLKNGRKISLDLQ

SEQ ID NO: 159 - protein, 20 aa, Artificial Sequence
  Description of Artificial Sequence Synthetic peptide
PHSPTAQLIATLKNGRKISL

SEQ ID NO: 160 - protein, 19 aa, Artificial Sequence
  Description of Artificial Sequence Synthetic peptide
PSPTAQLIATLKNGRKISL

(19) United States
(12) Patent Application Publication
(10) Pub. No.: US 2008/0305041 A1
(43) Pub. Date: Dec. 11, 2008

(54) Pf4 Pharmacophores and Their Uses

(75) Inventors: Philippe Manivet, Paris (FR); George Alexandre Guerin, Varennes Jarcy (FR); Monica Alemany, Barcelona (ES)

Correspondence Address: DARBY & DARBY P.C., P.O. BOX 770, Church Street Station, New York, NY, 10008-0770, US

(73) Assignee: BIOQUANTA CORP., Aurora, CO (US)

(21) Appl. No.: 11/719,614

(22) PCT Filed: Nov. 21, 2005
(86) PCT No.: PCT/US05/42386

§ 371 Date: Aug. 1, 2007

Related U.S. Application Data
(60) Provisional application No. 60/629,883, filed on Nov. 19, 2004.
(60) Provisional application No. 60/634,433, filed on Dec. 8, 2004.
(60) Provisional application No. 60/637,428, filed on Dec. 17, 2004.

(51) Int. Cl. A61K 51/08; C07K 14/00; G06F 19/00; C07D 401/12; C07D 235/16; C07C 233/01; C12N 9/00; A61K 49/14

(52) U.S. Cl. 424/1.69; 530/326; 702/19; 530/324; 546/199; 548/309.7; 546/211; 564/153; 435/183; 424/9.34

(57) ABSTRACT

The invention provides a novel PF4 pharmacophore that is useful, inter alia, for identifying peptidomimetics and other compounds capable of modulating PF4 activity (e.g., as inhibitors, agonists or antagonists). Mutant PF4 polypeptide sequences are also provided that modulate PF4 activity in cells.

### 1. FIELD OF THE INVENTION

The present invention generally relates to compositions and methods for modulating PF4 activity and, more specifically, to compositions and methods for modulating such PF4-mediated processes as angiogenesis, cell proliferation, cell migration and immune system processes. In particular, the invention relates to pharmacophore molecules that emulate the three-dimensional structure of a pharmacophore on the mature wild-type human PF4 molecule and to mutants or variants of such pharmacophore molecules, as well as to mimetic compounds (for example, peptidomimetics or small molecules) that have a pharmacophore or pharmacophore-like three-dimensional structure that is substantially the same as that of a PF4 ligand, or that differs in a function-determining aspect from a PF4 ligand and are capable of modulating PF4 activity. The invention also relates to methods of using such mimetic compounds to modulate PF4 activity, as well as to screening methods for identifying further mimetic compounds, including small molecules.

### 2. BACKGROUND OF THE INVENTION

Chemokines are a superfamily of structurally related, secreted, chemotactic peptides primarily affecting leukocyte migration during the inflammatory response. Their sequences are similar and are characterized by a 4-cysteine motif at the N-terminus. Structurally, all family members have a flexible N-terminal region followed by a loop, then three antiparallel beta strands and a single C-terminal alpha helix. One sub-class of chemokines, designated CXC, contain an intervening residue between the first two N-terminal cysteines. IL-8 is the most well-characterized CXC chemokine, but others include Gro-α and Gro-β, platelet factor-4 (PF4) and IL-10. CXC chemokines signal through receptors designated CXCR, where R designates an integer selected from the group of 1-6. All known CXCR are G-protein-coupled receptors having seven transmembrane-spanning alpha-helix domains.

The CXC chemokines have been implicated in human acute and chronic inflammatory diseases such as arthritis, respiratory diseases, and arteriosclerosis, and also in some acute disorders such as heparin-induced thrombocytopenia. Several CXC chemokines function as agonists of platelet function and stimulators of neutrophils. Recently, some chemokines have been shown to regulate endothelial cell migration and proliferation, suggesting a role in angiogenesis (Murdoch et al., Cytokine 1999; 9: 704-712).

Platelet factor 4 (PF4), which is also known as CXCL4, is a member of the CXC sub-family of chemokines derived from platelets. A preferred PF4 amino acid sequence has been described (see, e.g., Poncz et al, Blood 1987, 69:219-223) and is available from the GeneBank Database (Accession No. P02776). This full-length PF4 amino acid sequence is also provided here, in FIG. 1A (SEQ ID NO:32). The full-length PF4 amino acid sequence includes a signal peptide sequence that preferably comprises amino acid residues 1-31 of SEQ ID NO:32 (FIG. 1A). Typically, the signal peptide sequence is cleaved when the PF4 polypeptide is secreted by cells. Hence, preferred PF4 polypeptides of the invention are actually “mature” PF4 polypeptides, comprising amino acid residues 32-101 of SEQ ID NO:32 (FIG. 1A).

Other “variant” PF4 polypeptides are also known. For example, one preferred variant, referred to as PF4var1, has been described by Green et al (Mol. Cell. Biol. 1989, 9:1445-1451) and is available from the GeneBank Database (Accession No. P10720). This full-length PF4var1 sequence is also provided in FIG. 1B (SEQ ID NO:33). Like with wild-type PF4 (WTPF4) shown in FIG. 1A (SEQ ID NO:32), the PF4var1 includes a signal peptide sequence preferably comprising amino acid residues 1-34 of SEQ ID NO:33 (FIG. 1B), which is typically cleaved when the polypeptide is secreted by cells. Hence, preferred PF4var1 polypeptides are actually “mature” polypeptide that comprise amino acid residues 35-104 of SEQ ID NO:33 (FIG. 1B). For convenience, the PF4 polypeptides shown in FIGS. 1A and 1B (SEQ ID NOS:32-33) are referred to here as wild-type PF4 (WTPF4) and PF4var1, respectively. However, while the present invention is described (for convenience) primarily in terms of the mature WTPF4 sequence (i.e., residues 32-101 of SEQ ID NO:32), it is understood that both sequences represent polypeptide sequences of preferred, naturally occurring PF4 polypeptides. Similarly, other PF4 fragments, such as those fragments described in WO 99/41283 and the related peptides described in WO 01/46218 are also known.

PF4 is released from platelets during platelet aggregation, stimulates neutrophil adhesion to endothelial cells, and in the presence of co-stimulatory cytokines such as TNF, induces neutrophil degranulation in response to injury (Kasper et al, Blood 2003, 103:1602-1610). In addition, PF4 induces human natural killer cells to synthesize and release the related CXCL molecule IL-8, a potent neutrophil chemoattractant and activator (Marti et al., J Leukoc Biol. 2002; 72(3):590-7). PF4 also binds heparin with high affinity, resulting in the formation of immune complexes comprising PF4, heparin and IgG. These complexes lead to further platelet activation via binding of the IgG Fc to FcγRIIa receptors on platelets, resulting in thrombocytopenia and/or thrombosis in individuals receiving heparin.

Recently, PF4 was shown to bind directly to activated T cells and to inhibit their proliferation as well as the release of IFN gamma (Fleischer et al., J Immunol. 2002; 169(2):770-7). In addition, a peptide comprising amino acid residues 34-58 of PF4 produced a 30-40% inhibition of proliferation of murine hematopoietic progenitors (Lecompte-Raclet et al., Biochemistry. 2000; 39(31):9612-22). This activity has been attributed to the alpha helical motif at positions 34-58 of PF4, allowing a DLQ motif at position 54-56 to bind to the progenitor cells. Inhibition of human leukemic/megakaryocyte cell lines by PF4 was also dependent on certain C-terminal residues (residues 1-24 and 13-24 but not residues 16-24) (Lebeurier et al., J Lab Clin Med. 1996; 127(2):179-85). Abrogation or enhancement of PF4 inhibitory activity could be altered by mutations at specific residues within the 13-24 region.

Another important inhibitory activity of PF4, in particular of a C-terminal fragment comprising amino acid residues 47-70, is its anti-angiogenic activity. PF4 inhibits angiogenesis by binding to fibroblast growth factor 2 (FGF2) and preventing FGF-2 binding to vascular endothelial cells (Hagedorn et al., FASEB J. 200; 15(3):550-2). PF4 also disrupts binding of vascular endothelial cell growth factor, a mitogen for endothelial cells, thereby inhibiting its activity (Gengriniovitch et al., J. Biol. Chem. 1995; 270(25):15059-65). Modified C-terminal fragments of PF4 containing the sequence ELR (or the related modified motif DLR) had several times greater anti-angiogenic activity than the unmodified peptide (Hagedorn et al., Cancer Res. 2002; 62(23):6884-90). A single amino acid residue mutation at residue 52 (Cys52Ser) abolished all inhibitory activities (Hagedorn et al., 2001, supra). The conformation of the C-terminal inhibitory fragment in solution has been determined and has been found to be composed of two helical subdomains which interact with FGF in a specific 1:1 complex. Both subdomains are likely required for inhibition of fibroblast growth factor-driven mitogenesis (Lozano et al., J. Biol. Chem. 2001; 276(38):35723).

Recently, a splice variant of a previously known CXC receptor, CXCR3, was shown to bind PF4 with high affinity and act as a functional receptor for PF4 (Lasagni et al., J. Exp. Med. 2003; 197: 153749). Overexpression of this variant, designated CXCR3-B, in a human microvascular endothelial cell line, resulted in reduced DNA synthesis and in increased apoptosis.

NMR and crystal structures of PF4 demonstrate that the molecule exists as a homotetramer (Mayo et al., Biochemistry. 1995; 34(36):11399-409; and Zhang et al., Biochemistry. 1994; 33(27):8361-6). As described above, different residues from distinct structural motifs in the monomeric form of PF4 have been identified that confer specific activities to the molecule. However, there remains a need in the art for peptidomimetics, as well as for small molecule analogues that can mimic or preserve the functional groups on the amino acid residues within these motifs, for use as specific modulators of the immune response and angiogenesis.

Studies using a fluorescently labeled human recombinant PF4 purportedly show that the molecule preferentially binds at regions of active angiogenesis in vivo. Hansell et al., Am. J. Physiol. (1995) 269 (3 Pt 2):H829-836. This has led to the suggestion that PF4 might be useful as an imaging marker for angiogenesis in certain types of tumors, particularly in breast cancer tumors. Borgstrom et al. (Anticancer Res. (1998) 18(6A):4035-4041) and Moyer et al. (J. Nucl. Med. (1996) 37(4):673-679) describe using a ^{99m}Tc-labeled polypeptide that is said to contain the heparin-binding region of PF4. This peptide, which Moyer et al. refer to as P483H, is said to provide high contrast images of infection in vivo. The utility of PF4 as an imaging agent is limited, however, by the molecule's short half-life in blood plasma. Hence, there is a need for molecules, including peptidomimetics and small molecules, that can mimic or preserve the binding activity of PF4, and serve as useful imaging agents in vivo.

The development of such molecules requires elucidation of the entire pharmacophore structure for PF4 and/or PF4 variants (for example, PF4var1), and precise identification of essential and non-essential functional groups for a given activity.

The citation and/or discussion of a reference in this section and throughout the specification is provided merely to clarify the description of the present invention and is not an admission that any such reference is “prior art” to the invention described herein.

### 3. SUMMARY OF THE INVENTION

In response to one or more of the foregoing needs for PF4 activity modulation, the present invention provides novel pharmacophores that are useful, inter alia, for identifying novel compounds, such as novel peptidomimetics or small molecules, that are PF4 agonists or, alternatively, PF4 inhibitors. In particular, the invention provides a PF4 pharmacophore having at least 7 and preferably 10 functional groups, as set forth in Table 1, infra, and arranged in three-dimensional space in a manner that is substantially identical to the arrangement of corresponding functional groups in a PF4 polypeptide (see, for example, FIGS. 2A-2B); provided, however, that the pharmacophore is not PF4 itself nor any of the foregoing peptides discussed above as being in the prior art.

In a preferred aspect, the invention provides methods for identifying novel or existing compounds interacting with PF4 and/or having PF4-like or PF4 antagonistic activities. Such compounds include peptidomimetics and small molecules. Entities identified according to these methods can be either designed (e.g., in silico) and synthesized, or they can be selected from an existing compound library, e.g., by screening in silico. Entities identified according to these methods will modulate PF4 activity as agonists, antagonists, or inhibitors. In some embodiments, these methods comprise comparing a three-dimensional structure for a candidate compound to a three-dimensional structure of a PF4 pharmacophore (preferably a PF4 pharmacophore as substantially described herein). The three-dimensional structures for many compounds that can be screened according to these methods have already been elucidated and can be obtained, e.g., from publicly available databases or other sources. Alternatively, where the three-dimensional structure of a candidate compound has not yet been elucidated, its structure can often be determined using routine techniques (for example, X-Ray diffraction or NMR spectroscopy). Similarity between these three-dimensional structures and associated intramolecular characteristics (such as hydrogen bond forming properties as proton donors or acceptors, hydrophobic interactions, sulfide bond forming properties and electrostatic interactions) would predict that the candidate compound is a compound that modulates PF4 activity. In particular, the root-mean square deviation (RMSD) between the two three-dimensional structures is preferably not greater than about 1.0. The preselected compounds can then be tested as to whether they have the desired activity, in the presence of the pharmacophore molecule or in the presence of native PF4, the latter in vitro or in vivo. Alternatively, a PF4 mimic displaying the PF4 pharmacophore could be a “stand-in” for PF4 in in vitro screening libraries of compounds for those, if any, that have PF4 modulating activity.

In other embodiments, the invention provides PF4 mimetics, which can be mutant PF4 polypeptides that modulate (enhance or impede) PF4 activity in cells. The mutant PF4 polypeptides of the present invention preferably comprise the mature PF4 amino acid sequence set forth in FIG. 1C (SEQ ID NO:1) or a fragment thereof containing at least residues 5 to 23 with one or more amino acid substitutions in the 11 key residues that form the pharmacophore of the present invention. For example, and not by way of limitation, it is understood that wild-type PF4 (WTPF4) and variants thereof (e.g., PF4var1) interact with heparan sulfates through its lysine amino acid. In this predominant pathway, PF4 acts as an antiangiogenic agent by interacting with the surface of endothialial cells. Hence, in one embodiment, the amino acid substitutions include at least one substitution on the pharmacophore that affects PF4 binding to heparan sulfate, such as the amino acid substitutions Lys61→Gln, Lys62→Glu, Lys65→Gln and/or Lys66→Glu. Heparan sulfate binding can be preserved, lessened or increased. Particularly preferred examples of this embodiment are described in detail, below, and include a mutant that is referred to here as PF4-M1 (SEQ ID NO:2) described in the Examples, infra (see, in particular, Tables 3-4 below).

In other embodiments of PF4 mutants, the amino acid substitutions include substitutions in the DLQ sequence motif, such as one or more of the amino acid substitutions Gln9→Arg, Gln9→Ala, and Asp7→Ala. Other preferred amino acid substitutions include one or more of Leu11→Ser, Val13→Gln, Thr16→Ala, Gln18→Ala, Val19→Ser and His23→Ala. It should be noted that mimetics of these PF4 mutants are also within the invention, as long as the three-dimensional structure and intramolecular properties of the original and mutated key residues (including the modifications thereof) are preserved. There is also considerable freedom in linker structures present between key residues of the PF4 mutant or of its mimetic, again as long as the three-dimensional structure is preserved. For example, the invention additionally provides, within its scope, mutant PF4 polypeptides that comprise one or more amino acid additions or deletions, in addition to any of the key residue substitutions described above. Preferred mutant PF4 amino acid sequences of the invention comprise an amino acid sequence as set forth in any of SEQ ID NOS:2-30. See also, Table 3, infra.

Mutants used for validation of the pharmacophore are not active since the point of such mutagenesis is to replace one or more residues that are believed to be important for activity, with other residues that are believed to be unimportant for activity (i.e., the replacement of such residues is expected to abolish or modulate activity). If the mutant is deprived of all (or even some) biological activity compared to the wild type molecule, this means that the residue is crucial for biological activity and should be included in the pharmacophore definition.

The nature of the mutation can also be crucial. For example, it may not be beneficial to replace a hydrophilic residue with one that is hydrophobic (for example, alanine) since both will typically lead to the same type of interaction. The environment of the residue selected for mutation can also be crucial. For example, a mutation may give misleading positive or negative results because neighboring residues compensate (e.g., by conformational change) for the constraints imposed or released by the mutation. This can lead to erroneous interpretation of the results. In addition, the nature of the mutation is preferably chosen to avoid a shift of activity of PF4 toward IL8. Otherwise, the resulting mutant may have IL8-like properties.

The coordinates of the validation mutants described here are not important since the mutants have no interesting biological activity. The mimetics of PF4 can be readily determined with the pharmacophore. If the “candidate mimetic” fits on (i.e., is three-dimensionally superimposable with) the pharmacophore, it is a real mimetic. If the candidate contains only a part of the pharmacophore it can be an antagonist, capable of binding the protein target and competing with PF4 but not capable of activating the target. At least one such mimetic is provided in the present invention, and discussed in detail below.

In preferred embodiments, the present invention provides novel compositions that modulate PF4 activity, e.g., as PF4 agonists and/or antagonists. For example, the invention provides a compound having the following chemical formula:

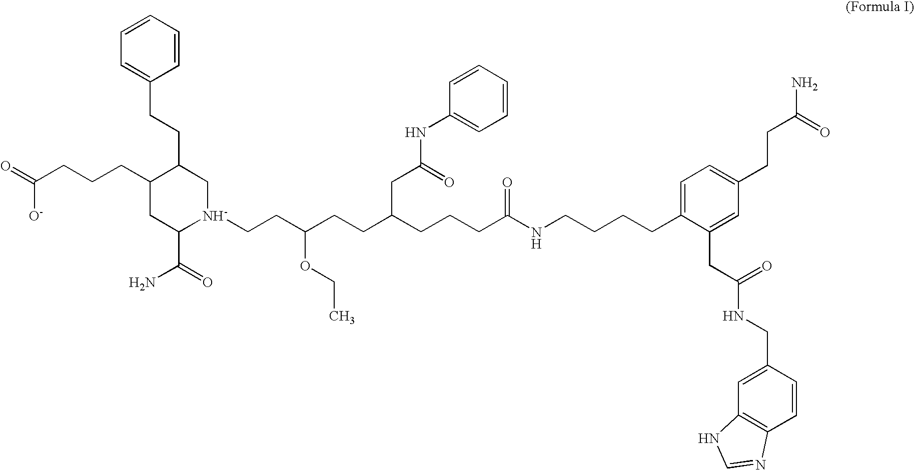

Still other compounds provided by the invention are set forth in Formulas II through VIII illustrated in FIGS. 8 and 9A-9B. In addition, peptide based compounds are provided that can be used, e.g., as PF4 agonists and/or antagonists in accordance with the invention. These include the peptides referred to in the Examples, infra, as P34-56 (SEQ ID NO:157), P37-56 (SEQ ID NO:158), P34-53 (SEQ ID NO:159) and P35-53 (SEQ ID NO:160). A particularly preferred PF4 agonist is the peptide moiety P34-56 (SEQ ID NO:157), whereas the peptide moiety P34-53 (SEQ ID NO:159) is a particularly preferred PF4 antagonist.

In other embodiments, the invention provides detectable markers that are useful for detecting PF4 binding sites, such as PF4 receptors. These detectable markers generally comprise a PF4 antagonist of the invention with a detectable label conjugated thereto. Generally speaking, these detectable markers can be used to detect PF4 binding sites in an individual (for example, in a medical imaging technique such as MRI) by (a) administering the detectable marker to an individual; and (b) detecting the detectable marker's presence in the individual. Previous reports have indicated that PF4 preferably binds to sites of infection and/or angiogenesis in individuals, and can be used to detect certain tumors such as breast cancer tumors. Hence, the methods of this invention can also be used to detect sites of infection and/or angiogenesis in an individual.

These and other aspects of the present invention are described in detail in the following sections.

### 4. BRIEF DESCRIPTION OF THE DRAWINGS

FIGS. 1A-C depict amino acid sequences of preferred PF4 polypeptides. FIG. 1A depicts the amino acid sequence (SEQ ID NO:32) of the full length PF4 polypeptide sequence from GenBank (Accession No. P02776). This full length PF4 polypeptide includes a “signal sequence” (residues 1-31) and a “mature” PF4 sequence comprising amino acid residues 32-101. FIG. 1B depicts the amino acid sequence (SEQ ID NO:33) of a preferred variant, PF4var1. This variant also includes a “signal sequence” (residues 1-34) and a “mature” sequence comprising residues 35-104. FIG. 1C depicts the amino acid sequence (SEQ ID NO:1) of a preferred, mature human PF4 polypeptide (residues 32-101 of SEQ ID NO:32). Dotted lines in FIG. 1C indicate covalent bonds between cysteine amino acid residues. Shaded portions of the sequence in FIG. 1C correspond to the DLQ binding motif (residues 7-9 and 54-56 of SEQ ID NO:1), which is part of the pharmacophore of the invention, and the heparan sulfate binding domain (residues 22-23, 49-50 and 61-66 of SEQ ID NO:1).

FIGS. 2A-2B illustrate the placement in three-dimensions of all ten key functional groups of the PF4 pharmacophore of the present invention. FIG. 2A shows the three-dimensional structure of the mature PF4 polypeptide backbone, based on the coordinates set forth in the Appendix, and highlights ten important functional groups, some of which are on the same residue. Amino acid residues containing functional groups of the pharmacophore as displayed on the native mature PF4 molecule are shown with each functional group of the pharmacophore circled and labeled with a roman numeral. The geometric arrangement of different functional groups in the native PF4 pharmacophore (or in a pharmacophore according to the invention that is a mimetic of PF4) is illustrated in FIG. 2B, with lines indicating the distances between each pair of functional groups, which are labeled with the same roman numerals used in FIG. 2A. Spheres designated with concentric circles indicate functional groups that are hydrogen bond acceptors, whereas grey spheres denote hydrogen bond donors. The black balls adjacent to these functional groups indicate a reference point A that gives the direction of an ideal hydrogen bond at each of these functional groups. For an explanation, see Example 6.2.5, infra. The wire mesh drawn around the hydrophobic functional groups VIII, IX and X indicates the preferred volume of a hydrophobic zone around those points.

FIGS. 3A-3B illustrate the placement and bonding potential in three-dimensions of the PF4 pharmacophore of the invention in Cartesian and spherical coordinate systems having the same origin. FIG. 3A illustrates the placement in three-dimensions of all ten key functional groups of the PF4 pharmacophore in Cartesian and spherical coordinate systems having the same origin. FIG. 3B illustrates the placement of the hydrophobic volume around pharmacophore point VI in the coordinate system of FIG. 3A as well as the direction of one of two potential hydrogen bonding vectors from pharmacophore point V and its corresponding hydrogen bonding potential surface area.

FIG. 4 illustrates hydrogen bond donating and hydrogen bond vectors and potential spheres. Ideal hydrogen bonding potential spherical caps are calculated and shown bisected at ¼ the length of the hydrogen bonding vector which corresponds to the ideal hydrogen bonding surface area for polar pharmacophore points

FIG. 5 illustrates the chemical structure of BQ-A01104, a particular compound which comprises all ten of the PF4 pharmacophore points listed in Table 5, below, held structurally rigid by a scaffold conceptualized as seven distinct subunits or “zones.” with each of the ten pharmacophore points indicated by the corresponding Roman numeral and each of the structural subunits indicated by a corresponding Arabic numeral.

FIGS. 6A-6G, illustrate the structural subunits or “zones” in the scaffold of BQ-A01104.

FIG. 7 illustrates certain exemplary modifications that can be made to optimize the compound BQ-A011004.

FIG. 8 illustrates the complete chemical structures of the modified compounds (Formulas II-VI).

FIGS. 9A-9B illustrate the complete chemical structures of exemplary PF4 agonists. FIG. 9A shows the complete chemical structure of one preferred example of a PF4 agonist (Formula VII). The chemical structure illustrated in FIG. 9B (Formula VIE) represents a preferred example of the PF4 agonist with a contrasting agent conjugated thereto for to detect PF4 polypeptides, e.g., in a medical imaging assay such as magnetic resonance imaging (MRI).

FIGS. 10A-10B compare three-dimensional structures of the peptides P34-56 (SEQ ID NO:157) and P34-53 (SEQ ID NO:159) to the three-dimensional structure of the pharmacophore points in wtPF4 (SEQ ID NO:1). In FIG. 10A, a representation of the P34-56 peptide's (SEQ ID NO:157) three-dimensional structure is shown in the bottom half of the figure. A representation of the three-dimensional structure of the region from Asp7-His23 in wtPF4 (SEQ ID NO:1) is depicted above the peptide. In FIG. 10B, a representation of the P34-53 peptide's (SEQ ID NO:159) three-dimensional structure is shown in the bottom half of the figure, beneath a representation of the wtPF4 (SEQ ID NO:1) three-dimensional structure in the region from Asp7-His23. Amino acid residues in the P34-56 and P34-53 peptides (SEQ ID NOS:157 and 159, respectively) are labeled to indicate the residue of the full-length WTPF4 amino acid sequence (SEQ ID NO:1) to which they correspond.

### 5. DETAILED DESCRIPTION

The present invention pertains to pharmacophore molecules for a cytokine that is referred to here as Platelet Factor 4 or “PF4”. The PF4 cytokine is also known as CXCL4. The PF4 amino acid sequence has been previously described (see, for example, Deuel et al, Proc. Natl. Acad. Sci. U.S.A. 1977, 74:2256-2258; Walz et al, Thromb. Res. 1977, 11:893-898; and Poncz et al., Blood 1987, 69:219-223). The sequence is also available, e.g., on the GenBank databases (Benson et al., Nucleic Acids Research 2003, 31:23-27) under the Accession No. P02776 (GI No. 130304).

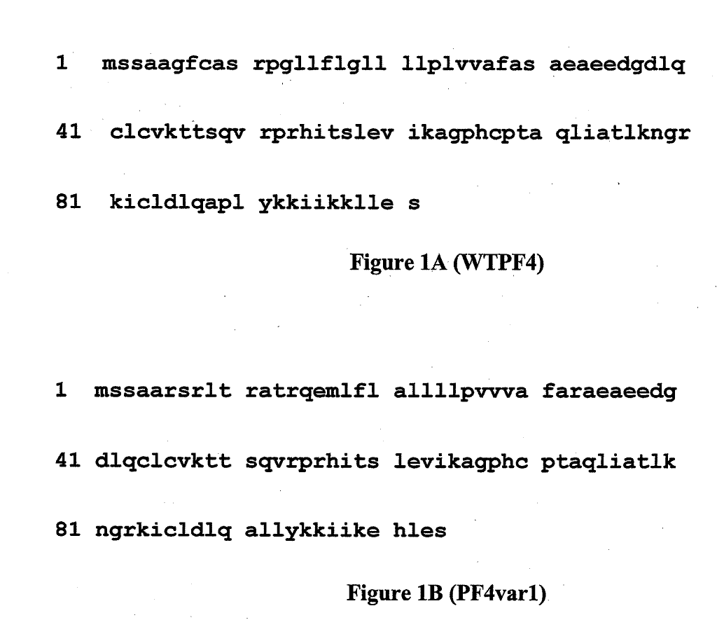

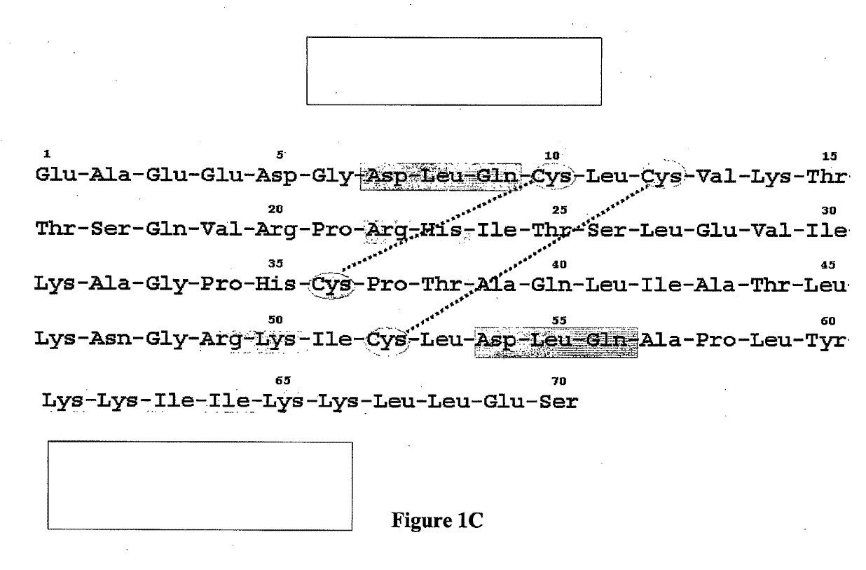

For convenience, the invention is described here primarily in terms of the mature PF4 polypeptide whose amino acid sequence is set forth in FIG. 1C (SEQ ID NO:1). This mature PF4 polypeptide is also referred to here as the mature wild-type PF4 or “WTPF4.” PF4 variants can also be used in the present invention. For example, the full length amino acid sequence of one known, preferred variant, which is referred to here as PF4var1, is depicted in FIG. 1B (SEQ ID NO:33). Preferably, the PF4 polypeptide used in the present invention is a “mature” PF4 polypeptide. Hence, in embodiments that use a variant PF4 polypeptide, such as PF4var1, the polypeptide preferably does not contain the signal peptide sequence (e.g. amino acid residues 1-34 of SEQ ID NO:33) but comprises the amino acid residues of the mature polypeptide (e.g., residues 35-104 of SEQ ID NO:33). Generally, the level of amino acid sequence identity between the mature sequence of a variant PF4 and WTPF4 (SEQ ID NO:1) will be high—e.g., at least 70% and more preferably at least 75, 80, 85, 90, or 95%. Also, any differences between a variant and a wild-type PF4 sequence (as opposed to the PF4 “mutants” described in the Examples, infra) preferably will not modify any points of the pharmacophore. Different PF4 polypeptide sequences can be aligned and their levels of sequence identity to each other determined using any of different known sequence alignment algorithms, such as BLAST, FASTA, DNA Strider, CLUSTAL, etc.

In the case of WTPF4, the full length PF4 cytokine (SEQ ID NO:32) is expressed as a polypeptide chain of 101 amino acid residues. The first 31 amino acid residues of this “full length” PF4 amino acid sequence correspond to a domain that is generally referred to as the “signal sequence domain,” whereas the remaining amino acid residues (i.e., residues 32-101 of SEQ ID NO:32) correspond to what is generally referred to as the “mature” PF4 amino acid sequence. On processing, the PF4 signal sequence domain is cleaved and the “mature” PF4 polypeptide, which exhibits PF4 cytokine activity, is secreted by cells. Hence, pharmacophore molecules of the present invention contain the pharmacophoric structure of the mature PF4. For convenience, a mature wild-type human PF4 amino acid sequence is provided in FIG. 1C (SEQ ID NO:1). As explained above, however, variants of this sequence can also be used in this invention. The full length sequence of one such variant, PF4var1, is provided in FIG. 1B (SEQ ID NO:33), of which amino acid residues 1-34 correspond to the signal sequence. Hence, a preferred mature, variant PF4 polypeptide comprises the sequence of amino acid residues 35-104 of the PF4var1 sequence depicted in FIG. 1B (SEQ ID NO:33).

The three-dimensional structure of PF4 has also been determined by both X-ray crystallography (Zhang et al, Biochemistry 1994, 33:8361-8366) and NMR spectroscopy (Mayo et al, Biochemistry 1995, 34:11399-11409). The coordinates of these structures are available on the Protein Data Bank (Berman et al., Nucleic Acids Research 2000, 28:235-242) under the Accession Numbers 1RHP and 1PFM, respectively. For convenience, a list of coordinates from a preferred three-dimensional structure for mature human PF4 is also provided here in a PDB file format, as an Appendix, infra.

5.1. PF4 Pharmacophores

The term “pharmacophore,” as it is used to describe the present invention, refers to a compound or molecule having a particular collection of functional groups (e.g., atoms) in a particular three-dimensional configuration. More specifically, the term pharmacophore refers to compounds possessing this collection of functional groups in a three-dimensional configuration that is substantially identical to their three-dimensional arrangement on a protein or other compound of interest (referred to here as the “prototype” protein or compound). The present invention concerns the prototype protein PF4. Hence, pharmacophores of the present invention preferably possess a collection of functional groups in a three-dimensional configuration that is substantially identical to their three-dimensional arrangement on PF4. For example, the RMSD between functional groups in a prototype compound of interest and in a pharmacophore should preferably be less than or equal to about one angstrom as calculated, e.g., using the Molecular Similarity module within a molecular modeling program such as QUANTA (available from Molecular Simulations, Inc., San Diego, Calif.).

Preferred pharmacophores are derived from the three-dimensional structure of the protein (preferably the mature or active form of the protein) or other prototype compound of interest that is experimentally determined, e.g., by X-ray crystallography or by nuclear magnetic resonance (NMR) spectroscopy. However, suitable pharmacophores can also be derived, e.g., from homology models based on the structures of related compounds, or from three-dimensional structure-activity relationships. For example, preferred pharmacophores of the present invention are derived from the analysis of point mutations in a PF4 polypeptide, and evaluation of the effects those mutations have on PF4 activity. Suitable PF4 pharmacophores can then be deduced or derived, e.g., by correlating the effects of such mutations to three-dimensional, homology models of a mature PF4.

In preferred embodiments of the invention, PF4 antagonists can be used to detect PF4 receptor molecules, or other PF4 binding sites. The usefulness of detecting such PF4 binding sites is well known in the art. For example, Moyer et al., (J. Nucl. Med. (1996) 37(4):673-679) have described a polypeptide, which they call P483H, that purportedly contains a heparin-binding domain of PF4. ^{99m}Tc-labeled versions of this polypeptide are said to provide high contrast images of infection in vivo. Others have suggested that PF4 might be useful as an imaging marker for angiogenesis in certain types of tumors—particularly in breast cancer tumors. Borgstrom et al., Anticancer Res. (1998) 18(6A):4035-4041.

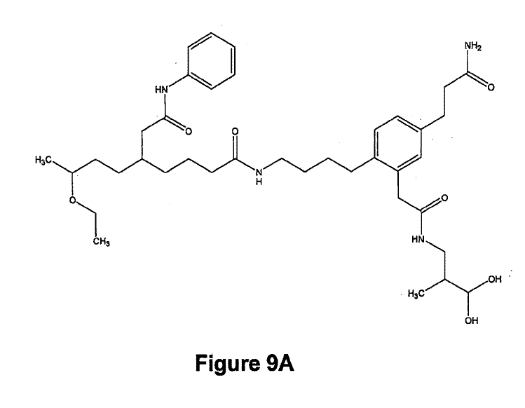

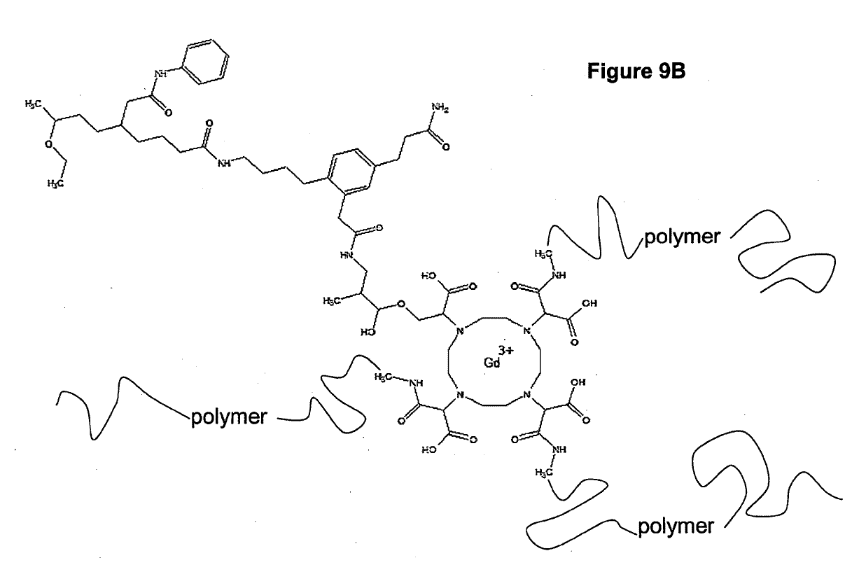

Accordingly, the present invention also provides detectable markers that can be used to detect PF4 binding molecules (for example, PF4 receptor molecules) and PF4 binding. Such detectable markers generally comprise a PF4 antagonist having a detectable label conjugated thereto. The PF4 antagonist can be any compound that binds to a PF4 receptor or binding site without activating the receptor or otherwise inducing PF4-mediated activity. An example of one small molecule antagonist is illustrated in FIG. 9A, whereas FIG. 9B illustrates an exemplary embodiment wherein the antagonist has a detectable label conjugated thereto, e.g., as a contrasting agent for magnetic resonance imaging.

While FIGS. 9A-9B illustrate any embodiment where the PF4 antagonist is a small molecule, PF4 antagonists that are peptides, polypeptides or peptidomimetics can also be used in accordance with these methods. Hence, the invention also includes detectable markers that comprise, as a PF4 antagonist, any of the PF4 polypeptides set forth in SEQ ID NOS:2-30, or any of the PF4 peptides described in international patent publication nos. WO 99/41283 and WO 01/46218. These include any of the peptides set forth in SEQ ID NOS:34-156, described infra. Still other PF4 antagonist peptides are provided in the Examples, infra, including the peptide designated P35-53 (SEQ ID NO:159).

The PF4 antagonist moiety can be readily conjugated to a detectable label according to any technique that is well known and routine to a person having ordinary skill in the art. In preferred embodiments, the detectable marker is used to detect PF4 binding sites in vivo, for example in a medical diagnostic or imaging assay such as magnetic resonance imaging (MRI) or computer assisted tomography (CAT). The PF4 antagonist can be conjugated to any of a variety of contrast or detection agents for such uses, including metals, radioactive isotopes, and radioopaque agents (e.g., gallium, technetium, indium, strontium, iodine, barium, bromine and phosphorus-containing compounds), radiolucent agents, contrast agents, dyes (e.g., fluorescent dyes and chromophores) and enzymes that catalyze a calorimetric or fluorometric reaction. In general, such agents can be attached using any of a variety of techniques known in the art, and in any orientation. See, for example, U.S. Pat. Nos. 5,330,742; 5,384,108; 5,618,513; 5,804,157; 5,952,464; and 6,797,255. One or more water soluble polymer moieties, such as poly-ethylene glycol or “PEG,” can also be conjugated to the PF4 antagonist, e.g., to increase solubility and/or bioavailability of the detectable marker.

As mentioned above, such detectable markers can be used to detect or identify the presence of PF4 binding sites, including the presence of PF4 receptors, in an individual. Generally, such methods comprise steps of administering the detectable marker to the individual, and detecting its presence, e.g. by detecting the presence of the detectable label. Previous reports have indicated that PF4 will preferably bind to sites of angiogenesis and/or infection in an individual. Hence, these methods can also be used to detect sites of angiogenesis and/or infection in individuals. The methods of detecting angiogenesis are particularly useful for detecting the sites of tumors or other cancers in individuals.

In preferred embodiments, these methods detect PF4 binding sites using known methods of medical imaging, such as magnetic resonance imaging (MRI). However, the methods can be practiced using any technique available to a person of ordinary skill for detecting the presence of the detectable label. For example, the methods can also be practiced by detecting the presence of the detectable label in situ (e.g., in a tissue sample from an individual), using, for example, a fluorescent moiety for the detectable label.

Pharmacophores of the present invention are particularly useful for identifying compounds, such as peptidomimetics or small molecules (i.e., organic or inorganic molecules that are preferably less than about 2 kDa in molecular weight, and are more preferably less than about 1 kDa in molecular weight), that modulate PF4 activity in cells (either in vitro or in vivo). For example, in certain embodiments pharmacophores of the present invention can be used to identify compounds that mimic the natural activity of PF4, e.g., by binding to a PF4 receptor. Such compounds, which are capable of increasing or enhancing PF4 activity, are referred to here as PF4 “agonists” or “agonist compounds.” In other embodiments, pharmacophores of the invention can be used to identify compounds that compete with PF4, e.g., for binding to a PF4 receptor, but do not themselves generate any PF4 activity. Such compounds therefore effectively inhibit or decrease PF4 activity, and are referred to here as PF4 “antagonists” or “antagonist compounds.”

Pharmacophore molecules of the present invention are generally more effective, and hence preferable, when the molecule consists essentially of those unique functional groups or elements that are necessary for PF4 activity, while having few if any functional groups or elements that do not affect such activity. Such pharmacophores thereby simplify the search for PF4 agonists and antagonists since the number of functional groups that must be compared between candidate compounds and the pharmacophore is greatly reduced. Accordingly, the present invention provides, in preferred embodiments, a PF4 pharmacophore that consists essentially of at least seven and not more than ten functional groups or “pharmacophore points” bearing the aforementioned spatial relationship Preferred pharmacophore points are given numbers and are set forth in Table I below. Each of these points corresponds to a particular amino acid side chain in the mature PF4 polypeptide sequence set forth in FIG. 1 (SEQ ID NO:1). More specifically, each point corresponds to a particular, unique atom or functional group on an amino acid side chain of that sequence. Accordingly, the pharmacophore points in Table 1 are set forth by specifying both the amino acid residue where they are located, and a particular atom or functional group of that residue side chain. Seven of the ten functional groups listed in Table 1 are essential for anti-angiogenic activity. The seven essential functional groups for anti-angiogenic activity include pharmacophore points I, II, III, IV and VIII, corresponding to the DLQ (Asp7-Leu8-Gln9) motif near the N-terminus of PF4; and pharmacophore points IX and X, corresponding to the hydrophobic centers of Leu11 and Val13. Preferable, but not essential, functional groups for anti-angiogenic activity include pharmacophore points V, VI and VII, corresponding to Gln18 and His23. If these latter points are omitted from a compound otherwise conforming to the pharmacophore, the compound will bind to endothelial cells, but does not activate those cells.

For consistency, the atoms and functional groups in Table 1 use the same notation that is used in the PDB file set forth as an Appendix, infra.

TABLE 1
PREFERRED PF4
PHARMACOPHORE POINTS

| Pharmacophore | Amino Acid | |
|---|---|---|
| Point | Residue | Atom/Functional Group |
| I | Asp7 | (Atom 15) OD1 |
| II | Asp7 | (Atom 16) OD2 |
| III | Gln9 | (Atom 49) NE2 |
| IV | Gln9 | (Atom 50) OE1 |
| V | Gln18 | (Atom 182) OE1 |
| VI | Gln18 | (Atom 183) NE2 |
| VII | His23 | (Atom 276) NE2 |
| VIII | Leu8 | (Atom 26) CG |
| IX | Val13 | (Atom 98) CB |
| X | Leu11 | (Atom 72) CG |

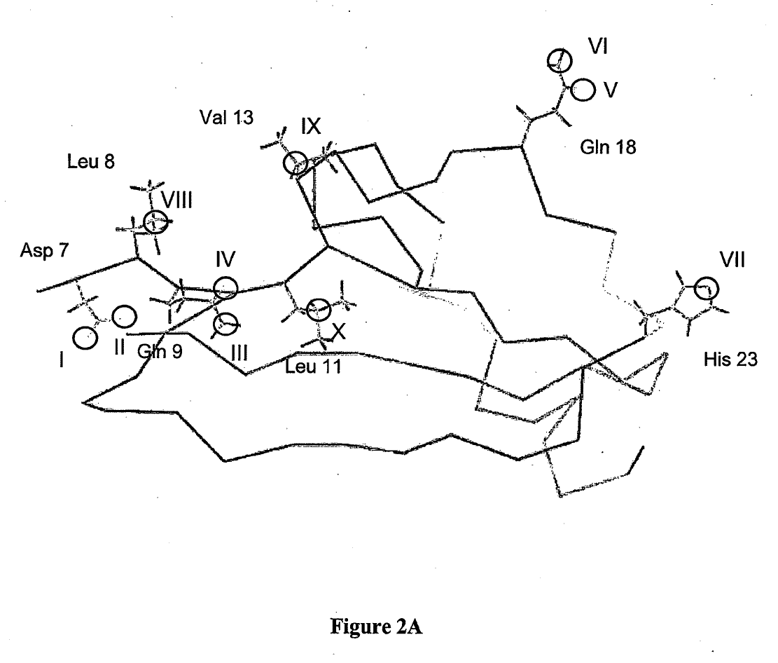

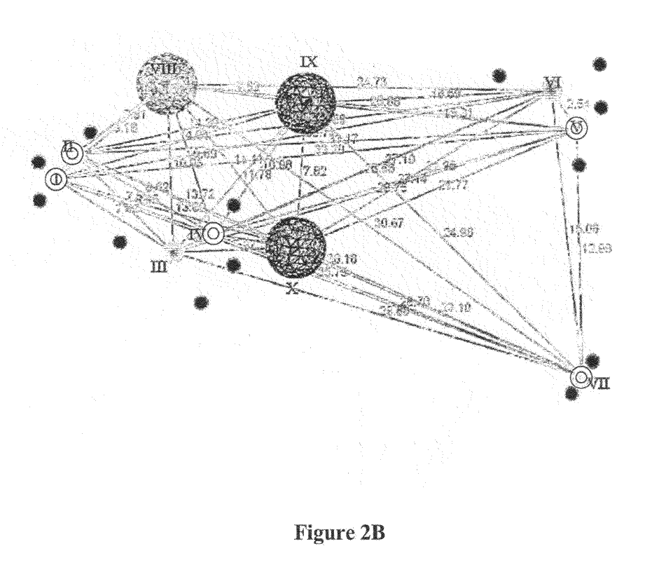

FIGS. 2A and 2B illustrate the pharmacophore points on mature PF4 itself. In particular, FIG. 2A shows an exemplary three-dimensional structure of the mature PF4 polypeptide backbone, based on the coordinates set forth in the Appendix, infra. Amino acid residues containing functional groups of the PF4 are shown with each functional group of the pharmacophore circled and labeled with the corresponding Roman numeral in Table 1, above. FIG. 2B shows the PF4 pharmacophore structure with each point corresponding to a particular functional group. Distances between these functional groups are indicated by lines drawn between the different functional groups in FIG. 2B. These distances can be readily determined and evaluated by a user, e.g., by measuring or calculating distances between the corresponding functional groups in the three-dimensional structure of mature PF4, such as the coordinates set forth in the Appendix, infra. For convenience, preferred distances between these functional groups are also set forth below in Table 2.

TABLE 2
PF4 PHARMACOPHORE DISTANCES

| Pharmacophore | Distance (Å) |
|---|---|
| Points | Mean ± SD |
| I-II | 2.25 ± 0.05 |
| I-III | 6.03 ± 1.37 |
| I-IV | 6.92 ± 1.60 |
| I-V | 30.27 ± 2.92 |
| I-VI | 29.94 ± 2.49 |
| I-VII | 30.41 ± 4.31 |
| I-VIII | 8.57 ± 2.60 |
| I-IX | 14.20 ± 1.53 |
| I-X | 12.54 ± 1.51 |
| II-III | 6.00 ± 2.43 |
| II-IV | 7.01 ± 1.84 |
| II-V | 30.83 ± 1.99 |
| II-VI | 30.33 ± 1.97 |
| II-VII | 31.24 ± 4.03 |
| II-VIII | 9.09 ± 1.22 |
| II-IX | 14.45 ± 0.24 |
| II-X | 13.28 ± 0.37 |
| III-IV | 2.31 ± 0.07 |
| III-V | 26.35 ± 2.76 |
| III-VI | 26.57 ± 2.02 |
| III-VII | 26.31 ± 3.05 |
| III-VIII | 9.19 ± 1.40 |
| III-IX | 10.91 ± 1.74 |
| III-X | 7.06 ± 2.49 |
| IV-V | 25.58 ± 1.40 |
| IV-VI | 25.80 ± 1.31 |
| IV-VII | 25.34 ± 2.81 |
| IV-VIII | 9.02 ± 0.63 |
| IV-IX | 10.46 ± 0.46 |
| IV-X | 6.52 ± 1.26 |
| V-VI | 3.85 ± 1.54 |
| V-VII | 10.21 ± 2.21 |
| V-VIII | 23.10 ± 2.21 |
| V-IX | 17.29 ± 1.68 |
| V-X | 19.25 ± 2.12 |
| VI-VII | 14.07 ± 0.94 |
| VI-VIII | 21.84 ± 2.74 |
| VI-IX | 16.42 ± 2.03 |
| VI-X | 19.95 ± 2.02 |
| VII-VIII | 25.38 ± 4.39 |
| VII-IX | 20.60 ± 3.57 |
| VII-X | 18.76 ± 3.72 |
| VIII-IX | 6.87 ± 0.96 |
| VIII-X | 9.84 ± 1.05 |
| IX-X | 7.25 ± 0.49 |

Preferably, a pharmacophore in the present invention is described using a coordinate system in which each point of the pharmacophore is described by a set of at least three coordinates representing and/or indicating its position in three-dimensional space. In this way, the arrangement of key points in the pharmacophore can be readily modeled and/or visualized (e.g. using various programs and algorithms for modeling molecular structure, such as INSIGHT II described infra). The coordinates of the pharmacophore can also be readily used to compare the pharmacophore structure, as described below, with points in a peptidomimetic or other candidate compound.

Additional parameters can and preferably are also used to describe other properties of the individual pharmacophore points. These can include, in the case of pharmacophore points that are hydrogen bond donors or acceptors, parameters indicating the preferred direction, orientation, size and/or distance of the hydrogen bond. Other parameters that can be used include, for hydrophobic pharmacophore points, a parameter indicating the size (e.g., the distance or volume) of the preferred hydrophobic interaction.

An example of a particularly preferred coordinate system and its use to describe the preferred PF4 pharmacophore is set forth in Example 6.2.5, below. This system can use either Cartesian or spherical coordinates to indicate the position of each pharmacophore point. Those skilled in the art will appreciate that the Cartesian coordinates for a given point can be readily converted into a set of spherical coordinates, and vice-versa, using well-known mathematical relationships between those two coordinate systems that are also set forth in the Example. To describe the preferred size and orientation of hydrogen bonds, the Example also provides, for each hydrogen bond donor and acceptor, coordinates for a hydrogen-bond vector, A, pointing in the direction of the preferred hydrogen bond. The surface area, S, of a preferred hydrogen bonding potential is also provided for each hydrogen bond donor and acceptor in the pharmacophore. This parameter defines the surface of a sphere cap around the hydrogen bonding vector, A, corresponding to the surface where hydrogen bond formation is preferable. For each hydrophobic pharmacophore point, the Example provides a point, m, indicating a point at the closest distance to the pharmacophore point at which undesirable interactions (e.g., interactions with hydrophilic or polar residues, or with polar solvent) should be avoided.

5.2. Peptidomimetics

As noted above, PF4 pharmacophores of the present invention are particularly useful as peptidomimetics and other compounds that are agonists and/or antagonists of PF4 activity. Accordingly, the invention also provides peptidomimetics that are agonists or antagonists of PF4 activity.

Peptidomimetics are described generally, e.g., in International Patent publication no. WO 01/5331 A2 by Gour et al. Such compounds can be, for example, peptides and peptide analogues that comprise a portion of a PF4 amino acid sequence (or an analogue thereof) which contain pharmacophore points substantially similar in configuration to the configuration of functional groups in a mature PF4 pharmacophore. However, one or more pharmacophore points in a peptidomimetic can be modified in a manner that affects PF4 activity (either as an agonist or antagonist), such as by replacement of an amino acid residue displaying that particular pharmacophore point. Alternatively, at least a portion of the peptidomimetics may be replaced by one or more non-peptide structures, such that the three-dimensional structure of functional groups in the pharmacophore is retained at least in part. In other words, one, two, three or more amino acid residues within a PF4 peptide may be replaced by a non-peptide structure. In addition, at least one key amino acid residue can be replaced by another having different characteristics (for example, different properties of hydrophobicity, hydrophilicity, proton donor or acceptor properties, electrostatic properties, etc.). Other portions of a peptide or peptidomimetic can also be replaced by a non-peptide structure.

Typically, peptidomimetics (both peptide and non-peptidyl analogues) may have improved properties (e.g., decreased proteolysis, increased retention or increased bioavailability) that make them more suitable for pharmaceutical compositions than a PF4 peptide. Peptidomimetics may also have improved oral availability. It should be noted that peptidomimetics of the invention may or may not have similar two-dimensional structures, such as sequences and structural formulas. However, all peptidomimetics within the invention with the same activity will share common three-dimensional structural features and geometry with one another, and all will be close to the three-dimensional structure of the pharmacophore of the native human PF4. Each peptidomimetic of the invention may further have one or more unique additional binding elements. The present invention provides methods (described infra) for identifying peptidomimetics.

All peptidomimetics provided herein have a three-dimensional structure that is substantially similar to a three-dimensional structure of a pharmacophore displayed on the native molecule as described above. Generally, the three-dimensional structure of a compound is considered substantially similar to that of a pharmacophore if the two structures have RMSD less than or equal to about one angstrom, as calculated, e.g., using the Molecular Similarity module with the QUANTA program (Biopolymer module of INSIGHT II program available from Accelrys, Inc., San Diego, Calif.) or using other molecular modeling programs and algorithms that are available to those skilled in the art. In preferred embodiments, compounds of the invention have a RMSD less than or equal to about 1.0 Angstrom. More preferably, compounds of the invention have an RMSD that is less than or equal to about 0.5 Angstrom, and still more preferably about 0.1 Angstroms. In particular, a peptidomimetic of the invention will have at least one low-energy three-dimensional structure that is or is predicted to be (e.g. by ab-initio modeling) substantially similar to the three-dimensional structure of a PP4 pharmacophore.

Lower energy conformations can be identified by conformational energy calculations using, for example, the CHARMM program (Brooks et al., J. Comput. Chem. 1983, 4:187-217). The energy terms include bonded and non-bonded terms, including bond length energy. It will be apparent that the conformational energy of a compound can also be calculated using any of a variety of other commercially available quantum mechanic or molecular mechanic programs. Generally, a low energy structure has a conformational energy that is within 50 kcal/mol of the global energy minimum.

As an example, and not by way of limitation, low energy conformations can be identified using combinations of two procedures. The first procedure involves a simulated annealing molecular dynamics approach. In this procedure, the system (which includes the designed peptidomimetics and water molecules) is heated up to above room temperature, preferably to around 600 degrees Kelvin (i.e., 600 K), and is simulated for a period for about 50 to 100 ps (e.g., for 70 ps) or longer. Gradually, the temperature of the system is reduced, e.g., to about 500 K and simulated for a period of about 100 ps or longer, then gradually reduced to 400 K and simulated for a period of 100 ps or longer. The system temperature is then reduced, again, to about 300 K and simulated for a period of about 500 ps or longer. During this analysis, the atom trajectories are recorded. Such simulated annealing procedures are well known in the art and are particularly advantageous, e.g., for their ability to efficiently search the conformational “space” of a protein or other compound. That is to say, using such procedures, it is possible to sample a large variety of possible conformations for a compound and rapidly identify those conformations having the lowest energy.

A second procedure involves the use of self-guided molecular dynamics (SGMD), as described by Wu & Wang, J. Physical Chem. 1998, 102:7238-7250. The SGMD method has been demonstrated to have an extremely enhanced conformational searching capability. Using the SGMD method, therefore, simulation may be performed at 300 K for 1000 ps or longer, and the atom trajectories recorded for analysis.

Conformational analysis of peptidomimetics and other compounds can also be carried out using the INSIGHT II molecular modeling package. First, cluster analysis may be performed using the trajectories generated from molecular dynamics simulations (as described above). From each cluster, the lowest energy conformation may be selected as the representative conformation for this cluster and can be compared to other conformational clusters. Upon cluster analysis, major conformational clusters may be identified and compared to the solution conformations of the cyclic peptide(s). Specifically, a peptidomimetic or other agonist/antagonist compound is optimally superimposed on the pharmacophore model using computational methods well known to those of skill in the art as implemented in, e.g., CATALYST™ (Molecular Simulations, Inc., San Diego, Calif.). A superposition of structures and the pharmacophore model is defined as a minimization of the root mean square distances between the centroids of the corresponding features of the molecule and the pharmacophore. A van der Waals surface is then calculated around the superimposed structures using a computer program such as CERIUS²™ (Molecular Simulations, Inca, San Diego, Calif.). The conformational comparison may also be carried out by using the Molecular Similarity module within the program INSIGHT II.

Similarity in structure can also be evaluated by visual comparison of the three-dimensional structures in graphical format, or by any of a variety of computational comparisons. For example, an atom equivalency may be defined in the peptidomimetic and pharmacophore three-dimensional structures, and a fitting operation used to establish the level of similarity. As used herein, an “atom equivalency” is a set of conserved atoms in the two structures. A “fitting operation” may be any process by which a candidate compound structure is translated and rotated to obtain an optimum fit with the cyclic peptide structure. A fitting operation may be a rigid fitting operation (e.g., the pharmacophore structure can be kept rigid and the three dimensional structure of the peptidomimetic can be translated and rotated to obtain an optimum fit with the pharmacophore structure). Alternatively, the fitting operation may use a least squares fitting algorithm that computes the optimum translation and rotation to be applied to the moving compound structure, such that the root mean square difference of the fit over the specified pairs of equivalent atoms is a minimum. Preferably, atom equivalencies may be established by the user and the fitting operation is performed using any of a variety of available software applications (e.g., INSIGHT II (available from Accelrys Inc. in San Diego, Calif.) or QUANTA, (available from Molecular Simulations)). Three-dimensional structures of candidate compounds for use in establishing substantial similarity can be determined experimentally (e.g., using NMR or X-ray crystallography techniques) or may be computer generated ab initio using, for example, methods provided herein. The use of such modeling and experimental methods to compare and identify peptidomimetics is well known in the art. See, for example, International Patent Publication Nos. WO 01/5331 and WO 98/02452, which are incorporated herein by reference in their entireties (see, Section 7 below).

As one example, and not by way of limitation, chemical libraries (containing, e.g., hydantoin and/or oxopiperazine compounds) may be made using combinatorial chemical techniques and initially screened, in silico, to identify compounds having elements of a PF4 pharmacophore of the invention, which are therefore likely to be either PF4 agonists or antagonists. Combinatorial chemical technology enables the parallel synthesis of organic compounds through the systematic addition of defined chemical components using highly reliable chemical reactions and robotic instrumentation. Large libraries of compounds result from the combination of all possible reactions that can be done at one site with all the possible reactions that can be done at a second, third or greater number of sites. Such methods have the potential to generate tens to hundreds of millions of new chemical compounds, either as mixtures attached to a solid support, or as individual, isolated compounds.

PF4 pharmacophores of the present invention can be used to greatly simplify and facilitate the screening of such chemical libraries to identify those compounds that are most likely to be effective agonists or antagonists of PF4. As a result, library synthesis can focus on those library members with the greatest likelihood of interacting with the target (e.g., a PF4 receptor or the PF4 polypeptide itself), and eliminate the need for synthesizing every possible member of a library (which often results in an unwieldy number of compounds). The integrated application of structure-based design and combinatorial chemical technologies can produce synergistic improvements in the efficiency of drug discovery. By way of example, hydantoin and oxopiperazine libraries may be limited to those compounds that involve only the addition of histidine and valine surrogates to a hydantoin or oxopiperazine backbone.

Peptidomimetic compounds of the present invention also include compounds that are or appear to be unrelated to the original PF4 peptide, but contain functional groups positioned on a nonpeptide scaffold that serve as topographical mimics. Such peptiomimetics are referred to here as “non-peptidyl analogues.” Non-peptidyl analogues can be identified, e.g., using library screens of large chemical databases. Such screens use the three-dimensional conformation of a pharmacophore to search such databases in three-dimensional space. A single three-dimensional structure can be used as a pharmacophore model in such a search. Alternatively, a pharmacophore model may be generated by considering the crucial chemical structural features present within multiple three-dimensional structures.

Any of a variety of databases of three-dimensional structures can be used for such searches. A database of three-dimensional structures can also be prepared by generating three-dimensional structures of compounds, and storing the three-dimensional structures in the form of data storage material encoded with machine-readable data. The three-dimensional structures can be displayed on a machine capable of displaying a graphical three-dimensional representation and programmed with instructions for using the data. Within preferred embodiments, three-dimensional structures are supplied as a set of coordinates that define the three-dimensional structure.

Preferably, the three-dimensional (3D) structure database contains at least 100,000 compounds, with small, non-peptidyl molecules having relatively simple chemical structures particularly preferred. It is also important that the 3D coordinates of compounds in the database be accurately and correctly represented. The National Cancer Institute (NCI) 3D-database (Milne et al., J. Chem. Inf. Comput. Sci. 1994, 34:1219-1224) and the Available Chemicals DIrector (ACD; available from MDL Information Systems, San Leandro, Calif.) are two exemplary databases that can be used to generate a database of three-dimensional structures, using molecular modeling methods such as those described, supra. For flexible molecules, which can have several low-energy conformations, it is desirable to store and search multiple conformations. The Chem-X program (Oxford Molecular Group PLC, Oxford, United Kingdom) is capable of searching thousands or even millions of conformations for a flexible compound. This capability of Chem-X provides a real advantage in dealing with compounds that can adopt multiple conformations. Using this approach, hundreds of millions of conformations can be searched in a 3D-pharmacophore searching process.

Typically, a pharmacophore search will involve at least three steps. The first of these is generation of a pharmacophore query. Such queries can be developed from an evaluation of distances in the three-dimensional structure of the pharmacophore. For example, FIG. 2A shows an exemplary three-dimensional structure of the mature PF4 polypeptide backbone, based on the coordinates set forth in the appendix, infra. Amino acid residues containing functional groups of the PF4 pharmacophore are shown with each functional group of the pharmacophore circled and labeled with a roman numeral corresponding to the numbering used in Table 1, supra. FIG. 2B shows the PF4 pharmacophore structure. In particular, each point in FIG. 2B corresponds to a particular functional group of the PF4 pharmacophore (indicated by roman numerals corresponding to the numbering used in Table 1, supra). Critical pharmacophore distances, which are preferably used in a pharmacophore search, are indicated by lines drawn between the different functional groups in FIG. 2B. These distances can be readily determined and evaluated by a user, e.g., by measuring distances between the corresponding functional groups in a three-dimensional structure of the mature PF4 polypeptide (for example, using the coordinates set forth in the Appendix, infra).

Using the pharmacophore query, a distance bit screening is preferably performed on a database to identify compounds that fulfill the required geometrical constraints. First, the candidate compounds are scanned in order to determine their important physical points (i.e., hydrogen bond donors, hydrogen bond acceptors, hydrophobic volumes, etc.) and important geometric parameters (i.e., relative distances between important physical points). Chemical groups (i.e., hydrophobic, NH₄⁺, carbonyl, carboxylate) are used to map the surface of each candidate compound, while interaction fields are utilized to extract the number and nature of key-points within candidate molecules. There are a number of well-known techniques in the art, such as the GRID program (Molecular Discovery Ltd., London, United Kingdom; Goodford, 1985), which automatically extract important physical points and geometric parameters from the candidate molecules.

Once key-points are extracted from candidate molecules, the candidate compounds and the pharmacophores of the present invention are superimposed or aligned. The degree of similarity between the pharmacophore points and the corresponding key-points of the candidate compound is calculated and utilized to determine a degree of similarity between the two molecules. Details of the superposition method that can be utilized to compare the candidate molecules and the pharmacophores of the present invention are found in the following publications, De Esch et al., J Med. Chem. 2001 24:1666-74 and Lemmen et al., J Med. Chem. 1998 41(23):4502-20. Fitting of a compound to the pharmacophore volume can be done using other computational methods well known in the art. Visual inspection and manual docking of compounds into the active site volume can be done using such programs as QUANTA (Molecular Simulations, Burlington, Mass., 1992), SYBYL (Molecular Modeling Software, Tripos Associates, Inc., St. Louis, Mo., 1992), AMBER (Weiner et al., J. Am. Chem. Soc., 106: 765-784, 1984), or CHARMM (Brooks et al., J. Comp. Chem., 4: 187-217, 1983). This modeling step may be followed by energy minimization using standard force fields, such as CHARMM or AMBER. Other more specialized modeling programs include GRID (Goodford et al., J. Med. Chem., 28: 849-857, 1985), MCSS (Miranker & Karplus, Function and Genetics, 11: 29-34, 1991), AUTODOCK (Goodsell & Olsen, Proteins: Structure, Function and Genetics, 8: 195-202, 1990), and DOCK (Kuntz et al., J. Mol. Biol., 161:269-288 (1982)). In addition, compounds may be constructed de novo in an empty active site or in an active site including some portions of a known inhibitor using computer programs such as LUDI (Bohm, J. Comp. Aid. Molec. Design, 6: 61-78, 1992), LEGEND (Nishibata & Itai, Tetrahedron, 47: 8985, 1991), and LeapFrog (Tripos Associates, St. Louis, Mo.).

After the superposition procedure, molecules with a high matching score or high degree of similarity are selected for further verification of their similarity. Programs, such as ANOVA (performed, for example, with Minitab Statistical Software (Minitab, State College, Pa.)), extract differences that are statistically significant for a defined p value (preferably p values are less than 0.05) between the pharmacophore of the present invention and the candidate molecule. Candidate molecules with a p value below the defined p value are rejected.

A number of different mathematical indices can be utilized to measure the similarity between pharmacophore and candidate molecules. The mathematical indices of interest for the present invention are generally incorporated in the software packages. The choice of mathematical indices will depend on a number of factors, such as the pharmacophore of interest, the library of candidate molecules, and the functional groups identified as essential for activity. For a review on this topic see, Frederique et al., Current Topics in Medicinal Chem. 2004, 4: 589-600.

Compounds that have at least one low energy conformation satisfying the geometric requirement can be considered “hits,” and are candidate compounds for PF4 agonists or antagonists. In a specific embodiment of the invention, compounds of the invention are not PF4, PF4 mutants, IL-8, or a peptide having the amino acid sequence selected from the group consisting of: PHSPTAQLIA TLKNGQKISL DLQAP (SEQ ID NO:34); PHSPTVQLIA TLKNGQKISL DLQAP (SEQ ID NO:35); PYSPTAQLIA TLKNGQKISL DLQEP (SEQ ID NO:36); PHSPQTELUV KLKNGQKISL DLQAP (SEQ ID NO:37); PHSPTAQLIA TLKNGQKISV DLQAP (SEQ ID NO:38); AHSPTAQLIA TLKNGQKISL DLQAP (SEQ ID NO:39); AHSPTVQLIA TLKNGQQISL DLQAP (SEQ ID NO:40); AYSPTAQLIA TLKNGQKISL DLQEP (SEQ ID NO:41); AHSPQTELIV KLKNGQKISL DLQAP (SEQ ID NO:42); AHSPTAQLIA TLKNGQKISV DLQAP (SEQ ID NO:43); PHSATAQLIA TLKNGQKISL DLQAP (SEQ ID NO:44); PHSATVQLIA TLKNGQKISL DLQAP (SEQ ID NO:45); PYSATAQLIA TLKNGQKISL DLQEP (SEQ ID NO:46); PHSAQTELIV KLKNGQKISL DLQAP (SEQ ID NO:47); PHSATAQLIA TLKNGQKISV DLQAP (SEQ ID NO:48); AHSATAQLIA TLKNGQKISL DLQAP (SEQ ID NO:49); AHSATVQLIA TLKNGQQISL DLQAP (SEQ ID NO:50); AYSATAQLIA TLKNGQKISL DLQEP (SEQ ID NO:51); AHSAQTELIV KLKNGQKISL DLQAP (SEQ ID NO:52); AHSATAQLIA TLKNGQKISV DLQAP (SEQ ID NO:53); PHSPTAQLIA TLKNGQKISL DLQAPLY (SEQ ID NO:54); PHSPTVQLIA TLKNGQKISL DLQAPLY (SEQ ID NO:55); AHSATAQLIA TLKNGQKISL DLQAPLY (SEQ ID NO:56); PHSPQTELIV KLKNGQKISL DLQAPRY (SEQ ID NO:57); PHSPTAQLIA TLKNGQKISL DLQAPRY (SEQ ID NO:58); PHSTAAQLIA TLKNGQKISL DLQAPLY (SEQ ID NO:59); PHCPTAQLIA TLKNGRKICL DLQAP (SEQ ID NO:60); PHSPTPQLIA TLKNGQKISL DLQAP (SEQ ID NO:61); PHSTAPQLIA TLKNGQKISL DLQAPLY (SEQ ID NO:62); PHSPTAQLIA TLKNGQKISL (SEQ ID NO:63); PHSPTVQLIA TLKNGQKISL (SEQ ID NO:64); PYSPTAQLIA TLKNGQKISL (SEQ ID NO:65); PHSPQTELIV KLKNGQKISL (SEQ ID NO:66); PHSPTAQLIA TLKNGQKISV (SEQ ID NO:67); AHSPTAQLIA TLKNGQKISL (SEQ ID NO:68); AHSPTVQLIA TLKNGQQISL (SEQ ID NO:69); AYSPTAQLIA TLKNGQKISL (SEQ ID NO:70); AHSPQTELIV KLKNGQKISL (SEQ ID NO:71); AHSPTAQLIA TLKNGQKISV (SEQ ID NO:72); PHSATAQLIA TLKNGQKISL (SEQ ID NO:73); PHSATVQLIA TLKNGQKISL (SEQ ID NO:74); PYSATAQLIA TLKNGQKISL (SEQ ID NO:75); PHSAQTELIV KLKNGQKISL (SEQ ID NO:76); PHSATAQLIA TLKNGQKISV (SEQ ID NO:77); AHSATAQLIA TLKNGQKISL (SEQ ID NO:78); AHSATVQLIA TLKNGQQISL (SEQ ID NO:79); AYSATAQLIA TLKNGQKISL (SEQ ID NO:80); AHSAQTELIV KLKNGQKISL (SEQ ID NO:81); AHSATAQLIA TLKNGQKISV (SEQ ID NO:82); PHSPTAQLIA TLKNGRKISL (SEQ ID NO:83); PHSPTVQLIA TLKNGRKISL (SEQ ID NO:84); PYSPTAQLIA TLKNGRKISL (SEQ ID NO:85); PHSPQTELIV KLKNGRKISL (SEQ ID NO:86); PHSPTAQLIA TLKNGRKISV (SEQ ID NO:87); AHSPTAQLIA TLKNGRKISL (SEQ ID NO:88); AHSPTVQLIA TLKNGRQISL (SEQ ID NO:89); AYSPTAQLIA TLKNGRKISL (SEQ ID NO:90); AHSPQTELIV KLKNGRKISL (SEQ ID NO:91); AHSPTAQLIA TLKNGRKISV (SEQ ID NO:92); PHSATAQLIA TLKNGRKISL (SEQ ID NO:93); PHSATVQLIA TLKNGRKISL (SEQ ID NO:94); PYSATAQLIA TLKNGRKISL (SEQ ID NO:95); PHSAQTELIV KLKNGRKISL (SEQ ID NO:96); PHSATAQLIA TLKNGRKISV (SEQ ID NO:97); AHSATAQLIA TLKNGRKISL (SEQ ID NO:98); AHSATVQLIA TLKNGRQISL (SEQ ID NO:99); AYSATAQLIA TLKNGRKISL (SEQ ID NO:100); AHSAQTELIV KLKNGRKISL (SEQ ID NO:101); AHSATAQLIA TLKNGRKISV (SEQ ID NO:102); PHSPTAQLIA TLKNGQKISL ELR (SEQ ID NO:103); PHSPTVQLIA TLKNGQKISL ELR (SEQ ID NO:104); PYSPTAQLIA TLKNGQKISL ELR (SEQ ID NO:105); PHSPQTELIV KLKNGQKISL ELR (SEQ ID NO:106); PHSPTAQLIA TLKNGQKISV ELR (SEQ ID NO:107); AHSPTAQLIA TLKNGQKISL ELR (SEQ ID NO:108); AHSPTVQLIA TLKNGQQISL ELR (SEQ ID NO:109); AYSPTAQLIA TLKNGQKISL ELR (SEQ ID NO:110); AHSPQTELIV KLKNGQKISL ELR (SEQ ID NO:111); AHSPTAQLIA TLKNGQKISV ELR (SEQ ID NO:112); PHSATAQLIA TLKNGQKISL ELR (SEQ ID NO:113); PHSATVQLIA TLKNGQKISL ELR (SEQ ID NO:114); PYSATAQLIA TLKNGQKISL ELR (SEQ ID NO:115); PHSAQTELIV KLKNGQKISL ELR (SEQ ID NO:116); PHSATAQLIA TLKNGQKISV ELR (SEQ ID NO:117); AHSATAQLIA TLKNGQKISL ELR (SEQ ID NO:118); AHSATVQLIA TLKNGQQISL ELR (SEQ ID NO:119); AYSATAQLIA TLKNGQKISL ELR (SEQ ID NO:120); AHSAQTELIV KLKNGQKISL ELR (SEQ ID NO:121); AHSATAQLIA TLKNGQKISV ELR (SEQ ID NO:122); PHSPTAQLIA TLKNGRKISL ELR (SEQ ID NO:123); PHSPTVQLIA TLKNGRKISL ELR (SEQ ID NO:124); DYSPTAQLIA TLKNGRKISL ELR (SEQ ID NO:125); PHSPQTELIV KLKNGRKISL ELR (SEQ ID NO:126); PHSPTAQLIA TLKNGRKISV ELR (SEQ ID NO:127); AHSPTAQLIA TLKNGRKISL ELR (SEQ ID NO:128); AHSPTVQLIA TLKNGRQISL ELR (SEQ ID NO:129); AYSPTAQLIA TLKNGRKISL ELR (SEQ ID NO:130); AHSPQTELIV KLKNGRKISL ELR (SEQ ID NO:131); AHSPTAQLIA TLKNGRKISV ELR (SEQ ID NO:132); PHSATAQLIA TLKNGRKISL ELR (SEQ ID NO:133); PHSATVQLIA TLKNGRKISL ELR (SEQ ID NO:134); PYSATAQLIA TLKNGRKISL ELR (SEQ ID NO:135); PHSAQTELIV KLKNGRKISL ELR (SEQ ID NO:136); PHSATAQLIA TLKNGRKISV ELR (SEQ ID NO:137); AHSATAQLIA TLKNGRKISL ELR (SEQ ID NO:138); AHSATVQLIA TLKNGRQISL ELR (SEQ ID NO:139); AYSATAQLIA TLKNGRKISL ELR (SEQ ID NO:140); AHSAQTELIV KLKNORKISL ELR (SEQ ID NO:141); AHSATAQLIA TLKNGRKISV ELR (SEQ ID NO:142); PHSPTAQLIA TLKNGQKISL ELRAPLY (SEQ. ID NO:143); PHSPTVQLIA TLKNGQKISL ELRAPLY (SEQ ID NO:144); AHSATAQLIA TLKNGQKISL ELRAPLY (SEQ ID NO:145); PHSPQTELIV KLKNGQKISL ELRAPRY (SEQ ID NO:146); PHSPTAQLIA TLKNGQKISL ELRAPRY (SEQ ID NO:147); PHSATAQLIA TLKNGQKISL ELRAPLY (SEQ ID NO:148); PHSPTAQLIA TLKNGRKISL ELRAPLY (SEQ ID NO:149); PHSPTVQLIA TLKNGRKISL ELRAPLY (SEQ ID NO:150); AHSATAQLIA TLKNGRKISL ELRAPLY (SEQ ID NO:151); PHSPQTELIV KLKNGRKISL ELRAPRY (SEQ ID NO:152); PHCPTAQLIA TLKNGRKICL DLQAP (SEQ ID NO:153); PHSPTPQLIA TLKNGQKISL DLQAP (SEQ ID NO:154); PHSTAPQLIA TLKNGQKISL ELRAPLY (SEQ ID NO:155) or PHSPTAQLIA TLKNGQKISL DLQAP (SEQ ID NO:156).

Those skilled in the art will appreciate that a compound structure may be optimized, e.g., using screens as provided herein. Within such screens, the effect of specific alterations of a candidate compound on three-dimensional structure may be evaluated, e.g., to optimize three-dimensional similarity to a PF4 pharmacophore. Such alterations include, for example, changes in hydrophobicity, steric bulk, electrostatic properties, size and bond angle. Biological testing of candidate agonists and antagonists identified by these methods is also preferably used to confirm their activity.

Once an active peptidomimetic has been identified, related analogues can also be identified, e.g., by two-dimensional similarity searching. Such searching can be performed, for example, using the program ISIS Base (Molecular Design Limited). Two-dimensional similarity searching permits the identification of other available, closely related compounds which may be readily screened to optimize biological activity.

### 6. EXAMPLES

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

6.1. Experimental Procedures

6.1.1 Recombinant PF4 Production

Recombinant PF4 was produced in E. coli as a protein containing a unique methionine residue immediately preceding the PF4 portion. More specifically, expression plasmids were constructed by cloning a synthetic gene encoding native sequence PF4 between the NcoI and XhoI sites in the multiple restriction site region of plasmid pET-15b (available from Novagen, Fontenay-sous-Bois, France). Mutant PF4 genes were generated using standard PCR amplification of synthetic oligonucleotide primers and the wild-type construct as template. All constructs were independently sequenced and verified (Génome Express, Grenoble, France).

BL21(DE) bacteria (available from Novagen, Fontenay-sous-Bois, France) carrying the PF4 plasmids were cultured at 37° C. in EZmix 2×YT medium containing 1 M glucose and appropriate antibiotics. Protein expression was induced in these cell cultures with 1 mM IPTG for 4 hours. Bacterial cells were harvested by centrifugation and were subjected to lysozyme treatment (1 mg/ml) and sonication. The resultant fusion protein was extracted from the lysis pellet with 6 M Urea in 50 mM Tris-HCl, pH 7.4, 5 mM EDTA, and 10 mM DTT. The extracts were then purified using ion-exchange chromatography, and the PF4 proteins were eluted with a gradient of 0-1 M NaCl followed by dialysis into PBS containing 0.5 NaCl. The final protein concentration was determined by use of a BCA Protein Assay Reagent. The homogeneity of recombinant PF4 proteins thus produced was verified by SDS-PAGE and Western blotting with polyclonal antibody against PF4.

6.1.2 Endothelial Cell Cultures

Human umbilical vein endothelial cells (HUVEC) were isolated by collagenase (Roche Diagnostics) digestion as described previously (Jaffe et al., J Clin Invest. 1973; 85(11):2745-56).

Cells were grown in M199 medium containing 15% fetal calf serum (FCS), 5% human serum, 2 mM glutamine, 50 U/ml penicillin, 50 μg/ml streptomycin, 2.5 μg/ml amphotericin B, and 15 mM HEPES. Cultures were maintained at 37° C. amd 5% CO₂ in humidified atmosphere. Every 3-4 days, the cultures were harvested by trypsin treatment, diluted, replated and grown to confluence. HUVEC grown until confluence from the second or third passage are preferably used for experiments described here.

6.1.3 Endothelial Cell Proliferation Assays

Inhibition of DNA synthesis was measured by [³H]-thymidine incorporation assay. Cells were plated at 15,000 cells per well in a 24 well-plate in 0.5 ml medium containing 2.5% FCS and allowed to attach for 4 hours at 37° C. Proliferation was then induced by addition of 10 ng/ml of FGF-2, VEGF₁₆₅ or VEGF₁₂₁. Increasing concentrations of purified recombinant PF4 proteins were added to some wells and HUVEC were further incubated for 48 hours. [³H]-thymidine (1 μCi/well) was added during the last 20 hours of incubation. Cells were washed twice with PBS and treated with ice-cold 10% (w/v) trichloroacetic acid for 30 minutes. The resulting precipitates were solubilized with 1 M NaOH and incorporated radioactivity was measured in a Beckman LS-6500 multi-purpose scintillation counter.

6.1.4 HUVEC Migration Assay

HUVEC migration was evaluated in a modified Boyden chamber assay. Transwell cell culture chamber inserts with porous polycarbonate filters (8 μM pore size) were coated with 0.2% gelatin. HUVEC suspended in medium supplemented with 2.5% FCS were added to the inserts at 4×10⁴ cells per well. The inserts were placed over chambers containing a chemotactic stimulus (10 ng/ml VEGF₁₆₅), and cells were allowed to migrate for 4 hours at 37° C. in a CO₂ incubator. For inhibition experiments, recombinant PF4 proteins were added to both the lower and upper chambers. After incubation, filters were rinsed with PBS, fixed with 1% paraformaldehyde and stained with hematoxyline of Harris (EMD Chemicals Inc. Gibbstown, N.J.).

The upper surfaces of the filters was scraped with a cotton swab to remove the nonmigrant cells. The upper surfaces of the filters were viewed in a optical microscope at high powered (×200) magnification, and the number of cells within the microscope visualization field was recorded. Each experimental point was performed in triplicate, and 20 visual fields were analyzed per filter.

6.1.5 Molecular Modeling

IL8 and PF4 polypeptide molecules were modeled in a molecular dynamics simulation that ran for 700 ps at 300 degrees Kelvin (i.e., 300 K). The molecules were modeled with periodic boundary conditions in a 62 Å×62 Å×62 Å box with approximately 8,000 water molecules. Seven Cl⁻ ions were included in simulations of the PF4 molecule and 4 Cl⁻ ions in simulations of the IL8 molecule, to neutralize electrostatic charges.

Molecular dynamics simulation of peptides ran for 700 ps at 900 K. The peptides were modeled with periodic boundary conditions in a 62 Å×62 Å×62 Å box with approximately 7680 water molecules and 720 trifluoroethanol molecules. Data from NMR analysis of the peptides were included in the molecular dynamics simulation. Harmonic distance constraints with coupling constants and velocities were adjusted to obtain a conformity between NMR experiments and simulation protocol when comparing coupling constants, relative population of different conformers of the same molecule, chemical shift anisotropy, dipole-dipole relaxation rates and other experimental factors to theoretical data.

Virtual peptides were modeled using Langevin dynamics, or other fast technique that avoids using periodic boundary condition with explicit water solvent, to increase the diversity of test peptides. Virtual peptides were randomly mutated at biologically active residues via computer manipulations. After molecular dynamics, virtual peptides were selected for probable activity using a QSAR filter and synthesized and tested on cell cultures (Grassy G, Calas B, Yasri A, Lahana R, Woo J, Iyer S, Kaczorek M, Floc'h R, Buelow R. Computer-assisted rational design of immunosuppressive compounds. Nat. Biotechnol. 1998; 16(8): 748-52).

Langevin dynamics simulations ran for 700 ps at 900 K under harmonic constraints on the peptide backbone. Quenched dynamics of certain density systems were used along with a distance-dependent dielectric constant (∈) to cool the simulated system to 300 K for re-equilibration. The last conformation obtained at the end of the quenched dynamics was finally submitted to 500 ps of molecular dynamics at 300 K.

6.1.6 Statistical Analysis

Triplicate determinations per experimental point were performed for most experiments, and the results are expressed as the mean±one standard deviation (SD) for the data combined from separate experiments. The significance of differences between groups was determined by a standard Student t-test for unpaired data.

6.2. Results

Peptide fragments of the mature PP4 polypeptide sequence depicted in FIG. 1C (SEQ ID NO:1) were generated and their angiogenic effects (cell migration and proliferation) on HUVEC cells evaluated using the assays described in Section 6.1, above. These peptides were investigated further using molecular modeling and quantitative structure activity relationship (QSAR) techniques to determine which conformation(s) and structural properties were common in peptides that exhibited anti-angiogenic activity.

Molecular dynamics calculations of full length PF4 and the related IL8 polypeptides were also performed. Active peptides were found to have a triad of amino acid residues Asp-Leu-Gln (DLQ) near the N-terminus with the same conformation as the Asp-Leu-Gln triad in full length PF4.

Next, the PF4 surface was mapped using site-directed mutagenesis. In particular, a series of mutant PF4 polypeptides was generated, and their angiogenic activity in HUVEC cells was investigated using assays such as those described in Section 6.1, above. Table 3 below lists amino acid sequences of the PF4 polypeptides generated, along with each polypeptide designation and sequence identification number (SEQ ID NO.). The first sequence, which is designated WTPF4, corresponds to the wild-type, mature PF4 amino acid sequence that is also depicted in FIG. 1C (SEQ ID NO:1). The other sequences depicted in Table 3 comprise one or more amino acid substitutions, indicated by bold-faced, underlined type in the amino acid sequence.

TABLE 3
MUTANT PF4 POLYPEPTIDES

| | |
|---|---|
| Mutant/WT | Sequence |

| | | |
|---|---|---|
| WTPF4 | EAEEDGDLQC LCVKTTSQVR PRHITSLEVI KAGPHCPTAQ | SEQ ID NO:1 |
| | LIATLKNGRK ICLDLQAPLY KKIIKKLLES | |
| PF4-M1 | EAEEDGDLQC LCVKTTSQVR PRHITSLEVI KAGPHCPTAQ | SEQ ID NO:2 |
| | LIATLKNGRK ICLDLQAPLY QEIIQELLES | |
| PF4-M2 | EAEEDGDLQS LCVKTTSQVR PRHITSLEVI KAGPHSPTAQ | SEQ ID NO:3 |
| | LIATLKNGRK ICLDLQAPLY KKIIKKLLES | |
| PF4-M2H | EAEEDGDLQS LCVKTTSQVR PRHITSLEVI KAGPHSPTAQ | SEQ ID NO:4 |
| | LIATLKNGRK ICLDLQAPLY QEIIQELLES | |
| PF4-M3 | EAEEDGDLRC LCVKTTSQVR PRHITSLEVI KAGPHCPTAQ | SEQ ID NO:5 |
| | LIATLKNGRK ICLDLQAPLY KKIIKKLLES | |
| PF4-M3H | EAEEDGDLRC LCVKTTSQVR PRHITSLEVI KAGPHCPTAQ | SEQ ID NO:6 |
| | LIATLKNGRK ICLDLQAPLY QEIIQELLES | |
| PF4-M4 | EAEEDGALAC LCVKTTSQVR PRHITSLEVI KAGPHCPTAQ | SEQ ID NO:7 |
| | LIATLKNGRK ICLDLQAPLY KKIIKKLLES | |
| PF4-M4H | EAEEDGALAC LCVKTTSQVR PRHITSLEVI KAGPHCPTAQ | SEQ ID NO:8 |
| | LIATLKNGRK ICLDLQAPLY QEIIQELLES | |
| PF4-M5 | EAEEDGDLQC SCQKTASQVR PRHITSLEVI KAGPHCPTAQ | SEQ ID NO:9 |
| | LIATLKNGRK ICLDLQAPLY KKIIKKLLES | |
| PF4-M5H | EAEEDGDLQC SCQKTASQVR PRHITSLEVI KAGPHCPTAQ | SEQ ID NO:10 |
| | LIATLKNGRK ICLDLQAPLY QEIIQELLES | |
| PF4-M6 | EAEEDGDLQC LCVKTTSASR PRAITSLEVI KAGPHCPTAQ | SEQ ID NO:11 |
| | LIATLKNGRK ICLDLQAPLY KKIIKKLLES | |
| PF4-M6H | EAEEDGDLQC LCVKTTSASR PRAITSLEVI KAGPHCPTAQ | SEQ ID NO:12 |
| | LIATLKNGRK ICLDLQAPLY QEIIQELLES | |
| PF4-M7H | EAEEDGDLQC LCVKTTSQVR PRHITSLEVI KAGPHCPTAQ | SEQ ID NO:13 |
| | LIATLKNGRK ICLDLQAPLY QEIIQELLES YYY | |
| PF4-M8 | EAEEDGDLQC SCVKTTSQVR PRHITSLEVI KAGPHCPTAQ | SEQ ID NO:14 |
| | LIATLKNGRK ICLDLQAPLY KKIIKKLLES | |
| PF4-M8H | EAEEDGDLQC SCVKTTSQVR PRHITSLEVI KAGPHCPTAQ | SEQ ID NO:15 |
| | LIATLKNGRK ICLDLQAPLY QEIIQELLES | |
| PF4-M9 | EAEEDGDLQC LCQKTTSQVR PRHITSLEVI KAGPHCPTAQ | SEQ ID NO:16 |
| | LIATLKNGRK ICLDLQAPLY KKIIKKLLES | |
| PF4-M9H | EAEEDGDLQC LCQKTTSQVR PRHITSLEVI KAGPHCPTAQ | SEQ ID NO:17 |
| | LIATLKNGRK ICLDLQAPLY QEIIQELLES | |
| PF4-M10 | EAEEDGDLQC LCVKTASQVR PRHITSLEVI KAGPHCPTAQ | SEQ ID NO:18 |
| | LIATLKNGRK ICLDLQAPLY KKIIKKLLES | |
| PF4M10H | EAEEDGDLQC LCVKTASQVR PRHITSLEVI KAGPHCPTAQ | SEQ ID NO:19 |
| | LIATLKNGRK ICLDLQAPLY QEIIQELLES | |
| PF4-M11 | EAEEDGDLQC LCVKTTSAVR PRHITSLEVI KAGPHCPTAQ | SEQ ID NO:20 |
| | LIATLKNGRK ICLDLQAPLY KKIIKKLLES | |
| PF4-M11H | EAEEDGDLQC LCVKTTSAVR PRHITSLEVI KAGPHCPTAQ | SEQ ID NO:21 |
| | LIATLKNGRK ICLDLQAPLY QEIIQELLES | |
| PF4-M12 | EAEEDGDLQC LCVKTTSQVR PRAITSLEVI KAGPHCPTAQ | SEQ ID NO:22 |
| | LIATLKNGRK ICLDLQAPLY KKIIKKLLES | |
| PF4-M12H | EAEEDGDLQC LCVKTTSQVR PRAITSLEVI KAGPHCPTAQ | SEQ ID NO:23 |
| | LIATLKNGRK ICLDLQAPLY QEIIQELLES | |
| PF4-M13 | EAEEDGDLQC LCVKTTSQSR PRHITSLEVI KAGPHCPTAQ | SEQ ID NO:24 |
| | LIATLKNGRK ICLDLQAPLY KKIIKKLLES | |
| PF4-M13H | EAEEDGDLQC LCVKTTSQSR PRHITSLEVI KAGPHCPTAQ | SEQ ID NO:25 |
| | LIATLKNGRK ICLDLQAPLY QEIIQELLES | |
| PF4-M14 | AC LCVKTTSQVR PRHITSLEVI KAGPHCPTAQ | SEQ ID NO:26 |
| | LIATLKNGRK ICLDLQAPLY KKIIKKLLES | |
| PF4-M14H | AC LCVKTTSQVR PRHITSLEVI KAGPHCPTAQ | SEQ ID NO:27 |
| | LIATLKNGRK ICLDLQAPLY QEIIQELLES | |
| PF4-M15 | AC SCQKTASQVR PRHITSLEVI KAGPHCPTAQ | SEQ ID NO:28 |
| | LIATLKNGRK ICLDLQAPLY KKIIKKLLES | |
| PF4-M15H | AC SCQKTASQVR PRHITSLEVI KAGPHCPTAQ | SEQ ID NO:29 |
| | LIATLKNGRK ICLDLQAPLY QEIIQELLES | |
| PF4-M17H | DLQC LCVKTTSQVR PRHITSLEVI KAGPHCPTAQ | SEQ ID NO:30 |
| | LIATLKNGRK ICLDLQAPLY QEIIQELLES | |

The significance of each mutation described in Table 3, above, is summarized in Table 4, infra, along with a description of the mutation's expected effect on PF4 activity.

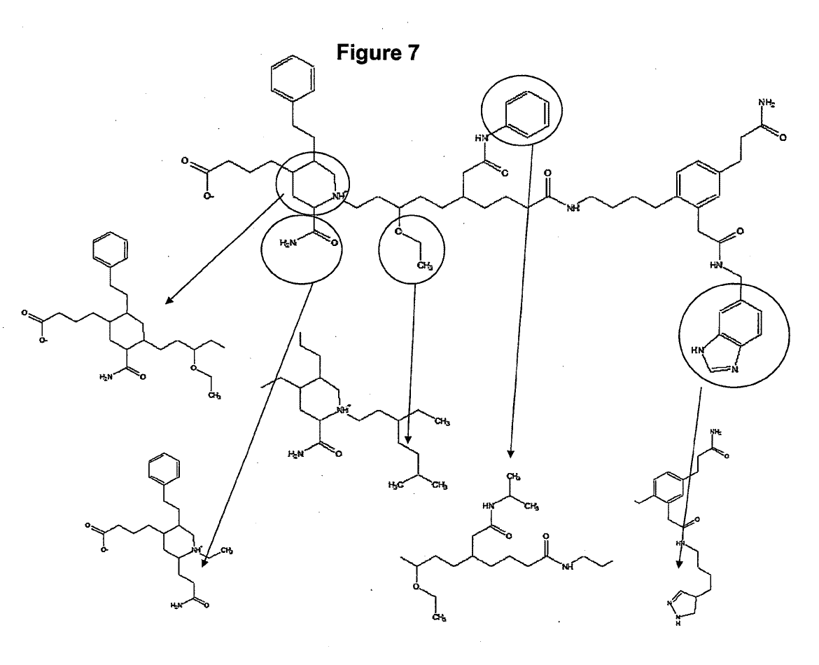

TABLE 4
PF4 MUTATIONS
AND THEIR SIGNIFICANCE

| Mutant(s) | Significance | Function |
|---|---|---|
| PF4-M1 | Lysine residues 61, 62, 65 and 66 allow PF4 to | These mutations prevent |
| | bind to heparan sulfate negative charges. This | interaction of PF4-M1 with |
| | motif is referred to as the Heparan sulfate | heparan sulfate and allow |
| | Binding Domain or “HBD”. By interacting | pF4-M1 to inhibit |
| | with heparan sulfate, PF4 activates growth | angiogenesis through a |
| | factors transduction pathway. In the mutant | separate pathway, for which a |
| | PF4-M1, the HBD is absent | pharmacophore is developed |
| | | in this invention. The mutant |
| | | does not interact with |
| | | heparan sulfate or its |
| | | receptor, and so does not |
| | | prevent VEGF₁₆₅ interaction |
| | | with heparan sulfate. |
| | | PF4-M1 is antiangiogenic |
| | | and inhibits the proliferation |
| | | of HUVECT stimulated by |
| | | VEGF₁₂₁ and VEGF₁₆₅. |
| | | PF4-M1 also inhibits cell |
| | | migration. |
| PF4-M2 and | To test the importance of Cys residues in the | Complete loss of activity due |
| PF4-M2H | stability of PF4. | to misfolding. |
| PF4-M3 | Test of the influence of DLQ motif | This DLQ mutant is still able |
| | replacement by an IL8-like motif (ELR), on | to inhibit angiogenesis but by |
| | the antiangiogenic activity of PF4. PF4-M3 is | interacting with heparan |
| | carrying HBD. | sulfate. It has the same effect |
| | | as WTPF4 on HUVEC. DLQ |
| | | seems to play a minor role in |
| | | the heparan sulfate- |
| | | dependent pathway. |
| PF4-M3H | PF4-M3H is a PF4-M3 mutant without HBD. | This mutant cannot inhibit |
| | | the proliferation and the |
| | | migration of HUVEC cells. |
| | | DLQ seems to play a major |
| | | role in the pathway |
| | | postulated in this |
| | | specification. Replacement of |
| | | only one residue (Q to R) |
| | | cancels the antiangiogenic |
| | | properties of PF4. |
| PF4-M4 | Test of the influence of DLQ motif | Mutation does not modify the |
| | suppression on the antiangiogenic activity of | antiangiogenic properties of |
| | PF4. M4 is able to bind to heparan sulfate | PF4 since those properties |
| | through the HBD. | are, heparan sulfate- |
| | | dependantfor PF4-M4. |
| PF4-M4H | DLQ motif suppression. | Inhibits nearly completely or |
| | | completely the |
| | | antiangiogenic properties of |
| | | PF4. |
| PF4-M5 | After alignment of PF4 and IL8 (FIG. 7), | Mutation decreases the |
| | residues of different physical nature were | antiangiogenic properties of |
| | pointed (arrows). This sequence difference | PF4. |
| | can explain the difference in receptor | |
| | specificity between IL8 and PF4. IL8 is | |
| | proangiogenic and PF4 is antiangiogenic. | |
| | Those loci were mutated. M5 is a triple | |
| | mutant. Amino acids in WT replaced by | |
| | residues with different physical properties: | |
| | L42S, V44Q, T47A. All mutants are carrying | |
| | HBD. | |
| PF4-M5H | Triple mutant: L42S, V44Q, T47A, without | Decreases the antiangiogenic |
| | HBD. | activity of the triple mutant. |
| PF4-M6 | M6 is a triple mutant. Amino acids in WT | Mutation decreases the |
| | replaced by residues with different physical | antiangiogenic properties of |
| | properties: Q49A, V50S, H54A. All mutants | PF4. |
| | are carrying the heparan sulfate-binding | |
| | domain (HBD). | |
| PF4-M6H | Triple mutant: Q49A, V50S, H54A, without | Mutations induce a loss of |
| | HBD. | antiangiogenic activity of |
| | | PF4. The mutated loci are |
| | | important for the |
| | | antiangiogenic activity. |
| PF4-M7H | This mutant is a PF4-M1 mutant with three | Repetition of tyrosine increases |
| | tyrosine residues added at the C-terminal end | the number of [¹²⁵I] atoms that |
| | of the PF4. The additional tyrosine residues | can be carried by PF4-M7. |
| | are useful to carry [¹²⁵I] label. | |
| PF4-M8 | Single mutant of PF4-M5 carrying HBD. The | Mutation does not modify the |
| | single mutation (L42S) controls formation of | antiangiogenic properties of |
| | the triple mutant. | PF4. |
| PF4-M8H | PF4-M8H is a single mutant: L42S, without | Mutation suppresses the |
| | HBD. | antiangiogenic properties of |
| | | PF4. This locus is necessary |
| | | for pharmacophore |
| | | definition. |
| PF4-M9 | Single mutant of PF4-M5 carrying HBD. The | Mutation does not modify the |
| | single mutation (V44Q) is used to control | antiangiogenic properties of |
| | triple mutant. | PF4. |
| PF4-M9H | PF4-M9H is a single mutant: V44Q, without | Mutation suppresses the |
| | HBD. | antiangiogenic properties of |
| | | PF4. This locus is necessary |
| | | for pharmacophore |
| | | definition. |
| PF4-M10 | Single mutant of PF4-M5 carrying HBD. The | Mutation does not modify the |
| | single mutation (T47A) controls formation of | antiangiogenic properties of |
| | the triple mutant. | PF4. |
| PF4-M10H | PF4-M10H is a single mutant: T47A, without | Mutation does not suppress |
| | HBD. | the antiangiogenic properties |
| | | of PF4. This locus is not |
| | | required for pharmacophore |
| | | definition. |
| PF4-M11 | Single mutant of PF4-M6 carrying HBD. The | Mutation does not modify the |
| | single mutation (Q49A) controls formation of | antiangiogenic properties of |
| | the triple mutant. | PF4. |
| PF4-M11H | PF4-M10H is a single mutant: Q49A, without | Mutation suppresses the |
| | HBD. | antiangiogenic properties of |
| | | PF4. This locus is necessary |
| | | for pharmacophore |
| | | definition. |
| PF4-M12 | Single mutant of PF4-M6 carrying HBD. The | Mutation does not modify the |
| | single mutation (H54A) controls formation of | antiangiogenic properties of |
| | the triple mutant. | PF4. |
| PF4-M12H | PF4-M12H is a single mutant: H54A, without | Mutation suppresses the |
| | HBD. | antiangiogenic properties of |
| | | PF4. This locus is necessary |
| | | for pharmacophore |
| | | definition. |
| PF4-M13 | Single mutant of PF4-M6 carrying HBD. The | Mutation does not modify the |
| | single mutation (V50S) controls formation of | antiangiogenic properties of |
| | the triple mutant. | PF4. |
| PF4-M13H | PF4-M13H is a single mutant: V50S, without | Mutation does not suppress |
| | HBD. | the antiangiogenic properties |
| | | of PF4. This locus is not |
| | | required for pharmacophore |
| | | definition. |
| PF4-M17H | Mutant lacking the N-terminal negative | Mutation does not modify the |
| | sequence EAEEDG (SEQ ID NO: 31) | antiangiogenic properties of |
| | | PF4. |

By characterizing the activity of these mutations and correlating the results with a three-dimensional structure of PF4, more complete pharmacophore structures for that molecule have been identified. In particular, this PF4 pharmacophore consists essentially of at least seven and up to ten key functional groups and of their spatial relationships that are believed to be critical for specific interactions of PF4 with a PF4-receptor. Each point in this pharmacophore structure corresponds to a particular, unique atom or functional group on an amino acid side chain of the mature PF4 sequence set forth in FIG. 1C (SEQ ID NO:1). These points are set forth in Table 1, above, and also in Table 5 below. In particular, Table 5 specifies the amino acid residue where each point in the PF4 pharmacophore is located, along with the particular atom or functional group of that side chain that corresponds to the pharmacophore point. The far left-hand column in Table 5 also provides a commentary describing the nature of possible interactions between the pharmacophore and a PF4-specific receptor.

TABLE 5
PREFERRED PF4 PHARMACOPHORE POINTS

| | Amino | | |
|---|---|---|---|
| Pharmacophore | Acid | Atom/Functional | |
| Point | Residue | Group | Comment(s) |
| I | Asp7 | (Atom 15) OD1 | Electrostatic interaction |
| | | | Hydrogen bond |
| | | | acceptor |
| II | Asp7 | (Atom 16) OD2 | Electrostatic interaction |
| | | | Hydrogen bond |
| | | | acceptor |
| III | Gln9 | (Atom 49) NE2 | Hydrogen bond donor |
| IV | Gln9 | (Atom 50) OE1 | Hydrogen bond |
| | | | acceptor |
| V | Gln18 | (Atom 182) OE1 | Hydrogen bond |
| | | | acceptor |
| VI | Gln18 | (Atom 183) NE2 | Hydrophobic donor |
| VII | His23 | (Atom 276) NE2 | Hydrogen bond |
| | | | acceptor |
| VIII | Leu8 | (Atom 26) CG | Hydrophobic |
| | | | interaction |
| IX | Val13 | (Atom 98) CB | Hydrophobic |
| | | | interaction |
| X | Leu11 | (Atom 72) CG | Hydrophobic |
| | | | interaction |

FIGS. 2A and 2B provide an illustration of this pharmacophore on the prototype molecule, native mature human PF4. In particular, FIG. 2A shows a three-dimensional structure of the mature PF4 polypeptide backbone, based on the coordinates set forth in the Appendix, infra. Amino acid residues containing functional groups of the PF4 pharmacophore are shown with each functional group of the pharmacophore circled and labeled with the corresponding roman numeral in Table 1, above. FIG. 2B shows the PF4 pharmacophore structure with each point corresponding to a particular functional group. Distances between these functional groups are indicated by lines drawn between the different functional groups in FIG. 2B. These distances can be readily determined and evaluated by a user, e.g. by measuring or calculating distances between the corresponding functional groups in the three-dimensional structure of mature PF4, such as the coordinates set forth in the Appendix, infra. For convenience, preferred distances between these functional groups are also set forth below in Table 6.

TABLE 6
PF4 PHARMACOPHORE DISTANCES

| Pharmacophore | Distance (Å) |
|---|---|
| Points | Mean ± SD |
| I-II | 2.25 ± 0.05 |
| I-III | 6.03 ± 1.37 |
| I-IV | 6.92 ± 1.60 |
| I-V | 30.27 ± 2.92 |
| I-VI | 29.94 ± 2.49 |
| I-VII | 30.41 ± 4.31 |
| I-VIII | 8.57 ± 2.60 |
| I-IX | 14.20 ± 1.53 |
| I-X | 12.54 ± 1.51 |
| II-III | 6.00 ± 2.43 |
| II-IV | 7.01 ± 1.84 |
| II-V | 30.83 ± 1.99 |
| II-VI | 30.33 ± 1.97 |
| II-VII | 31.24 ± 4.03 |
| II-VIII | 9.09 ± 1.22 |
| II-IX | 14.45 ± 0.24 |
| II-X | 13.28 ± 0.37 |
| III-IV | 2.31 ± 0.07 |
| III-V | 26.35 ± 2.76 |
| III-VI | 26.57 ± 2.02 |
| III-VII | 26.31 ± 3.05 |
| III-VIII | 9.19 ± 1.40 |
| III-IX | 10.91 ± 1.74 |
| III-X | 7.06 ± 2.49 |
| IV-V | 25.58 ± 1.40 |
| IV-VI | 25.80 ± 1.31 |
| IV-VII | 25.34 ± 2.81 |
| IV-VIII | 9.02 ± 0.63 |
| IV-IX | 10.46 ± 0.46 |
| IV-X | 6.52 ± 1.26 |
| V-VI | 3.85 ± 1.54 |
| V-VII | 10.21 ± 2.21 |
| V-VIII | 23.10 ± 2.21 |
| V-IX | 17.29 ± 1.68 |
| V-X | 19.25 ± 2.12 |
| VI-VII | 14.07 ± 0.94 |
| VI-VIII | 21.84 ± 2.74 |
| VI-IX | 16.42 ± 2.03 |
| VI-X | 19.95 ± 2.02 |
| VII-VIII | 25.38 ± 4.39 |
| VII-IX | 20.60 ± 3.57 |
| VII-X | 18.76 ± 3.72 |
| VIII-IX | 6.87 ± 0.96 |
| VIII-X | 9.84 ± 1.05 |
| IX-X | 7.25 ± 0.49 |

6.3. Coordinate System Visualization and Bonding Potentials

The PF4 pharmacophore of the invention was further visualized to elucidate bonding and hydrophobic potential around each of the pharmacophore points. As described above, each pharmacophore point is classified as either a hydrogen bond acceptor, a hydrogen bond donor, or as participating in a hydrophobic interaction. By visualizing these points onto a coordinate system, the hydrophobic volumes and hydrogen bonding spherical surface caps can be better understood for the purposes of agonist/antagonist design.

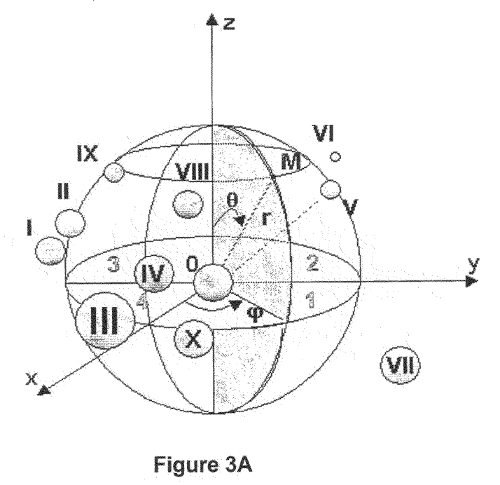

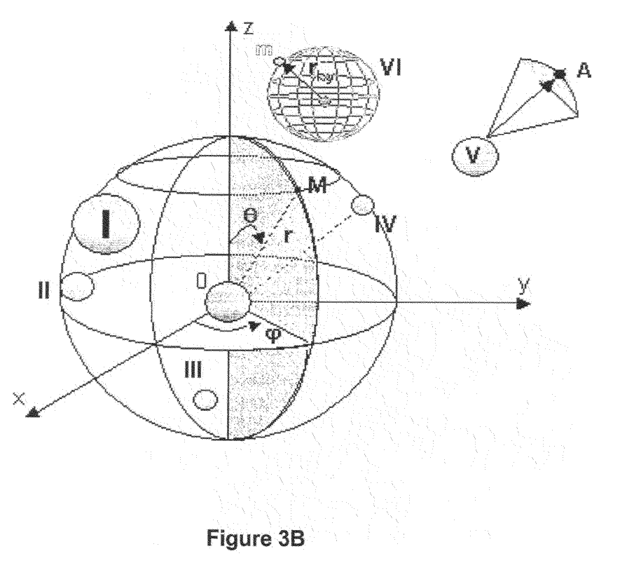

An origin was chosen and defined as 0 from which both Cartesian and spherical coordinate systems were drawn. The three dimensional figure from FIG. 2B was generated using the critical distances between the functional groups as described above. This figure was superimposed upon the Cartesian and spherical coordinate systems to account for hydrophobic volumes and hydrogen bonding vector directions. FIG. 3A provides an illustration of this pharmacophore in three dimensions. Each point in the pharmacophore is defined by the two geometric systems (Cartesian coordinates and spherical coordinates). Those skilled in the art can readily convert the Cartesian coordinates for a given point into spherical coordinates, and vice-versa, using well known mathematical relationships between these two coordinate systems. In particular, it is understood that the spherical coordinates, r, θ and φ, can be readily determined from given cartesian coordinates, x, y and z, using the relationships:

$$r = \sqrt{{x}^{2} + {y}^{2} + {z}^{2}}$$

$$ϕ = {tan}^{- 1} ( \frac{y}{x} ) , if x > 0$$

$$ϕ = {tan}^{- 1} ( \frac{y}{x} ) + 180 , if x < 0$$

$$θ = {cos}^{- 1} ( \frac{z}{r} )$$

Likewise, the cartesian coordinates, x, y and z, can be readily determined from given spherical coordinates, r, θ and φ, using the relationships:

x=r sin θ cos φ

y=r sin θ sin φ

z=r cos θ

For convenience, preferred Cartesian and spherical coordinates for the pharmacophore points are set fort below in Table 7.

TABLE 7
PF4 PHARMACOPHORE COORDINATES

| Pharmacophore | | | | | | |
|---|---|---|---|---|---|---|
| Points | x | y | z | r | θ | φ |
| I H. bond | −0.863 | −15.865 | 0.964 | 15.92 | 86.54 | 266.898 |
| acceptor | | | | | | |
| II H. bond | 0.452 | −15.619 | 2.749 | 15.87 | 80.036 | −88.355 |
| acceptor | | | | | | |
| III H. bond | 3.465 | −10.737 | −2.157 | 11.49 | 100.834 | −72.124 |
| donor | | | | | | |
| IV H. bond | 4.52 | −8.973 | −0.948 | 10.09 | 95.404 | −63.273 |
| acceptor | | | | | | |
| V H. bond | −1.662 | 17.444 | 0.654 | 17.53 | 87.874 | 95.431 |
| acceptor | | | | | | |
| VI H. bond | −3.49 | 16.751 | 2.271 | 17.26 | 82.451 | 101.758 |
| donor | | | | | | |
| VII H. bond | 1.2 | 17.361 | −12.069 | 21.18 | 124.756 | 86.058 |
| donor | | | | | | |
| VIII | −3.224 | −7.877 | 4.781 | 9.76 | 60.677 | 247.75 |
| hydrophobic | | | | | | |
| volume | | | | | | |
| IX hydrophobic | 0.564 | −1.39 | 4.395 | 4.74 | 21.998 | −67.924 |
| volume | | | | | | |
| X hydrophobic | 4.043 | −3.387 | −2.315 | 5.76 | 113.713 | −39.96 |
| volume | | | | | | |
| origin | 0.000 | 0.000 | 0.000 | 0.000 | 0.000 | 0.000 |

A point, M, was defined as the closest point to a hydrophobic pharmacophore point at which an undesirable interaction could be avoided. The hydrophobic volume around the pharmacophore point is defined as 4/3π(r_{hy})³ wherein r_{hy} is the distance between the pharmacophore point and point M on the surface of the hydrophobic volume. FIG. 3B provides an illustration of the hydrophobic volume around pharmacophore point VI. Preferred Cartesian and spherical coordinates for the hydrophobic volume outer sphere points (m points) are set forth below in Table 8.

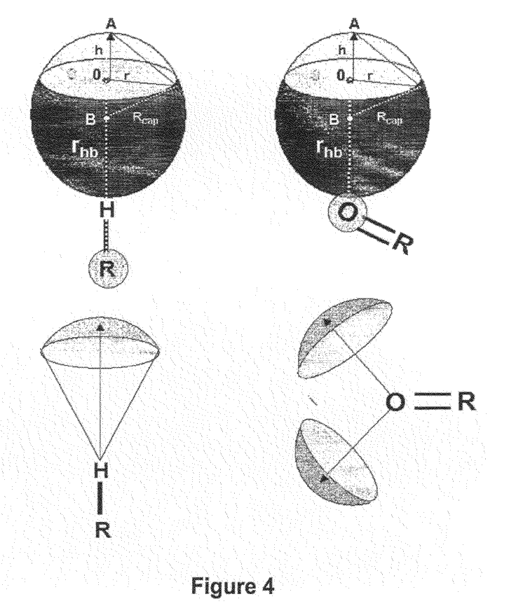

Furthermore, one or more hydrogen bond vectors, A, were calculated for each of the polar pharmacophore points using standard electronegativity data. FIG. 3B provides an illustration of one hydrogen bonding vector from pharmacophore point V. A hydrogen bonding potential spherical cap was then defined for each hydrogen bond vector as having a concave depth of ¼ the length of the hydrogen bonding vector in a sphere whose radius is ½ the length of the hydrogen bonding vector. FIG. 4 shows the graphical representation of both hydrogen bond donating and hydrogen bond accepting hydrogen bonding potential spherical caps.

The surface area of the hydrogen bond cap is defined as 2πR_{cap}h wherein R_{cap} is the radius of the sphere and h is concave depth of the spherical cap. For convenience, preferred Cartesian and spherical coordinates for the hydrogen bond vector points (A points) for this pharmacophore are set forth below in Table 8. Similarly, the hydrophobic volumes (“Vol”) and hydrogen bonding cap surface areas (“S”) for this pharmacophore are set forth below in Table 9.

TABLE 8
HYDROPHOBIC POINT AND
HYDROGEN BONDING VECTOR COORDINATES

| Pharmacophore | | | | | | |
|---|---|---|---|---|---|---|
| Points | x | y | z | r | θ | φ |
| origin | 0.000 | 0.000 | 0.000 | 0.000 | 0.000 | 0.000 |
| A_Ia | −2.128 | −15.922 | −0.612 | 16.07 | 92.195 | 262.399 |
| A_Ib | −0.428 | −17.301 | 2.154 | 17.44 | 82.917 | 268.595 |
| A_II | 1.677 | −15.542 | 4.299 | 16.21 | 74.631 | −83.853 |
| A_IIIa | 5.531 | −9.996 | −4.33 | 12.22 | 110.768 | −61.052 |
| A_IIIb | 1.381 | −12.838 | −2.067 | 13.08 | 99.106 | −83.872 |
| A_IVa | 4.281 | −7.704 | 0.472 | 8.83 | 86.948 | −60.948 |
| A_IVb | 6.071 | −8.527 | −2.208 | 10.7 | 101.923 | −54.558 |
| A_V | −1.005 | 17.349 | −1.135 | 17.41 | 93.751 | 93.303 |
| A_V | −0.689 | 18.181 | 2.074 | 18.31 | 83.508 | 92.158 |
| A_VI | −2.426 | 18.896 | 3.877 | 19.44 | 78.507 | 97.305 |
| A_VI | −5.475 | 14.573 | 2.612 | 15.78 | 80.483 | 110.581 |
| A_VII | −0.253 | 17.729 | −13.524 | 22.3 | 127.351 | 90.805 |
| A_VII | 2.637 | 17.019 | −10.78 | 20.32 | 122.057 | 81.204 |
| m_VIII | −5.281 | −7.342 | 3.491 | 9.69 | 68.893 | 234.281 |
| m_IX | 1.731 | −0.451 | 4.147 | 4.52 | 23.443 | −14.605 |
| m_X | 5.683 | −2.278 | −3.603 | 7.1 | 120.512 | −21.846 |

TABLE 9
PF4 PHARMACOPHORE POINT
HYDROPHOBIC VOLUMES
AND HYDROGEN BONDING CAP SURFACE AREAS

| Pharmacophore Point | Area (Å)² | Volume (Å)³ |
|---|---|---|
| S_Ia | 5 | |
| S_Ib | 5 | |
| S_II | 5 | |
| S_IIIa | 4 | |
| S_IIIb | 4 | |
| S_IVa | 5 | |
| S_IVb | 5 | |
| S_Va | 5 | |
| S_Vb | 5 | |
| S_VIa | 4 | |
| S_VIb | 4 | |
| S_VIIa | 4 | |
| S_VIIb | 4 | |
| Vol_VIII | | 64 ± 1 |
| Vol_IX | | 14.7 ± 1 |
| Vol_X | | 55 ± 1 |

6.4. Use of the Pharmacophore in Compound Design

This example demonstrates how a pharmacophore of this invention can be used to identify, design and synthesize compounds that can be either agonists or antagonists of the PF4 receptor. In particular, a lead compound, referred to here as BQ-A01104 (Formula I), is disclosed.

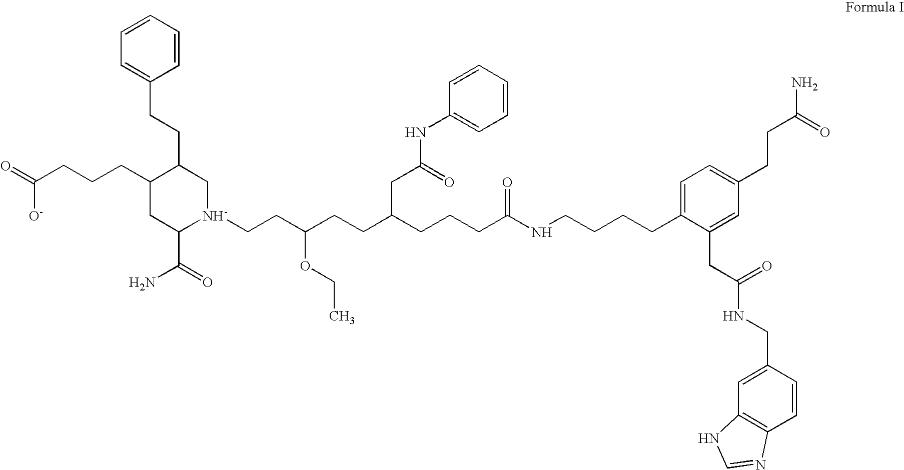

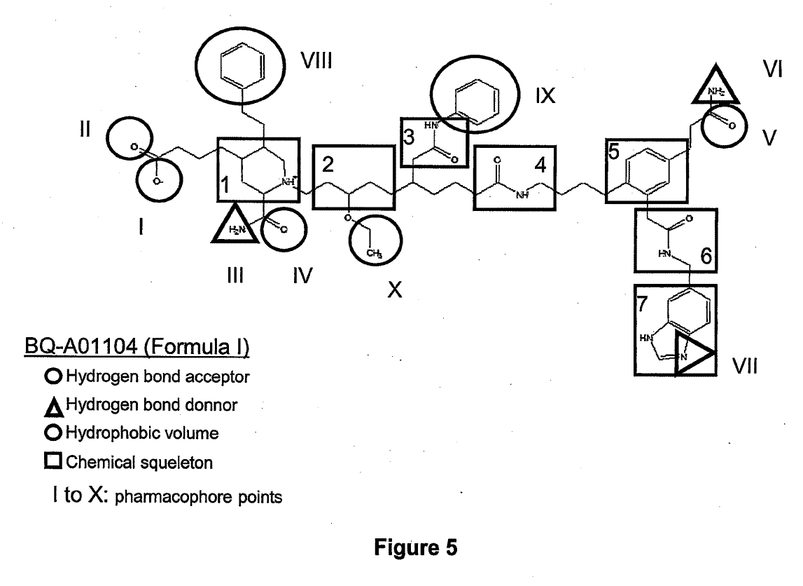

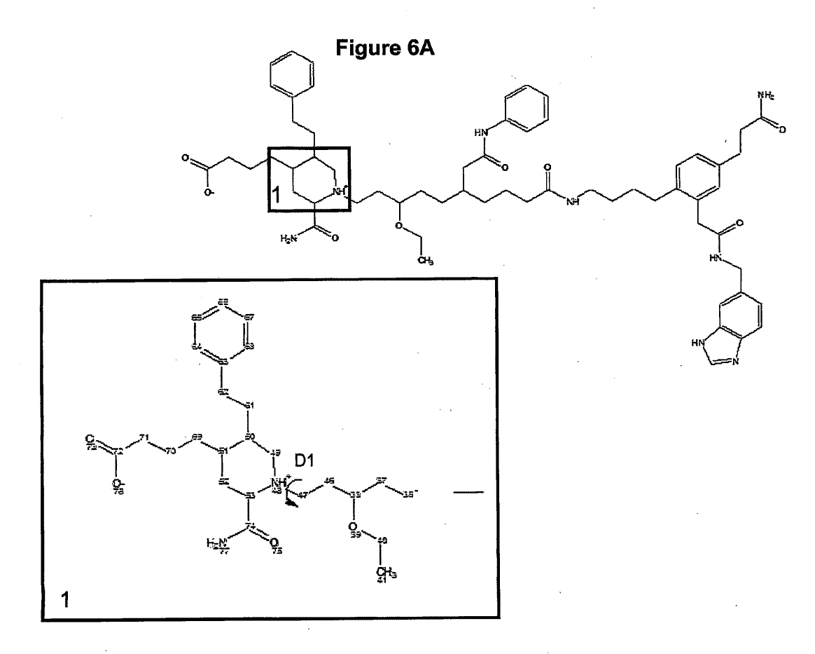

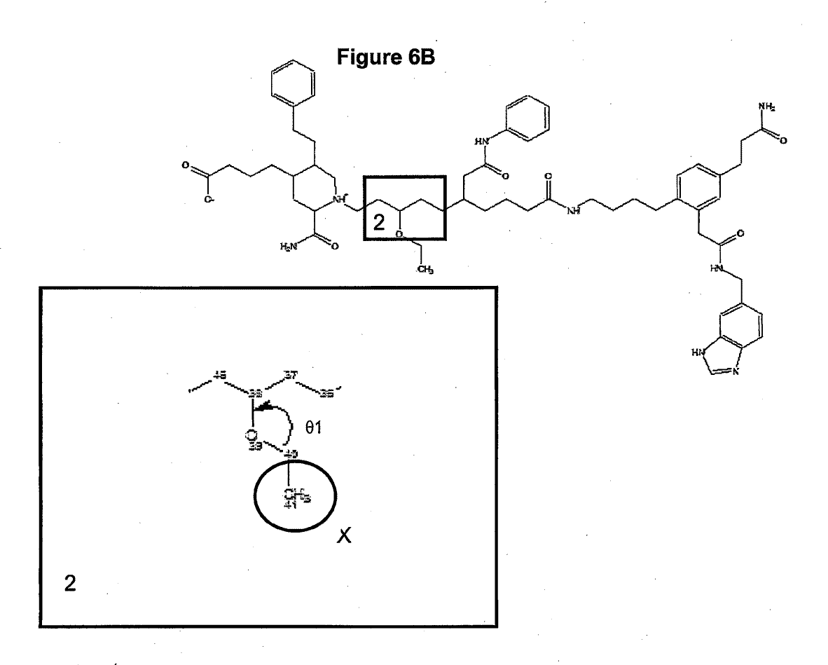

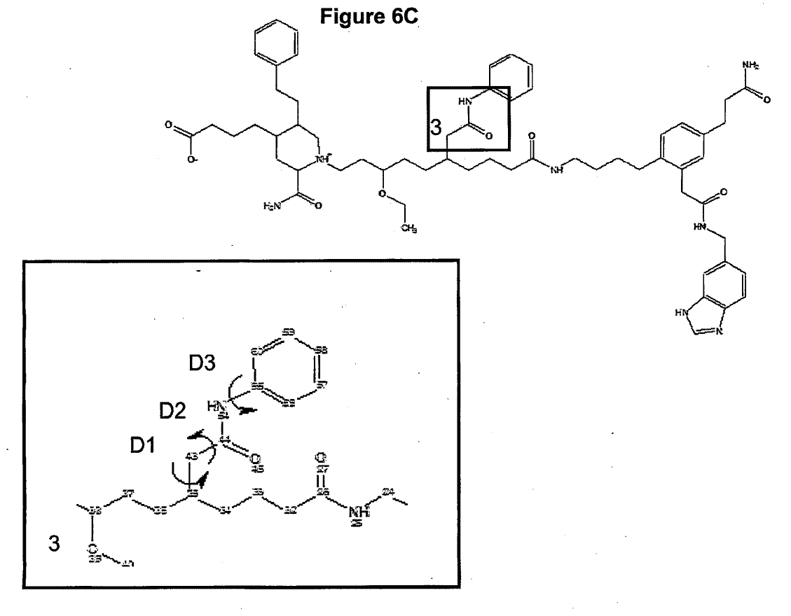

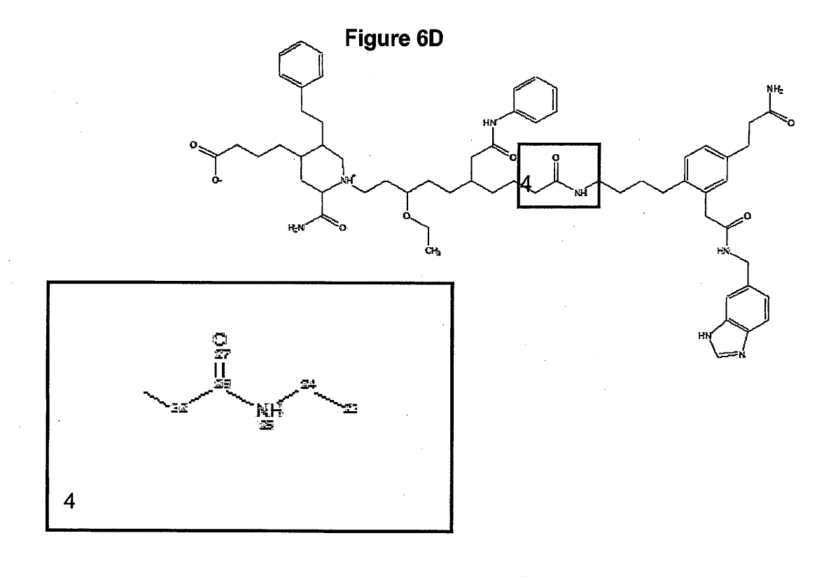

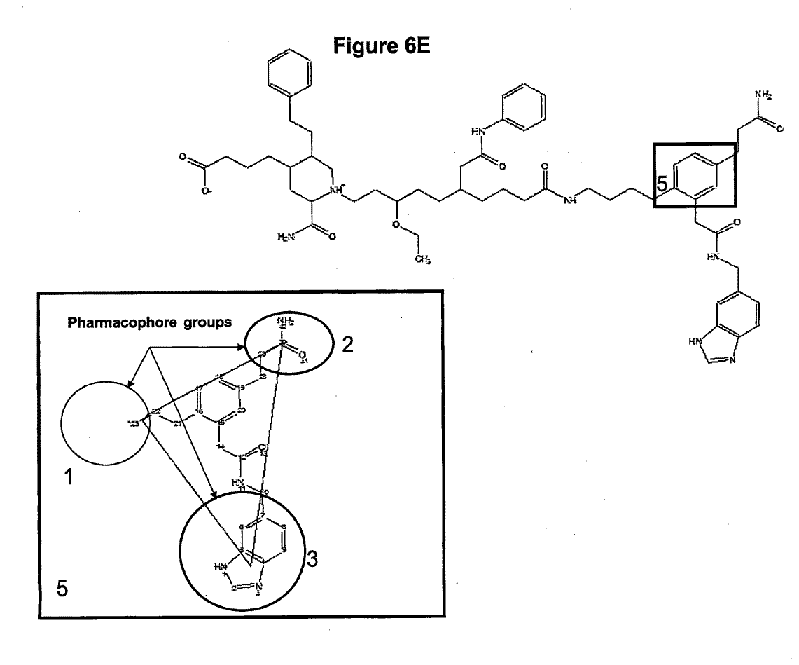

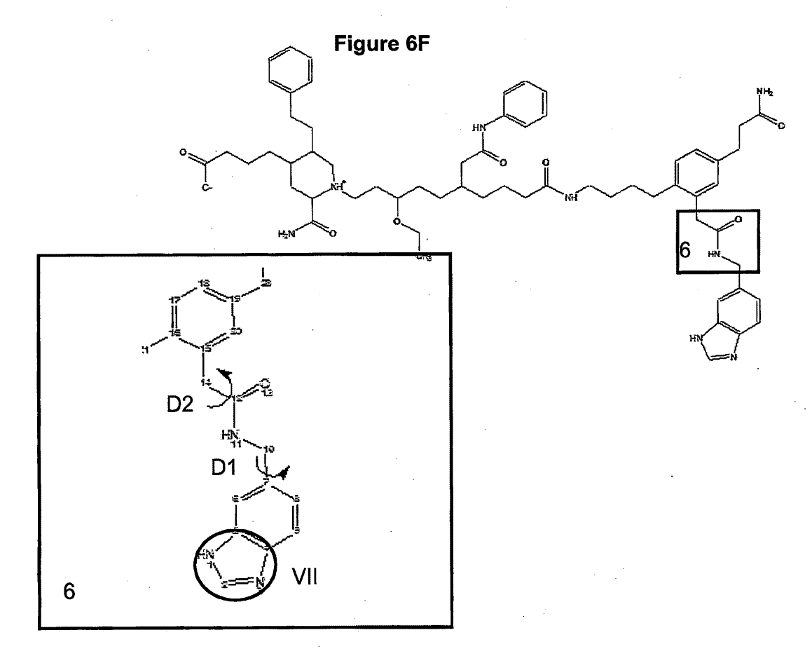

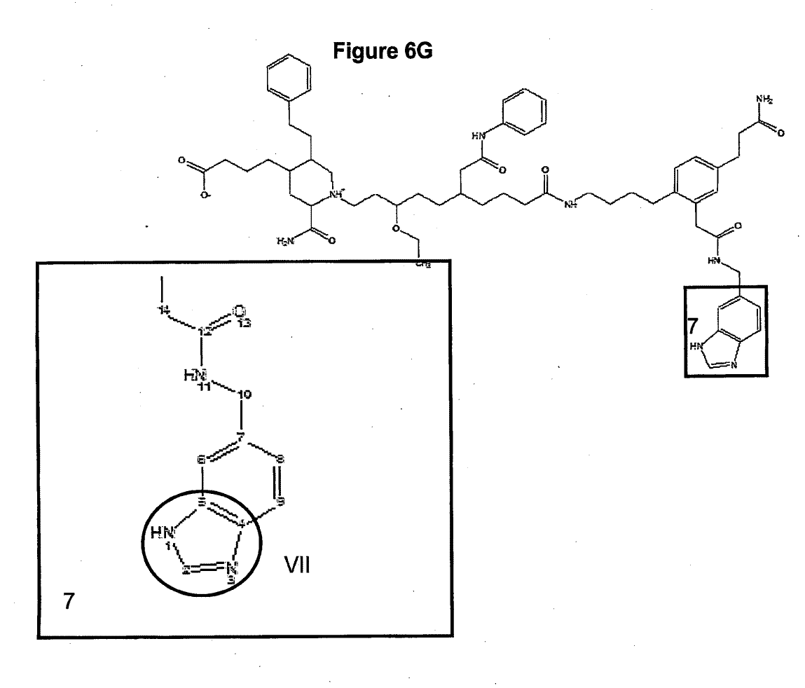

BQ-A01104 is a neutral molecule with one anionic group (a carboxylic acid group) and a cationic group (a quaternary amine in the piperidinium ring). The compound is soluble in an aqueous solution of sodium chloride. The compound comprises all ten of the PF4 pharmacophore points listed in Table 5, supra, held structurally rigid by a scaffold that, for convenience, can be conceptualized a seven distinct subunits or “zones.” The chemical structure of BQ-A01104 is illustrated in FIG. 5, with each of the ten pharmacophore points indicated by the corresponding Roman numeral listed in Table 5, above. Each of the structural subunits or “zones” is also indicated by a corresponding arabic numeral. These structural subunits are illustrated individually in FIGS. 6A-6G, and discussed below. High temperature molecular dynamics (MD) simulation of the molecule is water (1 nanosecond at 900 Kelvin) reveals that the molecule is structurally stable, and maintains all structural constraints. That is to say, the scaffold stays rigid along all MD trajectories.

Zone 1 (FIG. 6A), the first chemical subunit, comprises a piperidinium ring that carries the pharmacophore groups I through IV and VIII, linked to the ring by flexible chemical arms. The sp hybridization of the quaternary amine in this subunit allows good presentation of the pharmacophore points in three-dimensional space. Rotation about the dihedral angle D1 (shown in FIG. 6A), which joins Zone 1 and Zone 2, is limited due to the proximity of the nitrogen containing ring and aliphatic carbon (carbon 27). This dihedral angle has a value of about 46.9°, providing good presentation of the pharmacophore points.

Zone 2 (FIG. 6B) maintains the presentation of an ethyloxy side chain corresponding to pharmacophore point X via an sp³ carbon (C38) in the aliphatic backbone. The ketone oxygen gives a desirable bend to the bending angle, in order to correctly present the pharmacophore point X.

Zone 3 (FIG. 6C) comprises a peptide bond that gives some rigidity to the side chain carrying the pharmacophore point IX. The dihedral angles D1, D2 and D3 for this subunit (shown in FIG. 6C) have average values of −155.6°, 53.3° and 22.3°, respectively. This configuration allows the aromatic ring corresponding to the pharmacophore point IX to be oriented toward the above-described chemical subunits.

Zone 4 (FIG. 6D) links zones 3 and 5 to each other at a fixed angle, by means of a peptide bond that is rigid even during high temperature MD simulations.

Zone 5 (FIG. 6E) comprises an aromatic ring, which maintains an energetically favorable relative orientation between the pharmacophore points V and VI on one branch (labeled in FIG. 6E as Branch 2), pharmacophore point VII on the other branch (labeled in FIG. 6E as Branch 3), and the remaining pharmacophore points I-IV and VIII-X on the third branch (labeled in FIG. 6E as Branch 1).

Zone 6 (FIG. 61) comprises a peptide bond, giving rigidity to the side chain carrying the pharmacophore point VII. The average dihedral angle values D1 and D2 (shown in FIG. 6F) are −108° and 26°, respectively. This configuration allows the benzimidazole ring corresponding to pharmacophore point VII to be correctly oriented for efficient activity.

Zone 7 (FIG. 6G) comprises a benzimidazole ring that correctly orients the nitrogen three atom in order to fit the pharmacophore point VII.

Pharmacophore points I, II, V, VI and VIII are connected to backbone subunits in BQ-A011004 via flexible aliphatic chains. By contrast, pharmacophore points III, IV, VII, IX and X are connected to the backbone subunits of BQ-A011004 by chains that are relatively rigid and constrained. These latter pharmacophore points are therefore relatively constrained compared to the former. This reflects the relative flexibility of different pharmacophore points in the PF4 polypeptide itself. For example, restrained flexibility of pharmacophore points X and IX, which are located on the Ala43 and Leu45 amino acid residues of PF4 (SEQ ID NO:1), is imposed by the existence of an α-helix that is necessary for PF4 activity. The stability of this helix is maintained by a capping box present at its N-terminal end. In the PF4 polypeptide (SEQ ID NO:1), therefore, the movements of residues Val13 and Leu11 are restrained due to the rigidity of the PF4 skeleton imposed by two disulfide bridges.

6.4.1 Preparation of BQ-A01104

BQ-A01104 and other compounds identified and designed as either agonists or antagonists of the PF4 receptor can be obtained via standard, well-known synthetic methodology.

Various compounds identified and designed as either agonists or antagonists of the PF4 receptor contain one or more chiral centers, and can exist as racemic mixtures of enantiomers or mixtures of diastereomers. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, Ind., 1972).

Some convenient methods are illustrated in Schemes 14. These schemes are merely meant to be illustrative of one synthetic pathway, however, these synthetic pathways can be modified in ways that will be obvious to those skilled in the art to create a variety of compounds. Starting materials useful for preparing the compounds of the invention and intermediates therefore, are commercially available or can be prepared from commercially available materials using known synthetic methods and reagents.

Methods of synthesizing the compounds of the present invention are illustrated in the following schemes. Because of possible discrepancies in using chemical nomenclature, where structures are provided for compounds or moieties the structure, and not the chemical name, controls the definition of the compound or moiety.

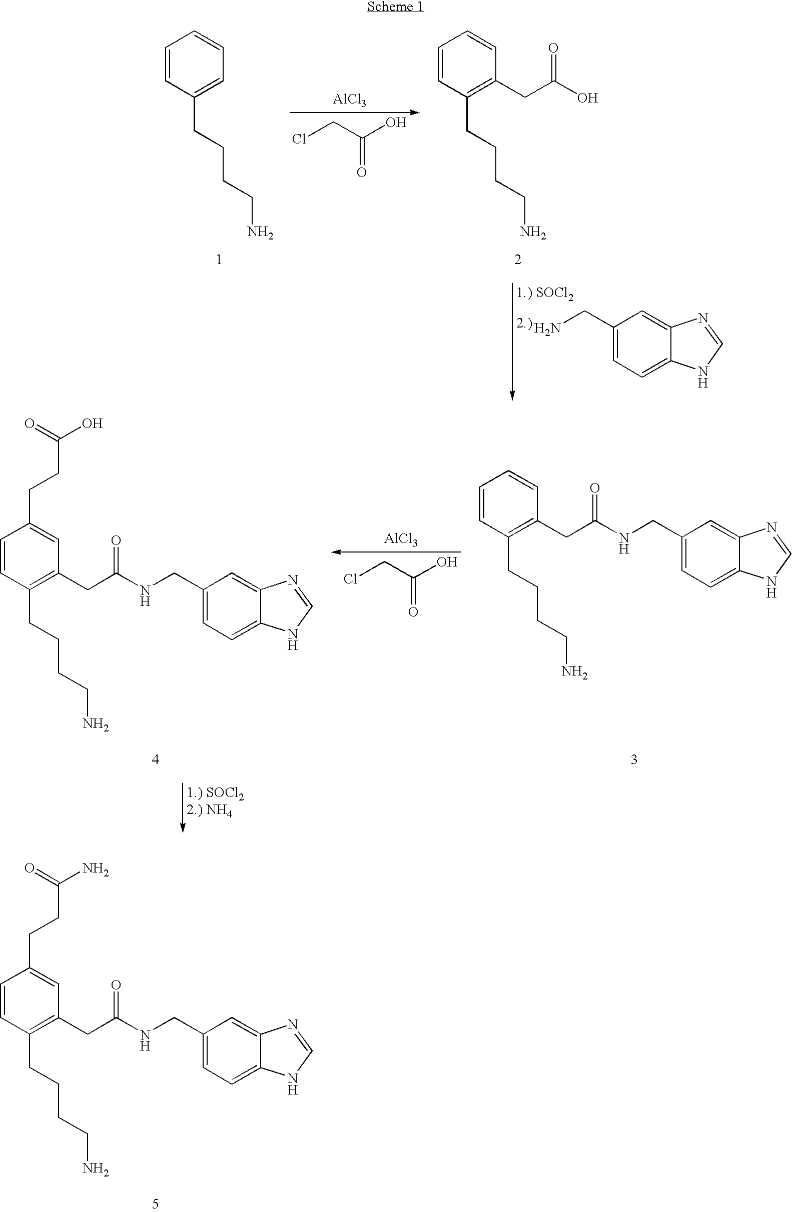

In scheme 1, intermediate 5 is produced by first alkylating 4-phenylbutylamine (1) (Aldrich Chemical Co.) with aluminum chloride in water with chloroacetic acid to produce phenylacetic acid compound 2. Compound 2 is reacted with thionyl chloride to produce the acid chloride which is reacted with the benzimidazol-5-yl-methylamine to form the amide compound 3. Benzimidazol-5-yl-methylamine is made in 3 steps from commercially available benzimidazole carboxylic acid (Aldrich Chemical Co.); (1) treatment of the carboxylic acid with thionyl chloride to form the acid chloride, (2) reaction of the acid chloride with ammonia to form the corresponding primary amide (See Beckwith et al. in Zabicky The Chemistry of Amides Wiley, NY, 1970, pg. 73), and (3) reduction of the amide with lithium aluminum hydride in THF to form the desired methyl amine (See Challis et al. in Zabicky The Chemistry of Amides Wiley, NY, 1970, pg. 795). Compound 3 is then alkylated again with 3-chloropropionic acid and aluminum chloride in water to produce the trisubstituted phenyl compound 4. Finally compound 4 is reacted with thionyl chloride and ammonia to convert the carboxylic acid to the amide intermediate 5.

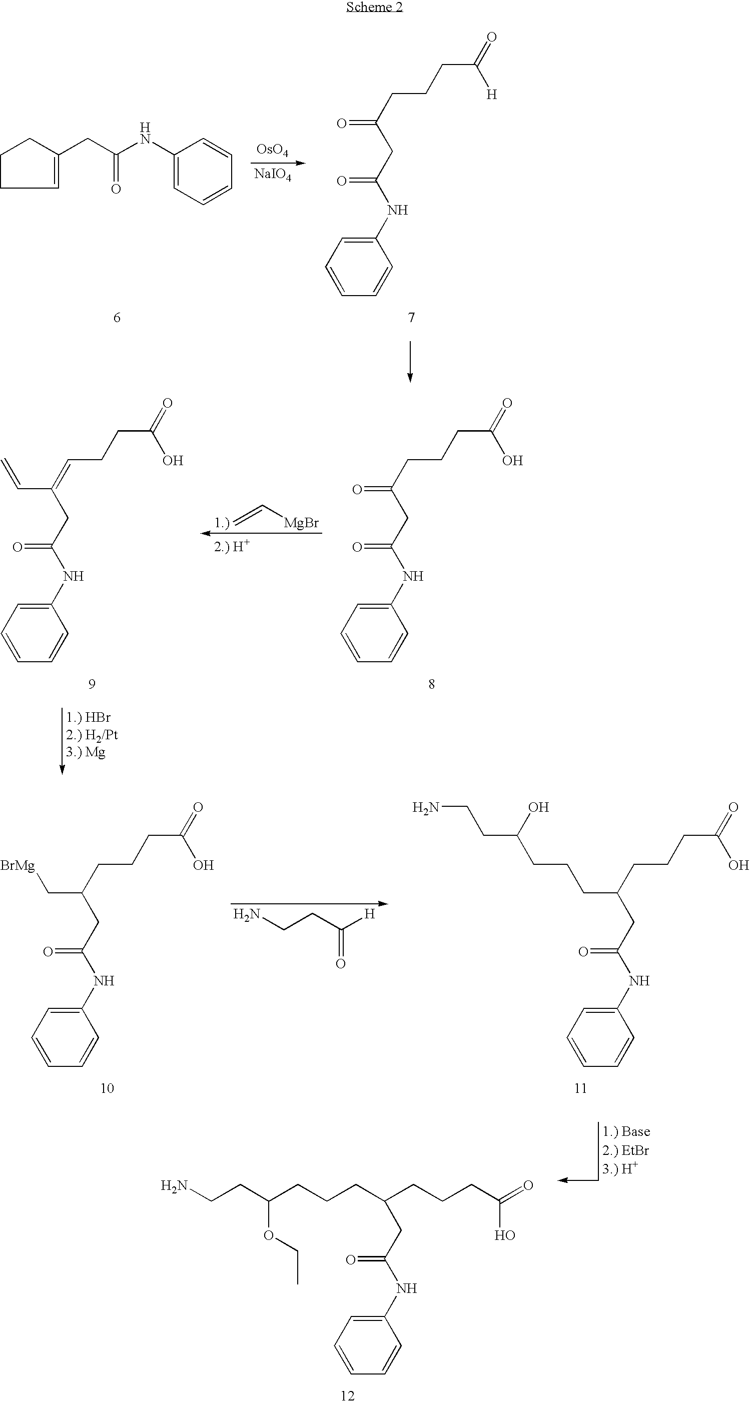

In scheme 2, intermediate 12 is produced by converting the cylcopentenyl amide compound (6) to the 1,3-dicarbonyl compound (7) with osmium tetroxide followed by treatment with sodium periodate and then treatment with water and a mild reducing agent such as NaHSO₃. Compound 6 is formed in 3 steps from commercially available cyclopentanone (Aldrich Chemical Co.); (1) an aldol reaction of cyclopentanone with the enolate of ethyl acetate, (2) dehydration of the resultant alcohol by treatment with acid, and (3) conversion of the resultant α,β-unsaturated ester to its corresponding amide upon reaction with the sodium or lithium salt of aniline (Majetich et al. Tetrahedron Lett. 1994, 35, 8727). Compound 7 is oxidized using standard techniques, for example treatment with KMnO₄, to the carboxylic acid compound 8. Compound 8 is treated with vinylmagnesium chloride and the resultant alcohol subsequently dehydrated with acid to produce the diene compound 9. The vinyl alkene of compound 9 is brominated with hydrogen bromide followed by hydrogenation of the heptenyl olefin with hydrogen gas in the presence of a catalytic amount of palladium on carbon. Finally the 1-bromoalkane is reacted with magnesium to produce the alkyl grignard reagent 10. Compound 10 is then reacted with 3-aminopropanal in ether to produce alcohol compound 11. Finally compound 11 is reacted with a base, followed by ethylbromide and then acid to form ethyl ether intermediate 12.

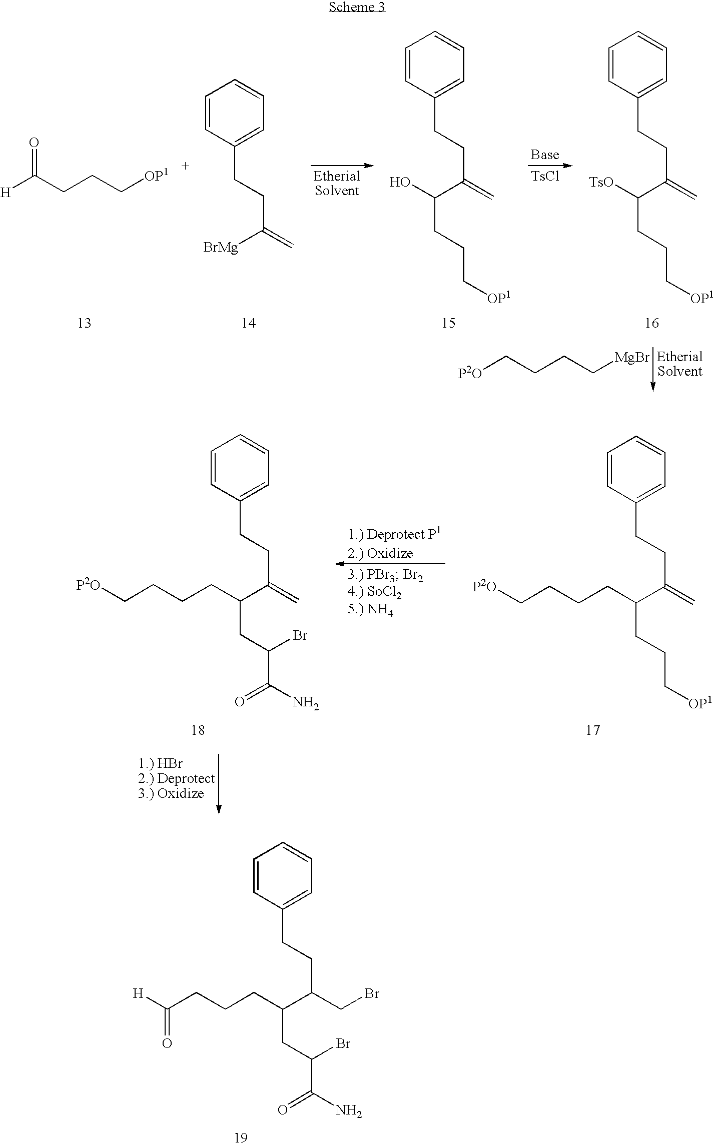

In scheme 3, intermediate 19 is produced in three steps from commercially available 3-butenal diethyl acetal (Aldrich Chemical Co.); (1) hydroboration with BH₃ followed by oxidation with NaOH/H₂O₂, 2) conversion of the diethyl acetal to the aldehyde with treatment of catalytic p-toluene sulfonic acid, and (3) protection of the alcohol of 4-hydroxy-butanal to form compound 13. The choice of appropriate protecting groups in this and other steps of the synthesis will be readily determined by one of ordinary skill in the art. Suitable protecting groups and standard techniques for choosing and synthesizing protecting groups can be found in T. W. Greene and P. G. M. Wuts, Protective Groups in Organic Synthesis (Wiley-Interscience, New York, 1999). Compound 13 is then reacted with the grignard reagent (14; forms in two steps, (1) addition of HBr to 4-phenyl-1-butyne (Aldrich Chemical Co.) under Markovnikov conditions, and (2) reaction of the resultant vinylic bromide with magnesium) in ether to produce alcohol compound 15. Compound 15 is reacted with tosyl chloride, or other suitable leaving group precursor, in the presence of base, e.g., NEt₃, to produce tosylate compound 16. Compound 16 is reacted with the Gringard reagent formed by protecting 4-bromobutanal (4-bromobutanal is made from 4-hydroxy-butanal (supra) upon treatment with 2,4,6-trichloro[1,3,5]triazine, NaBr and N,N-dimethylformamide in methylene chloride; de Luca et al. Org. Lett., 2002, 4, 553-555) with a protecting group that is orthogonal to P¹ and reacting the protected compound with magnesium to form compound 17. Compound 17 is deprotected to remove the original protecting group P¹ and the free alcohol is subsequently oxidized to the carboxylic acid with, for example, CrO₃. After oxidation, the intermediate is brominated with tribromophospine and bromine gas to form the α-bromo carboxylic acid. The carboxylic acid is then treated with thionyl chloride and the resultant acid chloride is treated with ammonia to produce amide compound 18. The olefin of compound 18 is brominated with hydrogen bromide to afford the primary bromide and the second protecting group (P²) is removed from the intermediate and the resultant alcohol oxidized to the aldehyde using standard methods, e.g. treatment with the Swern or Dess-Martin reagent, to form intermediate 19.

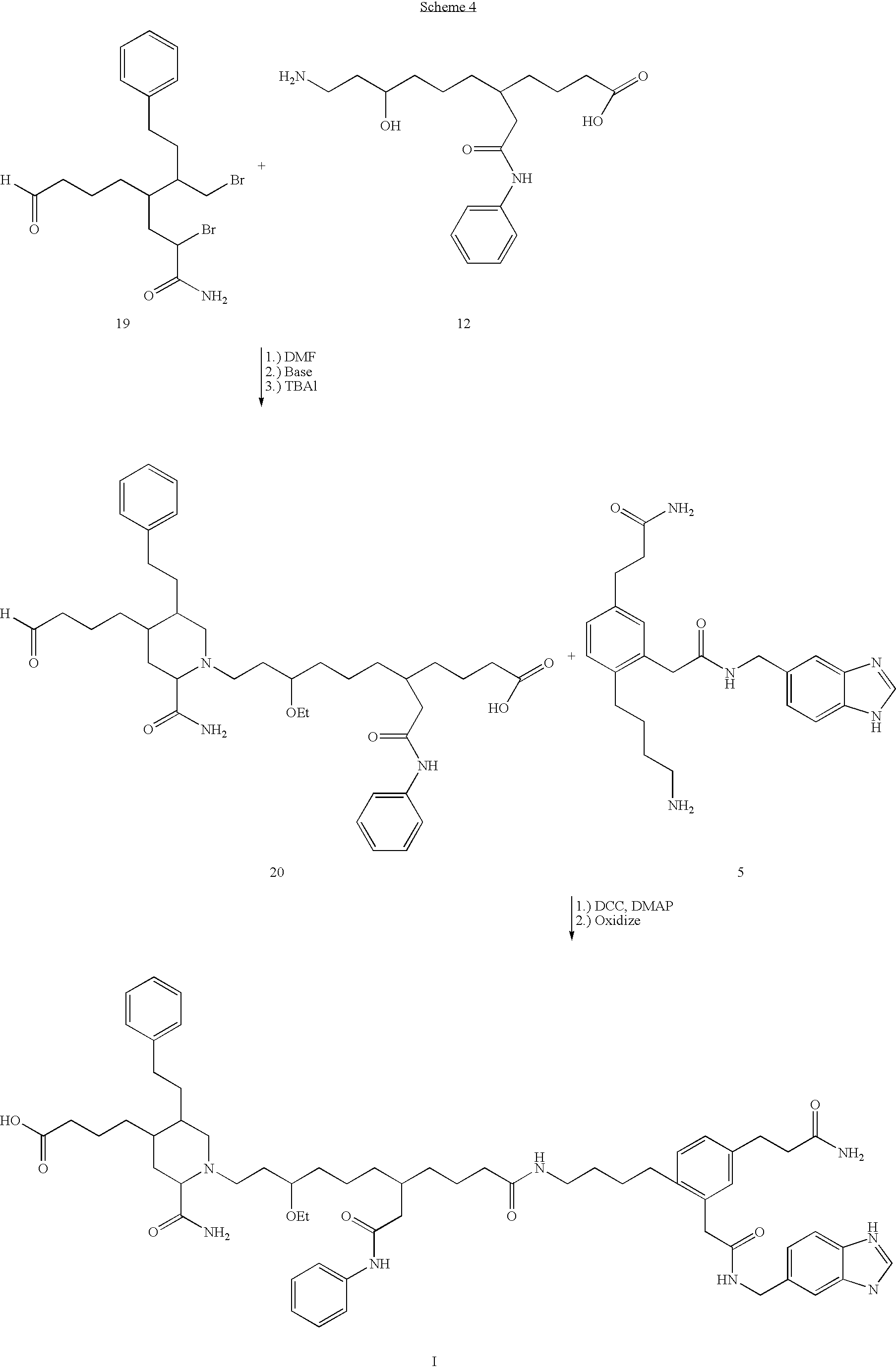

Finally, in scheme 4, the dibromo intermediate 19 is coupled with the amine intermediate 12 in the presence of a base and tert butyl-ammonium iodide (TBAI) to give the piperidine intermediate 20. The carboxylic acid of intermediate 20 is coupled with the amine of intermediate 5 in the presence of DCC and catalytic DMAP followed by oxidation of the remaining aldehyde with, for example KMnO₄, to afford title compound I, BQ-A01104.

6.5. Optimization of Activity

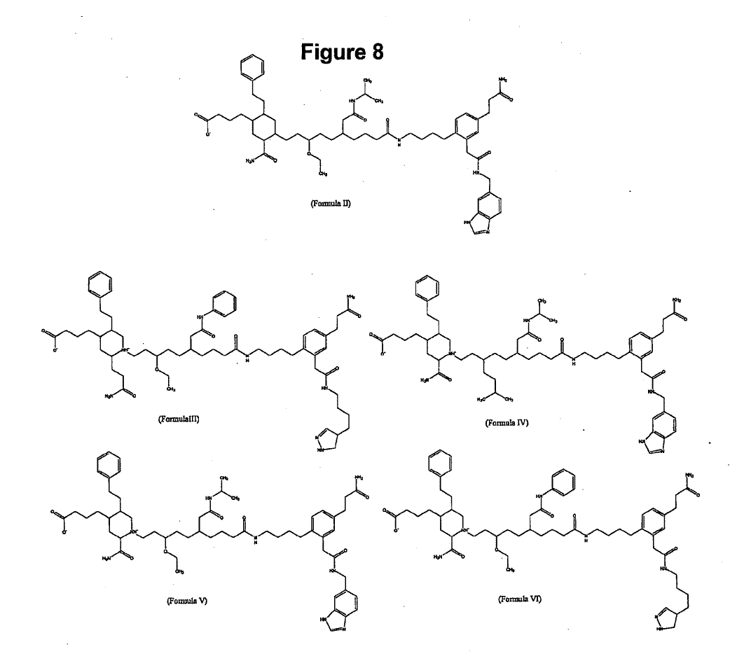

Using routine techniques of chemical synthesis and modification, it is possible to further optimize both the activity and Absorption, Distribution, Metabolism and Excretion (ADME) properties of compounds that are designed and/or identified using the pharmacophores of this invention. For example, candidate PF4 agonist or antagonist compounds can be modified either by modifying one or more functional groups that correspond to pharmacophore points, by modifying the scaffolding (e.g. the subunits or “zones” described, supra, for BQ-A011004), or both. FIG. 7 illustrates certain, exemplary modifications that can be made to optimize the compound BQ-A011004. The complete chemical structures of these modified compounds (Formulas II-VI) are shown in FIGS. 8A-8E. Such modifications, and compounds comprising them, therefore are also considered a part of the present invention, as is their use, e.g., as agonists or antagonists of PF4. Compounds II-VI can, for example, be prepared from the following synthetic protocols that are derivations of schemes 1-4.

The preparation of the compound of Formula II is illustrated in schemes 5-6. The key modifications to the BQ-A011004 scaffold are the substitution of a cyclohexyl ring for the piperazine ring, and the substitution of an isopropylamide group for the phenylamide group.

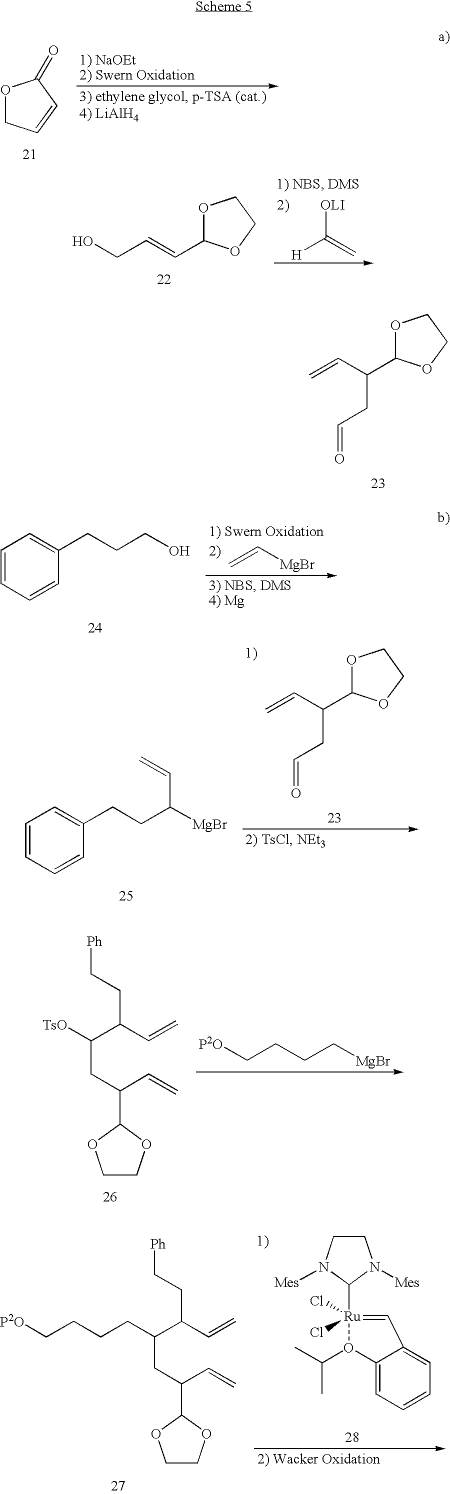

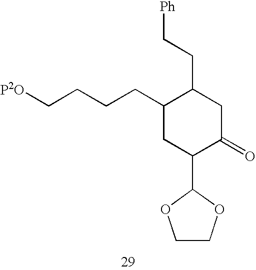

The substitution of the cyclohexyl fragment for the piperazine ring is accomplished according to Schemes 5a, 5b. First, γ-caprolactone (Aldrich Chemical Co.) is converted to the corresponding ring-opened ester with treatment of sodium ethoxide (Scheme 5A). The intermediate ester alcohol is oxidized under Swern conditions to the corresponding ester aldehyde, and the aldehyde protected as its dioxolane with ethylene glycol in the presence of catalytic p-TSA. The ester is then reduced to the alcohol with LiAlH₄ to provide compound 22. Treatment of compound 22 with N-bromosuccinimide and methylsulfide converts the allylic alcohol to its corresponding allyl bromide (Corey et al. Tetrahedron Lett. 1972, 4339). Reaction of the allyl bromide with the lithium enolate of acetaldehyde (formed from acetaldehyde and lithium diisopropylamide) provides alkene aldehyde compound 23.

As illustrated in Scheme 5B, 3-phenyl-1-propanol (24, Aldrich Chemical Co.) is first oxidized under Swern conditions to the aldehyde and the aldehyde is reacted with vinylmagnesium bromide which, upon reaction workup, affords the corresponding allylic alcohol. The allylic alcohol is first reacted with NBS and DMS to afford the allyl bromide and the bromide is converted to the corresponding Grignard reagent (25) with magnesium. Compound 25 is then added to aldehyde 23 and the resultant alcohol is converted to the corresponding tosylate (26) with tosyl chloride in the presence of base (e.g., NEt₃). The tosylate is displaced by treatment with a protected 4-hydroxybutyl Grignard reagent to form diene 27. Ring closing metathesis of compound 27 with a catalytic amount of Hoveyda-Grubbs 2^{nd} generation catalyst (28) (Hoveyda et al. J. Am. Chem. Soc. 121, 791, 1999) followed by a Wacker oxidation with PdCl₂ (Tsuji, J. Synthesis 1990, 739) affords cyclic ketone 29.

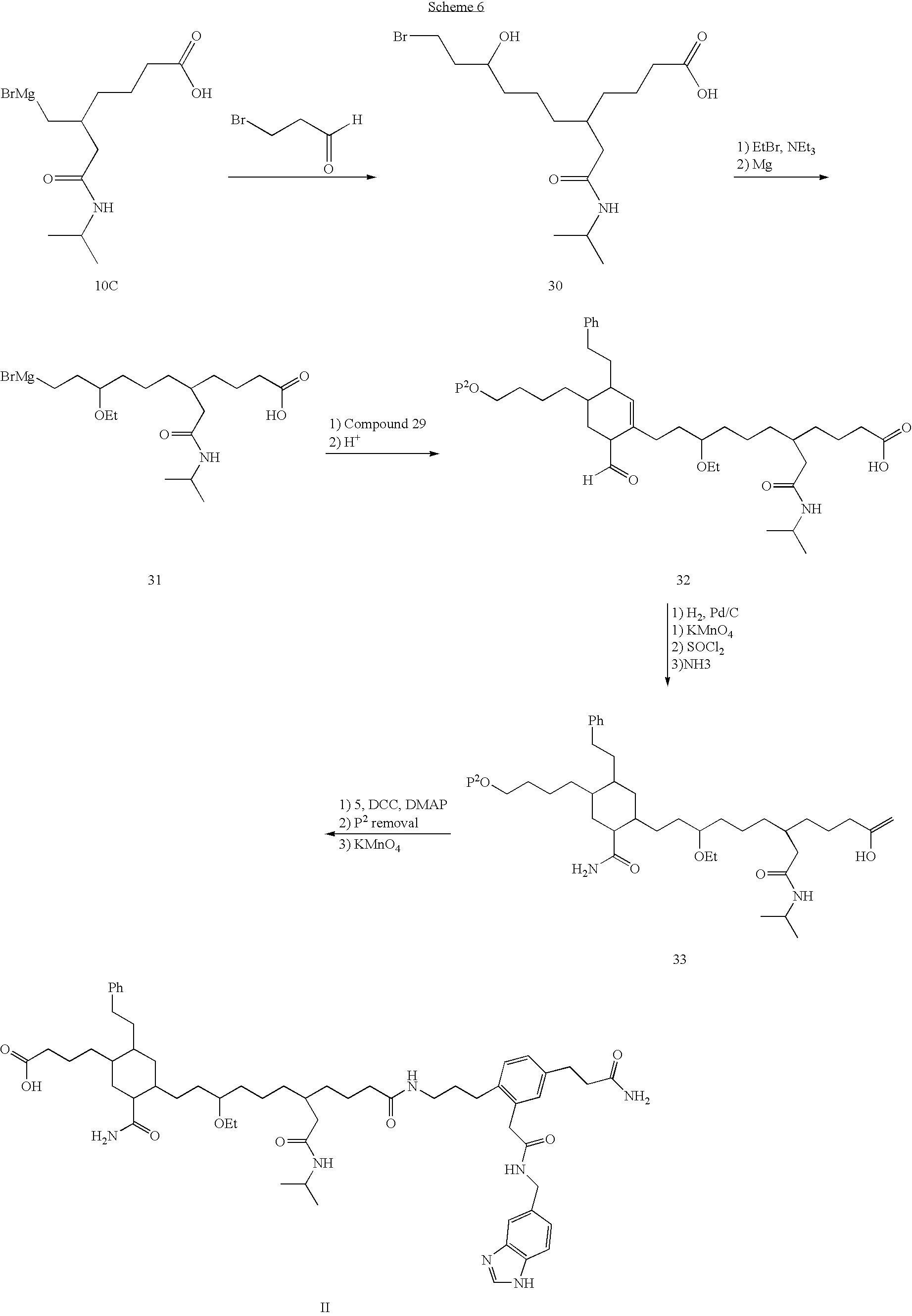

The remainder of the compound of Formula II is constructed as illustrated in Scheme 6. Compound 10C (See Scheme 9, infra) is reacted with 3-bromopropionaldehyde to afford alcohol 30. The alcohol is then converted to its corresponding ethyl ether upon treatment with ethyl bromide in the presence of base (e.g., NEt₃) and the intermediate compound is converted to its corresponding Grignard reagent (31) with magnesium metal. Grignard reagent 31 is then added to compound 29 and the resultant alcohol dehydrated to its corresponding alkene (32) upon treatment with acid (the dioxolane group is also removed in this step). Compound 32 is then hydrogenated in the presence of hydrogen and catalytic palladium on carbon and the aldehyde converted to its corresponding amide by 1) oxidation to the acid with KMnO₄, 2) conversion of the acid to the acid chloride with thionyl chloride, and 3) reaction of the acid chloride with ammonia. The resultant amide 33 is then coupled with compound 5 (See Scheme 1) in the presence of DCC and catalytic DMAP. Finally, the compound of Formula II is completed when the protecting group P² is removed and the resultant alcohol oxidized to its corresponding acid with KMnO₄.

The preparation of the compound of Formula III is illustrated in scheme 7-8. The key modifications to the BQ-A011004 scaffold are the substitution of an aminocarbonyl ethyl group for the aminocarbonyl group substituted on the piperazine ring, and the substitution of a 4-[4-aminobutyl]-4,5-dihydropyrazole for the aminomethylbenzimidazole fragment.

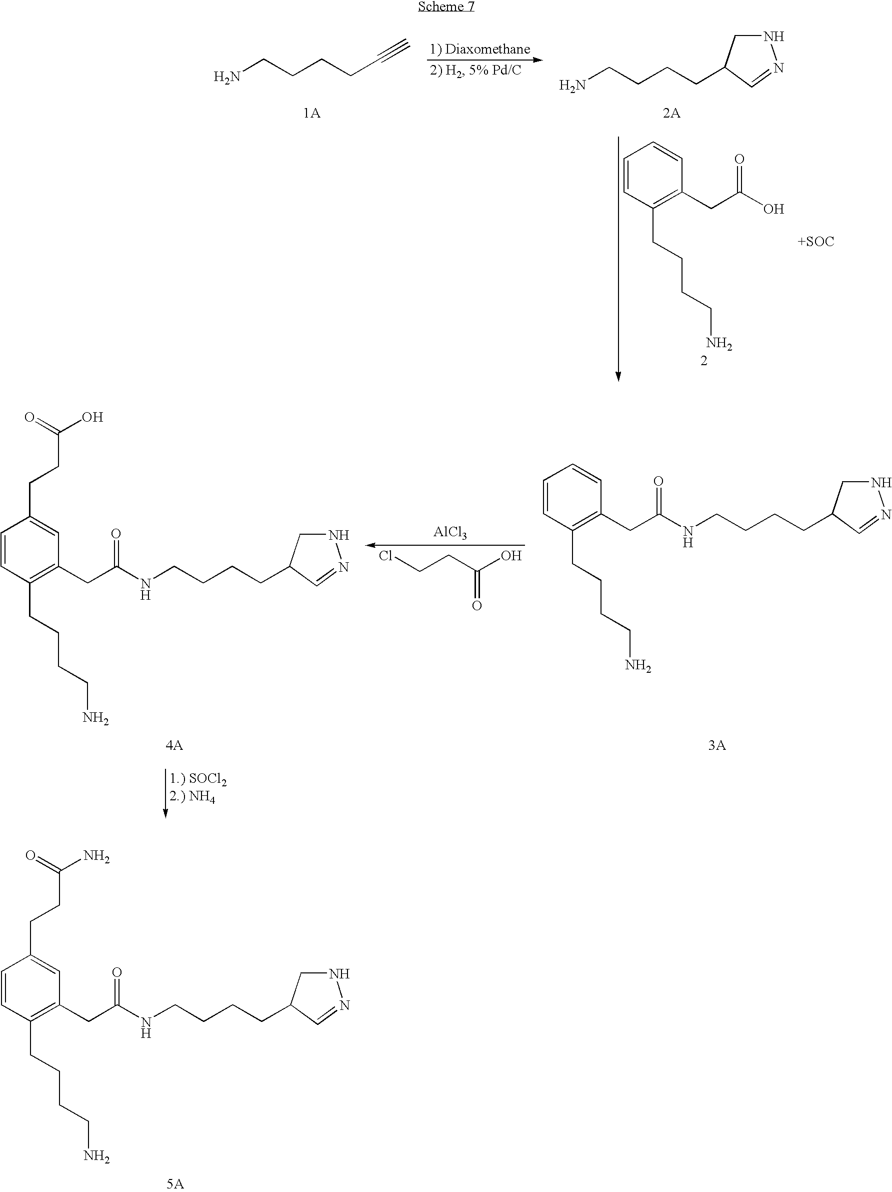

The substitution of an 4-[4-aminobutyl]-4,5-dihydropyrazole group is achieved through the synthesis of fragment 5A as illustrated in Scheme 7. Reaction of 6-amino heptyne (IA) with diazomethane under Pechmann conditions (T. L. Jacobs in R. C. Elderfield, Heterocyclic Compounds 5, 70 (New York, 1957)) affords 4-[4-aminobutyl]pyrazole which is reduced to the corresponding 4,5-dihydropyrazole (2A) with hydrogen in the presence of catalytic palladium on carbon. Dihydropyrazole 2A is then coupled with the acid chloride of compound 2 (i.e., reaction of compound 2 from Scheme 1 with thionyl chloride) to form amide 3A. Compound 3A is then alkylated again with 3-chloropropionic acid and aluminum chloride in water to produce the trisubstituted phenyl compound 4A. Finally compound 4A is reacted with thionyl chloride and ammonia to convert the carboxylic acid to the amide intermediate 5A.

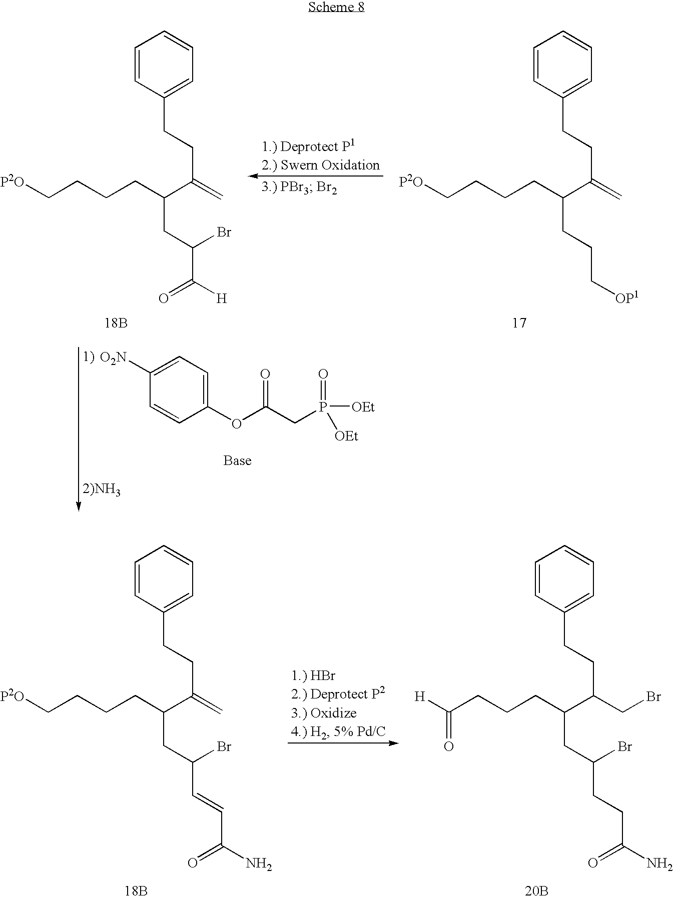

The substitution of an aminocarbonyl ethyl group is achieved from compound 17 (prepared in Scheme 3, above). As shown in Scheme 8, Compound 17 is deprotected to remove the original protecting group P¹ and the free alcohol is subsequently oxidized to the aldehyde under Swern conditions. After oxidation, the intermediate is brominated with tribromophospine and bromine gas to form the α-bromo aldehyde 18B. The α-bromo aldehyde is reacted with α-(p-nitrophenoxycarbonyl)methyldiethylphosphonate (prepared from the p-nitrophenyl ester of acetic acid and diethylchlorophosphonate in the presence of, for example, NEt₃) under Horner Wadworth Emmons conditions to form the corresponding α,β-unsaturated γ-bromo ester. The activated ester is then converted to the corresponding amide 19B by treatment with ammonia (See Beckwith, A. L. J., in Zabicky The Chemistry of Amides; Wiley: NY, 1970, p. 96). The olefin of compound 19B is brominated with hydrogen bromide to afford the primary bromide and the second protecting group (P²) is removed from the intermediate and the resultant alcohol oxidized to the aldehyde using standard methods, e.g., treatment with the Swern or Dess-Martin reagent Finally, the α,β-unsaturated amide is hydrogenated with hydrogen in the presence of catalytic palladium on carbon to afford fragment 20B. To complete the synthesis of the compound of Formula III, compound 20A is coupled with compound 12 under the conditions described in Scheme 4 above. The resultant product is then coupled with compound 5A (See Scheme 5) in the presence of DCC and catalytic DMAP and the aldehyde oxidized to the corresponding carboxylic acid with, for example, KMnO₄.

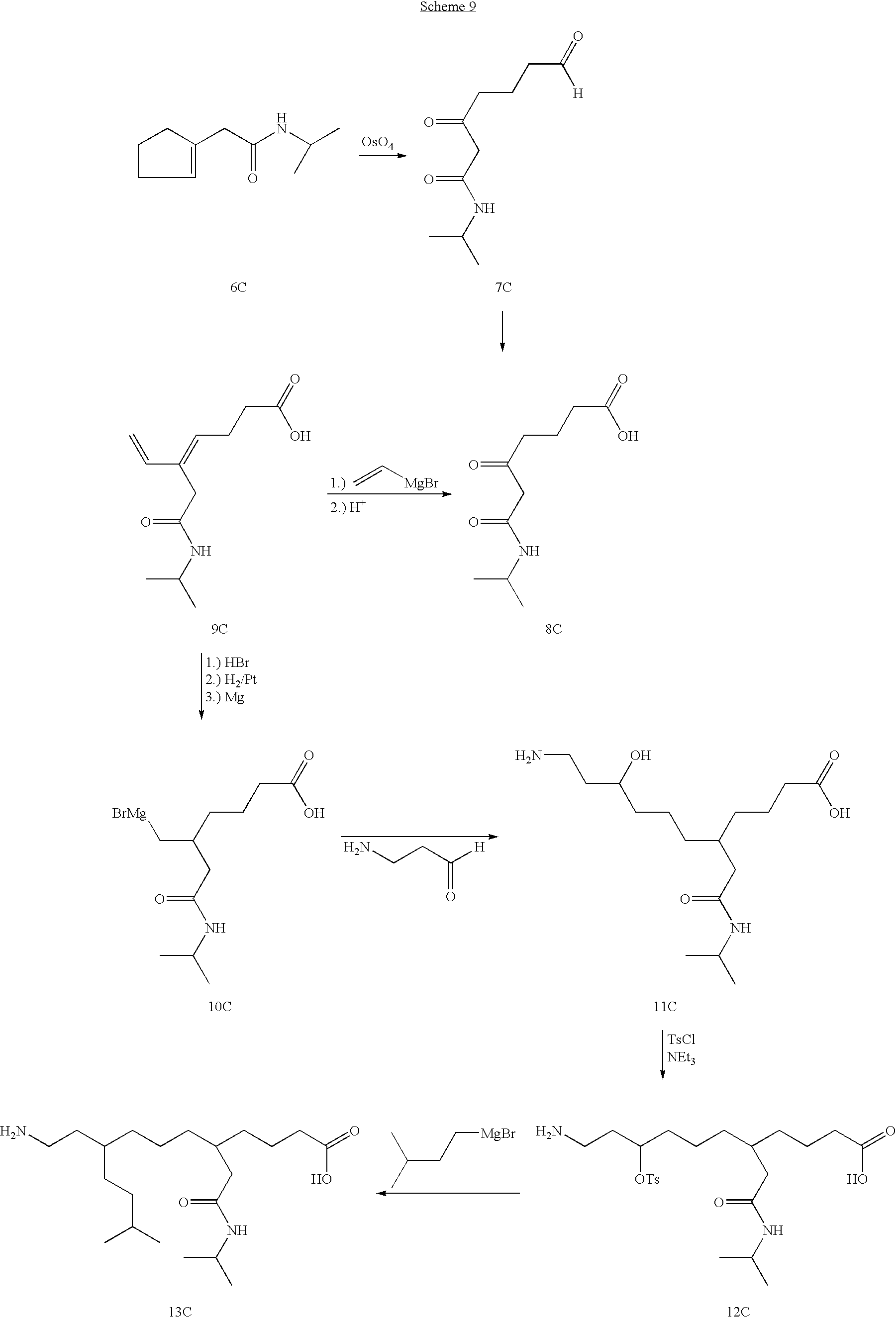

The preparation of the compound of Formula IV is illustrated in Scheme 9. The key modifications to the BQ-A011004 scaffold are the substitution of a 2-methylbutyl group for the ethoxy group y to the piperazine ring, and the substitution of an isopropoyl amide group for the phenyl amide group. The synthesis of a compound with these two modifications can be achieved via the synthesis of modified fragment 13C (Scheme 9). Fragment 13C is produced by converting the cyclopentenyl isopropylamide compound (6C) to the 1,3-dicarbonyl compound (7C) with osmium tetroxide followed by treatment with sodium periodate and then treatment with water and a mild reducing agent such as NaHSO₃. Compound 6C is formed in 3 steps form commercially available cyclopentanone (Aldrich Chemical Co.); (1) an aldol reaction of cyclopentanone with the enolate of ethyl acetate, (2) dehydration of the resultant alcohol by treatment with acid, and (3) conversion of the resultant α,β-unsaturated ester to its corresponding amide upon reaction with the lithium isopropylamide (Majetich et al. Tetrahedron Lett. 1994, 35, 8727). Compound 7C is oxidized using standard techniques, for example treatment with KMnO₄, to the carboxylic acid compound 8C. Compound 8C is treated with vinylmagnesium chloride and the resultant alcohol subsequently dehydrated with acid to produce the diene compound 9C. The vinyl alkene of compound 9C is brominated with hydrogen bromide followed by hydrogenation of the heptenyl olefin with hydrogen gas in the presence of a catalytic amount of palladium on carbon. Finally the 1-bromoalkane is reacted with magnesium to produce the alkyl grignard reagent 10C. Compound 10C is then reacted with 3-aminopropanal in ether, followed by treatment with mild acid to produce alcohol compound 11C. Compound 11C is converted to its corresponding tosylate 12C by treatment with tosyl chloride in the presence of base, for example NEt₃. Finally, treatment of tosylate 12C with 3-methylbutyl magnesium bromide (produced from 3-methyl-1-bromobutane and magnesium in ether) affords fragment 13C. The remainder of the synthesis of the compound of Formula IV can be achieved by substituting compound 13C for compound 12 in Scheme 4 (supra) and carrying out the appropriate coupling reactions with compounds 19 and 5.

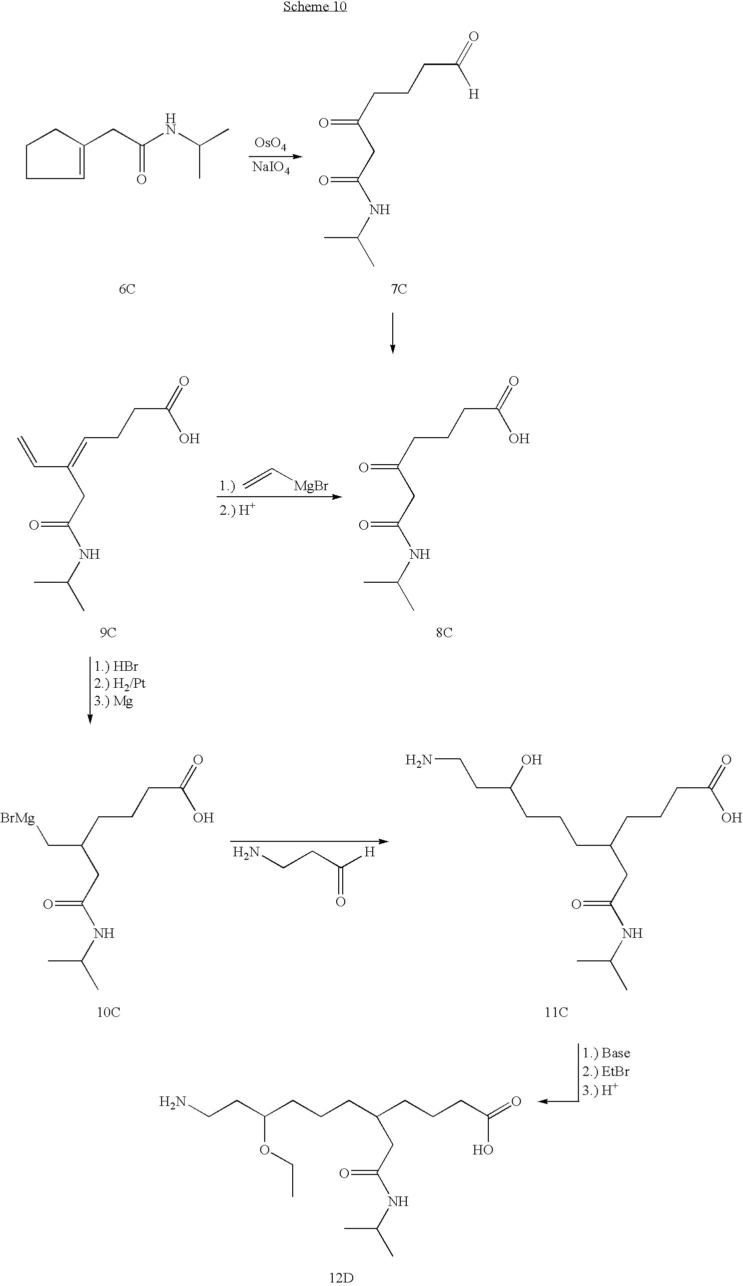

The preparation of the compound of Formula V is illustrated in Scheme 10. The key modification to the BQ-A011004 scaffold is the substitution of an isopropoyl amide group for the phenyl amide group. The synthesis of a compound with these two modifications can be achieved via the synthesis of modified fragment 12D. In scheme 10, intermediate 12D is produced by converting the cyclopentenyl isopropylamide compound (6C) to the 1,3-dicarbonyl compound (7C) with osmium tetroxide followed by treatment with sodium periodate and then treatment with water and a mild reducing agent such as NaHSO₃. Compound 7 is oxidized using standard techniques, for example treatment with KMnO₄, to the carboxylic acid compound 8C. Compound 8C is treated with vinylmagnesium chloride in the resultant alcohol subsequently dehydrated with acid to produce the diene compound 9C. The vinyl alkene of compound 9C is brominated with hydrogen bromide followed by hydrogenation of the heptenyl olefin with hydrogen gas in the presence of a catalytic amount of palladium on carbon. Finally the 1-bromoalkane is reacted with magnesium to produce the alkyl Grignard reagent 10C. Compound 10C is then reacted with 3-aminopropanal in ether, followed by treatment with mild acid to produce alcohol compound 11C. Finally compound 11C is reacted with a base, followed by ethylbromide and then acid to form ethyl ether intermediate 12D. The remainder of the synthesis of the compound of Formula IV can be achieved by substituting compound 12D for compound 12 in Scheme 4 (supra) and carrying out the appropriate coupling reactions with compounds 19 and 5.

The key modification to the BQ-A011004 scaffold for the compound of Formula VI is the substitution of a 4-[4-aminobutyl]-4,5-dihydropyrazole for the aminomethylbenzimidazole fragment. The synthesis is achieved by the coupling of compound 5A (See Scheme 7) with compound 20 (See Scheme 4) with DCC in the presence of catalytic DMAP followed by oxidation of the aldehyde to the corresponding carboxylic acid with, for example, KMnO₄.

Pharmacophore molecules of the invention can also be selected or modified by selecting or modifying molecules so that they include certain points of the PF4 pharmacophore while selectively excluding others. For example, without being limited to any particular theory or mechanism of action, lead PF4 antagonists (which bind to but do not activate PF4 receptor) can be selected and/or identified by identifying compounds that include certain pharmacophore points required and/or preferred for binding to the PF4 receptor, while selectively excluding other points that may be required or preferred for target (in this example PF4 receptor) activation. See also, Section 5.1, above.

The chemical structure of one such compound is illustrated in FIG. 9A (Formula VII). This compound includes functional groups corresponding to the PF4 pharmacophore points IX, X and VI (Tables 1 and 5, below), while functional groups corresponding to the remaining PF4 pharmacophore points (i.e., points I to V, VII and VIM) are not present. This compound is expected to compete with other molecules such as wild-type PF4 (SEQ ID NO:1) and BQ-A01004 (Formula I) for binding to the PF4 receptor without activating that target. Hence, a compound having this chemical structure is expected to be, and can be used as, a PF4 antagonist in accordance with the present invention.

In preferred embodiments, such PF4 agonist and/or antagonist compounds can be used to detect PF4 receptor polypeptides or fragments thereof. For example, a PF4 agonist or antagonist can be conjugated to a detectable label, and binding of the agonist molecule to PF4 receptor can be detected by detecting the detectable label. In particular embodiments, the PF4 agonist is conjugated to a contrasting agent, for detecting in a medical imaging application such as magnetic resonance imaging (MRI). For imaging purposes, any of a variety of diagnostic agents may be incorporated into a pharmaceutical composition, either linked to a modulating agent or free within the composition. Diagnostic agents include any substance administered to illuminate a physiological function within a patient, while leaving other physiological functions generally unaffected. Diagnostic agents include metals, radioactive isotopes and radioopaque agents (e.g., gallium, technetium, indium, strontium, iodine, barium, bromine and phosphorus-containing compounds), radiolucent agents, contrast agents, dyes (e.g., fluorescent dyes and chromophores) and enzymes that catalyze a calorimetric or fluorometric reaction. In general, such agents may be attached using a variety of techniques as described above, and may be present in any orientation. In such embodiments, one or more water soluble polymers (for example, polyethylene glycol or “PEG”) can also be conjugated to the PF4 agonist or antagonist.

One preferred, exemplary embodiment is illustrated in FIG. 9B. Here, a linker moiety can be used to attach a contrast agent or other detectable label, such as a lanthanide atom encaged inside a DOTA cycle.

6.6. Additional PF4-Derived Polypeptides

In still other embodiments, the present invention provides still other peptides that are derived from the amino acid sequence of PF4, and are useful, e.g. as PF4 agonists and/or antagonists according to methods described here. Particularly preferred polypeptides of these other embodiments include polypeptides having any one or more of the following amino acid sequences:

| | |
|---|---|
| Desig- | |
| nation | Sequence |

| | | |
|---|---|---|
| P34-56 | PHSPTAQLIATLKNGRKISLDLQ | (SEQ ID NO:157) |
| P37-56 | PTAQLIATLKNGRKISLDLQ | (SEQ ID NO:158) |
| P34-53 | PHSPTAQLIATLKNGRKISL | (SEQ ID NO:159) |
| P35-53 | PSPTAQLIATLKNGRKISL | (SEQ ID NO:160) |

The peptide designated P34-56 (SEQ ID NO:157), above, is so named because it is derived from the sequence of amino acids corresponding to residues 34-56 in the full-length, mature PF4 amino acid sequence set forth in FIG. 1C (SEQ ID NO:1). This peptide is understood to bind to and activate the PF4 receptor, and is therefore particularly useful as a PF4 agonist according to the methods of this invention. Without being limited to any particular theory or mechanism of action, the activity of P34-56 (SEQ ID NO:157) is believed to be mediated, at least in part, by residues in an alpha-helix region that comprises residues 5-13 of SEQ ID NO:157. This sequence is derived from and corresponds to an alpha-helix region of the mature PF4 polypeptide (FIG. 1C) comprising the sequence of amino acid residues 38-46 of SEQ ID NO:1. The alpha-helix in the P34-56 peptide (SEQ ID NO:157) is, in turn, understood to be stabilized at least in part by a “capping box” moiety corresponding to the sequence of amino acid residues 1-4 in that peptide. This capping box moiety is not present in the second peptide, designated P37-56 (SEQ ID NO:158), which is otherwise identical to the sequence of P34-56 (SEQ ID NO:157). The removal of this capping box is understood to destabilize the alpha-helix moiety, and thereby render the resulting peptide inactive. Hence, the P37-56 peptide (SEQ ID NO:158), supra, is understood to be inactive in that it does not activate the PF4 receptor.

The peptide designated P34-53 (SEQ ID NO:159) is likewise named because its sequence is derived from the sequence of amino acids corresponding to residues 34-53 of the full-length, mature PF4 amino acid sequence depicted in FIG. 1C (SEQ ID NO:1). The P34-53 peptide (SEQ ID NO:159) effectively competes against P34-56 (SEQ ID NO:157) for target binding, but does not activate the PF4 receptor. Hence, this peptide is particularly useful as a PF4 antagonist according to methods of the present invention. In preferred embodiments, a detectable label can be conjugated to the P34-53 peptide (SEQ ID NO:159), and the peptide can be used to detect PF4 receptor polypeptides, e.g., in a diagnostic assay. In particularly preferred embodiments, the P34-53 peptide (SEQ ID NO:159) can be used to detect PF4 receptor polypeptides (or fragments thereof) in vivo in an individual, for example as part of a magnetic resonance imaging (MRI) or other medical imaging and/or diagnostic assay.

The peptide designated P35-53 (SEQ ID NO:160) is identical to P34-53 (SEQ ID NO:159), except that the His2 residue of P34-53 (SEQ ID NO:159) has been removed. This modification is understood to abolish PF4 binding activity, so that the P35-53 peptide (SEQ ID NO:160) does not bind to or activate PF4 receptor.

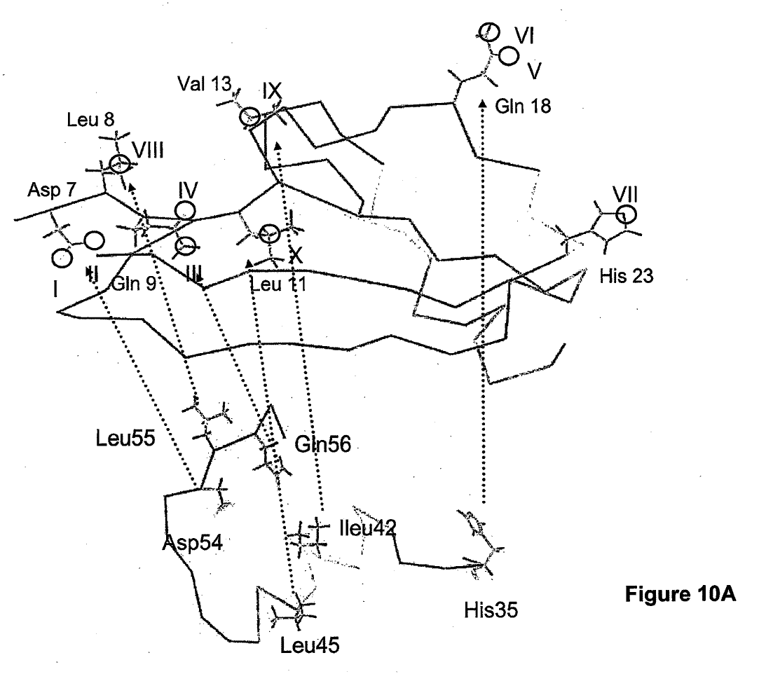

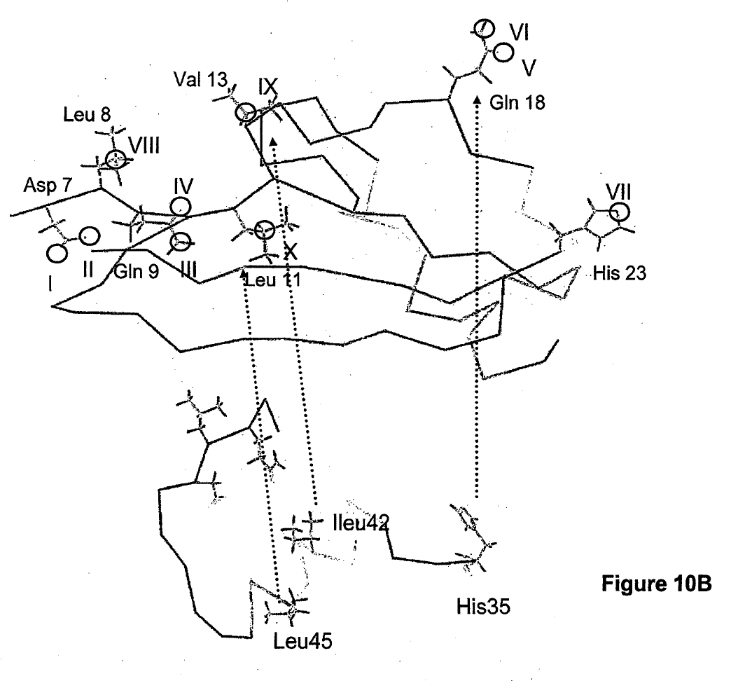

Without being limited to any particular theory or mechanism of action, it is believed that the activities of these peptides can be attributed to configurations of certain amino acid residues corresponding to some, but not necessarily all, of the PF4 pharmacophore points described, supra, in this application. This can be more readily seen by comparing three dimensional structures of the different peptides to the PF4 pharmacophore configuration. Two such exemplary comparisons are provided herein, in FIGS. 10A-10B.

Specifically, the bottom half of FIG. 10A provides a three-dimensional representation of the P34-56 peptide (SEQ ID NO:157) backbone, and compares it to the PF4 pharmacophore structure illustrated in FIG. 2A (which is also shown in the top half of FIG. 10A). For convenience, the P34-56 peptide (SEQ ID NO:157) amino acid residues are labeled in FIG. 10A with the numbers of corresponding residues in the full length, mature, wild-type PF4 amino acid sequence (SEQ ID NO:1).

The PF4 pharmacophore is partially present in the P34-56 peptide. Specifically, Gln23 in P34-56 (SEQ ID NO:157) mimics the position and orientation of Gln9 in wild-type, mature PF4 (SEQ ID NO:1) and, hence, provides functional groups corresponding to PF4 pharmacophore points III and IV listed in Table 1, supra. Leu22 in P34-56 (SEQ ID NO:157) mimics the position and orientation of Leu8 in WTPF4 (SEQ ID NO:1) and, hence, provides functional groups corresponding to PF4 pharmacophore point VIII. Asp21 in P34-56 (SEQ ID NO:157) mimics the position and orientation of Asp7 in wild-type PF4 (SEQ ID NO:1), and provides functional groups corresponding to PF4 pharmacophore points I and II. The P34-56 peptide (SEQ ID NO:157) residue Leu12 mimics the position and orientation of the Leu11 amino acid residue in WTPF4 (SEQ ID NO:1), and provides a functional group corresponding to pharmacophore point X. P34-56 peptide (SEQ ID NO:157) amino acid residue Ile9 mimics WTPF4 (SEQ ID NO:1) residue Val 13 and provides PF4 pharmacophore point IX. Finally, the His2 amino acid residue of P34-56 (SEQ ID NO:157) mimics Gln18 of WTPF4 (SEQ ID NO:1). This amino acid residue therefore provides a functional group corresponding to PF4 pharmacophore VI. Unlike glutamine, however, the histidine side chain does not comprise an oxygen. Hence, His2 and, by extension, the P34-56 peptide itself (SEQ ID NO:157) do not comprise a functional group corresponding to PF4 pharmacophore point V. A functional group corresponding to PF4 pharmacophore point VII also is not present in the P34-56 peptide (SEQ ID NO:157).

As explained, supra, the P34-56 peptide (SEQ ID NO:157) is derived from and corresponds to the sequence of amino acid residues 34-56 in the WTPF4 amino acid sequence set forth at SEQ ID NO:1. A person skilled in the art will therefore appreciate that amino acid residues His2, Ile9, Leu12, Asp21, Leu22 and Gln23 in that peptide (SEQ ID NO:157) correspond to residues His35, Ile42, Leu45, Asp54, Leu55 and Gln56, respectively, in SEQ ID NO:1. These residues are therefore identified in the bottom half of FIG. 10A according to those residues in WTPF4 (SEQ ID NO:1) from which they are derived and to which they correspond.

Further inspection of FIG. 10A provides further insight into the functional significance of points I through IV and VIII in the PF4 pharmacophore. These points are all located in the sequence of amino acid residues, Asp7-Leu8-Gln9, in the WTPF4 amino acid sequence (SEQ ID NO:1). The P34-56 peptide (SEQ ID NO:157) also comprises a DLQ motif, at residues 21-23. Without being limited to any particular theory or mechanism of action, this DLQ motif in P34-57 (SEQ ID NO:157) is believed to be stabilized by a network of hydrogen bonds, so that its conformation mimics the N-terminal folding of the DLQ motif at residues 7-9 in WTPF4.

FIG. 10B shows a similar comparison of the P34-53 peptide (SEQ ID NO:159) to the PF4 pharmacophore of FIG. 2A. Again, peptide residues in this figure are labeled according to the amino acid residues in full length WTPF4 (SEQ ID NO:1) to which they correspond. In particular, the P34-53 peptide (SEQ ID NO:159) comprises amino acid residues corresponding to His35, Ile42 and Leu45 in SEQ ID NO:1, and presents functional groups corresponding to points VI, IX and X of the PF4 pharmacophore. However, the DLQ residues, which are found in P34-56 (SEQ ID NO: 157), are not present in the P34-53 peptide (SEQ ID NO:159), and the peptide does not have any functional groups corresponding to pharmacophore points I through IV and VIII. The P34-53 peptide (SEQ ID NO:159) therefore effectively competes with PF4 for binding to the PF4 receptor, and can be used, e.g., in MRI imaging studies according to this invention. However, the peptide does not activate the PF4 receptor, and is not an effective PF4 agonist.

### 7. REFERENCES CITED

Numerous references, including patents, patent applications and various publications, are cited and discussed in the description of this invention. The citation and/or discussion of such references is provided merely to clarify the description of the present invention and is not an admission that any such reference is “prior art” to the invention described here. All references cited and/or discussed in this specification (including references, e.g., to biological sequences or structures in the GenBank, PDB or other public databases) are incorporated herein by reference in their entirety and to the same extent as if each reference was individually incorporated by reference.

### 8. APPENDIX

### Crystal Structure Coordinates for PF4

See also, Zhang et al., Biochemistry 1994, 33:8361-8366; and Accession No. 1RHP of the Protein Data Bank (both of which are hereby incorporated by reference and in their entireties).

| | | | | | |
|---|---|---|---|---|---|
| HEADER | PLATELET FACTOR | 16-SEP-94 | 1RHP | 1RHP | 2 |

| | | | | |
|---|---|---|---|---|
| COMPND | | PLATELET FACTOR 4 (HPF4) (HUMAN RECOMBINANT) | 1RHP | 3 |
| SOURCE | | HUMAN (HOMO SAPIENS) RECOMBINANT FORM EXPRESSED IN | 1RHP | 4 |
| SOURCE | 2 | (ESCHERICHIA COLI) | 1RHP | 5 |
| AUTHOR | | L. CHEN, X. ZHANG | 1RHP | 6 |
| REVDAT | 1 | 30-NOV-94 1RHP 0 | 1RHP | 7 |
| JRNL | | AUTH X. ZHANG, L. CHEN, D.P. BANCROFT, C.K. LAI, | 1RHP | 8 |
| | | T.E. MAIONE | | |
| JRNL | | TITL CRYSTAL STRUCTURE OF RECOMBINANT HUMAN PLATELET | 1RHP | 9 |
| JRNL | | TITL 2 FACTOR 4 | 1RHP | 10 |

| | | | | |
|---|---|---|---|---|
| JRNL | REF BIOCHEMISTRY | V. 33 8361 1994 | 1RHP | 11 |

| | | | | |
|---|---|---|---|---|
| JRNL | REFN ASTM BICHAW US ISSN 0006-2960 | 0033 | 1RHP | 12 |

| | | | | |
|---|---|---|---|---|
| REMARK | 1 | | 1RHP | 13 |
| REMARK | 2 | | 1RHP | 14 |
| REMARK | 2 | RESOLUTION. 2.4 ANGSTROMS. | 1RHP | 15 |
| REMARK | 3 | | 1RHP | 16 |
| REMARK | 3 | REFINEMENT. | 1RHP | 17 |

| | | | | | |
|---|---|---|---|---|---|
| REMARK | 3 | PROGRAM | X-PLOR | 1RHP | 18 |
| REMARK | 3 | AUTHORS | BRUNGER | 1RHP | 19 |
| REMARK | 3 | R VALUE | 0.241 | 1RHP | 20 |

| | | | | | | |
|---|---|---|---|---|---|---|
| REMARK | 3 | RMSD BOND DISTANCES | 0.016 | ANGSTROMS | 1RHP | 21 |
| REMARK | 3 | RMSD BOND ANGLES | 3.89 | DEGREES | 1RHP | 22 |
| REMARK | 3 | | | | 1RHP | 23 |
| REMARK | 3 | NUMBER OF REFLECTIONS | 11037 | | 1RHP | 24 |
| REMARK | 3 | RESOLUTION RANGE | 8.-2.4 | ANGSTROMS | 1RHP | 25 |
| REMARK | 3 | DATA CUTOFF | 2. | SIGMA(F) | 1RHP | 26 |
| REMARK | 3 | PERCENT COMPLETION | 94.4 | | 1RHP | 27 |
| REMARK | 3 | | | | 1RHP | 28 |

| | | | | | |
|---|---|---|---|---|---|
| REMARK | 3 | NUMBER OF PROTEIN ATOMS | 1988 | 1RHP | 29 |
| REMARK | 3 | NUMBER OF NUCLEIC ACID ATOMS | 0 | 1RHP | 30 |
| REMARK | 3 | NUMBER OF SOLVENT ATOMS | 91 | 1RHP | 31 |

| | | | | |
|---|---|---|---|---|
| REMARK | 3 | | 1RHP | 32 |
| REMARK | 3 | R-FACTOR 22.0% FOR RESOLUTION RANGE 8-3.0 ANGSTROMS. | 1RHP | 33 |
| REMARK | 4 | | 1RHP | 34 |
| REMARK | 4 | THE ASYMMETRIC UNIT CONSISTS OF FOUR IDENTICAL | 1RHP | 35 |
| REMARK | 4 | CHAINS AND EACH OF THE FOUR IDENTICAL CHAINS IS | 1RHP | 36 |
| REMARK | 4 | MISSING THE FIRST SIX RESIDUES DUE TO DISORDER. | 1RHP | 37 |
| REMARK | 5 | | 1RHP | 38 |
| REMARK | 5 | CROSS REFERENCE TO SEQUENCE DATABASE | 1RHP | 39 |

| | | | | | |
|---|---|---|---|---|---|
| REMARK | 5 | SWISS-PROT ENTRY NAME | PDB ENTRY CHAIN NAME | 1RHP | 40 |
| REMARK | 5 | PLF4_HUMAN | A | 1RHP | 41 |
| REMARK | 5 | PLF4_HUMAN | B | 1RHP | 42 |
| REMARK | 5 | PLF4_HUMAN | C | 1RHP | 43 |
| REMARK | 5 | PLF4_HUMAN | D | 1RHP | 44 |
| REMARK | 5 | | | 1RHP | 45 |

| | | | | |
|---|---|---|---|---|
| REMARK | 5 | THE FOLLOWING RESIDUES APE MISSING FROM THE | 1RHP | 46 |
| REMARK | 5 | N-TERMINUS OF CHAINS A, B, C, AND D: | 1RHP | 47 |
| REMARK | 5 | SEQUENCE NUMBER IS THAT FROM SWISS-PROT ENTRY | 1RHP | 48 |

| | | | | | |
|---|---|---|---|---|---|
| REMARK | 5 | GLU | 32 | 1RHP | 49 |
| REMARK | 5 | ALA | 33 | 1RHP | 50 |
| REMARK | 5 | GLU | 34 | 1RHP | 51 |
| REMARK | 5 | GLU | 35 | 1RHP | 52 |
| REMARK | 5 | ASP | 36 | 1RHP | 53 |
| REMARK | 5 | GLY | 37 | 1RHP | 54 |

| | | | | | | |
|---|---|---|---|---|---|---|
| SEQRES | 1 | A | 70 | GLU ALA GLU GLU ASP GLY ASP LEU GLN CYS LEU CYS VAL | 1RHP | 55 |
| SEQRES | 2 | A | 70 | LYS THR THR SER GLN VAL ARG PRO ARG HIS ILE THR SER | 1RHP | 56 |
| SEQRES | 3 | A | 70 | LEU GLU VAL ILE LYS ALA GLY PRO HIS CYS PRO THR ALA | 1RHP | 57 |
| SEQRES | 4 | A | 70 | GLN LEU ILE ALA THR LEU LYS ASN GLY ARG LYS ILE CYS | 1RHP | 58 |
| SEQRES | 5 | A | 70 | LEU ASP LEU GLN ALA PRO LEU TYR LYS LYS ILE ILE LYS | 1RHP | 59 |
| SEQRES | 6 | A | 70 | LYS LEU LEU GLU SER | 1RHP | 60 |
| SEQRES | 1 | B | 70 | GLU ALA GLU GLU ASP GLY ASP LEU GLN CYS LEU CYS VAL | 1RHP | 61 |
| SEQRES | 2 | B | 70 | LYS THR THR SER GLN VAL ARG PRO ARG HIS ILE THR SER | 1RHP | 62 |
| SEQRES | 3 | B | 70 | LEU GLU VAL ILE LYS ALA GLY PRO HIS CYS PRO THR ALA | 1RHP | 63 |
| SEQRES | 4 | B | 70 | GLN LEU ILE ALA THR LEU LYS ASN GLY ARG LYS ILE CYS | 1RHP | 64 |
| SEQRES | 5 | B | 70 | LEU ASP LEU GLN ALA PRO LEU TYR LYS LYS ILE ILE LYS | 1RHP | 65 |
| SEQRES | 6 | B | 70 | LYS LEU LEU GLU SER | 1RHP | 66 |
| SEQRES | 1 | C | 70 | GLU ALA GLU GLU ASP GLY ASP LEU GLN CYS LEU CYS VAL | 1RHP | 67 |
| SEQRES | 2 | C | 70 | LYS THR THR SER GLN VAL ARG PRO ARG HIS ILE THR SER | 1RHP | 68 |
| SEQRES | 3 | C | 70 | LEU GLU VAL ILE LYS ALA GLY PRO HIS CYS PRO THR ALA | 1RHP | 69 |
| SEQRES | 4 | C | 70 | GLN LEU ILE ALA THR LEU LYS ASN GLY ARG LYS ILE CYS | 1RHP | 70 |
| SEQRES | 5 | C | 70 | LEU ASP LEU GLN ALA PRO LEU TYR LYS LYS ILE ILE LYS | 1RHP | 71 |
| SEQRES | 6 | C | 70 | LYS LEU LEU GLU SER | 1RHP | 72 |
| SEQRES | 1 | D | 70 | GLU ALA GLU GLU ASP GLY ASP LEU GLN CYS LEU CYS VAL | 1RHP | 73 |
| SEQRES | 2 | D | 70 | LYS THR THR SER GLN VAL ARG PRO ARG HIS ILE THR SER | 1RHP | 74 |
| SEQRES | 3 | D | 70 | LEU GLU VAL ILE LYS ALA GLY PRO HIS CYS PRO THR ALA | 1RHP | 75 |
| SEQRES | 4 | D | 70 | GLN LEU ILE ALA THR LEU LYS ASN GLY ARG LYS ILE CYS | 1RHP | 76 |
| SEQRES | 5 | D | 70 | LEU ASP LEU GLN ALA PRO LEU TYR LYS LYS ILE ILE LYS | 1RHP | 77 |
| SEQRES | 6 | D | 70 | LYS LEU LEU GLU SER | 1RHP | 78 |

| | | | | | |
|---|---|---|---|---|---|
| FORMUL | 5 | HOH | *91(H2 O1) | 1RHP | 79 |

| | | | | | | | | | |
|---|---|---|---|---|---|---|---|---|---|
| HELIX | 1 | H1 | ALA A | 57 | SER A | 70 | 1 | 1RHP | 80 |
| HELIX | 2 | H1 | ALA B | 57 | SER B | 70 | 1 | 1RHP | 81 |
| HELIX | 3 | H1 | ALA C | 57 | SER C | 70 | 1 | 1RHP | 82 |
| HELIX | 4 | H1 | ALA D | 57 | SER D | 70 | 1 | 1RHP | 83 |

| | | | | | | | | | | |
|---|---|---|---|---|---|---|---|---|---|---|
| SHEET | 1 | A | 3 | THR A | 25 | GLY A | 33 | 0 | 1RHP | 84 |

| | | | | | | | | | | | | | | | | |
|---|---|---|---|---|---|---|---|---|---|---|---|---|---|---|---|---|
| SHEET | 2 | A | 3 | PRO A | 37 | LYS A | 46 | −1 | O | GLN A | 40 | N | ILE A | 30 | 1RHP | 85 |
| SHEET | 3 | A | 3 | GLY A | 48 | LEU A | 53 | −1 | O | ILE A | 51 | N | ALA A | 43 | 1RHP | 86 |
| SHEET | 1 | B | 3 | THR B | 25 | GLY B | 33 | 0 | | | | | | | 1RHP | 87 |
| SHEET | 2 | B | 3 | PRO B | 37 | LYS B | 46 | −1 | O | GLN B | 40 | N | ILE B | 30 | 1RHP | 88 |
| SHEET | 3 | B | 3 | GLY B | 48 | LEU B | 53 | −1 | O | ILE B | 51 | N | ALA B | 43 | 1RHP | 89 |
| SHEET | 1 | C | 3 | THR C | 25 | GLY C | 33 | 0 | | | | | | | 1RHP | 90 |
| SHEET | 2 | C | 3 | PRO C | 37 | LYS C | 46 | −1 | O | GLN C | 40 | N | ILE C | 30 | 1RHP | 91 |
| SHEET | 3 | C | 3 | GLY C | 48 | LEU C | 53 | −1 | O | ILE C | 51 | N | ALA C | 43 | 1RHP | 92 |
| SHEET | 1 | D | 3 | THR D | 25 | GLY D | 33 | 0 | | | | | | | 1RHP | 93 |
| SHEET | 2 | D | 3 | PRO D | 37 | LYS D | 46 | −1 | O | GLN D | 40 | N | ILE D | 30 | 1RHP | 94 |
| SHEET | 3 | D | 3 | GLY D | 48 | LEU D | 53 | −1 | O | ILE D | 51 | N | ALA D | 43 | 1RHP | 95 |

| | | | | | | | |
|---|---|---|---|---|---|---|---|
| SSBOND | 1 | CYS A | 10 | CYS A | 36 | 1RHP | 96 |
| SSBOND | 2 | CYS A | 12 | CYS A | 52 | 1RHP | 97 |
| SSBOND | 3 | CYS B | 10 | CYS B | 36 | 1RHP | 98 |
| SSBOND | 4 | CYS B | 12 | CYS B | 52 | 1RHP | 99 |
| SSBOND | 5 | CYS C | 10 | CYS C | 36 | 1RHP | 100 |
| SSBOND | 6 | CYS C | 12 | CYS C | 52 | 1RHP | 101 |
| SSBOND | 7 | CYS D | 10 | CYS D | 36 | 1RHP | 102 |
| SSBOND | 8 | CYS D | 12 | CYS D | 52 | 1RHP | 103 |

| | | | | | | | | | | |
|---|---|---|---|---|---|---|---|---|---|---|
| CRYST1 | 78.200 | 86.200 | 43.400 | 90.00 | 90.00 | 90.00 | P 21 21 21 | 16 | 1RHP | 104 |

| | | | | | | |
|---|---|---|---|---|---|---|
| ORIGX1 | 1.000000 | 0.000000 | 0.000000 | 0.00000 | 1RHP | 105 |
| ORIGX2 | 0.000000 | 1.000000 | 0.000000 | 0.00000 | 1RHP | 106 |
| ORIGX3 | 0.000000 | 0.000000 | 1.000000 | 0.00000 | 1RHP | 107 |
| SCALE1 | 0.012788 | 0.000000 | 0.000000 | 0.00000 | 1RHP | 108 |
| SCALE2 | 0.000000 | 0.011601 | 0.000000 | 0.00000 | 1RHP | 109 |
| SCALE3 | 0.000000 | 0.000000 | 0.023041 | 0.00000 | 1RHP | 110 |

| | | | | | | | | | | | |
|---|---|---|---|---|---|---|---|---|---|---|---|
| ATOM | 1 | N | ASP A | 7 | 5.920 | 31.818 | 67.142 | 1.00 | 32.27 | 1RHP | 111 |
| ATOM | 2 | CA | ASP A | 7 | 4.777 | 31.780 | 66.231 | 1.00 | 31.32 | 1RHP | 112 |
| ATOM | 3 | C | ASP A | 7 | 5.295 | 30.941 | 65.064 | 1.00 | 28.47 | 1RHP | 113 |
| ATOM | 4 | O | ASP A | 7 | 6.467 | 30.537 | 65.143 | 1.00 | 27.01 | 1RHP | 114 |
| ATOM | 5 | CB | ASP A | 7 | 4.411 | 33.190 | 65.717 | 1.00 | 36.54 | 1RHP | 115 |
| ATOM | 6 | CG | ASP A | 7 | 5.466 | 33.875 | 64.816 | 1.00 | 38.80 | 1RHP | 116 |
| ATOM | 7 | OD1 | ASP A | 7 | 6.527 | 34.293 | 65.337 | 1.00 | 41.06 | 1RHP | 117 |
| ATOM | 8 | OD2 | ASP A | 7 | 5.205 | 33.985 | 63.598 | 1.00 | 41.38 | 1RHP | 118 |
| ATOM | 9 | N | LEU A | 8 | 4.445 | 30.661 | 64.076 | 1.00 | 24.38 | 1RHP | 119 |
| ATOM | 10 | CA | LEU A | 8 | 4.820 | 30.027 | 62.838 | 1.00 | 23.92 | 1RHP | 120 |
| ATOM | 11 | C | LEU A | 8 | 3.617 | 29.436 | 62.149 | 1.00 | 22.50 | 1RHP | 121 |
| ATOM | 12 | O | LEU A | 8 | 2.903 | 30.207 | 61.513 | 1.00 | 22.74 | 1RHP | 122 |
| ATOM | 13 | CB | LEU A | 8 | 5.856 | 28.891 | 62.973 | 1.00 | 23.90 | 1RHP | 123 |
| ATOM | 14 | CG | LEU A | 8 | 7.014 | 29.129 | 61.988 | 1.00 | 24.46 | 1RHP | 124 |
| ATOM | 15 | CD1 | LEU A | 8 | 7.712 | 30.503 | 62.220 | 1.00 | 22.75 | 1RHP | 125 |
| ATOM | 16 | CD2 | LEU A | 8 | 7.959 | 27.936 | 62.139 | 1.00 | 22.11 | 1RHP | 126 |
| ATOM | 17 | N | GLN A | 9 | 3.313 | 28.138 | 62.303 | 1.00 | 22.60 | 1RHP | 127 |
| ATOM | 18 | CA | GLN A | 9 | 2.327 | 27.424 | 61.482 | 1.00 | 22.44 | 1RHP | 128 |
| ATOM | 19 | C | GLN A | 9 | 2.315 | 27.896 | 60.051 | 1.00 | 20.54 | 1RHP | 129 |
| ATOM | 20 | O | GLN A | 9 | 1.358 | 28.414 | 59.467 | 1.00 | 20.00 | 1RHP | 130 |
| ATOM | 21 | CB | GLN A | 9 | 0.877 | 27.541 | 61.995 | 1.00 | 23.30 | 1RHP | 131 |
| ATOM | 22 | CG | GLN A | 9 | 0.344 | 26.299 | 62.744 | 1.00 | 25.06 | 1RHP | 132 |
| ATOM | 23 | CD | GLN A | 9 | 0.460 | 24.941 | 62.049 | 1.00 | 28.56 | 1RHP | 133 |
| ATOM | 24 | OE1 | GLN A | 9 | 1.431 | 24.655 | 61.348 | 1.00 | 26.97 | 1RHP | 134 |
| ATOM | 25 | NE2 | GLN A | 9 | −0.493 | 24.027 | 62.184 | 1.00 | 27.14 | 1RHP | 135 |
| ATOM | 26 | N | CYS A | 10 | 3.506 | 27.806 | 59.513 | 1.00 | 17.73 | 1RHP | 136 |
| ATOM | 27 | CA | CYS A | 10 | 3.602 | 28.161 | 58.136 | 1.00 | 17.90 | 1RHP | 137 |
| ATOM | 28 | C | CYS A | 10 | 3.115 | 26.980 | 57.376 | 1.00 | 15.84 | 1RHP | 138 |
| ATOM | 29 | O | CYS A | 10 | 3.000 | 27.123 | 56.167 | 1.00 | 16.62 | 1RHP | 139 |
| ATOM | 30 | CB | CYS A | 10 | 5.009 | 28.405 | 57.683 | 1.00 | 21.20 | 1RHP | 140 |
| ATOM | 31 | SG | CYS A | 10 | 5.602 | 30.061 | 58.036 | 1.00 | 24.89 | 1RHP | 141 |
| ATOM | 32 | N | LEU A | 11 | 2.890 | 25.835 | 58.019 | 1.00 | 11.26 | 1RHP | 142 |
| ATOM | 33 | CA | LEU A | 11 | 2.552 | 24.614 | 57.340 | 1.00 | 12.16 | 1RHP | 143 |
| ATOM | 34 | C | LEU A | 11 | 3.815 | 24.123 | 56.604 | 1.00 | 12.52 | 1RHP | 144 |
| ATOM | 35 | O | LEU A | 11 | 4.406 | 23.132 | 57.061 | 1.00 | 12.83 | 1RHP | 145 |
| ATOM | 36 | CB | LEU A | 11 | 1.341 | 24.865 | 56.386 | 1.00 | 12.17 | 1RHP | 146 |
| ATOM | 37 | CG | LEU A | 11 | 0.773 | 23.719 | 55.556 | 1.00 | 12.31 | 1RHP | 147 |
| ATOM | 38 | CD1 | LEU A | 11 | 0.187 | 22.683 | 56.487 | 1.00 | 12.97 | 1RHP | 148 |
| ATOM | 39 | CD2 | LEU A | 11 | −0.280 | 24.235 | 54.599 | 1.00 | 13.84 | 1RHP | 149 |
| ATOM | 40 | N | CYS A | 12 | 4.313 | 24.756 | 55.526 | 1.00 | 12.29 | 1RHP | 150 |
| ATOM | 41 | CA | CYS A | 12 | 5.545 | 24.328 | 54.849 | 1.00 | 14.66 | 1RHP | 151 |
| ATOM | 42 | C | CYS A | 12 | 6.761 | 24.766 | 55.647 | 1.00 | 17.40 | 1RHP | 152 |
| ATOM | 43 | O | CYS A | 12 | 7.006 | 25.982 | 55.712 | 1.00 | 18.24 | 1RHP | 153 |
| ATOM | 44 | CB | CYS A | 12 | 5.708 | 24.963 | 53.505 | 1.00 | 9.85 | 1RHP | 154 |
| ATOM | 45 | SG | CYS A | 12 | 4.211 | 24.752 | 52.569 | 1.00 | 6.57 | 1RHP | 155 |
| ATOM | 46 | N | VAL A | 13 | 7.493 | 23.885 | 56.319 | 1.00 | 18.97 | 1RHP | 156 |
| ATOM | 47 | CA | VAL A | 13 | 8.703 | 24.339 | 56.951 | 1.00 | 19.18 | 1RHP | 157 |
| ATOM | 48 | C | VAL A | 13 | 9.800 | 23.593 | 56.249 | 1.00 | 19.49 | 1RHP | 158 |
| ATOM | 49 | O | VAL A | 13 | 10.780 | 24.234 | 55.876 | 1.00 | 21.46 | 1RHP | 159 |
| ATOM | 50 | CB | VAL A | 13 | 8.688 | 24.086 | 58.477 | 1.00 | 19.46 | 1RHP | 160 |
| ATOM | 51 | CG1 | VAL A | 13 | 8.604 | 22.624 | 58.868 | 1.00 | 19.26 | 1RHP | 161 |
| ATOM | 52 | CG2 | VAL A | 13 | 9.939 | 24.806 | 58.999 | 1.00 | 20.35 | 1RHP | 162 |
| ATOM | 53 | N | LYS A | 14 | 9.758 | 22.298 | 56.008 | 1.00 | 17.21 | 1RHP | 163 |
| ATOM | 54 | CA | LYS A | 14 | 10.778 | 21.766 | 55.134 | 1.00 | 16.20 | 1RHP | 164 |
| ATOM | 55 | C | LYS A | 14 | 10.207 | 22.028 | 53.735 | 1.00 | 13.59 | 1RHP | 165 |
| ATOM | 56 | O | LYS A | 14 | 8.991 | 21.917 | 53.577 | 1.00 | 12.58 | 1RHP | 166 |
| ATOM | 57 | CB | LYS A | 14 | 10.951 | 20.310 | 55.478 | 1.00 | 16.82 | 1RHP | 167 |
| ATOM | 58 | CG | LYS A | 14 | 11.622 | 20.216 | 56.836 | 1.00 | 18.16 | 1RHP | 168 |
| ATOM | 59 | CD | LYS A | 14 | 12.135 | 18.793 | 57.015 | 1.00 | 22.66 | 1RHP | 169 |
| ATOM | 60 | CE | LYS A | 14 | 13.080 | 18.591 | 58.207 | 1.00 | 23.56 | 1RHP | 170 |
| ATOM | 61 | NZ | LYS A | 14 | 13.698 | 17.269 | 58.124 | 1.00 | 25.59 | 1RHP | 171 |
| ATOM | 62 | N | THR A | 15 | 10.910 | 22.511 | 52.728 | 1.00 | 9.85 | 1RHP | 172 |
| ATOM | 63 | CA | THR A | 15 | 10.338 | 22.605 | 51.397 | 1.00 | 9.27 | 1RHP | 173 |
| ATOM | 64 | C | THR A | 15 | 10.921 | 21.508 | 50.520 | 1.00 | 10.05 | 1RHP | 174 |
| ATOM | 65 | O | THR A | 15 | 11.402 | 20.489 | 51.028 | 1.00 | 8.41 | 1RHP | 175 |
| ATOM | 66 | CB | THR A | 15 | 10.645 | 23.944 | 50.740 | 1.00 | 10.64 | 1RHP | 176 |
| ATOM | 67 | OG1 | THR A | 15 | 11.980 | 24.164 | 51.109 | 1.00 | 12.44 | 1RHP | 177 |
| ATOM | 68 | CG2 | THR A | 15 | 9.866 | 25.135 | 51.159 | 1.00 | 10.83 | 1RHP | 178 |
| ATOM | 69 | N | THR A | 16 | 10.905 | 21.667 | 49.190 | 1.00 | 11.84 | 1RHP | 179 |
| ATOM | 70 | CA | THR A | 16 | 11.430 | 20.697 | 48.259 | 1.00 | 10.42 | 1RHP | 180 |
| ATOM | 71 | C | THR A | 16 | 11.473 | 21.218 | 46.829 | 1.00 | 11.95 | 1RHP | 181 |
| ATOM | 72 | O | THR A | 16 | 10.854 | 22.209 | 46.443 | 1.00 | 15.68 | 1RHP | 182 |
| ATOM | 73 | CB | THR A | 16 | 10.579 | 19.488 | 48.285 | 1.00 | 9.22 | 1RHP | 183 |
| ATOM | 74 | OG1 | THR A | 16 | 11.486 | 18.472 | 47.943 | 1.00 | 11.04 | 1RHP | 184 |
| ATOM | 75 | CG2 | THR A | 16 | 9.361 | 19.543 | 47.369 | 1.00 | 11.47 | 1RHP | 185 |
| ATOM | 76 | N | SER A | 17 | 12.165 | 20.407 | 46.051 | 1.00 | 13.29 | 1RHP | 186 |
| ATOM | 77 | CA | SER A | 17 | 12.355 | 20.554 | 44.624 | 1.00 | 13.44 | 1RHP | 187 |
| ATOM | 78 | C | SER A | 17 | 12.264 | 19.173 | 44.064 | 1.00 | 13.47 | 1RHP | 188 |
| ATOM | 79 | O | SER A | 17 | 12.310 | 19.009 | 42.853 | 1.00 | 15.02 | 1RHP | 189 |
| ATOM | 80 | CB | SER A | 17 | 13.740 | 21.082 | 44.255 | 1.00 | 14.59 | 1RHP | 190 |
| ATOM | 81 | OG | SER A | 17 | 14.836 | 20.665 | 45.089 | 1.00 | 14.64 | 1RHP | 191 |
| ATOM | 82 | N | GLN A | 18 | 12.106 | 18.144 | 44.887 | 1.00 | 13.15 | 1RHP | 192 |
| ATOM | 83 | CA | GLN A | 18 | 12.166 | 16.791 | 44.392 | 1.00 | 13.65 | 1RHP | 193 |
| ATOM | 84 | C | GLN A | 18 | 10.815 | 16.391 | 43.829 | 1.00 | 15.30 | 1RHP | 194 |
| ATOM | 85 | O | GLN A | 18 | 10.226 | 15.381 | 44.246 | 1.00 | 17.24 | 1RHP | 195 |
| ATOM | 86 | CB | GLN A | 18 | 12.593 | 15.916 | 45.541 | 1.00 | 9.92 | 1RHP | 196 |
| ATOM | 87 | CG | GLN A | 18 | 13.908 | 16.385 | 46.123 | 1.00 | 9.21 | 1RHP | 197 |
| ATOM | 88 | CD | GLN A | 18 | 14.805 | 15.203 | 46.298 | 1.00 | 9.39 | 1RHP | 198 |
| ATOM | 89 | OE1 | GLN A | 18 | 14.832 | 14.503 | 47.308 | 1.00 | 8.91 | 1RHP | 199 |
| ATOM | 90 | NE2 | GLN A | 18 | 15.485 | 14.921 | 45.218 | 1.00 | 8.23 | 1RHP | 200 |
| ATOM | 91 | N | VAL A | 19 | 10.320 | 17.162 | 42.849 | 1.00 | 14.83 | 1RHP | 201 |
| ATOM | 92 | CA | VAL A | 19 | 8.997 | 16.910 | 42.346 | 1.00 | 13.99 | 1RHP | 202 |
| ATOM | 93 | C | VAL A | 19 | 8.992 | 16.754 | 40.852 | 1.00 | 15.12 | 1RHP | 203 |
| ATOM | 94 | O | VAL A | 19 | 9.413 | 17.619 | 40.092 | 1.00 | 16.00 | 1RHP | 204 |
| ATOM | 95 | CB | VAL A | 19 | 8.083 | 18.032 | 42.774 | 1.00 | 11.56 | 1RHP | 205 |
| ATOM | 96 | CG1 | VAL A | 19 | 8.618 | 19.350 | 42.268 | 1.00 | 11.83 | 1RHP | 206 |
| ATOM | 97 | CG2 | VAL A | 19 | 6.681 | 17.690 | 42.313 | 1.00 | 9.87 | 1RHP | 207 |
| ATOM | 98 | N | ARG A | 20 | 8.524 | 15.576 | 40.446 | 1.00 | 16.24 | 1RHP | 208 |
| ATOM | 99 | CA | ARG A | 20 | 8.415 | 15.204 | 39.041 | 1.00 | 15.47 | 1RHP | 209 |
| ATOM | 100 | C | ARG A | 20 | 7.262 | 16.104 | 38.549 | 1.00 | 16.00 | 1RHP | 210 |
| ATOM | 101 | O | ARG A | 20 | 6.110 | 15.930 | 38.996 | 1.00 | 15.69 | 1RHP | 211 |
| ATOM | 102 | CB | ARG A | 20 | 8.105 | 13.688 | 39.016 | 1.00 | 17.77 | 1RHP | 212 |
| ATOM | 103 | CG | ARG A | 20 | 8.363 | 12.820 | 37.776 | 1.00 | 17.58 | 1RHP | 213 |
| ATOM | 104 | CD | ARG A | 20 | 9.854 | 12.432 | 37.507 | 1.00 | 19.04 | 1RHP | 214 |
| ATOM | 105 | NE | ARG A | 20 | 10.497 | 13.196 | 36.432 | 1.00 | 21.73 | 1RHP | 215 |
| ATOM | 106 | CZ | ARG A | 20 | 11.216 | 12.655 | 35.408 | 1.00 | 23.83 | 1RHP | 216 |
| ATOM | 107 | NH1 | ARG A | 20 | 11.467 | 11.337 | 35.268 | 1.00 | 20.82 | 1RHP | 217 |
| ATOM | 108 | NH2 | ARG A | 20 | 11.732 | 13.465 | 34.467 | 1.00 | 22.90 | 1RHP | 218 |
| ATOM | 109 | N | PRO A | 21 | 7.477 | 17.107 | 37.685 | 1.00 | 14.55 | 1RHP | 219 |
| ATOM | 110 | CA | PRO A | 21 | 6.450 | 18.059 | 37.270 | 1.00 | 12.40 | 1RHP | 220 |
| ATOM | 111 | C | PRO A | 21 | 5.179 | 17.423 | 36.719 | 1.00 | 11.88 | 1RHP | 221 |
| ATOM | 112 | O | PRO A | 21 | 4.078 | 17.880 | 36.986 | 1.00 | 11.60 | 1RHP | 222 |
| ATOM | 113 | CB | PRO A | 21 | 7.171 | 18.935 | 36.265 | 1.00 | 12.55 | 1RHP | 223 |
| ATOM | 114 | CG | PRO A | 21 | 8.582 | 18.926 | 36.769 | 1.00 | 13.34 | 1RHP | 224 |
| ATOM | 115 | CD | PRO A | 21 | 8.758 | 17.454 | 37.087 | 1.00 | 13.99 | 1RHP | 225 |
| ATOM | 116 | N | ARG A | 22 | 5.255 | 16.309 | 36.002 | 1.00 | 11.94 | 1RHP | 226 |
| ATOM | 117 | CA | ARG A | 22 | 4.074 | 15.736 | 35.396 | 1.00 | 11.80 | 1RHP | 227 |
| ATOM | 118 | C | ARG A | 22 | 3.197 | 14.949 | 36.342 | 1.00 | 10.17 | 1RHP | 228 |
| ATOM | 119 | O | ARG A | 22 | 2.360 | 14.173 | 35.915 | 1.00 | 9.69 | 1RHP | 229 |
| ATOM | 120 | CB | ARG A | 22 | 4.555 | 14.899 | 34.204 | 1.00 | 16.50 | 1RHP | 230 |
| ATOM | 121 | CG | ARG A | 22 | 4.663 | 13.373 | 34.169 | 1.00 | 21.54 | 1RHP | 231 |
| ATOM | 122 | CD | ARG A | 22 | 3.648 | 12.864 | 33.126 | 1.00 | 27.26 | 1RHP | 232 |
| ATOM | 123 | NE | ARG A | 22 | 4.164 | 12.009 | 32.059 | 1.00 | 29.40 | 1RHP | 233 |
| ATOM | 124 | CZ | ARG A | 22 | 3.958 | 12.303 | 30.757 | 1.00 | 29.28 | 1RHP | 234 |
| ATOM | 125 | NH1 | ARG A | 22 | 3.334 | 13.428 | 30.344 | 1.00 | 30.90 | 1RHP | 235 |
| ATOM | 126 | NH2 | ARG A | 22 | 4.443 | 11.459 | 29.839 | 1.00 | 32.62 | 1RHP | 236 |
| ATOM | 127 | N | HIS A | 23 | 3.310 | 15.084 | 37.651 | 1.00 | 9.21 | 1RHP | 237 |
| ATOM | 128 | CA | HIS A | 23 | 2.483 | 14.290 | 38.533 | 1.00 | 9.07 | 1RHP | 238 |
| ATOM | 129 | C | HIS A | 23 | 1.778 | 15.196 | 39.483 | 1.00 | 8.72 | 1RHP | 239 |
| ATOM | 130 | O | HIS A | 23 | 1.192 | 14.684 | 40.428 | 1.00 | 9.28 | 1RHP | 240 |
| ATOM | 131 | CB | HIS A | 23 | 3.307 | 13.314 | 39.354 | 1.00 | 13.04 | 1RHP | 241 |
| ATOM | 132 | CG | HIS A | 23 | 4.031 | 12.223 | 38.576 | 1.00 | 17.54 | 1RHP | 242 |
| ATOM | 133 | ND1 | HIS A | 23 | 3.837 | 11.800 | 37.325 | 1.00 | 20.70 | 1RHP | 243 |
| ATOM | 134 | CD2 | HIS A | 23 | 5.057 | 11.477 | 39.108 | 1.00 | 17.68 | 1RHP | 244 |
| ATOM | 135 | CE1 | HIS A | 23 | 4.686 | 10.840 | 37.063 | 1.00 | 19.46 | 1RHP | 245 |
| ATOM | 136 | NE2 | HIS A | 23 | 5.413 | 10.662 | 38.148 | 1.00 | 17.19 | 1RHP | 246 |
| ATOM | 137 | N | ILE A | 24 | 1.839 | 16.507 | 39.285 | 1.00 | 9.76 | 1RHP | 247 |
| ATOM | 138 | CA | ILE A | 24 | 1.215 | 17.473 | 40.182 | 1.00 | 12.32 | 1RHP | 248 |
| ATOM | 139 | C | ILE A | 24 | −0.066 | 17.989 | 39.521 | 1.00 | 11.73 | 1RHP | 249 |
| ATOM | 140 | O | ILE A | 24 | −0.083 | 18.492 | 38.387 | 1.00 | 14.22 | 1RHP | 250 |
| ATOM | 141 | CB | ILE A | 24 | 2.255 | 18.639 | 40.528 | 1.00 | 12.41 | 1RHP | 251 |
| ATOM | 142 | CG1 | ILE A | 24 | 1.564 | 19.756 | 41.248 | 1.00 | 13.69 | 1RHP | 252 |
| ATOM | 143 | CG2 | ILE A | 24 | 2.866 | 19.247 | 39.294 | 1.00 | 13.68 | 1RHP | 253 |
| ATOM | 144 | CD1 | ILE A | 24 | 2.516 | 20.917 | 41.556 | 1.00 | 13.22 | 1RHP | 254 |
| ATOM | 145 | N | THR A | 25 | −1.167 | 17.775 | 40.227 | 1.00 | 10.49 | 1RHP | 255 |
| ATOM | 146 | CA | THR A | 25 | −2.475 | 18.091 | 39.719 | 1.00 | 9.49 | 1RHP | 256 |
| ATOM | 147 | C | THR A | 25 | −2.967 | 19.410 | 40.195 | 1.00 | 8.70 | 1RHP | 257 |
| ATOM | 148 | O | THR A | 25 | −3.884 | 19.918 | 39.565 | 1.00 | 11.30 | 1RHP | 258 |
| ATOM | 149 | CB | THR A | 25 | −3.501 | 17.047 | 40.130 | 1.00 | 10.90 | 1RHP | 259 |
| ATOM | 150 | OG1 | THR A | 25 | −3.015 | 16.424 | 41.317 | 1.00 | 13.26 | 1RHP | 260 |
| ATOM | 151 | CG2 | THR A | 25 | −3.763 | 16.060 | 39.024 | 1.00 | 10.43 | 1RHP | 261 |
| ATOM | 152 | N | SER A | 26 | −2.434 | 20.008 | 41.244 | 1.00 | 8.82 | 1RHP | 262 |
| ATOM | 153 | CA | SER A | 26 | −2.984 | 21.237 | 41.733 | 1.00 | 9.11 | 1RHP | 263 |
| ATOM | 154 | C | SER A | 26 | −1.891 | 21.947 | 42.458 | 1.00 | 9.15 | 1RHP | 264 |
| ATOM | 155 | O | SER A | 26 | −1.072 | 21.265 | 43.074 | 1.00 | 10.35 | 1RHP | 265 |
| ATOM | 156 | CB | SER A | 26 | −4.115 | 20.914 | 42.678 | 1.00 | 11.50 | 1RHP | 266 |
| ATOM | 157 | OG | SER A | 26 | −4.569 | 22.018 | 43.455 | 1.00 | 12.47 | 1RHP | 267 |
| ATOM | 158 | N | LEU A | 27 | −1.851 | 23.269 | 42.384 | 1.00 | 6.51 | 1RHP | 268 |
| ATOM | 159 | CA | LEU A | 27 | −0.907 | 24.041 | 43.152 | 1.00 | 7.96 | 1RHP | 269 |
| ATOM | 160 | C | LEU A | 27 | −1.757 | 25.047 | 43.887 | 1.00 | 8.42 | 1RHP | 270 |
| ATOM | 161 | O | LEU A | 27 | −2.814 | 25.448 | 43.407 | 1.00 | 8.51 | 1RHP | 271 |
| ATOM | 162 | CB | LEU A | 27 | 0.066 | 24.841 | 42.309 | 1.00 | 7.51 | 1RHP | 272 |
| ATOM | 163 | CG | LEU A | 27 | 1.587 | 24.669 | 42.337 | 1.00 | 8.75 | 1RHP | 273 |
| ATOM | 164 | CD1 | LEU A | 27 | 2.084 | 25.935 | 41.700 | 1.00 | 6.32 | 1RHP | 274 |
| ATOM | 165 | CD2 | LEU A | 27 | 2.251 | 24.618 | 43.701 | 1.00 | 7.89 | 1RHP | 275 |
| ATOM | 166 | N | GLU A | 28 | −1.336 | 25.428 | 45.067 | 1.00 | 7.36 | 1RHP | 276 |
| ATOM | 167 | CA | GLU A | 28 | −1.991 | 26.446 | 45.821 | 1.00 | 8.63 | 1RHP | 277 |
| ATOM | 166 | C | GLU A | 28 | −0.849 | 27.423 | 46.053 | 1.00 | 11.10 | 1RHP | 278 |
| ATOM | 169 | O | GLU A | 28 | 0.174 | 26.983 | 46.591 | 1.00 | 13.03 | 1RHP | 279 |
| ATOM | 170 | CB | GLU A | 28 | −2.440 | 25.983 | 47.182 | 1.00 | 7.72 | 1RHP | 280 |
| ATOM | 171 | CG | GLU A | 28 | −3.551 | 24.972 | 47.399 | 1.00 | 6.93 | 1RHP | 281 |
| ATOM | 172 | CD | GLU A | 28 | −4.027 | 25.007 | 48.847 | 1.00 | 7.80 | 1RHP | 282 |
| ATOM | 173 | OE1 | GLU A | 28 | −4.347 | 26.097 | 49.352 | 1.00 | 7.76 | 1RHP | 283 |
| ATOM | 174 | OE2 | GLU A | 28 | −4.068 | 23.942 | 49.455 | 1.00 | 4.78 | 1RHP | 284 |
| ATOM | 175 | N | VAL A | 29 | −0.923 | 28.700 | 45.691 | 1.00 | 8.25 | 1RHP | 285 |
| ATOM | 176 | CA | VAL A | 29 | 0.106 | 29.679 | 45.977 | 1.00 | 8.98 | 1RHP | 296 |
| ATOM | 177 | C | VAL A | 29 | −0.426 | 30.383 | 47.227 | 1.00 | 9.95 | 1RHP | 287 |
| ATOM | 178 | O | VAL A | 29 | −1.162 | 31.352 | 47.049 | 1.00 | 12.68 | 1RHP | 285 |
| ATOM | 179 | CB | VAL A | 29 | 0.201 | 30.639 | 44.773 | 1.00 | 7.55 | 1RHP | 289 |
| ATOM | 180 | CG1 | VAL A | 29 | 1.260 | 31.673 | 45.026 | 1.00 | 8.26 | 1RHP | 290 |
| ATOM | 181 | CG2 | VAL A | 29 | 0.548 | 29.882 | 43.510 | 1.00 | 8.40 | 1RHP | 291 |
| ATOM | 182 | N | ILE A | 30 | −0.212 | 30.061 | 48.497 | 1.00 | 9.87 | 1RHP | 292 |
| ATOM | 183 | CA | ILE A | 30 | −0.895 | 30.848 | 49.525 | 1.00 | 11.90 | 1RHP | 293 |
| ATOM | 184 | C | ILE A | 30 | −0.067 | 32.077 | 49.797 | 1.00 | 13.74 | 1RHP | 294 |
| ATOM | 185 | O | ILE A | 30 | 1.143 | 31.942 | 49.851 | 1.00 | 15.29 | 1RHP | 295 |
| ATOM | 186 | CB | ILE A | 30 | −1.187 | 29.978 | 50.842 | 1.00 | 9.72 | 1RHP | 296 |
| ATOM | 187 | CG1 | ILE A | 30 | −1.198 | 30.888 | 52.043 | 1.00 | 12.46 | 1RHP | 297 |
| ATOM | 188 | CG2 | ILE A | 30 | −0.248 | 28.826 | 50.989 | 1.00 | 9.43 | 1RHP | 298 |
| ATOM | 189 | CD1 | ILE A | 30 | −1.185 | 30.136 | 53.394 | 1.00 | 14.02 | 1RHP | 299 |
| ATOM | 190 | N | LYS A | 31 | −0.675 | 33.267 | 49.825 | 1.00 | 16.87 | 1RHP | 300 |
| ATOM | 191 | CA | LYS A | 31 | −0.025 | 34.551 | 50.127 | 1.00 | 18.85 | 1RHP | 301 |
| ATOM | 192 | C | LYS A | 31 | 0.598 | 34.467 | 51.507 | 1.00 | 20.23 | 1RHP | 302 |
| ATOM | 193 | O | LYS A | 31 | 0.043 | 33.761 | 52.358 | 1.00 | 22.18 | 1RHP | 303 |
| ATOM | 194 | CB | LYS A | 31 | −1.063 | 35.685 | 50.127 | 1.00 | 19.16 | 1RHP | 304 |
| ATOM | 195 | CG | LYS A | 31 | −0.760 | 37.143 | 50.534 | 1.00 | 16.15 | 1RHP | 305 |
| ATOM | 196 | CD | LYS A | 31 | −0.336 | 38.044 | 49.374 | 1.00 | 14.86 | 1RHP | 306 |
| ATOM | 197 | CE | LYS A | 31 | −0.665 | 39.520 | 49.666 | 1.00 | 14.11 | 1RHP | 307 |
| ATOM | 198 | NZ | LYS A | 31 | −0.667 | 40.291 | 48.430 | 1.00 | 12.44 | 1RHP | 308 |
| ATOM | 199 | N | ALA A | 32 | 1.712 | 35.167 | 51.731 | 1.00 | 18.98 | 1RHP | 309 |
| ATOM | 200 | CA | ALA A | 32 | 2.342 | 35.183 | 53.045 | 1.00 | 15.84 | 1RHP | 310 |
| ATOM | 201 | C | ALA A | 32 | 1.544 | 36.105 | 53.949 | 1.00 | 13.39 | 1RHP | 311 |
| ATOM | 202 | O | ALA A | 32 | 0.823 | 36.952 | 53.437 | 1.00 | 13.10 | 1RHP | 312 |
| ATOM | 203 | CB | ALA A | 32 | 3.744 | 35.743 | 52.962 | 1.00 | 16.26 | 1RHP | 313 |
| ATOM | 204 | N | GLY A | 33 | 1.628 | 36.045 | 55.257 | 1.00 | 11.11 | 1RHP | 314 |
| ATOM | 205 | CA | GLY A | 33 | 0.920 | 36.998 | 56.055 | 1.00 | 12.95 | 1RHP | 315 |
| ATOM | 206 | C | GLY A | 33 | 1.357 | 36.900 | 57.492 | 1.00 | 17.73 | 1RHP | 316 |
| ATOM | 207 | O | GLY A | 33 | 2.303 | 36.189 | 57.839 | 1.00 | 16.02 | 1RHP | 317 |
| ATOM | 208 | N | PRO A | 34 | 0.643 | 37.570 | 58.400 | 1.00 | 20.24 | 1RHP | 318 |
| ATOM | 209 | CA | PRO A | 34 | 0.919 | 37.533 | 59.825 | 1.00 | 22.34 | 1RHP | 319 |
| ATOM | 210 | C | PRO A | 34 | 0.942 | 36.067 | 60.211 | 1.00 | 24.98 | 1RHP | 320 |
| ATOM | 211 | O | PRO A | 34 | 1.693 | 35.655 | 61.077 | 1.00 | 26.54 | 1RHP | 321 |
| ATOM | 212 | CB | PRO A | 34 | −0.222 | 38.337 | 60.420 | 1.00 | 22.09 | 1RHP | 322 |
| ATOM | 213 | CG | PRO A | 34 | −1.395 | 38.112 | 59.482 | 1.00 | 20.22 | 1RHP | 323 |
| ATOM | 214 | CD | PRO A | 34 | −0.652 | 38.223 | 58.148 | 1.00 | 22.42 | 1RHP | 324 |
| ATOM | 215 | N | HIS A | 35 | 0.111 | 35.262 | 59.558 | 1.00 | 27.98 | 1RHP | 325 |
| ATOM | 216 | CA | HIS A | 35 | 0.173 | 33.814 | 59.672 | 1.00 | 30.67 | 1RHP | 326 |
| ATOM | 217 | C | HIS A | 35 | 1.376 | 33.509 | 58.780 | 1.00 | 31.78 | 1RHP | 327 |
| ATOM | 218 | O | HIS A | 35 | 1.108 | 33.485 | 57.563 | 1.00 | 35.19 | 1RHP | 328 |
| ATOM | 219 | CB | HIS A | 35 | −1.123 | 33.177 | 59.069 | 1.00 | 29.24 | 1RHP | 329 |
| ATOM | 220 | CG | HIS A | 35 | −1.094 | 31.666 | 58.769 | 1.00 | 28.40 | 1RHP | 330 |
| ATOM | 221 | ND1 | HIS A | 35 | −1.695 | 30.720 | 59.484 | 1.00 | 28.16 | 1RHP | 331 |
| ATOM | 222 | CD2 | HIS A | 35 | −0.462 | 31.035 | 57.703 | 1.00 | 28.45 | 1RHP | 332 |
| ATOM | 223 | CE1 | HIS A | 35 | −1.445 | 29.568 | 58.898 | 1.00 | 28.38 | 1RHP | 333 |
| ATOM | 224 | NE2 | HIS A | 35 | −0.712 | 29.761 | 57.829 | 1.00 | 30.01 | 1RHP | 334 |
| ATOM | 225 | N | CYS A | 36 | 2.641 | 33.364 | 59.218 | 1.00 | 30.63 | 1RHP | 335 |
| ATOM | 226 | CA | CYS A | 36 | 3.728 | 32.933 | 58.306 | 1.00 | 27.47 | 1RHP | 336 |
| ATOM | 227 | C | CYS A | 36 | 4.249 | 33.946 | 57.284 | 1.00 | 23.26 | 1RHP | 337 |
| ATOM | 228 | O | CYS A | 36 | 3.568 | 34.247 | 56.299 | 1.00 | 19.53 | 1RHP | 338 |
| ATOM | 229 | CB | CYS A | 36 | 3.299 | 31.690 | 57.517 | 1.00 | 26.70 | 1RHP | 339 |
| ATOM | 230 | SG | CYS A | 36 | 4.637 | 30.995 | 56.546 | 1.00 | 24.84 | 1RHP | 340 |
| ATOM | 231 | N | PRO A | 37 | 5.516 | 34.395 | 57.419 | 1.00 | 20.18 | 1RHP | 341 |
| ATOM | 232 | CA | PRO A | 37 | 6.175 | 35.441 | 56.609 | 1.00 | 20.07 | 1RHP | 342 |
| ATOM | 233 | C | PRO A | 37 | 6.481 | 35.131 | 55.156 | 1.00 | 18.93 | 1RHP | 343 |
| ATOM | 234 | O | PRO A | 37 | 7.020 | 35.967 | 54.426 | 1.00 | 19.14 | 1RHP | 344 |
| ATOM | 235 | CB | PRO A | 37 | 7.449 | 35.771 | 57.327 | 1.00 | 16.70 | 1RHP | 345 |
| ATOM | 236 | CG | PRO A | 37 | 7.184 | 35.275 | 58.730 | 1.00 | 18.17 | 1RHP | 346 |
| ATOM | 237 | CD | PRO A | 37 | 6.414 | 33.985 | 58.492 | 1.00 | 20.40 | 1RHP | 347 |
| ATOM | 238 | N | THR A | 38 | 6.090 | 33.963 | 54.681 | 1.00 | 18.67 | 1RHP | 348 |
| ATOM | 239 | CA | THR A | 38 | 6.482 | 33.515 | 53.379 | 1.00 | 17.88 | 1RHP | 349 |
| ATOM | 240 | C | THR A | 38 | 5.284 | 32.772 | 52.799 | 1.00 | 18.35 | 1RHP | 350 |
| ATOM | 241 | O | THR A | 38 | 4.488 | 32.107 | 53.469 | 1.00 | 17.93 | 1RHP | 351 |
| ATOM | 242 | CB | THR A | 38 | 7.728 | 32.637 | 53.602 | 1.00 | 19.13 | 1RHP | 352 |
| ATOM | 243 | OG1 | THR A | 38 | 8.380 | 32.642 | 52.353 | 1.00 | 18.55 | 1RHP | 353 |
| ATOM | 244 | CG2 | THR A | 38 | 7.454 | 31.200 | 54.068 | 1.00 | 19.49 | 1RHP | 354 |
| ATOM | 245 | N | ALA A | 39 | 5.139 | 32.937 | 51.503 | 1.00 | 17.25 | 1RHP | 355 |
| ATOM | 246 | CA | ALA A | 39 | 4.123 | 32.234 | 50.774 | 1.00 | 15.24 | 1RHP | 356 |
| ATOM | 247 | C | ALA A | 39 | 4.464 | 30.762 | 50.860 | 1.00 | 13.86 | 1RHP | 357 |
| ATOM | 248 | O | ALA A | 39 | 5.572 | 30.385 | 51.245 | 1.00 | 13.92 | 1RHP | 358 |
| ATOM | 249 | CB | ALA A | 39 | 4.154 | 32.591 | 49.310 | 1.00 | 17.14 | 1RHP | 359 |
| ATOM | 250 | N | GLN A | 40 | 3.543 | 29.893 | 50.538 | 1.00 | 12.35 | 1RHP | 360 |
| ATOM | 251 | CA | GLN A | 40 | 3.904 | 28.521 | 50.366 | 1.00 | 12.47 | 1RHP | 361 |
| ATOM | 252 | C | GLN A | 40 | 3.491 | 28.142 | 48.949 | 1.00 | 12.13 | 1RHP | 362 |
| ATOM | 253 | O | GLN A | 40 | 2.702 | 28.840 | 48.314 | 1.00 | 10.90 | 1RHP | 363 |
| ATOM | 254 | CB | GLN A | 40 | 3.195 | 27.723 | 51.409 | 1.00 | 14.36 | 1RHP | 364 |
| ATOM | 255 | CG | GLN A | 40 | 3.806 | 28.017 | 52.765 | 1.00 | 15.18 | 1RHP | 365 |
| ATOM | 256 | CD | GLN A | 40 | 2.959 | 28.817 | 53.732 | 1.00 | 17.98 | 1RHP | 366 |
| ATOM | 257 | OE1 | GLN A | 40 | 1.742 | 28.820 | 53.665 | 1.00 | 17.09 | 1RHP | 367 |
| ATOM | 258 | NE2 | GLN A | 40 | 3.516 | 29.461 | 54.728 | 1.00 | 18.48 | 1RHP | 368 |
| ATOM | 259 | N | LEU A | 41 | 4.086 | 27.133 | 48.351 | 1.00 | 9.76 | 1RHP | 369 |
| ATOM | 260 | CA | LEU A | 41 | 3.613 | 26.663 | 47.079 | 1.00 | 10.44 | 1RHP | 370 |
| ATOM | 261 | C | LEU A | 41 | 3.250 | 25.240 | 47.468 | 1.00 | 10.98 | 1RHP | 371 |
| ATOM | 262 | O | LEU A | 41 | 4.120 | 24.381 | 47.539 | 1.00 | 12.68 | 1RHP | 372 |
| ATOM | 263 | CB | LEU A | 41 | 4.728 | 26.687 | 46.037 | 1.00 | 10.60 | 1RHP | 373 |
| ATOM | 264 | CG | LEU A | 41 | 5.193 | 27.934 | 45.291 | 1.00 | 9.44 | 1RHP | 374 |
| ATOM | 265 | CD1 | LEU A | 41 | 4.109 | 28.405 | 44.381 | 1.00 | 12.88 | 1RHP | 375 |
| ATOM | 266 | CD2 | LEU A | 41 | 5.568 | 29.022 | 46.255 | 1.00 | 10.00 | 1RHP | 376 |
| ATOM | 267 | N | ILE A | 42 | 2.001 | 24.946 | 47.797 | 1.00 | 12.65 | 1RHP | 377 |
| ATOM | 268 | CA | ILE A | 42 | 1.575 | 23.627 | 48.295 | 1.00 | 12.42 | 1RHP | 378 |
| ATOM | 269 | C | ILE A | 42 | 1.255 | 22.846 | 47.021 | 1.00 | 16.06 | 1RHP | 379 |
| ATOM | 270 | O | ILE A | 42 | 0.448 | 23.339 | 46.222 | 1.00 | 19.20 | 1RHP | 380 |
| ATOM | 271 | CB | ILE A | 42 | 0.313 | 23.822 | 49.236 | 1.00 | 9.65 | 1RHP | 381 |
| ATOM | 272 | CG1 | ILE A | 42 | 0.679 | 24.728 | 50.402 | 1.00 | 8.60 | 1RHP | 382 |
| ATOM | 273 | CG2 | ILE A | 42 | −0.133 | 22.548 | 49.867 | 1.00 | 3.96 | 1RHP | 383 |
| ATOM | 274 | CD1 | ILE A | 42 | −0.501 | 25.149 | 51.266 | 1.00 | 8.11 | 1RHP | 384 |
| ATOM | 275 | N | ALA A | 43 | 1.825 | 21.683 | 46.740 | 1.00 | 13.77 | 1RHP | 385 |
| ATOM | 276 | CA | ALA A | 43 | 1.591 | 20.993 | 45.491 | 1.00 | 12.85 | 1RHP | 386 |
| ATOM | 277 | C | ALA A | 43 | 0.753 | 19.798 | 45.867 | 1.00 | 13.98 | 1RHP | 387 |
| ATOM | 278 | O | ALA A | 43 | 1.083 | 19.148 | 46.861 | 1.00 | 16.28 | 1RHP | 388 |
| ATOM | 279 | CB | ALA A | 43 | 2.878 | 20.494 | 44.907 | 1.00 | 8.50 | 1RHP | 389 |
| ATOM | 280 | N | THR A | 44 | −0.318 | 19.476 | 45.163 | 1.00 | 11.50 | 1RHP | 390 |
| ATOM | 281 | CA | THR A | 44 | −1.105 | 18.316 | 45.476 | 1.00 | 10.77 | 1RHP | 391 |
| ATOM | 282 | C | THR A | 44 | −0.799 | 17.471 | 44.265 | 1.00 | 10.22 | 1RHP | 392 |
| ATOM | 283 | O | THR A | 44 | −0.807 | 17.982 | 43.137 | 1.00 | 11.88 | 1RHP | 393 |
| ATOM | 284 | CB | THR A | 44 | −2.565 | 18.766 | 45.589 | 1.00 | 9.12 | 1RHP | 394 |
| ATOM | 285 | OG1 | THR A | 44 | −2.603 | 19.824 | 46.550 | 1.00 | 10.81 | 1RHP | 395 |
| ATOM | 286 | CG2 | THR A | 44 | −3.486 | 17.656 | 46.075 | 1.00 | 9.87 | 1RHP | 396 |
| ATOM | 287 | N | LEU A | 45 | −0.448 | 16.214 | 44.482 | 1.00 | 10.20 | 1RHP | 397 |
| ATOM | 288 | CA | LEU A | 45 | −0.014 | 15.330 | 43.413 | 1.00 | 11.61 | 1RHP | 398 |
| ATOM | 289 | C | LEU A | 45 | −1.105 | 14.312 | 43.124 | 1.00 | 14.08 | 1RHP | 399 |
| ATOM | 290 | O | LEU A | 45 | −1.942 | 14.042 | 43.990 | 1.00 | 17.83 | 1RHP | 400 |
| ATOM | 291 | CB | LEU A | 45 | 1.261 | 14.548 | 43.807 | 1.00 | 11.22 | 1RHP | 401 |
| ATOM | 292 | CG | LEU A | 45 | 2.537 | 15.133 | 44.412 | 1.00 | 8.97 | 1RHP | 402 |
| ATOM | 293 | CD1 | LEU A | 45 | 3.318 | 14.026 | 45.021 | 1.00 | 8.15 | 1RHP | 403 |
| ATOM | 294 | CD2 | LEU A | 45 | 3.416 | 15.749 | 43.383 | 1.00 | 8.74 | 1RHP | 404 |
| ATOM | 295 | N | LYS A | 46 | −1.048 | 13.625 | 41.984 | 1.00 | 13.60 | 1RHP | 405 |
| ATOM | 296 | CA | LYS A | 46 | −2.052 | 12.663 | 41.583 | 1.00 | 13.97 | 1RHP | 406 |
| ATOM | 297 | C | LYS A | 46 | −2.229 | 11.557 | 42.592 | 1.00 | 13.96 | 1RHP | 407 |
| ATOM | 298 | O | LYS A | 46 | −3.339 | 11.095 | 42.755 | 1.00 | 15.19 | 1RHP | 408 |
| ATOM | 299 | CB | LYS A | 46 | −1.702 | 12.051 | 40.230 | 1.00 | 14.76 | 1RHP | 409 |
| ATOM | 300 | CG | LYS A | 46 | −1.866 | 13.033 | 39.065 | 1.00 | 17.13 | 1RHP | 410 |
| ATOM | 301 | CD | LYS A | 46 | −1.250 | 12.460 | 37.748 | 1.00 | 20.99 | 1RHP | 411 |
| ATOM | 302 | CE | LYS A | 46 | −0.759 | 13.458 | 36.626 | 1.00 | 22.33 | 1RHP | 412 |
| ATOM | 303 | NZ | LYS A | 46 | −1.776 | 14.097 | 35.794 | 1.00 | 22.26 | 1RHP | 413 |
| ATOM | 304 | N | ASN A | 47 | −1.280 | 11.124 | 43.394 | 1.00 | 13.76 | 1RHP | 414 |
| ATOM | 305 | CA | ASN A | 47 | −1.600 | 10.042 | 44.318 | 1.00 | 15.20 | 1RHP | 415 |
| ATOM | 306 | C | ASN A | 47 | −2.176 | 10.589 | 45.613 | 1.00 | 15.26 | 1RHP | 416 |
| ATOM | 307 | O | ASN A | 47 | −2.129 | 9.930 | 46.646 | 1.00 | 15.75 | 1RHP | 417 |
| ATOM | 308 | CB | ASN A | 47 | −0.378 | 9.202 | 44.686 | 1.00 | 15.96 | 1RHP | 418 |
| ATOM | 309 | CG | ASN A | 47 | −0.817 | 7.792 | 45.074 | 1.00 | 19.04 | 1RHP | 419 |
| ATOM | 310 | OD1 | ASN A | 47 | −0.659 | 7.308 | 46.203 | 1.00 | 18.63 | 1RHP | 420 |
| ATOM | 311 | ND2 | ASN A | 47 | −1.369 | 7.086 | 44.096 | 1.00 | 18.26 | 1RHP | 421 |
| ATOM | 312 | N | GLY A | 48 | −2.701 | 11.810 | 45.687 | 1.00 | 14.17 | 1RHP | 422 |
| ATOM | 313 | CA | GLY A | 48 | −3.213 | 12.321 | 46.954 | 1.00 | 11.43 | 1RHP | 423 |
| ATOM | 314 | C | GLY A | 48 | −2.195 | 13.099 | 47.769 | 1.00 | 10.76 | 1RHP | 424 |
| ATOM | 315 | O | GLY A | 48 | −2.554 | 14.153 | 48.257 | 1.00 | 9.32 | 1RHP | 425 |
| ATOM | 316 | N | ARG A | 49 | −0.965 | 12.610 | 47.943 | 1.00 | 11.85 | 1RHP | 426 |
| ATOM | 317 | CA | ARG A | 49 | 0.135 | 13.221 | 48.702 | 1.00 | 13.22 | 1RHP | 427 |
| ATOM | 318 | C | ARG A | 49 | 0.314 | 14.672 | 48.370 | 1.00 | 11.78 | 1RHP | 428 |
| ATOM | 319 | O | ARG A | 49 | 0.318 | 15.018 | 47.189 | 1.00 | 12.00 | 1RHP | 429 |
| ATOM | 320 | CB | ARG A | 49 | 1.449 | 12.511 | 48.387 | 1.00 | 15.17 | 1RHP | 430 |
| ATOM | 321 | CG | ARG A | 49 | 2.281 | 11.940 | 49.537 | 1.00 | 20.18 | 1RHP | 431 |
| ATOM | 322 | CD | ARG A | 49 | 1.582 | 10.975 | 50.528 | 1.00 | 21.59 | 1RHP | 432 |
| ATOM | 323 | NE | ARG A | 49 | 1.168 | 9.676 | 49.992 | 1.00 | 20.23 | 1RHP | 433 |
| ATOM | 324 | CZ | ARG A | 49 | 0.080 | 9.028 | 50.441 | 1.00 | 19.78 | 1RHP | 434 |
| ATOM | 325 | NH1 | ARG A | 49 | −0.698 | 9.572 | 51.381 | 1.00 | 19.11 | 1RHP | 435 |
| ATOM | 326 | NH2 | ARG A | 49 | −0.239 | 7.837 | 49.905 | 1.00 | 17.52 | 1RHP | 436 |
| ATOM | 327 | N | LYS A | 50 | 0.555 | 15.472 | 49.394 | 1.00 | 10.80 | 1RHP | 437 |
| ATOM | 328 | CA | LYS A | 50 | 0.672 | 16.910 | 49.232 | 1.00 | 11.69 | 1RHP | 438 |
| ATOM | 329 | C | LYS A | 50 | 2.050 | 17.317 | 49.732 | 1.00 | 9.93 | 1RHP | 439 |
| ATOM | 330 | O | LYS A | 50 | 2.426 | 16.787 | 50.776 | 1.00 | 12.77 | 1RHP | 440 |
| ATOM | 331 | CB | LYS A | 50 | −0.419 | 17.581 | 50.060 | 1.00 | 11.76 | 1RHP | 441 |
| ATOM | 332 | CG | LYS A | 50 | −0.756 | 19.024 | 49.731 | 1.00 | 8.99 | 1RHP | 442 |
| ATOM | 333 | CD | LYS A | 50 | −1.843 | 19.485 | 50.661 | 1.00 | 8.23 | 1RHP | 443 |
| ATOM | 334 | CE | LYS A | 50 | −3.090 | 18.633 | 50.494 | 1.00 | 8.38 | 1RHP | 444 |
| ATOM | 335 | NZ | LYS A | 50 | −3.682 | 18.764 | 49.176 | 1.00 | 8.05 | 1RHP | 445 |
| ATOM | 336 | N | ILE A | 51 | 2.795 | 18.232 | 49.094 | 1.00 | 8.81 | 1RHP | 446 |
| ATOM | 337 | CA | ILE A | 51 | 4.161 | 18.623 | 49.447 | 1.00 | 6.35 | 1RHP | 447 |
| ATOM | 338 | C | ILE A | 51 | 4.343 | 20.099 | 49.242 | 1.00 | 4.95 | 1RHP | 448 |
| ATOM | 339 | O | ILE A | 51 | 3.728 | 20.643 | 48.352 | 1.00 | 3.08 | 1RHP | 449 |
| ATOM | 340 | CB | ILE A | 51 | 5.227 | 17.946 | 48.572 | 1.00 | 7.68 | 1RHP | 450 |
| ATOM | 341 | CG1 | ILE A | 51 | 4.690 | 17.783 | 47.149 | 1.00 | 7.68 | 1RHP | 451 |
| ATOM | 342 | CG2 | ILE A | 51 | 5.663 | 16.655 | 49.221 | 1.00 | 7.95 | 1RHP | 452 |
| ATOM | 343 | CD1 | ILE A | 51 | 5.619 | 17.112 | 46.158 | 1.00 | 8.20 | 1RHP | 453 |
| ATOM | 344 | N | CYS A | 52 | 5.152 | 20.798 | 50.002 | 1.00 | 2.53 | 1RHP | 454 |
| ATOM | 345 | CA | CYS A | 52 | 5.404 | 22.219 | 49.787 | 1.00 | 5.14 | 1RHP | 455 |
| ATOM | 346 | C | CYS A | 52 | 6.647 | 22.453 | 48.935 | 1.00 | 7.95 | 1RHP | 456 |
| ATOM | 347 | O | CYS A | 52 | 7.777 | 22.245 | 49.380 | 1.00 | 12.65 | 1RHP | 457 |
| ATOM | 348 | CB | CYS A | 52 | 5.686 | 22.996 | 51.053 | 1.00 | 5.68 | 1RHP | 458 |
| ATOM | 349 | SG | CYS A | 52 | 4.550 | 22.812 | 52.423 | 1.00 | 10.46 | 1RHP | 459 |
| ATOM | 350 | N | LEU A | 53 | 6.542 | 22.911 | 47.715 | 1.00 | 9.86 | 1RHP | 460 |
| ATOM | 351 | CA | LEU A | 53 | 7.706 | 23.191 | 46.913 | 1.00 | 11.72 | 1RHP | 461 |
| ATOM | 352 | C | LEU A | 53 | 8.528 | 24.360 | 47.474 | 1.00 | 13.93 | 1RHP | 462 |
| ATOM | 353 | O | LEU A | 53 | 8.194 | 25.048 | 48.453 | 1.00 | 14.45 | 1RHP | 463 |
| ATOM | 354 | CB | LEU A | 53 | 7.230 | 23.503 | 45.531 | 1.00 | 14.03 | 1RHP | 464 |
| ATOM | 355 | CG | LEU A | 53 | 6.339 | 22.460 | 44.946 | 1.00 | 13.85 | 1RHP | 465 |
| ATOM | 356 | CD1 | LEU A | 53 | 5.583 | 23.020 | 43.776 | 1.00 | 12.70 | 1RHP | 466 |
| ATOM | 357 | CD2 | LEU A | 53 | 7.173 | 21.279 | 44.591 | 1.00 | 12.91 | 1RHP | 467 |
| ATOM | 358 | N | ASP A | 54 | 9.654 | 24.602 | 46.829 | 1.00 | 15.11 | 1RHP | 468 |
| ATOM | 359 | CA | ASP A | 54 | 10.563 | 25.649 | 47.191 | 1.00 | 13.41 | 1RHP | 469 |
| ATOM | 360 | C | ASP A | 54 | 10.282 | 26.899 | 46.453 | 1.00 | 10.64 | 1RHP | 470 |
| ATOM | 361 | O | ASP A | 54 | 9.979 | 26.823 | 45.267 | 1.00 | 7.83 | 1RHP | 471 |
| ATOM | 362 | CB | ASP A | 54 | 11.935 | 25.176 | 46.905 | 1.00 | 18.99 | 1RHP | 472 |
| ATOM | 363 | CG | ASP A | 54 | 12.612 | 24.949 | 48.223 | 1.00 | 22.87 | 1RHP | 473 |
| ATOM | 364 | OD1 | ASP A | 54 | 12.538 | 25.855 | 49.083 | 1.00 | 25.87 | 1RHP | 474 |
| ATOM | 365 | OD2 | ASP A | 54 | 13.182 | 23.870 | 48.379 | 1.00 | 25.23 | 1RHP | 475 |
| ATOM | 366 | N | LEU A | 55 | 10.457 | 28.005 | 47.160 | 1.00 | 8.17 | 1RHP | 476 |
| ATOM | 367 | CA | LEU A | 55 | 10.010 | 29.293 | 46.668 | 1.00 | 9.24 | 1RHP | 477 |
| ATOM | 368 | C | LEU A | 55 | 10.735 | 29.824 | 45.456 | 1.00 | 8.86 | 1RHP | 478 |
| ATOM | 369 | O | LEU A | 55 | 11.291 | 29.021 | 44.735 | 1.00 | 7.38 | 1RHP | 479 |
| ATOM | 370 | CB | LEU A | 55 | 10.115 | 30.297 | 47.789 | 1.00 | 10.49 | 1RHP | 480 |
| ATOM | 371 | CG | LEU A | 55 | 8.892 | 30.808 | 48.533 | 1.00 | 11.79 | 1RHP | 481 |
| ATOM | 372 | CD1 | LEU A | 55 | 9.394 | 31.663 | 49.671 | 1.00 | 9.03 | 1RHP | 482 |
| ATOM | 373 | CD2 | LEU A | 55 | 7.994 | 31.649 | 47.650 | 1.00 | 11.79 | 1RHP | 483 |
| ATOM | 374 | N | GLN A | 56 | 10.629 | 31.144 | 45.175 | 1.00 | 16.82 | 1RHP | 484 |
| ATOM | 375 | CA | GLN A | 56 | 11.337 | 31.983 | 44.176 | 1.00 | 21.72 | 1RHP | 485 |
| ATOM | 376 | C | GLN A | 56 | 12.129 | 31.506 | 42.963 | 1.00 | 24.95 | 1RHP | 486 |
| ATOM | 377 | O | GLN A | 56 | 12.797 | 32.356 | 42.340 | 1.00 | 27.82 | 1RHP | 487 |
| ATOM | 378 | CB | GLN A | 56 | 12.259 | 32.910 | 44.918 | 1.00 | 23.23 | 1RHP | 488 |
| ATOM | 379 | CG | GLN A | 56 | 11.407 | 34.107 | 45.202 | 1.00 | 23.55 | 1RHP | 489 |
| ATOM | 380 | CD | GLN A | 56 | 11.753 | 34.832 | 46.499 | 1.00 | 23.23 | 1RHP | 490 |
| ATOM | 381 | OE1 | GLN A | 56 | 11.472 | 36.032 | 46.669 | 1.00 | 23.31 | 1RHP | 491 |
| ATOM | 382 | NE2 | GLN A | 56 | 12.362 | 34.117 | 47.460 | 1.00 | 25.27 | 1RHP | 492 |
| ATOM | 383 | N | ALA A | 57 | 11.964 | 30.231 | 42.576 | 1.00 | 23.51 | 1RHP | 493 |
| ATOM | 384 | CA | ALA A | 57 | 12.760 | 29.535 | 41.587 | 1.00 | 20.41 | 1RHP | 494 |
| ATOM | 385 | C | ALA A | 57 | 12.129 | 29.564 | 40.205 | 1.00 | 17.80 | 1RHP | 495 |
| ATOM | 386 | O | ALA A | 57 | 10.948 | 29.869 | 40.089 | 1.00 | 19.01 | 1RHP | 496 |
| ATOM | 387 | CB | ALA A | 57 | 12.919 | 28.086 | 42.084 | 1.00 | 18.09 | 1RHP | 497 |
| ATOM | 388 | N | PRO A | 58 | 12.807 | 29.146 | 39.141 | 1.00 | 15.05 | 1RHP | 498 |
| ATOM | 389 | CA | PRO A | 58 | 12.202 | 28.563 | 37.964 | 1.00 | 11.47 | 1RHP | 499 |
| ATOM | 390 | C | PRO A | 58 | 11.294 | 27.377 | 38.184 | 1.00 | 11.03 | 1RHP | 500 |
| ATOM | 391 | O | PRO A | 58 | 10.688 | 26.871 | 37.244 | 1.00 | 12.47 | 1RHP | 501 |
| ATOM | 392 | CB | PRO A | 58 | 13.388 | 28.228 | 37.096 | 1.00 | 12.77 | 1RHP | 502 |
| ATOM | 393 | CG | PRO A | 58 | 14.421 | 29.237 | 37.510 | 1.00 | 14.91 | 1RHP | 503 |
| ATOM | 394 | CD | PRO A | 58 | 14.251 | 29.253 | 39.011 | 1.00 | 15.12 | 1RHP | 504 |
| ATOM | 395 | N | LEU A | 59 | 11.149 | 26.862 | 39.402 | 1.00 | 9.80 | 1RHP | 505 |
| ATOM | 396 | CA | LEU A | 59 | 10.329 | 25.673 | 39.612 | 1.00 | 9.59 | 1RHP | 506 |
| ATOM | 397 | C | LEU A | 59 | 8.918 | 26.113 | 39.436 | 1.00 | 9.71 | 1RHP | 507 |
| ATOM | 398 | O | LEU A | 59 | 8.217 | 25.446 | 38.676 | 1.00 | 9.71 | 1RHP | 508 |
| ATOM | 399 | CB | LEU A | 59 | 10.539 | 25.122 | 40.999 | 1.00 | 7.77 | 1RHP | 509 |
| ATOM | 400 | CG | LEU A | 59 | 10.145 | 23.723 | 41.333 | 1.00 | 5.57 | 1RHP | 510 |
| ATOM | 401 | CD1 | LEU A | 59 | 10.672 | 22.782 | 40.299 | 1.00 | 8.78 | 1RHP | 511 |
| ATOM | 402 | CD2 | LEU A | 59 | 10.710 | 23.369 | 42.685 | 1.00 | 4.36 | 1RHP | 512 |
| ATOM | 403 | N | TYR A | 60 | 8.531 | 27.251 | 40.027 | 1.00 | 9.61 | 1RHP | 513 |
| ATOM | 404 | CA | TYR A | 60 | 7.179 | 27.686 | 39.795 | 1.00 | 10.86 | 1RHP | 514 |
| ATOM | 405 | C | TYR A | 60 | 6.782 | 28.095 | 38.396 | 1.00 | 12.85 | 1RHP | 515 |
| ATOM | 406 | O | TYR A | 60 | 5.645 | 27.806 | 38.042 | 1.00 | 17.68 | 1RHP | 516 |
| ATOM | 407 | CB | TYR A | 60 | 6.753 | 28.820 | 40.643 | 1.00 | 10.43 | 1RHP | 517 |
| ATOM | 408 | CG | TYR A | 60 | 7.525 | 30.086 | 40.905 | 1.00 | 10.88 | 1RHP | 518 |
| ATOM | 409 | CD1 | TYR A | 60 | 7.668 | 31.120 | 40.020 | 1.00 | 10.18 | 1RHP | 519 |
| ATOM | 410 | CD2 | TYR A | 60 | 7.904 | 30.229 | 42.220 | 1.00 | 11.56 | 1RHP | 520 |
| ATOM | 411 | CE1 | TYR A | 60 | 8.182 | 32.311 | 40.508 | 1.00 | 14.29 | 1RHP | 521 |
| ATOM | 412 | CE2 | TYR A | 60 | 8.409 | 31.396 | 42.704 | 1.00 | 14.15 | 1RHP | 522 |
| ATOM | 413 | CZ | TYR A | 60 | 8.537 | 32.444 | 41.850 | 1.00 | 15.73 | 1RHP | 523 |
| ATOM | 414 | OH | TYR A | 60 | 9.008 | 33.617 | 42.428 | 1.00 | 16.68 | 1RHP | 524 |
| ATOM | 415 | N | LYS A | 61 | 7.547 | 28.713 | 37.510 | 1.00 | 10.88 | 1RHP | 525 |
| ATOM | 416 | CA | LYS A | 61 | 7.071 | 28.866 | 36.141 | 1.00 | 8.59 | 1RHP | 526 |
| ATOM | 417 | C | LYS A | 61 | 7.006 | 27.494 | 35.468 | 1.00 | 8.97 | 1RHP | 527 |
| ATOM | 418 | O | LYS A | 61 | 6.167 | 27.257 | 34.615 | 1.00 | 8.97 | 1RHP | 528 |
| ATOM | 419 | CB | LYS A | 61 | 8.003 | 29.700 | 35.340 | 1.00 | 8.98 | 1RHP | 529 |
| ATOM | 420 | CG | LYS A | 61 | 8.208 | 31.107 | 35.794 | 1.00 | 9.67 | 1RHP | 530 |
| ATOM | 421 | CD | LYS A | 61 | 9.543 | 31.372 | 35.176 | 1.00 | 10.69 | 1RHP | 531 |
| ATOM | 422 | CE | LYS A | 61 | 9.920 | 32.802 | 35.407 | 1.00 | 12.58 | 1RHP | 532 |
| ATOM | 423 | NZ | LYS A | 61 | 9.697 | 33.191 | 36.788 | 1.00 | 11.42 | 1RHP | 533 |
| ATOM | 424 | N | LYS A | 62 | 7.866 | 26.539 | 35.816 | 1.00 | 12.30 | 1RHP | 534 |
| ATOM | 425 | CA | LYS A | 62 | 7.867 | 25.205 | 35.221 | 1.00 | 13.09 | 1RHP | 535 |
| ATOM | 426 | C | LYS A | 62 | 6.565 | 24.462 | 35.482 | 1.00 | 13.14 | 1RHP | 536 |
| ATOM | 427 | O | LYS A | 62 | 5.787 | 24.156 | 34.577 | 1.00 | 12.78 | 1RHP | 537 |
| ATOM | 428 | CB | LYS A | 62 | 9.019 | 24.391 | 35.798 | 1.00 | 14.18 | 1RHP | 538 |
| ATOM | 429 | CG | LYS A | 62 | 9.224 | 22.991 | 35.222 | 1.00 | 14.28 | 1RHP | 539 |
| ATOM | 430 | CD | LYS A | 62 | 9.853 | 23.007 | 33.821 | 1.00 | 15.27 | 1RHP | 540 |
| ATOM | 431 | CE | LYS A | 62 | 10.143 | 21.568 | 33.477 | 1.00 | 13.02 | 1RHP | 541 |
| ATOM | 432 | NZ | LYS A | 62 | 11.245 | 21.549 | 32.556 | 1.00 | 13.10 | 1RHP | 542 |
| ATOM | 433 | N | ILE A | 63 | 6.301 | 24.217 | 36.758 | 1.00 | 11.80 | 1RHP | 543 |
| ATOM | 434 | CA | ILE A | 63 | 5.121 | 23.508 | 37.192 | 1.00 | 9.60 | 1RHP | 544 |
| ATOM | 435 | C | ILE A | 63 | 3.882 | 24.251 | 36.742 | 1.00 | 8.86 | 1RHP | 545 |
| ATOM | 436 | O | ILE A | 63 | 3.032 | 23.642 | 36.101 | 1.00 | 9.55 | 1RHP | 546 |
| ATOM | 437 | CB | ILE A | 63 | 5.182 | 23.388 | 38.692 | 1.00 | 8.16 | 1RHP | 547 |
| ATOM | 438 | CG1 | ILE A | 63 | 6.450 | 22.656 | 39.107 | 1.00 | 10.34 | 1RHP | 548 |
| ATOM | 439 | CG2 | ILE A | 63 | 3.972 | 22.664 | 39.139 | 1.00 | 8.73 | 1RHP | 549 |
| ATOM | 440 | CD1 | ILE A | 63 | 6.694 | 22.307 | 40.578 | 1.00 | 13.69 | 1RHP | 550 |
| ATOM | 441 | N | ILE A | 64 | 3.799 | 25.558 | 36.987 | 1.00 | 7.59 | 1RHP | 551 |
| ATOM | 442 | CA | ILE A | 64 | 2.634 | 26.306 | 36.611 | 1.00 | 9.19 | 1RHP | 552 |
| ATOM | 443 | C | ILE A | 64 | 2.388 | 26.193 | 35.133 | 1.00 | 10.55 | 1RHP | 553 |
| ATOM | 444 | O | ILE A | 64 | 1.229 | 25.962 | 34.820 | 1.00 | 12.93 | 1RHP | 554 |
| ATOM | 445 | CB | ILE A | 64 | 2.805 | 27.773 | 37.061 | 1.00 | 7.50 | 1RHP | 555 |
| ATOM | 446 | CG1 | ILE A | 64 | 2.518 | 27.768 | 38.576 | 1.00 | 8.52 | 1RHP | 556 |
| ATOM | 447 | CG2 | ILE A | 64 | 1.920 | 28.750 | 36.294 | 1.00 | 6.18 | 1RHP | 557 |
| ATOM | 448 | CD1 | ILE A | 64 | 2.803 | 29.046 | 39.420 | 1.00 | 8.38 | 1RHP | 558 |
| ATOM | 449 | N | LYS A | 65 | 3.298 | 26.234 | 34.177 | 1.00 | 12.10 | 1RHP | 559 |
| ATOM | 450 | CA | LYS A | 65 | 2.837 | 26.150 | 32.821 | 1.00 | 14.02 | 1RHP | 560 |
| ATOM | 451 | C | LYS A | 65 | 2.628 | 24.716 | 32.459 | 1.00 | 15.11 | 1RHP | 561 |
| ATOM | 452 | O | LYS A | 65 | 2.005 | 24.500 | 31.426 | 1.00 | 15.60 | 1RHP | 562 |
| ATOM | 453 | CB | LYS A | 65 | 3.803 | 26.767 | 31.839 | 1.00 | 17.12 | 1RHP | 563 |
| ATOM | 454 | CG | LYS A | 65 | 3.034 | 27.539 | 30.717 | 1.00 | 21.77 | 1RHP | 564 |
| ATOM | 455 | CD | LYS A | 65 | 2.299 | 26.673 | 29.654 | 1.00 | 24.17 | 1RHP | 565 |
| ATOM | 456 | CE | LYS A | 65 | 1.231 | 27.395 | 28.822 | 1.00 | 23.70 | 1RHP | 566 |
| ATOM | 457 | NZ | LYS A | 65 | 0.326 | 26.419 | 28.223 | 1.00 | 24.58 | 1RHP | 567 |
| ATOM | 458 | N | LYS A | 66 | 3.044 | 23.683 | 33.194 | 1.00 | 15.89 | 1RHP | 568 |
| ATOM | 459 | CA | LYS A | 66 | 2.617 | 22.342 | 32.789 | 1.00 | 16.58 | 1RHP | 569 |
| ATOM | 460 | C | LYS A | 66 | 1.150 | 22.218 | 33.221 | 1.00 | 17.67 | 1RHP | 570 |
| ATOM | 461 | O | LYS A | 66 | 0.394 | 21.432 | 32.639 | 1.00 | 21.96 | 1RHP | 571 |
| ATOM | 462 | CB | LYS A | 66 | 3.449 | 21.222 | 33.447 | 1.00 | 17.02 | 1RHP | 572 |
| ATOM | 463 | CG | LYS A | 66 | 4.983 | 21.322 | 33.249 | 1.00 | 20.36 | 1RHP | 573 |
| ATOM | 464 | CD | LYS A | 66 | 5.432 | 21.487 | 31.785 | 1.00 | 21.07 | 1RHP | 574 |
| ATOM | 465 | CE | LYS A | 66 | 6.484 | 22.605 | 31.641 | 1.00 | 20.00 | 1RHP | 575 |
| ATOM | 466 | NZ | LYS A | 66 | 6.436 | 23.278 | 30.346 | 1.00 | 20.18 | 1RHP | 576 |
| ATOM | 467 | N | LEU A | 67 | 0.707 | 23.032 | 34.202 | 1.00 | 16.85 | 1RHP | 577 |
| ATOM | 468 | CA | LEU A | 67 | −0.685 | 23.097 | 34.659 | 1.00 | 13.25 | 1RHP | 578 |
| ATOM | 469 | C | LEU A | 67 | −1.542 | 23.997 | 33.779 | 1.00 | 12.29 | 1RHP | 579 |
| ATOM | 470 | O | LEU A | 67 | −2.682 | 23.657 | 33.515 | 1.00 | 13.15 | 1RHP | 580 |
| ATOM | 471 | CB | LEU A | 67 | −0.803 | 23.641 | 36.092 | 1.00 | 7.96 | 1RHP | 581 |
| ATOM | 472 | CG | LEU A | 67 | −0.093 | 22.944 | 37.237 | 1.00 | 3.92 | 1RHP | 562 |
| ATOM | 473 | CD1 | LEU A | 67 | −0.401 | 23.676 | 38.487 | 1.00 | 2.52 | 1RHP | 583 |
| ATOM | 474 | CD2 | LEU A | 67 | −0.575 | 21.537 | 37.435 | 1.00 | 2.93 | 1RHP | 584 |
| ATOM | 475 | N | LEU A | 68 | −1.099 | 25.138 | 33.290 | 1.00 | 10.42 | 1RHP | 585 |
| ATOM | 476 | CA | LEU A | 68 | −1.929 | 26.029 | 32.504 | 1.00 | 10.73 | 1RHP | 586 |
| ATOM | 477 | C | LEU A | 68 | −2.078 | 25.609 | 31.058 | 1.00 | 12.75 | 1RHP | 587 |
| ATOM | 478 | O | LEU A | 68 | −1.946 | 26.370 | 30.082 | 1.00 | 10.65 | 1RHP | 588 |
| ATOM | 479 | CB | LEU A | 68 | −1.338 | 27.396 | 32.584 | 1.00 | 11.18 | 1RHP | 589 |
| ATOM | 480 | CG | LEU A | 68 | −1.865 | 28.415 | 33.560 | 1.00 | 12.10 | 1RHP | 590 |
| ATOM | 481 | CD1 | LEU A | 68 | −2.062 | 27.801 | 34.914 | 1.00 | 13.70 | 1RHP | 591 |
| ATOM | 482 | CD2 | LEU A | 68 | −0.867 | 29.561 | 33.647 | 1.00 | 12.88 | 1RHP | 582 |
| ATOM | 483 | N | GLU A | 69 | −2.516 | 24.365 | 30.979 | 1.00 | 16.13 | 1RHP | 593 |
| ATOM | 484 | CA | GLU A | 69 | −2.643 | 23.615 | 29.748 | 1.00 | 23.50 | 1RHP | 594 |
| ATOM | 485 | C | GLU A | 69 | −3.186 | 22.251 | 30.223 | 1.00 | 26.75 | 1RHP | 595 |
| ATOM | 486 | O | GLU A | 69 | −4.124 | 22.260 | 31.039 | 1.00 | 28.16 | 1RHP | 596 |
| ATOM | 487 | CB | GLU A | 69 | −1.285 | 23.510 | 29.180 | 1.00 | 24.43 | 1RHP | 597 |
| ATOM | 488 | CG | GLU A | 69 | −1.239 | 23.349 | 27.710 | 1.00 | 26.48 | 1RHP | 598 |
| ATOM | 489 | CD | GLU A | 69 | 0.114 | 22.771 | 27.367 | 1.00 | 28.27 | 1RHP | 599 |
| ATOM | 490 | OE1 | GLU A | 69 | 1.134 | 23.310 | 27.837 | 1.00 | 27.88 | 1RHP | 600 |
| ATOM | 491 | OE2 | GLU A | 69 | 0.132 | 21.760 | 26.656 | 1.00 | 30.17 | 1RHP | 601 |
| ATOM | 492 | N | SER A | 70 | −2.624 | 21.073 | 29.881 | 1.00 | 27.97 | 1RHP | 602 |
| ATOM | 493 | CA | SER A | 70 | −3.177 | 19.785 | 30.310 | 1.00 | 26.87 | 1RHP | 603 |
| ATOM | 494 | C | SER A | 70 | −2.057 | 18.800 | 30.668 | 1.00 | 27.50 | 1RHP | 604 |
| ATOM | 495 | O | SER A | 70 | −1.019 | 18.762 | 29.974 | 1.00 | 27.68 | 1RHP | 605 |
| ATOM | 496 | CB | SER A | 70 | −4.025 | 19.221 | 29.172 | 1.00 | 27.57 | 1RHP | 606 |
| ATOM | 497 | OG | SER A | 70 | −4.609 | 20.314 | 28.452 | 1.00 | 26.61 | 1RHP | 607 |
| TER | 498 | | SER A | 70 | | | | | | 1RHP | 608 |
| HETATM | 499 | O | HOH A | 71 | 1.441 | 9.706 | 30.512 | 1.00 | 23.59 | 1RHP | 609 |
| HETATM | 500 | O | HOH A | 72 | 7.286 | 26.889 | 50.542 | 1.00 | 27.41 | 1RHP | 610 |
| HETATM | 501 | O | HOH A | 73 | −3.388 | 30.794 | 61.718 | 1.00 | 15.03 | 1RHP | 611 |
| HETATM | 502 | O | HOH A | 74 | 7.376 | 36.705 | 41.431 | 1.00 | 21.13 | 1RHP | 612 |
| HETATM | 503 | O | HOH A | 75 | −7.221 | 20.396 | 27.869 | 1.00 | 15.40 | 1RHP | 613 |
| HETATM | 504 | O | HOH A | 76 | 14.249 | 16.834 | 42.220 | 1.00 | 39.10 | 1RHP | 614 |
| HETATM | 505 | O | HOH A | 77 | 1.991 | 6.425 | 47.231 | 1.00 | 17.03 | 1RHP | 615 |
| HETATM | 506 | O | HOH A | 78 | 10.865 | 16.120 | 59.043 | 1.00 | 29.88 | 1RHP | 616 |
| HETATM | 507 | O | HOH A | 79 | −6.335 | 16.868 | 43.397 | 1.00 | 44.71 | 1RHP | 617 |
| HETATM | 508 | O | HOH A | 80 | 10.601 | 36.531 | 38.480 | 1.00 | 20.86 | 1RHP | 618 |
| HETATM | 509 | O | HOH A | 81 | 2.395 | 13.017 | 27.637 | 1.00 | 35.17 | 1RHP | 619 |
| HETATM | 510 | O | HOH A | 82 | 1.629 | 20.208 | 28.804 | 1.00 | 29.20 | 1RHP | 620 |
| HETATM | 511 | O | HOH A | 83 | 0.848 | 14.249 | 33.742 | 1.00 | 34.71 | 1RHP | 621 |
| HETATM | 512 | O | HOH A | 84 | 16.874 | 15.525 | 59.033 | 1.00 | 29.01 | 1RHP | 622 |
| HETATM | 513 | O | HOH A | 85 | 4.996 | 35.535 | 60.902 | 1.00 | 9.39 | 1RHP | 623 |
| HETATM | 514 | O | HOH A | 86 | 8.076 | 29.220 | 51.759 | 1.00 | 27.50 | 1RHP | 624 |
| HETATM | 515 | O | HOH A | 87 | −4.320 | 14.479 | 42.695 | 1.00 | 40.77 | 1RHP | 625 |
| HETATM | 516 | O | HOH A | 88 | 1.991 | 4.000 | 48.464 | 1.00 | 33.25 | 1RHP | 626 |
| HETATM | 517 | O | HOH A | 89 | 11.385 | 18.541 | 53.057 | 1.00 | 29.34 | 1RHP | 627 |
| HETATM | 518 | O | HOH A | 90 | 1.082 | 19.686 | 35.468 | 1.00 | 23.94 | 1RHP | 628 |
| HETATM | 519 | O | HOH A | 91 | 2.102 | 11.541 | 35.327 | 1.00 | 24.90 | 1RHP | 629 |
| HETATM | 520 | O | HOH A | 92 | −1.591 | 29.019 | 29.987 | 1.00 | 39.76 | 1RHP | 630 |
| HETATM | 521 | O | HOH A | 93 | 12.054 | 9.498 | 37.355 | 1.00 | 42.50 | 1RHP | 631 |
| HETATM | 522 | O | HOH A | 94 | −0.812 | 4.693 | 44.959 | 1.00 | 30.68 | 1RHP | 632 |
| HETATM | 523 | O | HOH A | 95 | 14.214 | 11.730 | 33.809 | 1.00 | 35.05 | 1RHP | 633 |
| ATOM | 524 | N | ASP B | 7 | −16.599 | 14.751 | 41.674 | 1.00 | 6.71 | 1RHP | 634 |
| ATOM | 525 | CA | ASP B | 7 | −16.628 | 13.780 | 40.622 | 1.00 | 6.88 | 1RHP | 635 |
| ATOM | 526 | C | ASP B | 7 | −16.717 | 14.647 | 39.404 | 1.00 | 7.82 | 1RHP | 636 |
| ATOM | 527 | O | ASP B | 7 | −15.961 | 15.605 | 39.505 | 1.00 | 7.86 | 1RHP | 637 |
| ATOM | 528 | CB | ASP B | 7 | −18.207 | 13.152 | 40.677 | 1.00 | 6.72 | 1RHP | 638 |
| ATOM | 529 | CG | ASP B | 7 | −18.187 | 11.717 | 40.148 | 1.00 | 9.29 | 1RHP | 639 |
| ATOM | 530 | OD1 | ASP B | 7 | −17.925 | 11.507 | 38.946 | 1.00 | 7.94 | 1RHP | 640 |
| ATOM | 531 | OD2 | ASP B | 7 | −18.433 | 10.807 | 40.958 | 1.00 | 7.47 | 1RHP | 641 |
| ATOM | 532 | N | LEU B | 8 | −17.410 | 14.431 | 38.314 | 1.00 | 7.26 | 1RHP | 642 |
| ATOM | 533 | CA | LEU B | 8 | −17.258 | 15.225 | 37.119 | 1.00 | 9.93 | 1RHP | 643 |
| ATOM | 534 | C | LEU B | 8 | −17.343 | 16.755 | 37.198 | 1.00 | 13.16 | 1RHP | 644 |
| ATOM | 535 | O | LEU B | 8 | −17.337 | 17.373 | 36.125 | 1.00 | 15.46 | 1RHP | 645 |
| ATOM | 536 | CB | LEU B | 8 | −18.288 | 14.602 | 36.161 | 1.00 | 9.86 | 1RHP | 646 |
| ATOM | 537 | CG | LEU B | 8 | −19.030 | 15.233 | 34.963 | 1.00 | 9.12 | 1RHP | 647 |
| ATOM | 538 | CD1 | LEU B | 8 | −19.510 | 14.113 | 34.059 | 1.00 | 10.31 | 1RHP | 648 |
| ATOM | 539 | CD2 | LEU B | 8 | −20.213 | 16.076 | 35.418 | 1.00 | 10.66 | 1RHP | 649 |
| ATOM | 540 | N | GLN B | 9 | −17.385 | 17.502 | 38.302 | 1.00 | 14.34 | 1RHP | 650 |
| ATOM | 541 | CA | GLN B | 9 | −17.674 | 18.905 | 38.082 | 1.00 | 17.97 | 1RHP | 651 |
| ATOM | 542 | C | GLN B | 9 | −16.502 | 19.824 | 38.023 | 1.00 | 17.32 | 1RHP | 652 |
| ATOM | 543 | O | GLN B | 9 | −15.448 | 19.702 | 38.660 | 1.00 | 18.64 | 1RHP | 653 |
| ATOM | 544 | CB | GLN B | 9 | −18.621 | 19.509 | 39.125 | 1.00 | 19.36 | 1RHP | 654 |
| ATOM | 545 | CG | GLN B | 9 | −20.067 | 18.997 | 39.169 | 1.00 | 21.09 | 1RHP | 655 |
| ATOM | 546 | CD | GLN B | 9 | −21.028 | 19.399 | 38.059 | 1.00 | 22.41 | 1RHP | 656 |
| ATOM | 547 | OE1 | GLN B | 9 | −22.125 | 19.822 | 38.405 | 1.00 | 24.70 | 1RHP | 657 |
| ATOM | 548 | NE2 | GLN B | 9 | −20.866 | 19.237 | 36.746 | 1.00 | 21.91 | 1RHP | 658 |
| ATOM | 549 | N | CYS B | 10 | −16.792 | 20.758 | 37.150 | 1.00 | 16.30 | 1RHP | 659 |
| ATOM | 550 | CA | CYS B | 10 | −15.970 | 21.912 | 37.007 | 1.00 | 14.44 | 1RHP | 660 |
| ATOM | 551 | C | CYS B | 10 | −16.412 | 22.725 | 38.203 | 1.00 | 14.54 | 1RHP | 661 |
| ATOM | 552 | O | CYS B | 10 | −17.598 | 22.808 | 38.503 | 1.00 | 12.26 | 1RHP | 662 |
| ATOM | 553 | CB | CYS B | 10 | −16.319 | 22.605 | 35.739 | 1.00 | 14.58 | 1RHP | 663 |
| ATOM | 554 | SG | CYS B | 10 | −15.736 | 21.662 | 34.314 | 1.00 | 18.74 | 1RHP | 664 |
| ATOM | 555 | N | LEU B | 11 | −15.466 | 23.247 | 38.965 | 1.00 | 14.29 | 1RHP | 665 |
| ATOM | 556 | CA | LEU B | 11 | −15.830 | 24.120 | 40.036 | 1.00 | 12.18 | 1RHP | 666 |
| ATOM | 557 | C | LEU B | 11 | −16.330 | 25.396 | 39.388 | 1.00 | 14.39 | 1RHP | 667 |
| ATOM | 558 | O | LEU B | 11 | −17.463 | 25.808 | 39.647 | 1.00 | 16.26 | 1RHP | 668 |
| ATOM | 559 | CB | LEU B | 11 | −14.623 | 24.368 | 40.891 | 1.00 | 9.14 | 1RHP | 669 |
| ATOM | 560 | CG | LEU B | 11 | −14.486 | 23.331 | 41.964 | 1.00 | 9.44 | 1RHP | 670 |
| ATOM | 561 | CD1 | LEU B | 11 | −13.203 | 23.445 | 42.713 | 1.00 | 5.83 | 1RHP | 671 |
| ATOM | 562 | CD2 | LEU B | 11 | −15.612 | 23.559 | 42.945 | 1.00 | 9.77 | 1RHP | 672 |
| ATOM | 563 | N | CYS B | 12 | −15.564 | 26.025 | 38.499 | 1.00 | 14.94 | 1RHP | 673 |
| ATOM | 564 | CA | CYS B | 12 | −15.975 | 27.294 | 37.938 | 1.00 | 14.83 | 1RHP | 674 |
| ATOM | 565 | C | CYS B | 12 | −17.016 | 27.143 | 36.874 | 1.00 | 15.04 | 1RHP | 675 |
| ATOM | 566 | O | CYS B | 12 | −16.757 | 26.586 | 35.813 | 1.00 | 13.79 | 1RHP | 676 |
| ATOM | 567 | CB | CYS B | 12 | −14.792 | 28.001 | 37.357 | 1.00 | 13.61 | 1RHP | 677 |
| ATOM | 568 | SG | CYS B | 12 | −13.619 | 28.035 | 38.710 | 1.00 | 12.15 | 1RHP | 678 |
| ATOM | 569 | N | VAL B | 13 | −18.208 | 27.631 | 37.223 | 1.00 | 17.68 | 1RHP | 679 |
| ATOM | 570 | CA | VAL B | 13 | −19.361 | 27.689 | 36.324 | 1.00 | 16.43 | 1RHP | 680 |
| ATOM | 571 | C | VAL B | 13 | −19.330 | 28.970 | 35.529 | 1.00 | 16.01 | 1RHP | 681 |
| ATOM | 572 | O | VAL B | 13 | −19.831 | 29.010 | 34.414 | 1.00 | 15.17 | 1RHP | 682 |
| ATOM | 573 | CB | VAL B | 13 | −20.652 | 27.570 | 37.177 | 1.00 | 15.62 | 1RHP | 683 |
| ATOM | 574 | CG1 | VAL B | 13 | −21.698 | 28.661 | 37.052 | 1.00 | 13.84 | 1RHP | 684 |
| ATOM | 575 | CG2 | VAL B | 13 | −21.237 | 26.277 | 36.674 | 1.00 | 16.66 | 1RHP | 685 |
| ATOM | 576 | N | LYS B | 14 | −18.790 | 30.024 | 36.135 | 1.00 | 14.99 | 1RHP | 686 |
| ATOM | 577 | CA | LYS B | 14 | −18.645 | 31.326 | 35.502 | 1.00 | 17.11 | 1RHP | 687 |
| ATOM | 578 | C | LYS B | 14 | −17.412 | 31.932 | 36.132 | 1.00 | 14.55 | 1RHP | 688 |
| ATOM | 579 | O | LYS B | 14 | −17.235 | 31.696 | 37.335 | 1.00 | 14.45 | 1RHP | 689 |
| ATOM | 580 | CB | LYS B | 14 | −19.784 | 32.322 | 35.794 | 1.00 | 20.91 | 1RHP | 690 |
| ATOM | 581 | CG | LYS B | 14 | −21.141 | 32.184 | 35.106 | 1.00 | 24.68 | 1RHP | 691 |
| ATOM | 582 | CD | LYS B | 14 | −22.018 | 33.283 | 35.714 | 1.00 | 29.58 | 1RHP | 692 |
| ATOM | 583 | CE | LYS B | 14 | −23.504 | 32.970 | 35.428 | 1.00 | 32.18 | 1RHP | 693 |
| ATOM | 584 | NZ | LYS B | 14 | −24.423 | 33.691 | 36.319 | 1.00 | 36.38 | 1RHP | 694 |
| ATOM | 585 | N | THR B | 15 | −16.638 | 32.737 | 35.393 | 1.00 | 11.59 | 1RHP | 695 |
| ATOM | 586 | CA | THR B | 15 | −15.407 | 33.326 | 35.857 | 1.00 | 11.41 | 1RHP | 696 |
| ATOM | 587 | C | THR B | 15 | −15.728 | 34.557 | 36.676 | 1.00 | 14.81 | 1RHP | 697 |
| ATOM | 588 | O | THR B | 15 | −16.787 | 34.567 | 37.345 | 1.00 | 17.29 | 1RHP | 698 |
| ATOM | 589 | CB | THR B | 15 | −14.579 | 33.664 | 34.654 | 1.00 | 11.02 | 1RHP | 699 |
| ATOM | 590 | OG1 | THR B | 15 | −15.370 | 34.543 | 33.860 | 1.00 | 8.03 | 1RHP | 700 |
| ATOM | 591 | CG2 | THR B | 15 | −14.101 | 32.405 | 33.945 | 1.00 | 9.03 | 1RHP | 701 |
| ATOM | 592 | N | THR B | 16 | −14.793 | 35.521 | 36.754 | 1.00 | 13.55 | 1RHP | 702 |
| ATOM | 593 | CA | THR B | 16 | −15.032 | 36.758 | 37.451 | 1.00 | 13.04 | 1RHP | 703 |
| ATOM | 594 | C | THR B | 16 | −13.966 | 37.746 | 37.067 | 1.00 | 13.11 | 1RHP | 704 |
| ATOM | 595 | O | THR B | 16 | −13.022 | 38.042 | 37.785 | 1.00 | 14.65 | 1RHP | 705 |
| ATOM | 596 | CB | THR B | 16 | −15.020 | 36.563 | 38.960 | 1.00 | 15.18 | 1RHP | 706 |
| ATOM | 597 | OG1 | THR B | 16 | −14.956 | 35.175 | 39.309 | 1.00 | 14.67 | 1RHP | 707 |
| ATOM | 598 | CG2 | THR B | 16 | −16.276 | 37.201 | 39.509 | 1.00 | 13.87 | 1RHP | 708 |
| ATOM | 599 | N | SER B | 17 | −14.182 | 38.308 | 35.895 | 1.00 | 15.59 | 1RHP | 709 |
| ATOM | 600 | CA | SER B | 17 | −13.316 | 39.266 | 35.234 | 1.00 | 17.24 | 1RHP | 710 |
| ATOM | 601 | C | SER B | 17 | −12.747 | 40.478 | 35.978 | 1.00 | 17.71 | 1RHP | 711 |
| ATOM | 602 | O | SER B | 17 | −12.144 | 41.324 | 35.316 | 1.00 | 21.74 | 1RHP | 712 |
| ATOM | 603 | CB | SER B | 17 | −14.057 | 39.757 | 33.947 | 1.00 | 19.44 | 1RHP | 713 |
| ATOM | 604 | OG | SER B | 17 | −14.784 | 38.766 | 33.166 | 1.00 | 21.82 | 1RHP | 714 |
| ATOM | 605 | N | GLN B | 18 | −12.824 | 40.742 | 37.270 | 1.00 | 15.85 | 1RHP | 715 |
| ATOM | 606 | CA | GLN B | 18 | −12.114 | 41.888 | 37.819 | 1.00 | 18.33 | 1RHP | 716 |
| ATOM | 607 | C | GLN B | 18 | −12.062 | 41.589 | 39.286 | 1.00 | 18.02 | 1RHP | 717 |
| ATOM | 608 | O | GLN B | 18 | −13.002 | 40.997 | 39.821 | 1.00 | 17.08 | 1RHP | 718 |
| ATOM | 609 | CB | GLN B | 18 | −12.848 | 43.214 | 37.641 | 1.00 | 22.53 | 1RHP | 719 |
| ATOM | 610 | CG | GLN B | 18 | −12.058 | 44.482 | 38.076 | 1.00 | 28.01 | 1RHP | 720 |
| ATOM | 611 | CD | GLN B | 18 | −10.948 | 45.055 | 37.148 | 1.00 | 28.58 | 1RHP | 721 |
| ATOM | 612 | OE1 | GLN B | 18 | −10.335 | 46.087 | 37.477 | 1.00 | 31.50 | 1RHP | 722 |
| ATOM | 613 | NE2 | GLN B | 18 | −10.602 | 44.528 | 35.958 | 1.00 | 33.23 | 1RHP | 723 |
| ATOM | 614 | N | VAL B | 19 | −10.937 | 41.951 | 39.887 | 1.00 | 17.73 | 1RHP | 724 |
| ATOM | 615 | CA | VAL B | 19 | −10.675 | 41.751 | 41.300 | 1.00 | 19.22 | 1RHP | 725 |
| ATOM | 616 | C | VAL B | 19 | −9.359 | 42.461 | 41.567 | 1.00 | 22.93 | 1RHP | 726 |
| ATOM | 617 | O | VAL B | 19 | −8.525 | 42.541 | 40.656 | 1.00 | 24.40 | 1RHP | 727 |
| ATOM | 618 | CB | VAL B | 19 | −10.541 | 40.252 | 41.616 | 1.00 | 18.45 | 1RHP | 728 |
| ATOM | 619 | CG1 | VAL B | 19 | −9.417 | 39.572 | 40.838 | 1.00 | 16.26 | 1RHP | 729 |
| ATOM | 620 | CG2 | VAL B | 19 | −10.267 | 40.130 | 43.090 | 1.00 | 18.16 | 1RHP | 730 |
| ATOM | 621 | N | ARG B | 20 | −9.124 | 42.955 | 42.780 | 1.00 | 24.01 | 1RHP | 731 |
| ATOM | 622 | CA | ARG B | 20 | −7.859 | 43.625 | 43.077 | 1.00 | 24.80 | 1RHP | 732 |
| ATOM | 623 | C | ARG B | 20 | −6.749 | 42.648 | 43.555 | 1.00 | 22.48 | 1RHP | 733 |
| ATOM | 624 | O | ARG B | 20 | −6.958 | 42.040 | 44.617 | 1.00 | 23.56 | 1RHP | 734 |
| ATOM | 625 | CB | ARG B | 20 | −8.244 | 44.714 | 44.114 | 1.00 | 26.07 | 1RHP | 735 |
| ATOM | 626 | CG | ARG B | 20 | −7.155 | 45.739 | 44.509 | 1.00 | 28.36 | 1RHP | 736 |
| ATOM | 627 | CD | ARG B | 20 | −7.631 | 46.952 | 45.373 | 1.00 | 30.29 | 1RHP | 737 |
| ATOM | 628 | NE | ARG B | 20 | −8.331 | 47.957 | 44.580 | 1.00 | 28.61 | 1RHP | 738 |
| ATOM | 629 | CZ | ARG B | 20 | −8.682 | 49.168 | 45.044 | 1.00 | 26.73 | 1RHP | 739 |
| ATOM | 630 | NH1 | ARG B | 20 | −8.397 | 49.615 | 46.269 | 1.00 | 24.77 | 1RHP | 740 |
| ATOM | 631 | NH2 | ARG B | 20 | −9.313 | 49.988 | 44.206 | 1.00 | 27.79 | 1RHP | 741 |
| ATOM | 632 | N | PRO B | 21 | −5.552 | 42.441 | 42.957 | 1.00 | 20.67 | 1RHP | 742 |
| ATOM | 633 | CA | PRO B | 21 | −4.487 | 41.499 | 43.412 | 1.00 | 20.34 | 1RHP | 743 |
| ATOM | 634 | C | PRO B | 21 | −4.042 | 41.734 | 44.855 | 1.00 | 20.31 | 1RHP | 744 |
| ATOM | 635 | O | PRO B | 21 | −3.468 | 40.900 | 45.534 | 1.00 | 21.08 | 1RHP | 745 |
| ATOM | 636 | CB | PRO B | 21 | −3.331 | 41.690 | 42.460 | 1.00 | 17.36 | 1RHP | 746 |
| ATOM | 637 | CG | PRO B | 21 | −3.935 | 42.366 | 41.248 | 1.00 | 18.63 | 1RHP | 747 |
| ATOM | 638 | CD | PRO B | 21 | −5.044 | 43.238 | 41.857 | 1.00 | 19.44 | 1RHP | 748 |
| ATOM | 639 | N | ARG B | 22 | −4.320 | 42.914 | 45.391 | 1.00 | 23.94 | 1RHP | 749 |
| ATOM | 640 | CA | ARG B | 22 | −4.069 | 43.213 | 46.795 | 1.00 | 25.25 | 1RHP | 750 |
| ATOM | 641 | C | ARG B | 22 | −5.208 | 42.566 | 47.624 | 1.00 | 25.15 | 1RHP | 751 |
| ATOM | 642 | O | ARG B | 22 | −5.410 | 42.924 | 48.794 | 1.00 | 24.31 | 1RHP | 752 |
| ATOM | 643 | CB | ARG B | 22 | −3.946 | 44.802 | 46.867 | 1.00 | 28.32 | 1RHP | 753 |
| ATOM | 644 | CG | ARG B | 22 | −4.775 | 45.792 | 47.742 | 1.00 | 27.20 | 1RHP | 754 |
| ATOM | 645 | CD | ARG B | 22 | −3.925 | 46.817 | 48.555 | 1.00 | 27.40 | 1RHP | 755 |
| ATOM | 646 | NE | ARG B | 22 | −3.077 | 46.078 | 49.495 | 1.00 | 25.56 | 1RHP | 756 |
| ATOM | 647 | CZ | ARG B | 22 | −3.014 | 46.290 | 50.811 | 1.00 | 23.63 | 1RHP | 757 |
| ATOM | 648 | NH1 | ARG B | 22 | −3.699 | 47.274 | 51.390 | 1.00 | 24.89 | 1RHP | 758 |
| ATOM | 649 | NH2 | ARG B | 22 | −2.262 | 45.467 | 51.557 | 1.00 | 24.37 | 1RHP | 759 |
| ATOM | 650 | N | HIS B | 23 | −5.998 | 41.607 | 47.073 | 1.00 | 23.30 | 1RHP | 760 |
| ATOM | 651 | CA | HIS B | 23 | −7.116 | 41.029 | 47.810 | 1.00 | 22.41 | 1RHP | 761 |
| ATOM | 652 | C | HIS B | 23 | −7.066 | 39.533 | 48.017 | 1.00 | 18.77 | 1RHP | 762 |
| ATOM | 653 | O | HIS B | 23 | −7.518 | 39.032 | 49.047 | 1.00 | 15.42 | 1RHP | 763 |
| ATOM | 654 | CB | HIS B | 23 | −8.491 | 41.348 | 47.145 | 1.00 | 24.58 | 1RHP | 764 |
| ATOM | 655 | CG | HIS B | 23 | −9.124 | 42.723 | 47.499 | 1.00 | 28.15 | 1RHP | 765 |
| ATOM | 656 | ND1 | HIS B | 23 | −9.858 | 43.547 | 46.729 | 1.00 | 28.62 | 1RHP | 766 |
| ATOM | 657 | CD2 | HIS B | 23 | −9.014 | 43.377 | 48.735 | 1.00 | 30.22 | 1RHP | 767 |
| ATOM | 658 | CE1 | HIS B | 23 | −10.178 | 44.633 | 47.441 | 1.00 | 32.42 | 1RHP | 768 |
| ATOM | 659 | NE2 | HIS B | 23 | −9.659 | 44.525 | 48.651 | 1.00 | 30.14 | 1RHP | 769 |
| ATOM | 660 | N | ILE B | 24 | −6.471 | 38.824 | 47.079 | 1.00 | 14.30 | 1RHP | 770 |
| ATOM | 661 | CA | ILE B | 24 | −6.487 | 37.375 | 47.024 | 1.00 | 12.03 | 1RHP | 771 |
| ATOM | 662 | C | ILE B | 24 | −5.532 | 36.736 | 48.017 | 1.00 | 13.90 | 1RHP | 772 |
| ATOM | 663 | O | ILE B | 24 | −4.374 | 37.143 | 47.983 | 1.00 | 14.55 | 1RHP | 773 |
| ATOM | 664 | CB | ILE B | 24 | −6.118 | 37.018 | 45.601 | 1.00 | 11.19 | 1RHP | 774 |
| ATOM | 665 | CG1 | ILE B | 24 | −7.124 | 37.651 | 44.682 | 1.00 | 12.67 | 1RHP | 775 |
| ATOM | 666 | CG2 | ILE B | 24 | −6.052 | 35.522 | 45.428 | 1.00 | 12.84 | 1RHP | 776 |
| ATOM | 667 | CD1 | ILE B | 24 | −6.896 | 37.461 | 43.189 | 1.00 | 11.71 | 1RHP | 777 |
| ATOM | 668 | N | THR B | 25 | −5.878 | 35.786 | 48.895 | 1.00 | 15.57 | 1RHP | 778 |
| ATOM | 669 | CA | THR B | 25 | −4.873 | 35.145 | 49.741 | 1.00 | 15.27 | 1RHP | 779 |
| ATOM | 670 | C | THR B | 25 | −4.287 | 33.931 | 49.001 | 1.00 | 14.01 | 1RHP | 780 |
| ATOM | 671 | O | THR B | 25 | −3.114 | 33.889 | 48.635 | 1.00 | 13.94 | 1RHP | 781 |
| ATOM | 672 | CB | THR B | 25 | −5.430 | 34.597 | 51.075 | 1.00 | 18.14 | 1RHP | 782 |
| ATOM | 673 | OG1 | THR B | 25 | −6.736 | 35.076 | 51.329 | 1.00 | 19.37 | 1RHP | 783 |
| ATOM | 674 | CG2 | THR B | 25 | −4.494 | 34.992 | 52.192 | 1.00 | 20.19 | 1RHP | 784 |
| ATOM | 675 | N | SER B | 26 | −5.116 | 32.910 | 48.782 | 1.00 | 10.07 | 1RHP | 785 |
| ATOM | 676 | CA | SER B | 26 | −4.729 | 31.650 | 48.183 | 1.00 | 9.61 | 1RHP | 786 |
| ATOM | 677 | C | SER B | 26 | −5.030 | 31.797 | 46.724 | 1.00 | 8.16 | 1RHP | 787 |
| ATOM | 678 | O | SER B | 26 | −5.880 | 32.608 | 46.385 | 1.00 | 8.76 | 1RHP | 788 |
| ATOM | 679 | CB | SER B | 26 | −5.571 | 30.545 | 48.786 | 1.00 | 11.70 | 1RHP | 789 |
| ATOM | 680 | OG | SER B | 26 | −4.980 | 29.236 | 48.814 | 1.00 | 19.77 | 1RHP | 790 |
| ATOM | 681 | N | LEU B | 27 | −4.393 | 31.116 | 45.816 | 1.00 | 7.23 | 1RHP | 791 |
| ATOM | 682 | CA | LEU B | 27 | −4.794 | 31.192 | 44.437 | 1.00 | 9.33 | 1RHP | 792 |
| ATOM | 683 | C | LEU B | 27 | −4.427 | 29.800 | 44.000 | 1.00 | 12.71 | 1RHP | 793 |
| ATOM | 684 | O | LEU B | 27 | −3.292 | 29.345 | 44.187 | 1.00 | 14.92 | 1RHP | 794 |
| ATOM | 685 | CB | LEU B | 27 | −4.001 | 32.235 | 43.709 | 1.00 | 8.62 | 1RHP | 795 |
| ATOM | 686 | CG | LEU B | 27 | −4.286 | 32.468 | 42.237 | 1.00 | 9.60 | 1RHP | 796 |
| ATOM | 687 | CD1 | LEU B | 27 | −3.985 | 33.898 | 41.894 | 1.00 | 12.46 | 1RHP | 797 |
| ATOM | 688 | CD2 | LEU B | 27 | −3.405 | 31.583 | 41.374 | 1.00 | 10.99 | 1RHP | 798 |
| ATOM | 689 | N | GLU B | 28 | −5.400 | 29.118 | 43.416 | 1.00 | 12.03 | 1RHP | 799 |
| ATOM | 690 | CA | GLU B | 28 | −5.281 | 27.715 | 43.140 | 1.00 | 12.56 | 1RHP | 800 |
| ATOM | 691 | C | GLU B | 28 | −5.320 | 27.466 | 41.658 | 1.00 | 15.35 | 1RHP | 801 |
| ATOM | 692 | O | GLU B | 28 | −6.287 | 27.853 | 40.989 | 1.00 | 16.84 | 1RHP | 802 |
| ATOM | 693 | CB | GLU B | 28 | −6.425 | 27.020 | 43.839 | 1.00 | 11.80 | 1RHP | 803 |
| ATOM | 694 | CG | GLU B | 28 | −6.309 | 25.491 | 43.891 | 1.00 | 11.43 | 1RHP | 804 |
| ATOM | 695 | CD | GLU B | 28 | −7.341 | 24.801 | 44.743 | 1.00 | 12.19 | 1RHP | 805 |
| ATOM | 696 | OE1 | GLU B | 28 | −7.953 | 25.433 | 45.597 | 1.00 | 10.13 | 1RHP | 806 |
| ATOM | 697 | OE2 | GLU B | 28 | −7.525 | 23.614 | 44.542 | 1.00 | 12.36 | 1RHP | 807 |
| ATOM | 698 | N | VAL B | 29 | −4.278 | 26.813 | 41.155 | 1.00 | 15.27 | 1RHP | 808 |
| ATOM | 699 | CA | VAL B | 29 | −4.212 | 26.474 | 39.757 | 1.00 | 12.56 | 1RHP | 809 |
| ATOM | 700 | C | VAL B | 29 | −4.541 | 24.987 | 39.729 | 1.00 | 9.90 | 1RHP | 810 |
| ATOM | 701 | O | VAL B | 29 | −3.906 | 24.204 | 40.424 | 1.00 | 9.03 | 1RHP | 811 |
| ATOM | 702 | CB | VAL B | 29 | −2.796 | 26.835 | 39.272 | 1.00 | 12.48 | 1RHP | 812 |
| ATOM | 703 | CG1 | VAL B | 29 | −2.629 | 26.365 | 37.877 | 1.00 | 12.53 | 1RHP | 813 |
| ATOM | 704 | CG2 | VAL B | 29 | −2.581 | 28.344 | 39.197 | 1.00 | 10.30 | 1RHP | 814 |
| ATOM | 705 | N | ILE B | 30 | −5.605 | 24.616 | 39.022 | 1.00 | 9.52 | 1RHP | 815 |
| ATOM | 706 | CA | ILE B | 30 | −6.110 | 23.254 | 38.907 | 1.00 | 11.83 | 1RHP | 816 |
| ATOM | 707 | C | ILE B | 30 | −6.031 | 22.853 | 37.430 | 1.00 | 14.07 | 1RHP | 817 |
| ATOM | 708 | O | ILE B | 30 | −6.516 | 23.536 | 36.512 | 1.00 | 14.01 | 1RHP | 818 |
| ATOM | 709 | CB | ILE B | 30 | −7.589 | 23.155 | 39.392 | 1.00 | 10.60 | 1RHP | 819 |
| ATOM | 710 | CG1 | ILE B | 30 | −7.803 | 23.738 | 40.785 | 1.00 | 9.90 | 1RHP | 820 |
| ATOM | 711 | CG2 | ILE B | 30 | −7.944 | 21.695 | 39.436 | 1.00 | 10.95 | 1RHP | 821 |
| ATOM | 712 | CD1 | ILE B | 30 | −9.087 | 23.269 | 41.486 | 1.00 | 8.55 | 1RHP | 822 |
| ATOM | 713 | N | LYS B | 31 | −5.409 | 21.713 | 37.188 | 1.00 | 14.56 | 1RHP | 823 |
| ATOM | 714 | CA | LYS B | 31 | −5.141 | 21.288 | 35.836 | 1.00 | 14.90 | 1RHP | 824 |
| ATOM | 715 | C | LYS B | 31 | −6.268 | 20.385 | 35.421 | 1.00 | 15.83 | 1RHP | 825 |
| ATOM | 716 | O | LYS B | 31 | −6.937 | 19.762 | 36.255 | 1.00 | 15.93 | 1RHP | 826 |
| ATOM | 717 | CB | LYS B | 31 | −3.824 | 20.536 | 35.803 | 1.00 | 14.69 | 1RHP | 827 |
| ATOM | 718 | CG | LYS B | 31 | −3.202 | 20.382 | 34.433 | 1.00 | 13.07 | 1RHP | 828 |
| ATOM | 719 | CD | LYS B | 31 | −2.281 | 19.201 | 34.485 | 1.00 | 12.65 | 1RHP | 829 |
| ATOM | 720 | CE | LYS B | 31 | −3.074 | 17.932 | 34.765 | 1.00 | 14.37 | 1RHP | 830 |
| ATOM | 721 | NZ | LYS B | 31 | −2.200 | 16.777 | 34.917 | 1.00 | 15.82 | 1RHP | 831 |
| ATOM | 722 | N | ALA B | 32 | −6.418 | 20.321 | 34.101 | 1.00 | 15.07 | 1RHP | 832 |
| ATOM | 723 | CA | ALA B | 32 | −7.388 | 19.496 | 33.412 | 1.00 | 14.31 | 1RHP | 833 |
| ATOM | 724 | C | ALA B | 32 | −7.282 | 18.074 | 33.832 | 1.00 | 14.88 | 1RHP | 034 |
| ATOM | 725 | O | ALA B | 32 | −6.333 | 17.708 | 34.508 | 1.00 | 16.92 | 1RHP | 835 |
| ATOM | 726 | CB | ALA B | 32 | −7.155 | 19.512 | 31.936 | 1.00 | 14.87 | 1RHP | 836 |
| ATOM | 727 | N | GLY B | 33 | −8.228 | 17.270 | 33.398 | 1.00 | 18.70 | 1RHP | 837 |
| ATOM | 728 | CA | GLY B | 33 | −8.292 | 15.839 | 33.736 | 1.00 | 21.94 | 1RHP | 838 |
| ATOM | 729 | C | GLY B | 33 | −9.763 | 15.393 | 33.606 | 1.00 | 23.37 | 1RHP | 839 |
| ATOM | 730 | O | GLY B | 33 | −10.552 | 16.167 | 33.025 | 1.00 | 27.46 | 1RHP | 840 |
| ATOM | 731 | N | PRO B | 34 | −10.279 | 14.290 | 34.169 | 1.00 | 19.65 | 1RHP | 841 |
| ATOM | 732 | CA | PRO B | 34 | −11.676 | 13.848 | 34.016 | 1.00 | 18.30 | 1RHP | 842 |
| ATOM | 733 | C | PRO B | 34 | −12.795 | 14.720 | 34.645 | 1.00 | 16.74 | 1RHP | 843 |
| ATOM | 734 | O | PRO B | 34 | −13.929 | 14.282 | 34.838 | 1.00 | 17.33 | 1RHP | 844 |
| ATOM | 735 | CB | PRO B | 34 | −11.627 | 12.445 | 34.597 | 1.00 | 18.80 | 1RHP | 845 |
| ATOM | 736 | CG | PRO B | 34 | −10.173 | 12.134 | 34.875 | 1.00 | 16.67 | 1RHP | 846 |
| ATOM | 737 | CD | PRO B | 34 | −9.589 | 13.496 | 35.158 | 1.00 | 19.46 | 1RHP | 847 |
| ATOM | 738 | N | HIS B | 35 | −12.594 | 15.978 | 35.010 | 1.00 | 14.83 | 1RHP | 848 |
| ATOM | 739 | CA | HIS B | 35 | −13.635 | 16.704 | 35.720 | 1.00 | 15.41 | 1RHP | 849 |
| ATOM | 740 | C | HIS B | 35 | −13.996 | 18.076 | 35.163 | 1.00 | 14.14 | 1RHP | 850 |
| ATOM | 741 | O | HIS B | 35 | −14.917 | 18.787 | 35.578 | 1.00 | 14.88 | 1RHP | 851 |
| ATOM | 742 | CB | HIS B | 35 | −13.189 | 16.834 | 37.157 | 1.00 | 17.57 | 1RHP | 852 |
| ATOM | 743 | CG | HIS B | 35 | −11.793 | 17.433 | 37.281 | 1.00 | 21.36 | 1RHP | 853 |
| ATOM | 744 | ND1 | HIS B | 35 | −10.668 | 16.764 | 37.536 | 1.00 | 22.07 | 1RHP | 854 |
| ATOM | 745 | CD2 | HIS B | 35 | −11.445 | 18.761 | 37.116 | 1.00 | 23.26 | 1RHP | 855 |
| ATOM | 746 | CE1 | HIS B | 35 | −9.665 | 17.625 | 37.523 | 1.00 | 22.24 | 1RHP | 856 |
| ATOM | 747 | NE2 | HIS B | 35 | −10.149 | 18.813 | 37.274 | 1.00 | 24.14 | 1RHP | 857 |
| ATOM | 748 | N | CYS B | 36 | −13.207 | 18.489 | 34.191 | 1.00 | 11.64 | 1RHP | 858 |
| ATOM | 749 | CA | CYS B | 36 | −13.292 | 19.817 | 33.668 | 1.00 | 11.54 | 1RHP | 859 |
| ATOM | 750 | C | CYS B | 36 | −12.153 | 19.680 | 32.695 | 1.00 | 12.55 | 1RHP | 860 |
| ATOM | 751 | O | CYS B | 36 | −11.016 | 19.462 | 33.120 | 1.00 | 13.11 | 1RHP | 861 |
| ATOM | 752 | CB | CYS B | 36 | −12.962 | 20.825 | 34.738 | 1.00 | 10.73 | 1RHP | 862 |
| ATOM | 753 | SG | CYS B | 36 | −13.849 | 22.352 | 34.423 | 1.00 | 13.16 | 1RHP | 863 |
| ATOM | 754 | N | PRO B | 37 | −12.359 | 19.676 | 31.394 | 1.00 | 12.41 | 1RHP | 864 |
| ATOM | 755 | CA | PRO B | 37 | −11.304 | 19.769 | 30.413 | 1.00 | 14.05 | 1RHP | 865 |
| ATOM | 756 | C | PRO B | 37 | −10.665 | 21.142 | 30.377 | 1.00 | 13.26 | 1RHP | 866 |
| ATOM | 757 | O | PRO B | 37 | −10.088 | 21.503 | 29.357 | 1.00 | 14.90 | 1RHP | 867 |
| ATOM | 758 | CB | PRO B | 37 | −12.008 | 19.387 | 29.156 | 1.00 | 13.74 | 1RHP | 868 |
| ATOM | 759 | CG | PRO B | 37 | −13.369 | 19.984 | 29.372 | 1.00 | 16.34 | 1RHP | 869 |
| ATOM | 760 | CD | PRO B | 37 | −13.642 | 19.445 | 30.752 | 1.00 | 14.85 | 1RHP | 870 |
| ATOM | 761 | N | THR B | 38 | −10.651 | 21.893 | 31.467 | 1.00 | 12.60 | 1RHP | 871 |
| ATOM | 762 | CA | THR B | 38 | −10.186 | 23.255 | 31.455 | 1.00 | 14.10 | 1RHP | 872 |
| ATOM | 763 | C | THR B | 38 | −9.420 | 23.496 | 32.721 | 1.00 | 14.48 | 1RHP | 873 |
| ATOM | 764 | O | THR B | 38 | −9.778 | 23.038 | 33.809 | 1.00 | 16.21 | 1RHP | 874 |
| ATOM | 765 | CB | THR B | 38 | −11.385 | 24.158 | 31.387 | 1.00 | 14.43 | 1RHP | 875 |
| ATOM | 766 | OG1 | THR B | 38 | −11.924 | 23.841 | 30.109 | 1.00 | 15.57 | 1RHP | 876 |
| ATOM | 767 | CG2 | THR B | 38 | −11.112 | 25.643 | 31.566 | 1.00 | 14.47 | 1RHP | 877 |
| ATOM | 768 | N | ALA B | 39 | −8.315 | 24.186 | 32.580 | 1.00 | 15.51 | 1RHP | 878 |
| ATOM | 769 | CA | ALA B | 39 | −7.584 | 24.548 | 33.755 | 1.00 | 14.33 | 1RHP | 879 |
| ATOM | 770 | C | ALA B | 39 | −8.501 | 25.584 | 34.363 | 1.00 | 14.26 | 1RHP | 880 |
| ATOM | 771 | O | ALA B | 39 | −9.208 | 26.304 | 33.658 | 1.00 | 12.03 | 1RHP | 881 |
| ATOM | 772 | CB | ALA B | 39 | −6.265 | 25.141 | 33.367 | 1.00 | 16.08 | 1RHP | 882 |
| ATOM | 773 | N | GLN B | 40 | −8.572 | 25.537 | 35.675 | 1.00 | 14.67 | 1RHP | 883 |
| ATOM | 774 | CA | GLN B | 40 | −9.423 | 26.422 | 36.438 | 1.00 | 14.71 | 1RHP | 884 |
| ATOM | 775 | C | GLN B | 40 | −8.473 | 27.181 | 37.359 | 1.00 | 14.67 | 1RHP | 885 |
| ATOM | 776 | O | GLN B | 40 | −7.500 | 26.567 | 37.798 | 1.00 | 13.26 | 1RHP | 886 |
| ATOM | 777 | CB | GLN B | 40 | −10.402 | 25.587 | 37.245 | 1.00 | 15.63 | 1RHP | 887 |
| ATOM | 778 | CG | GLN B | 40 | −11.158 | 24.563 | 36.428 | 1.00 | 14.87 | 1RHP | 888 |
| ATOM | 779 | CD | GLN B | 40 | −12.024 | 23.745 | 37.354 | 1.00 | 17.01 | 1RHP | 889 |
| ATOM | 780 | OE1 | GLN B | 40 | −13.086 | 24.191 | 37.789 | 1.00 | 17.42 | 1RHP | 890 |
| ATOM | 781 | NE2 | GLN B | 40 | −11.605 | 22.544 | 37.735 | 1.00 | 17.23 | 1RHP | 891 |
| ATOM | 782 | N | LEU B | 41 | −8.697 | 28.441 | 37.727 | 1.00 | 15.12 | 1RHP | 892 |
| ATOM | 783 | CA | LEU B | 41 | −7.789 | 29.198 | 38.564 | 1.00 | 14.02 | 1RHP | 893 |
| ATOM | 784 | C | LEU B | 41 | −8.624 | 29.872 | 39.662 | 1.00 | 14.67 | 1RHP | 894 |
| ATOM | 785 | O | LEU B | 41 | −9.025 | 31.044 | 39.598 | 1.00 | 16.79 | 1RHP | 895 |
| ATOM | 786 | CB | LEU B | 41 | −7.106 | 30.190 | 37.641 | 1.00 | 15.99 | 1RHP | 896 |
| ATOM | 787 | CG | LEU B | 41 | −5.600 | 30.258 | 37.396 | 1.00 | 15.92 | 1RHP | 897 |
| ATOM | 788 | CD1 | LEU B | 41 | −5.005 | 29.070 | 35.675 | 1.00 | 16.60 | 1RHP | 898 |
| ATOM | 789 | CD2 | LEU B | 41 | −5.399 | 31.383 | 36.436 | 1.00 | 16.85 | 1RHP | 899 |
| ATOM | 790 | N | ILE B | 42 | −8.963 | 29.133 | 40.704 | 1.00 | 11.77 | 1RHP | 900 |
| ATOM | 791 | CA | ILE B | 42 | −9.817 | 29.595 | 41.814 | 1.00 | 10.15 | 1RHP | 901 |
| ATOM | 792 | C | ILE B | 42 | −9.121 | 30.667 | 42.653 | 1.00 | 9.73 | 1RHP | 902 |
| ATOM | 793 | O | ILE B | 42 | −7.904 | 30.543 | 42.796 | 1.00 | 14.00 | 1RHP | 903 |
| ATOM | 794 | CB | ILE B | 42 | −10.140 | 28.282 | 42.558 | 1.00 | 8.44 | 1RHP | 904 |
| ATOM | 795 | CG1 | ILE B | 42 | −11.154 | 27.593 | 41.722 | 1.00 | 7.82 | 1RHP | 905 |
| ATOM | 796 | CG2 | ILE B | 42 | −10.641 | 28.454 | 43.955 | 1.00 | 8.78 | 1RHP | 906 |
| ATOM | 797 | CD1 | ILE B | 42 | −10.754 | 26.139 | 41.638 | 1.00 | 6.30 | 1RHP | 907 |
| ATOM | 798 | N | ALA B | 43 | −9.731 | 31.693 | 43.235 | 1.00 | 7.13 | 1RHP | 908 |
| ATOM | 799 | CA | ALA B | 43 | −9.008 | 32.694 | 44.004 | 1.00 | 5.37 | 1RHP | 909 |
| ATOM | 800 | C | ALA B | 43 | −9.805 | 32.913 | 45.237 | 1.00 | 4.98 | 1RHP | 910 |
| ATOM | 801 | O | ALA B | 43 | −10.931 | 33.324 | 45.062 | 1.00 | 8.14 | 1RHP | 911 |
| ATOM | 802 | CB | ALA B | 43 | −8.956 | 33.991 | 43.259 | 1.00 | 2.74 | 1RHP | 912 |
| ATOM | 803 | N | THR B | 44 | −9.392 | 32.649 | 46.453 | 1.00 | 5.38 | 1RHP | 913 |
| ATOM | 804 | CA | THR B | 44 | −10.166 | 32.870 | 47.671 | 1.00 | 7.46 | 1RHP | 914 |
| ATOM | 805 | C | THR B | 44 | −9.866 | 34.289 | 48.114 | 1.00 | 9.32 | 1RHP | 915 |
| ATOM | 806 | O | THR B | 44 | −8.753 | 34.533 | 48.575 | 1.00 | 14.27 | 1RHP | 916 |
| ATOM | 807 | CB | THR B | 44 | −9.732 | 31.910 | 48.822 | 1.00 | 8.95 | 1RHP | 917 |
| ATOM | 808 | OG1 | THR B | 44 | −9.967 | 30.586 | 48.355 | 1.00 | 11.12 | 1RHP | 918 |
| ATOM | 809 | CG2 | THR B | 44 | −10.453 | 32.159 | 50.144 | 1.00 | 10.01 | 1RHP | 919 |
| ATOM | 810 | N | LEU B | 45 | −10.751 | 35.273 | 47.995 | 1.00 | 11.21 | 1RHP | 920 |
| ATOM | 811 | CA | LEU B | 45 | −10.466 | 36.640 | 48.423 | 1.00 | 9.85 | 1RHP | 921 |
| ATOM | 812 | C | LEU B | 45 | −10.167 | 36.778 | 49.914 | 1.00 | 7.93 | 1RHP | 922 |
| ATOM | 813 | O | LEU B | 45 | −10.373 | 35.870 | 50.707 | 1.00 | 4.49 | 1RHP | 923 |
| ATOM | 814 | CB | LEU B | 45 | −11.669 | 37.493 | 48.000 | 1.00 | 11.08 | 1RHP | 924 |
| ATOM | 815 | CG | LEU B | 45 | −11.612 | 38.407 | 46.776 | 1.00 | 14.78 | 1RHP | 925 |
| ATOM | 816 | CD1 | LEU B | 45 | −10.474 | 37.977 | 45.875 | 1.00 | 15.54 | 1RHP | 926 |
| ATOM | 817 | CD2 | LEU B | 45 | −12.969 | 38.383 | 46.055 | 1.00 | 14.25 | 1RHP | 927 |
| ATOM | 818 | N | LYS B | 46 | −9.715 | 37.942 | 50.333 | 1.00 | 9.27 | 1RHP | 928 |
| ATOM | 819 | CA | LYS B | 46 | −9.393 | 38.187 | 51.719 | 1.00 | 13.08 | 1RHP | 929 |
| ATOM | 820 | C | LYS B | 46 | −10.486 | 37.924 | 52.760 | 1.00 | 15.31 | 1RHP | 930 |
| ATOM | 821 | O | LYS B | 46 | −10.200 | 37.868 | 53.965 | 1.00 | 16.74 | 1RHP | 931 |
| ATOM | 822 | CB | LYS B | 46 | −8.954 | 39.620 | 51.828 | 1.00 | 13.07 | 1RHP | 932 |
| ATOM | 823 | CG | LYS B | 46 | −7.706 | 39.839 | 52.678 | 1.00 | 16.19 | 1RHP | 933 |
| ATOM | 824 | CD | LYS B | 46 | −6.411 | 39.746 | 51.860 | 1.00 | 18.42 | 1RHP | 934 |
| ATOM | 825 | CE | LYS B | 46 | −6.091 | 40.952 | 50.978 | 1.00 | 16.53 | 1RHP | 935 |
| ATOM | 826 | NZ | LYS B | 46 | −6.037 | 42.195 | 51.720 | 1.00 | 17.26 | 1RHP | 936 |
| ATOM | 827 | N | ASN B | 47 | −11.763 | 37.817 | 52.349 | 1.00 | 18.41 | 1RHP | 937 |
| ATOM | 828 | CA | ASN B | 47 | −12.859 | 37.659 | 53.304 | 1.00 | 18.10 | 1RHP | 938 |
| ATOM | 829 | C | ASN B | 47 | −13.334 | 36.239 | 53.438 | 1.00 | 15.94 | 1RHP | 939 |
| ATOM | 830 | O | ASN B | 47 | −13.514 | 35.791 | 54.558 | 1.00 | 17.03 | 1RHP | 940 |
| ATOM | 831 | CB | ASN B | 47 | −14.064 | 38.545 | 52.940 | 1.00 | 19.72 | 1RHP | 941 |
| ATOM | 832 | CG | ASN B | 47 | −14.782 | 38.334 | 51.612 | 1.00 | 20.57 | 1RHP | 942 |
| ATOM | 833 | OD1 | ASN B | 47 | −14.770 | 37.265 | 50.987 | 1.00 | 23.37 | 1RHP | 943 |
| ATOM | 834 | ND2 | ASN B | 47 | −15.473 | 39.373 | 51.170 | 1.00 | 21.71 | 1RHP | 944 |
| ATOM | 835 | N | GLY B | 48 | −13.563 | 35.496 | 52.387 | 1.00 | 14.44 | 1RHP | 945 |
| ATOM | 836 | CA | GLY B | 48 | −13.897 | 34.112 | 52.531 | 1.00 | 11.29 | 1RHP | 946 |
| ATOM | 837 | C | GLY B | 48 | −14.351 | 33.576 | 51.205 | 1.00 | 12.87 | 1RHP | 947 |
| ATOM | 838 | O | GLY B | 48 | −14.043 | 32.439 | 50.841 | 1.00 | 11.93 | 1RHP | 948 |
| ATOM | 839 | N | ARG B | 49 | −15.021 | 34.392 | 50.408 | 1.00 | 15.68 | 1RHP | 949 |
| ATOM | 840 | CA | ARG B | 49 | −15.615 | 33.817 | 49.228 | 1.00 | 22.27 | 1RHP | 950 |
| ATOM | 841 | C | ARG B | 49 | −14.776 | 33.856 | 47.963 | 1.00 | 22.09 | 1RHP | 951 |
| ATOM | 842 | O | ARG B | 49 | −14.163 | 34.866 | 47.601 | 1.00 | 23.53 | 1RHP | 952 |
| ATOM | 843 | CB | ARG B | 49 | −16.994 | 34.491 | 49.033 | 1.00 | 26.40 | 1RHP | 953 |
| ATOM | 844 | CG | ARG B | 49 | −18.198 | 33.946 | 49.940 | 1.00 | 31.14 | 1RHP | 954 |
| ATOM | 845 | CD | ARG B | 49 | −18.079 | 34.028 | 51.506 | 1.00 | 31.76 | 1RHP | 955 |
| ATOM | 846 | NE | ARG B | 49 | −17.463 | 35.289 | 51.961 | 1.00 | 32.10 | 1RHP | 956 |
| ATOM | 847 | CZ | ARG B | 49 | −17.095 | 35.534 | 53.237 | 1.00 | 31.04 | 1RHP | 957 |
| ATOM | 848 | NH1 | ARG B | 49 | −17.363 | 34.710 | 54.270 | 1.00 | 28.06 | 1RHP | 958 |
| ATOM | 849 | NH2 | ARG B | 49 | −16.491 | 36.689 | 53.482 | 1.00 | 29.81 | 1RHP | 959 |
| ATOM | 850 | N | LYS B | 50 | −14.734 | 32.664 | 47.367 | 1.00 | 18.08 | 1RHP | 960 |
| ATOM | 851 | CA | LYS B | 50 | −13.960 | 32.352 | 46.177 | 1.00 | 13.52 | 1RHP | 981 |
| ATOM | 852 | C | LYS B | 50 | −14.457 | 32.976 | 44.905 | 1.00 | 12.03 | 1RHP | 982 |
| ATOM | 853 | O | LYS B | 50 | −15.665 | 33.002 | 44.719 | 1.00 | 10.78 | 1RHP | 963 |
| ATOM | 854 | CB | LYS B | 50 | −13.930 | 30.844 | 45.922 | 1.00 | 13.13 | 1RHP | 964 |
| ATOM | 855 | CG | LYS B | 50 | −13.249 | 29.990 | 46.977 | 1.00 | 14.43 | 1RHP | 965 |
| ATOM | 856 | CD | LYS B | 50 | −13.812 | 28.559 | 47.020 | 1.00 | 15.02 | 1RHP | 966 |
| ATOM | 857 | CE | LYS B | 50 | −12.751 | 27.527 | 47.461 | 1.00 | 14.95 | 1RHP | 967 |
| ATOM | 858 | NZ | LYS B | 50 | −12.134 | 27.874 | 48.729 | 1.00 | 16.11 | 1RHP | 968 |
| ATOM | 859 | N | ILE B | 51 | −13.595 | 33.445 | 44.018 | 1.00 | 10.56 | 1RHP | 969 |
| ATOM | 860 | CA | ILE B | 51 | −13.988 | 33.799 | 42.655 | 1.00 | 12.15 | 1RHP | 970 |
| ATOM | 861 | C | ILE B | 51 | −13.039 | 33.039 | 41.722 | 1.00 | 13.58 | 1RHP | 971 |
| ATOM | 862 | O | ILE B | 51 | −11.893 | 32.801 | 42.104 | 1.00 | 14.88 | 1RHP | 972 |
| ATOM | 863 | CB | ILE B | 51 | −13.841 | 35.294 | 42.262 | 1.00 | 12.72 | 1RHP | 973 |
| ATOM | 864 | CG1 | ILE B | 51 | −12.425 | 35.786 | 42.469 | 1.00 | 17.18 | 1RHP | 974 |
| ATOM | 865 | CG2 | ILE B | 51 | −14.858 | 36.086 | 43.032 | 1.00 | 11.19 | 1RHP | 975 |
| ATOM | 866 | CD1 | ILE B | 51 | −12.206 | 37.108 | 41.746 | 1.00 | 18.52 | 1RHP | 976 |
| ATOM | 867 | N | CYS B | 52 | −13.419 | 32.633 | 40.512 | 1.00 | 11.26 | 1RHP | 977 |
| ATOM | 868 | CA | CYS B | 52 | −12.498 | 31.940 | 39.627 | 1.00 | 10.87 | 1RHP | 978 |
| ATOM | 869 | C | CYS B | 52 | −11.949 | 32.957 | 38.640 | 1.00 | 14.16 | 1RHP | 979 |
| ATOM | 870 | O | CYS B | 52 | −12.734 | 33.586 | 37.907 | 1.00 | 15.50 | 1RHP | 980 |
| ATOM | 871 | CB | CYS B | 52 | −13.151 | 30.868 | 38.769 | 1.00 | 11.97 | 1RHP | 981 |
| ATOM | 872 | SG | CYS B | 52 | −14.137 | 29.691 | 39.700 | 1.00 | 16.03 | 1RHP | 982 |
| ATOM | 873 | N | LEU B | 53 | −10.636 | 33.169 | 38.604 | 1.00 | 13.62 | 1RHP | 983 |
| ATOM | 874 | CA | LEU B | 53 | −10.033 | 34.018 | 37.574 | 1.00 | 13.12 | 1RHP | 984 |
| ATOM | 875 | C | LEU B | 53 | −10.073 | 33.278 | 36.236 | 1.00 | 12.30 | 1RHP | 985 |
| ATOM | 876 | O | LEU B | 53 | −10.387 | 32.074 | 36.213 | 1.00 | 11.22 | 1RHP | 986 |
| ATOM | 877 | CB | LEU B | 53 | −8.610 | 34.298 | 37.927 | 1.00 | 14.18 | 1RHP | 987 |
| ATOM | 878 | CG | LEU B | 53 | −8.347 | 34.855 | 39.282 | 1.00 | 13.56 | 1RHP | 988 |
| ATOM | 879 | CD1 | LEU B | 53 | −6.844 | 34.961 | 39.455 | 1.00 | 16.34 | 1RHP | 989 |
| ATOM | 880 | CD2 | LEU B | 53 | −9.027 | 36.201 | 39.422 | 1.00 | 16.96 | 1RHP | 990 |
| ATOM | 881 | N | ASP B | 54 | −9.750 | 33.863 | 35.108 | 1.00 | 9.76 | 1RHP | 991 |
| ATOM | 882 | CA | ASP B | 54 | −9.842 | 33.074 | 33.902 | 1.00 | 15.76 | 1RHP | 992 |
| ATOM | 883 | C | ASP B | 54 | −8.531 | 32.920 | 33.205 | 1.00 | 19.09 | 1RHP | 993 |
| ATOM | 884 | O | ASP B | 54 | −7.674 | 33.794 | 33.350 | 1.00 | 20.58 | 1RHP | 994 |
| ATOM | 885 | CB | ASP B | 54 | −10.781 | 33.692 | 32.918 | 1.00 | 18.44 | 1RHP | 995 |
| ATOM | 886 | CG | ASP B | 54 | −10.385 | 35.100 | 32.524 | 1.00 | 20.80 | 1RHP | 996 |
| ATOM | 887 | OD1 | ASP B | 54 | −9.971 | 35.867 | 33.403 | 1.00 | 20.52 | 1RHP | 997 |
| ATOM | 888 | OD2 | ASP B | 54 | −10.485 | 35.406 | 31.328 | 1.00 | 21.92 | 1RHP | 998 |
| ATOM | 889 | N | LEU B | 55 | −8.340 | 31.847 | 32.435 | 1.00 | 22.16 | 1RHP | 999 |
| ATOM | 890 | CA | LEU B | 55 | −7.119 | 31.739 | 31.622 | 1.00 | 24.05 | 1RHP | 1000 |
| ATOM | 891 | C | LEU B | 55 | −7.282 | 32.670 | 30.400 | 1.00 | 25.69 | 1RHP | 1001 |
| ATOM | 892 | O | LEU B | 55 | −8.282 | 33.418 | 30.318 | 1.00 | 26.52 | 1RHP | 1002 |
| ATOM | 893 | CB | LEU B | 55 | −6.902 | 30.309 | 31.123 | 1.00 | 23.82 | 1RHP | 1003 |
| ATOM | 894 | CG | LEU B | 55 | −7.069 | 29.154 | 32.114 | 1.00 | 23.30 | 1RHP | 1004 |
| ATOM | 895 | CD1 | LEU B | 55 | −6.677 | 27.880 | 31.382 | 1.00 | 22.40 | 1RHP | 1005 |
| ATOM | 896 | CD2 | LEU B | 55 | −6.233 | 29.355 | 33.371 | 1.00 | 22.31 | 1RHP | 1006 |
| ATOM | 897 | N | GLN B | 56 | −6.331 | 32.556 | 29.433 | 1.00 | 27.52 | 1RHP | 1007 |
| ATOM | 898 | CA | GLN B | 56 | −6.217 | 33.408 | 28.240 | 1.00 | 28.19 | 1RHP | 1008 |
| ATOM | 899 | C | GLN B | 56 | −6.587 | 34.858 | 28.590 | 1.00 | 29.51 | 1RHP | 1009 |
| ATOM | 900 | O | GLN B | 56 | −7.543 | 35.401 | 28.026 | 1.00 | 29.52 | 1RHP | 1010 |
| ATOM | 901 | CB | GLN B | 56 | −7.112 | 32.811 | 27.127 | 1.00 | 27.09 | 1RHP | 1011 |
| ATOM | 902 | CG | GLN B | 56 | −7.091 | 33.528 | 25.761 | 1.00 | 27.62 | 1RHP | 1012 |
| ATOM | 903 | CD | GLN B | 56 | −5.744 | 33.605 | 25.050 | 1.00 | 27.03 | 1RHP | 1013 |
| ATOM | 904 | OE1 | GLN B | 56 | −5.388 | 34.609 | 24.408 | 1.00 | 30.46 | 1RHP | 1014 |
| ATOM | 905 | NE2 | GLN B | 56 | −4.922 | 32.556 | 25.151 | 1.00 | 29.38 | 1RHP | 1015 |
| ATOM | 906 | N | ALA B | 57 | −5.878 | 35.497 | 29.559 | 1.00 | 29.94 | 1RHP | 1016 |
| ATOM | 907 | CA | ALA B | 57 | −6.253 | 36.853 | 29.965 | 1.00 | 31.90 | 1RHP | 1017 |
| ATOM | 908 | C | ALA B | 57 | −5.286 | 37.606 | 30.881 | 1.00 | 32.93 | 1RHP | 1018 |
| ATOM | 909 | O | ALA B | 57 | −4.824 | 36.972 | 31.851 | 1.00 | 34.58 | 1RHP | 1019 |
| ATOM | 910 | CB | ALA B | 57 | −7.581 | 36.843 | 30.695 | 1.00 | 32.44 | 1RHP | 1020 |
| ATOM | 911 | N | PRO B | 58 | −4.990 | 38.936 | 30.687 | 1.00 | 31.20 | 1RHP | 1021 |
| ATOM | 912 | CA | PRO B | 58 | −3.938 | 39.666 | 31.407 | 1.00 | 28.77 | 1RHP | 1022 |
| ATOM | 913 | C | PRO B | 58 | −3.982 | 39.484 | 32.909 | 1.00 | 27.71 | 1RHP | 1023 |
| ATOM | 914 | O | PRO B | 58 | −2.905 | 39.183 | 33.417 | 1.00 | 29.57 | 1RHP | 1024 |
| ATOM | 915 | CB | PRO B | 58 | −4.077 | 41.131 | 31.021 | 1.00 | 28.73 | 1RHP | 1025 |
| ATOM | 916 | CG | PRO B | 58 | −5.447 | 41.224 | 30.368 | 1.00 | 29.51 | 1RHP | 1026 |
| ATOM | 917 | CD | PRO B | 58 | −5.664 | 39.835 | 29.738 | 1.00 | 29.90 | 1RHP | 1027 |
| ATOM | 918 | N | LEU B | 59 | −5.113 | 39.512 | 33.649 | 1.00 | 24.61 | 1RHP | 1028 |
| ATOM | 919 | CA | LEU B | 59 | −5.023 | 39.353 | 35.111 | 1.00 | 22.91 | 1RHP | 1029 |
| ATOM | 920 | C | LEU B | 59 | −4.280 | 38.153 | 35.788 | 1.00 | 23.86 | 1RHP | 1030 |
| ATOM | 921 | O | LEU B | 59 | −3.657 | 38.376 | 36.849 | 1.00 | 23.40 | 1RHP | 1031 |
| ATOM | 922 | CB | LEU B | 59 | −6.440 | 39.398 | 35.746 | 1.00 | 20.62 | 1RHP | 1032 |
| ATOM | 923 | CG | LEU B | 59 | −6.820 | 40.596 | 36.665 | 1.00 | 17.79 | 1RHP | 1033 |
| ATOM | 924 | CD1 | LEU B | 59 | −7.694 | 40.133 | 37.814 | 1.00 | 17.99 | 1RHP | 1034 |
| ATOM | 925 | CD2 | LEU B | 59 | −5.614 | 41.129 | 37.400 | 1.00 | 19.38 | 1RHP | 1035 |
| ATOM | 926 | N | TYR B | 60 | −4.182 | 36.902 | 35.298 | 1.00 | 22.26 | 1RHP | 1036 |
| ATOM | 927 | CA | TYR B | 60 | −3.598 | 35.897 | 36.182 | 1.00 | 22.28 | 1RHP | 1037 |
| ATOM | 928 | C | TYR B | 60 | −2.062 | 35.975 | 36.367 | 1.00 | 23.64 | 1RHP | 1038 |
| ATOM | 929 | O | TYR B | 60 | −1.560 | 35.805 | 37.496 | 1.00 | 24.05 | 1RHP | 1039 |
| ATOM | 930 | CB | TYR B | 60 | −4.055 | 34.536 | 35.691 | 1.00 | 20.06 | 1RHP | 1040 |
| ATOM | 931 | CG | TYR B | 60 | −3.620 | 34.066 | 34.329 | 1.00 | 19.91 | 1RHP | 1041 |
| ATOM | 932 | CD1 | TYR B | 60 | −4.238 | 34.493 | 33.165 | 1.00 | 21.89 | 1RHP | 1042 |
| ATOM | 933 | CD2 | TYR B | 60 | −2.610 | 33.148 | 34.291 | 1.00 | 20.96 | 1RHP | 1043 |
| ATOM | 934 | CE1 | TYR B | 60 | −3.860 | 33.965 | 31.928 | 1.00 | 22.70 | 1RHP | 1044 |
| ATOM | 935 | CE2 | TYR B | 60 | −2.236 | 32.617 | 33.076 | 1.00 | 20.76 | 1RHP | 1045 |
| ATOM | 936 | CZ | TYR B | 60 | −2.843 | 33.015 | 31.900 | 1.00 | 22.51 | 1RHP | 1046 |
| ATOM | 937 | OH | TYR B | 60 | −2.441 | 32.365 | 30.736 | 1.00 | 23.69 | 1RHP | 1047 |
| ATOM | 938 | N | LYS B | 61 | −1.304 | 36.317 | 35.302 | 1.00 | 22.09 | 1RHP | 1048 |
| ATOM | 939 | CA | LYS B | 61 | 0.140 | 36.599 | 35.381 | 1.00 | 21.78 | 1RHP | 1049 |
| ATOM | 940 | C | LYS B | 61 | 0.418 | 37.627 | 36.512 | 1.00 | 21.96 | 1RHP | 1050 |
| ATOM | 941 | O | LYS B | 61 | 1.271 | 37.508 | 37.407 | 1.00 | 20.96 | 1RHP | 1051 |
| ATOM | 942 | CB | LYS B | 61 | 0.643 | 37.218 | 34.057 | 1.00 | 22.99 | 1RHP | 1052 |
| ATOM | 943 | CG | LYS B | 61 | 0.514 | 36.423 | 32.760 | 1.00 | 23.56 | 1RHP | 1053 |
| ATOM | 944 | CD | LYS B | 61 | 1.800 | 35.625 | 32.494 | 1.00 | 23.41 | 1RHP | 1054 |
| ATOM | 945 | CE | LYS B | 61 | 2.879 | 36.543 | 31.964 | 1.00 | 21.19 | 1RHP | 1055 |
| ATOM | 946 | NZ | LYS B | 61 | 2.411 | 37.078 | 30.699 | 1.00 | 21.14 | 1RHP | 1056 |
| ATOM | 947 | N | LYS B | 62 | −0.430 | 38.655 | 36.486 | 1.00 | 21.57 | 1RHP | 1057 |
| ATOM | 948 | CA | LYS B | 62 | −0.316 | 39.762 | 37.414 | 1.00 | 22.24 | 1RHP | 1058 |
| ATOM | 949 | C | LYS B | 62 | −0.604 | 39.277 | 38.830 | 1.00 | 22.14 | 1RHP | 1059 |
| ATOM | 950 | O | LYS B | 62 | 0.207 | 39.561 | 39.719 | 1.00 | 21.39 | 1RHP | 1060 |
| ATOM | 951 | CB | LYS B | 62 | −1.299 | 40.892 | 37.017 | 1.00 | 20.29 | 1RHP | 1061 |
| ATOM | 952 | CG | LYS B | 62 | −1.204 | 41.242 | 35.527 | 1.00 | 20.83 | 1RHP | 1062 |
| ATOM | 953 | CD | LYS B | 62 | −1.768 | 42.606 | 35.168 | 1.00 | 20.68 | 1RHP | 1063 |
| ATOM | 954 | CE | LYS B | 62 | −1.693 | 42.845 | 33.654 | 1.00 | 22.39 | 1RHP | 1064 |
| ATOM | 955 | NZ | LYS B | 62 | −2.047 | 44.227 | 33.343 | 1.00 | 23.06 | 1RHP | 1065 |
| ATOM | 956 | N | ILE B | 63 | −1.659 | 38.482 | 39.063 | 1.00 | 21.58 | 1RHP | 1066 |
| ATOM | 957 | CA | ILE B | 63 | −2.000 | 38.089 | 40.419 | 1.00 | 18.73 | 1RHP | 1067 |
| ATOM | 958 | C | ILE B | 63 | −0.893 | 37.187 | 40.952 | 1.00 | 20.98 | 1RHP | 1068 |
| ATOM | 959 | O | ILE B | 63 | −0.394 | 37.386 | 42.071 | 1.00 | 22.01 | 1RHP | 1069 |
| ATOM | 960 | CB | ILE B | 63 | −3.294 | 37.276 | 40.533 | 1.00 | 17.62 | 1RHP | 1070 |
| ATOM | 961 | CG1 | ILE B | 63 | −4.506 | 37.757 | 39.735 | 1.00 | 18.31 | 1RHP | 1071 |
| ATOM | 962 | CG2 | ILE B | 63 | −3.625 | 37.380 | 41.980 | 1.00 | 13.61 | 1RHP | 1072 |
| ATOM | 963 | CD1 | ILE B | 63 | −5.496 | 38.833 | 40.281 | 1.00 | 16.63 | 1RHP | 1073 |
| ATOM | 964 | N | ILE B | 64 | −0.457 | 36.192 | 40.163 | 1.00 | 20.69 | 1RHP | 1074 |
| ATOM | 965 | CA | ILE B | 64 | 0.549 | 35.251 | 40.639 | 1.00 | 20.83 | 1RHP | 1075 |
| ATOM | 966 | C | ILE B | 64 | 1.827 | 36.018 | 40.992 | 1.00 | 22.24 | 1RHP | 1076 |
| ATOM | 967 | O | ILE B | 64 | 2.480 | 35.677 | 41.985 | 1.00 | 23.04 | 1RHP | 1077 |
| ATOM | 968 | CB | ILE B | 64 | 0.799 | 34.168 | 39.537 | 1.00 | 18.99 | 1RHP | 1078 |
| ATOM | 969 | CG1 | ILE B | 64 | −0.492 | 33.353 | 39.313 | 1.00 | 18.27 | 1RHP | 1079 |
| ATOM | 970 | CG2 | ILE B | 64 | 1.969 | 33.254 | 39.952 | 1.00 | 18.99 | 1RHP | 1080 |
| ATOM | 971 | CD1 | ILE B | 64 | −0.364 | 32.141 | 38.361 | 1.00 | 13.64 | 1RHP | 1081 |
| ATOM | 972 | N | LYS B | 65 | 2.182 | 37.084 | 40.253 | 1.00 | 23.66 | 1RHP | 1082 |
| ATOM | 973 | CA | LYS B | 65 | 3.338 | 37.889 | 40.602 | 1.00 | 22.41 | 1RHP | 1083 |
| ATOM | 974 | C | LYS B | 65 | 3.140 | 38.434 | 41.994 | 1.00 | 22.16 | 1RHP | 1084 |
| ATOM | 975 | O | LYS B | 65 | 3.863 | 38.038 | 42.909 | 1.00 | 18.23 | 1RHP | 1085 |
| ATOM | 976 | CB | LYS B | 65 | 3.538 | 39.092 | 39.661 | 1.00 | 26.89 | 1RHP | 1086 |
| ATOM | 977 | CG | LYS B | 65 | 4.077 | 38.669 | 38.285 | 1.00 | 28.35 | 1RHP | 1087 |
| ATOM | 978 | CD | LYS B | 65 | 4.577 | 39.794 | 37.338 | 1.00 | 30.68 | 1RHP | 1088 |
| ATOM | 979 | CE | LYS B | 65 | 5.122 | 39.163 | 36.026 | 1.00 | 32.00 | 1RHP | 1089 |
| ATOM | 980 | NZ | LYS B | 65 | 4.962 | 40.050 | 34.877 | 1.00 | 28.71 | 1RHP | 1090 |
| ATOM | 981 | N | LYS B | 66 | 2.089 | 39.236 | 42.166 | 1.00 | 22.29 | 1RHP | 1091 |
| ATOM | 982 | CA | LYS B | 66 | 1.820 | 39.970 | 43.396 | 1.00 | 23.00 | 1RHP | 1092 |
| ATOM | 983 | C | LYS B | 66 | 2.003 | 39.065 | 44.579 | 1.00 | 21.48 | 1RHP | 1093 |
| ATOM | 984 | O | LYS B | 66 | 2.828 | 39.306 | 45.451 | 1.00 | 20.58 | 1RHP | 1094 |
| ATOM | 985 | CB | LYS B | 66 | 0.373 | 40.516 | 43.415 | 1.00 | 24.56 | 1RHP | 1095 |
| ATOM | 986 | CG | LYS B | 66 | 0.042 | 41.534 | 44.538 | 1.00 | 26.18 | 1RHP | 1096 |
| ATOM | 987 | CD | LYS B | 66 | 0.770 | 42.848 | 44.192 | 1.00 | 28.03 | 1RHP | 1097 |
| ATOM | 988 | CE | LYS B | 66 | 0.526 | 43.955 | 45.201 | 1.00 | 28.23 | 1RHP | 1098 |
| ATOM | 989 | NZ | LYS B | 66 | 1.349 | 45.096 | 44.845 | 1.00 | 27.88 | 1RHP | 1099 |
| ATOM | 990 | N | LEU B | 67 | 1.327 | 37.939 | 44.419 | 1.00 | 20.36 | 1RHP | 1100 |
| ATOM | 991 | CA | LEU B | 67 | 1.274 | 36.941 | 45.444 | 1.00 | 19.29 | 1RHP | 1101 |
| ATOM | 992 | C | LEU B | 67 | 2.671 | 36.445 | 45.751 | 1.00 | 20.23 | 1RHP | 1102 |
| ATOM | 993 | O | LEU B | 67 | 3.077 | 36.545 | 46.912 | 1.00 | 20.14 | 1RHP | 1103 |
| ATOM | 994 | CB | LEU B | 67 | 0.382 | 35.787 | 44.982 | 1.00 | 15.33 | 1RHP | 1104 |
| ATOM | 995 | CG | LEU B | 67 | −1.101 | 35.754 | 45.204 | 1.00 | 13.27 | 1RHP | 1105 |
| ATOM | 996 | CD1 | LEU B | 67 | −1.661 | 34.556 | 44.515 | 1.00 | 10.83 | 1RHP | 1106 |
| ATOM | 997 | CD2 | LEU B | 67 | −1.421 | 35.540 | 46.647 | 1.00 | 10.64 | 1RHP | 1107 |
| ATOM | 998 | N | LEU B | 68 | 3.439 | 35.986 | 44.754 | 1.00 | 21.24 | 1RHP | 1108 |
| ATOM | 999 | CA | LEU B | 68 | 4.734 | 35.412 | 45.069 | 1.00 | 22.54 | 1RHP | 1109 |
| ATOM | 1000 | C | LEU B | 68 | 5.784 | 36.336 | 45.736 | 1.00 | 23.67 | 1RHP | 1110 |
| ATOM | 1001 | O | LEU B | 68 | 6.500 | 35.872 | 46.642 | 1.00 | 24.01 | 1RHP | 1111 |
| ATOM | 1002 | CB | LEU B | 68 | 5.302 | 34.785 | 43.789 | 1.00 | 19.06 | 1RHP | 1112 |
| ATOM | 1003 | CG | LEU B | 68 | 5.155 | 33.256 | 43.562 | 1.00 | 19.29 | 1RHP | 1113 |
| ATOM | 1004 | CD1 | LEU B | 68 | 5.128 | 32.547 | 44.903 | 1.00 | 15.82 | 1RHP | 1114 |
| ATOM | 1005 | CD2 | LEU B | 68 | 3.894 | 32.937 | 42.791 | 1.00 | 16.56 | 1RHP | 1115 |
| ATOM | 1006 | N | GLU B | 69 | 5.911 | 37.634 | 45.387 | 1.00 | 23.00 | 1RHP | 1116 |
| ATOM | 1007 | CA | GLU B | 69 | 6.872 | 38.516 | 46.053 | 1.00 | 21.74 | 1RHP | 1117 |
| ATOM | 1008 | C | GLU B | 69 | 6.531 | 38.500 | 47.532 | 1.00 | 22.47 | 1RHP | 1118 |
| ATOM | 1009 | O | GLU B | 69 | 7.418 | 38.166 | 48.320 | 1.00 | 22.61 | 1RHP | 1119 |
| ATOM | 1010 | CB | GLU B | 69 | 6.770 | 39.965 | 45.619 | 1.00 | 23.39 | 1RHP | 112O |
| ATOM | 1011 | CG | GLU B | 69 | 6.770 | 40.255 | 44.101 | 1.00 | 26.10 | 1RHP | 1121 |
| ATOM | 1012 | CD | GLU B | 69 | 6.388 | 41.699 | 43.704 | 1.00 | 26.82 | 1RHP | 1122 |
| ATOM | 1013 | OE1 | GLU B | 69 | 5.849 | 42.452 | 44.539 | 1.00 | 27.65 | 1RHP | 1123 |
| ATOM | 1014 | OE2 | GLU B | 69 | 6.631 | 42.054 | 42.538 | 1.00 | 28.39 | 1RHP | 1124 |
| ATOM | 1015 | N | SER B | 70 | 5.253 | 38.775 | 47.870 | 1.00 | 22.94 | 1RHP | 1125 |
| ATOM | 1016 | CA | SER B | 70 | 4.727 | 38.778 | 49.233 | 1.00 | 23.87 | 1RHP | 1126 |
| ATOM | 1017 | C | SER B | 70 | 5.518 | 39.666 | 50.186 | 1.00 | 23.17 | 1RHP | 1127 |
| ATOM | 1018 | O | SER B | 70 | 4.904 | 40.518 | 50.839 | 1.00 | 25.59 | 1RHP | 1128 |
| ATOM | 1019 | CB | SER B | 70 | 4.693 | 37.328 | 49.814 | 1.00 | 24.60 | 1RHP | 1129 |
| ATOM | 1020 | OG | SER B | 70 | 3.534 | 36.510 | 49.576 | 1.00 | 24.06 | 1RHP | 1130 |
| TER | 1021 | | SER B | 70 | | | | | | 1RHP | 1131 |
| HETATM | 1022 | O | HOH B | 71 | −24.354 | 19.768 | 40.329 | 1.00 | 13.29 | 1RHP | 1132 |
| HETATM | 1023 | O | HOH B | 72 | −10.696 | 29.595 | 35.287 | 1.00 | 31.41 | 1RHP | 1133 |
| HETATM | 1024 | O | HOH B | 73 | −8.819 | 28.083 | 46.927 | 1.00 | 21.22 | 1RHP | 1134 |
| HETATM | 1025 | O | HOH B | 74 | 0.142 | 17.556 | 32.927 | 1.00 | 26.43 | 1RHP | 1135 |
| HETATM | 1026 | O | HOH B | 75 | −9.383 | 38.863 | 56.334 | 1.00 | 39.17 | 1RHP | 1136 |
| HETATM | 1027 | O | HOH B | 76 | −3.284 | 32.871 | 28.332 | 1.00 | 43.54 | 1RHP | 1137 |
| HETATM | 1028 | O | HOH B | 77 | −18.816 | 21.587 | 33.721 | 1.00 | 23.40 | 1RHP | 1138 |
| HETATM | 1029 | O | HOH B | 78 | −12.760 | 33.792 | 56.930 | 1.00 | 19.07 | 1RHP | 1139 |
| HETATM | 1030 | O | HOH B | 79 | −5.833 | 48.682 | 43.094 | 1.00 | 31.24 | 1RHP | 1140 |
| HETATM | 1031 | O | HOH B | 80 | −12.389 | 19.778 | 39.679 | 1.00 | 19.93 | 1RHP | 1141 |
| HETATM | 1032 | O | HOH B | 81 | −14.666 | 40.987 | 41.947 | 1.00 | 37.58 | 1RHP | 1142 |
| HETATM | 1033 | O | HOH B | 82 | −17.064 | 39.620 | 34.643 | 1.00 | 36.95 | 1RHP | 1143 |
| HETATM | 1034 | O | HOH B | 83 | −11.187 | 36.810 | 35.641 | 1.00 | 25.15 | 1RHP | 1144 |
| HETATM | 1035 | O | HOH B | 84 | −11.876 | 37.706 | 33.178 | 1.00 | 28.47 | 1RHP | 1145 |
| HETATM | 1036 | O | HOH B | 85 | −25.295 | 37.298 | 36.065 | 1.00 | 20.34 | 1RHP | 1146 |
| HETATM | 1037 | O | HOH B | 86 | −2.291 | 31.588 | 25.969 | 1.00 | 27.96 | 1RHP | 1147 |
| HETATM | 1038 | O | HOH B | 87 | 8.306 | 35.400 | 48.739 | 1.00 | 14.94 | 1RHP | 1148 |
| HETATM | 1039 | O | HOH B | 88 | −13.066 | 29.041 | 33.612 | 1.00 | 16.27 | 1RHP | 1149 |
| HETATM | 1040 | O | HOH B | 89 | −16.939 | 33.190 | 31.717 | 1.00 | 25.65 | 1RHP | 115O |
| HETATM | 1041 | O | HOH B | 90 | −4.929 | 48.818 | 40.338 | 1.00 | 16.04 | 1RHP | 1151 |
| HETATM | 1042 | O | HOH B | 91 | −17.260 | 23.347 | 32.284 | 1.00 | 34.49 | 1RHP | 1152 |
| HETATM | 1043 | O | HOH B | 92 | −7.802 | 47.434 | 38.776 | 1.00 | 26.98 | 1RHP | 1153 |
| HETATM | 1044 | O | HOH B | 93 | −3.557 | 34.876 | 26.628 | 1.00 | 28.47 | 1RHP | 1154 |
| HETATM | 1045 | O | HOH B | 94 | −17.438 | 19.376 | 33.177 | 1.00 | 35.28 | 1RHP | 1155 |
| ATOM | 1046 | N | ASP C | 7 | 12.167 | 11.391 | 46.389 | 1.00 | 27.00 | 1RHP | 1156 |
| ATOM | 1047 | CA | ASP C | 7 | 12.246 | 11.387 | 47.847 | 1.00 | 27.74 | 1RHP | 1157 |
| ATOM | 1048 | C | ASP C | 7 | 11.091 | 12.367 | 48.199 | 1.00 | 28.14 | 1RHP | 1158 |
| ATOM | 1049 | O | ASP C | 7 | 10.057 | 12.045 | 47.592 | 1.00 | 30.35 | 1RHP | 1159 |
| ATOM | 1050 | CB | ASP C | 7 | 13.673 | 11.878 | 48.333 | 1.00 | 26.85 | 1RHP | 1160 |
| ATOM | 1051 | CG | ASP C | 7 | 14.971 | 11.110 | 47.939 | 1.00 | 26.10 | 1RHP | 1161 |
| ATOM | 1052 | OD1 | ASP C | 7 | 14.915 | 10.047 | 47.299 | 1.00 | 28.98 | 1RHP | 1162 |
| ATOM | 1053 | OD2 | ASP C | 7 | 16.060 | 11.586 | 48.294 | 1.00 | 22.57 | 1RHP | 1163 |
| ATOM | 1054 | N | LEU C | 8 | 11.136 | 13.494 | 48.958 | 1.00 | 27.62 | 1RHP | 1164 |
| ATOM | 1055 | CA | LEU C | 8 | 10.020 | 14.388 | 49.355 | 1.00 | 26.87 | 1RHP | 1165 |
| ATOM | 1056 | C | LEU C | 8 | 9.486 | 14.017 | 50.731 | 1.00 | 25.75 | 1RHP | 1166 |
| ATOM | 1057 | O | LEU C | 8 | 9.440 | 12.825 | 51.032 | 1.00 | 25.30 | 1RHP | 1167 |
| ATOM | 1058 | CB | LEU C | 8 | 8.780 | 14.346 | 48.470 | 1.00 | 29.82 | 1RHP | 1168 |
| ATOM | 1059 | CG | LEU C | 8 | 9.021 | 14.770 | 47.033 | 1.00 | 31.49 | 1RHP | 1169 |
| ATOM | 1060 | CD1 | LEU C | 8 | 8.034 | 14.065 | 46.075 | 1.00 | 33.26 | 1RHP | 1170 |
| ATOM | 1061 | CD2 | LEU C | 8 | 9.009 | 16.282 | 47.026 | 1.00 | 31.74 | 1RHP | 1171 |
| ATOM | 1062 | N | GLN C | 9 | 8.941 | 14.997 | 51.465 | 1.00 | 23.85 | 1RHP | 1172 |
| ATOM | 1063 | CA | GLN C | 9 | 8.672 | 14.902 | 52.905 | 1.00 | 25.61 | 1RHP | 1173 |
| ATOM | 1064 | C | GLN C | 9 | 7.227 | 14.956 | 53.449 | 1.00 | 26.46 | 1RHP | 1174 |
| ATOM | 1065 | O | GLN C | 9 | 6.222 | 15.031 | 52.714 | 1.00 | 29.00 | 1RHP | 1175 |
| ATOM | 1066 | CB | GLN C | 9 | 9.504 | 16.039 | 53.563 | 1.00 | 23.45 | 1RHP | 1176 |
| ATOM | 1067 | CG | GLN C | 9 | 8.808 | 17.424 | 53.789 | 1.00 | 21.38 | 1RHP | 1177 |
| ATOM | 1068 | CD | GLN C | 9 | 8.174 | 18.224 | 52.636 | 1.00 | 20.67 | 1RHP | 1178 |
| ATOM | 1069 | OE1 | GLN C | 9 | 7.738 | 17.705 | 51.611 | 1.00 | 18.40 | 1RHP | 1179 |
| ATOM | 1070 | NE2 | GLN C | 9 | 8.093 | 19.538 | 52.693 | 1.00 | 17.94 | 1RHP | 1180 |
| ATOM | 1071 | N | CYS C | 10 | 7.157 | 14.966 | 54.794 | 1.00 | 25.67 | 1RHP | 1181 |
| ATOM | 1072 | CA | CYS C | 10 | 5.931 | 15.279 | 55.505 | 1.00 | 23.73 | 1RHP | 1182 |
| ATOM | 1073 | C | CYS C | 10 | 5.509 | 16.741 | 55.357 | 1.00 | 22.68 | 1RHP | 1183 |
| ATOM | 1074 | O | CYS C | 10 | 6.349 | 17.629 | 55.512 | 1.00 | 21.79 | 1RHP | 1184 |
| ATOM | 1075 | CB | CYS C | 10 | 6.096 | 15.000 | 56.999 | 1.00 | 23.32 | 1RHP | 1185 |
| ATOM | 1076 | SG | CYS C | 10 | 6.376 | 13.237 | 57.308 | 1.00 | 26.25 | 1RHP | 1186 |
| ATOM | 1077 | N | LEU C | 11 | 4.255 | 17.096 | 55.081 | 1.00 | 20.26 | 18RHP | 1187 |
| ATOM | 1078 | CA | LEU C | 11 | 3.867 | 18.492 | 55.169 | 1.00 | 16.64 | 1RHP | 1188 |
| ATOM | 1079 | C | LEU C | 11 | 3.635 | 18.751 | 56.668 | 1.00 | 17.78 | 1RHP | 1189 |
| ATOM | 1080 | O | LEU C | 11 | 4.349 | 19.551 | 57.277 | 1.00 | 16.76 | 1RHP | 1190 |
| ATOM | 1081 | CB | LEU C | 11 | 2.610 | 18.682 | 54.397 | 1.00 | 12.85 | 1RHP | 1191 |
| ATOM | 1082 | CG | LEU C | 11 | 2.266 | 20.030 | 53.877 | 1.00 | 9.54 | 1RHP | 1192 |
| ATOM | 1083 | CD1 | LEU C | 11 | 2.697 | 20.113 | 52.433 | 1.00 | 9.46 | 1RHP | 1193 |
| ATOM | 1084 | CD2 | LEU C | 11 | 0.762 | 20.238 | 53.950 | 1.00 | 10.01 | 1RHP | 1194 |
| ATOM | 1085 | N | CYS C | 12 | 2.734 | 18.006 | 57.336 | 1.00 | 16.99 | 1RHP | 1195 |
| ATOM | 1086 | CA | CYS C | 12 | 2.400 | 18.262 | 58.736 | 1.00 | 17.81 | 1RHP | 1196 |
| ATOM | 1087 | C | CYS C | 12 | 3.287 | 17.784 | 59.859 | 1.00 | 18.19 | 1RHP | 1197 |
| ATOM | 1088 | O | CYS C | 12 | 3.313 | 16.600 | 60.239 | 1.00 | 18.90 | 1RHP | 1198 |
| ATOM | 1089 | CB | CYS C | 12 | 1.045 | 17.730 | 59.150 | 1.00 | 16.39 | 1RHP | 1199 |
| ATOM | 1090 | SG | CYS C | 12 | −0.238 | 18.672 | 58.335 | 1.00 | 15.19 | 1RHP | 1200 |
| ATOM | 1091 | N | VAL C | 13 | 4.046 | 18.755 | 60.345 | 1.00 | 18.14 | 1RHP | 1201 |
| ATOM | 1092 | CA | VAL C | 13 | 4.737 | 18.565 | 61.590 | 1.00 | 19.30 | 1RHP | 1202 |
| ATOM | 1093 | C | VAL C | 13 | 4.351 | 19.678 | 62.572 | 1.00 | 20.29 | 1RHP | 1203 |
| ATOM | 1094 | O | VAL C | 13 | 4.154 | 19.427 | 63.757 | 1.00 | 22.46 | 1RHP | 1204 |
| ATOM | 1095 | CB | VAL C | 13 | 6.243 | 18.549 | 61.341 | 1.00 | 17.50 | 1RHP | 1205 |
| ATOM | 1096 | CG1 | VAL C | 13 | 6.825 | 19.903 | 60.902 | 1.00 | 17.16 | 1RHP | 1206 |
| ATOM | 1097 | CG2 | VAL C | 13 | 6.821 | 18.015 | 62.646 | 1.00 | 19.37 | 1RHP | 1207 |
| ATOM | 1098 | N | LYS C | 14 | 4.184 | 20.930 | 62.181 | 1.00 | 21.26 | 1RHP | 1208 |
| ATOM | 1099 | CA | LYS C | 14 | 3.913 | 21.945 | 63.183 | 1.00 | 22.54 | 1RHP | 1209 |
| ATOM | 1100 | C | LYS C | 14 | 2.393 | 21.895 | 63.297 | 1.00 | 22.88 | 1RHP | 1210 |
| ATOM | 1101 | O | LYS C | 14 | 1.625 | 22.049 | 62.324 | 1.00 | 24.98 | 1RHP | 1211 |
| ATOM | 1102 | CB | LYS C | 14 | 4.478 | 23.236 | 62.644 | 1.00 | 23.30 | 1RHP | 1212 |
| ATOM | 1103 | CG | LYS C | 14 | 5.974 | 23.067 | 62.344 | 1.00 | 26.29 | 1RHP | 1213 |
| ATOM | 1104 | CD | LYS C | 14 | 6.804 | 23.050 | 63.610 | 1.00 | 26.58 | 1RHP | 1214 |
| ATOM | 1105 | CE | LYS C | 14 | 7.402 | 24.462 | 63.597 | 1.00 | 27.18 | 1RHP | 1215 |
| ATOM | 1106 | NZ | LYS C | 14 | 8.149 | 24.818 | 64.800 | 1.00 | 29.62 | 1RHP | 1216 |
| ATOM | 1107 | N | THR C | 15 | 1.924 | 21.611 | 64.493 | 1.00 | 19.82 | 1RHP | 1217 |
| ATOM | 1108 | CA | THR C | 15 | 0.528 | 21.309 | 64.654 | 1.00 | 15.66 | 1RHP | 1218 |
| ATOM | 1109 | C | THR C | 15 | −0.161 | 22.215 | 65.648 | 1.00 | 18.53 | 1RHP | 1219 |
| ATOM | 1110 | O | THR C | 15 | 0.362 | 22.332 | 66.767 | 1.00 | 21.38 | 1RHP | 1220 |
| ATOM | 1111 | CB | THR C | 15 | 0.656 | 19.854 | 64.977 | 1.00 | 12.83 | 1RHP | 1221 |
| ATOM | 1112 | OG1 | THR C | 15 | 0.592 | 19.254 | 63.692 | 1.00 | 11.08 | 1RHP | 1222 |
| ATOM | 1113 | CG2 | THR C | 15 | −0.290 | 19.335 | 65.985 | 1.00 | 7.85 | 1RHP | 1223 |
| ATOM | 1114 | N | THR C | 16 | −1.304 | 22.856 | 65.287 | 1.00 | 18.15 | 1RHP | 1224 |
| ATOM | 1115 | CA | THR C | 16 | −1.977 | 23.780 | 66.200 | 1.00 | 16.66 | 1RHP | 1225 |
| ATOM | 1116 | C | THR C | 16 | −3.330 | 23.272 | 66.687 | 1.00 | 17.77 | 1RHP | 1226 |
| ATOM | 1117 | O | THR C | 16 | −3.916 | 22.306 | 66.175 | 1.00 | 16.61 | 1RHP | 1227 |
| ATOM | 1118 | CB | THR C | 16 | −2.081 | 25.185 | 65.494 | 1.00 | 15.70 | 1RHP | 1228 |
| ATOM | 1119 | OG1 | THR C | 16 | −2.551 | 26.147 | 66.437 | 1.00 | 15.98 | 1RHP | 1229 |
| ATOM | 1120 | CG2 | THR C | 16 | −3.002 | 25.168 | 64.332 | 1.00 | 14.32 | 1RHP | 1230 |
| ATOM | 1121 | N | SER C | 17 | −3.700 | 23.903 | 67.807 | 1.00 | 19.29 | 1RHP | 1231 |
| ATOM | 1122 | CA | SER C | 17 | −4.953 | 23.708 | 68.494 | 1.00 | 18.26 | 1RHP | 1232 |
| ATOM | 1123 | C | SER C | 17 | −5.555 | 25.028 | 68.925 | 1.00 | 18.09 | 1RHP | 1233 |
| ATOM | 1124 | O | SER C | 17 | −6.429 | 25.061 | 69.792 | 1.00 | 18.96 | 1RHP | 1234 |
| ATOM | 1125 | CB | SER C | 17 | −4.769 | 22.826 | 69.720 | 1.00 | 19.54 | 1RHP | 1235 |
| ATOM | 1126 | OG | SER C | 17 | −4.693 | 21.459 | 69.311 | 1.00 | 22.25 | 1RHP | 1236 |
| ATOM | 1127 | N | GLN C | 18 | −5.146 | 26.162 | 68.359 | 1.00 | 18.24 | 1RHP | 1237 |
| ATOM | 1128 | CA | GLN C | 18 | −5.868 | 27.398 | 68.656 | 1.00 | 20.51 | 1RHP | 1238 |
| ATOM | 1129 | O | GLN C | 18 | −6.643 | 27.780 | 67.363 | 1.00 | 21.73 | 1RHP | 1239 |
| ATOM | 1130 | O | GLN C | 18 | −6.312 | 28.682 | 66.578 | 1.00 | 21.55 | 1RHP | 1240 |
| ATOM | 1131 | CB | GLN C | 18 | −4.855 | 28.500 | 69.144 | 1.00 | 18.57 | 1RHP | 1241 |
| ATOM | 1132 | CG | GLN C | 18 | −4.255 | 28.119 | 70.531 | 1.00 | 16.31 | 1RHP | 1242 |
| ATOM | 1133 | CD | GLN C | 18 | −4.117 | 29.224 | 71.584 | 1.00 | 14.73 | 1RHP | 1243 |
| ATOM | 1134 | OE1 | GLN C | 18 | −4.186 | 30.431 | 71.334 | 1.00 | 13.97 | 1RHP | 1244 |
| ATOM | 1135 | NE2 | GLN C | 18 | −3.963 | 28.807 | 72.827 | 1.00 | 13.01 | 1RHP | 1245 |
| ATOM | 1136 | N | VAL C | 19 | −7.717 | 27.001 | 67.157 | 1.00 | 21.87 | 1RHP | 1246 |
| ATOM | 1137 | CA | VAL C | 19 | −8.564 | 27.081 | 65.986 | 1.00 | 19.95 | 1RHP | 1247 |
| ATOM | 1138 | C | VAL C | 19 | −10.026 | 27.289 | 66.367 | 1.00 | 23.44 | 1RHP | 1248 |
| ATOM | 1139 | O | VAL C | 19 | −10.504 | 26.629 | 67.313 | 1.00 | 25.69 | 1RHP | 1249 |
| ATOM | 1140 | CB | VAL C | 19 | −8.394 | 25.778 | 65.193 | 1.00 | 19.87 | 1RHP | 1250 |
| ATOM | 1141 | CG1 | VAL C | 19 | −8.914 | 24.501 | 65.878 | 1.00 | 15.93 | 1RHP | 1251 |
| ATOM | 1142 | CG2 | VAL C | 19 | −9.114 | 26.057 | 63.911 | 1.00 | 17.97 | 1RHP | 1252 |
| ATOM | 1143 | N | ARG C | 20 | −10.776 | 28.173 | 65.680 | 1.00 | 24.01 | 1RHP | 1253 |
| ATOM | 1144 | CA | ARG C | 20 | −12.207 | 28.270 | 66.003 | 1.00 | 24.09 | 1RHP | 1254 |
| ATOM | 1145 | C | ARG C | 20 | −12.901 | 27.247 | 65.087 | 1.00 | 24.47 | 1RHP | 1255 |
| ATOM | 1146 | O | ARG C | 20 | −13.012 | 27.616 | 63.903 | 1.00 | 26.75 | 1RHP | 1256 |
| ATOM | 1147 | CB | ARG C | 20 | −12.781 | 29.696 | 65.722 | 1.00 | 23.75 | 1RHP | 1257 |
| ATOM | 1148 | CG | ARG C | 20 | −12.225 | 30.771 | 66.640 | 1.00 | 24.28 | 1RHP | 1258 |
| ATOM | 1149 | CD | ARG C | 20 | −12.998 | 32.130 | 66.738 | 1.00 | 27.61 | 1RHP | 1259 |
| ATOM | 1150 | NE | ARG C | 20 | −13.211 | 32.971 | 65.527 | 1.00 | 30.14 | 1RHP | 1260 |
| ATOM | 1151 | CZ | ARG C | 20 | −13.434 | 34.325 | 65.567 | 1.00 | 32.49 | 1RHP | 1261 |
| ATOM | 1152 | NH1 | ARG C | 20 | −13.301 | 35.026 | 66.712 | 1.00 | 35.34 | 1RHP | 1262 |
| ATOM | 1153 | NH2 | ARG C | 20 | −13.657 | 35.069 | 64.454 | 1.00 | 33.58 | 1RHP | 1263 |
| ATOM | 1154 | N | PRO C | 21 | −13.418 | 26.022 | 65.413 | 1.00 | 23.95 | 1RHP | 1264 |
| ATOM | 1155 | CA | PRO C | 21 | −14.154 | 25.154 | 64.466 | 1.00 | 23.04 | 1RHP | 1265 |
| ATOM | 1156 | C | PRO C | 21 | −15.201 | 25.946 | 63.701 | 1.00 | 25.37 | 1RHP | 1266 |
| ATOM | 1157 | O | PRO C | 21 | −15.479 | 25.589 | 62.557 | 1.00 | 26.02 | 1RHP | 1267 |
| ATOM | 1158 | CB | PRO C | 21 | −14.781 | 24.027 | 65.274 | 1.00 | 22.03 | 1RHP | 1268 |
| ATOM | 1159 | CG | PRO C | 21 | −14.724 | 24.534 | 66.690 | 1.00 | 20.18 | 1RHP | 1269 |
| ATOM | 1160 | CD | PRO C | 21 | −13.430 | 25.388 | 66.725 | 1.00 | 22.71 | 1RHP | 1270 |
| ATOM | 1161 | N | ARG C | 22 | −15.666 | 27.104 | 64.241 | 1.00 | 25.93 | 1RHP | 1271 |
| ATOM | 1162 | CA | ARG C | 22 | −16.628 | 27.973 | 63.585 | 1.00 | 24.90 | 1RHP | 1272 |
| ATOM | 1163 | C | ARG C | 22 | −15.975 | 28.842 | 62.527 | 1.00 | 23.89 | 1RHP | 1273 |
| ATOM | 1164 | O | ARG C | 22 | −16.435 | 29.967 | 62.294 | 1.00 | 22.00 | 1RHP | 1274 |
| ATOM | 1165 | CB | ARG C | 22 | −17.374 | 28.840 | 64.663 | 1.00 | 24.99 | 1RHP | 1275 |
| ATOM | 1166 | CG | ARG C | 22 | −18.722 | 28.142 | 65.034 | 1.00 | 26.31 | 1RHP | 1276 |
| ATOM | 1167 | CD | ARG C | 22 | −18.955 | 27.628 | 66.512 | 1.00 | 26.29 | 1RHP | 1277 |
| ATOM | 1168 | NE | ARG C | 22 | −19.535 | 28.593 | 67.473 | 1.00 | 27.93 | 1RHP | 1278 |
| ATOM | 1169 | CZ | ARG C | 22 | −20.038 | 28.252 | 68.694 | 1.00 | 27.26 | 1RHP | 1279 |
| ATOM | 1170 | NH1 | ARG C | 22 | −20.061 | 26.997 | 69.177 | 1.00 | 24.63 | 1RHP | 1280 |
| ATOM | 1171 | NH2 | ARG C | 22 | −20.512 | 29.213 | 69.501 | 1.00 | 25.48 | 1RHP | 1281 |
| ATOM | 1172 | N | HIS C | 23 | −14.931 | 28.334 | 61.852 | 1.00 | 23.14 | 1RHP | 1282 |
| ATOM | 1173 | CA | HIS C | 23 | −14.238 | 29.038 | 60.775 | 1.00 | 25.16 | 1RHP | 1283 |
| ATOM | 1174 | C | HIS C | 23 | −13.406 | 28.107 | 59.864 | 1.00 | 23.32 | 1RHP | 1284 |
| ATOM | 1175 | O | HIS C | 23 | −12.482 | 28.566 | 59.189 | 1.00 | 23.91 | 1RHP | 1285 |
| ATOM | 1176 | CB | HIS C | 23 | −13.325 | 30.161 | 61.391 | 1.00 | 25.80 | 1RHP | 1286 |
| ATOM | 1177 | CG | HIS C | 23 | −13.956 | 31.559 | 61.565 | 1.00 | 25.80 | 1RHP | 1287 |
| ATOM | 1178 | ND1 | HIS C | 23 | −15.130 | 32.004 | 61.099 | 1.00 | 25.91 | 1RHP | 1288 |
| ATOM | 1179 | CD2 | HIS C | 23 | −13.361 | 32.643 | 62.186 | 1.00 | 27.67 | 1RHP | 1289 |
| ATOM | 1180 | CE1 | HIS C | 23 | −15.259 | 33.279 | 61.389 | 1.00 | 25.02 | 1RHP | 1290 |
| ATOM | 1181 | NE2 | HIS C | 23 | −14.187 | 33.653 | 62.039 | 1.00 | 26.38 | 1RHP | 1291 |
| ATOM | 1182 | N | ILE C | 24 | −13.707 | 26.810 | 59.756 | 1.00 | 21.17 | 1RHP | 1292 |
| ATOM | 1183 | CA | ILE C | 24 | −12.971 | 25.846 | 58.929 | 1.00 | 18.86 | 1RHP | 1293 |
| ATOM | 1184 | C | ILE C | 24 | −13.911 | 25.445 | 57.777 | 1.00 | 18.28 | 1RHP | 1294 |
| ATOM | 1185 | O | ILE C | 24 | −15.080 | 25.081 | 57.927 | 1.00 | 22.14 | 1RHP | 1295 |
| ATOM | 1186 | CB | ILE C | 24 | −12.533 | 24.604 | 59.821 | 1.00 | 13.21 | 1RHP | 1296 |
| ATOM | 1187 | CG1 | ILE C | 24 | −11.439 | 25.001 | 60.845 | 1.00 | 9.90 | 1RHP | 1297 |
| ATOM | 1188 | CG2 | ILE C | 24 | −12.010 | 23.505 | 58.937 | 1.00 | 11.82 | 1RHP | 1298 |
| ATOM | 1189 | CD1 | ILE C | 24 | −10.828 | 23.823 | 61.627 | 1.00 | 2.35 | 1RHP | 1299 |
| ATOM | 1190 | N | THR C | 25 | −13.321 | 25.486 | 56.610 | 1.00 | 17.57 | 1RHP | 1300 |
| ATOM | 1191 | CA | THR C | 25 | −13.987 | 25.336 | 55.354 | 1.00 | 14.81 | 1RHP | 1301 |
| ATOM | 1192 | C | THR C | 25 | −13.778 | 23.941 | 54.805 | 1.00 | 15.91 | 1RHP | 1302 |
| ATOM | 1193 | O | THR C | 25 | −14.693 | 23.365 | 54.220 | 1.00 | 19.78 | 1RHP | 1303 |
| ATOM | 1194 | CB | THR C | 25 | −13.355 | 26.515 | 54.618 | 1.00 | 13.81 | 1RHP | 1304 |
| ATOM | 1195 | OG1 | THR C | 25 | −14.137 | 27.605 | 55.087 | 1.00 | 11.52 | 1RHP | 1305 |
| ATOM | 1196 | CG2 | THR C | 25 | −13.240 | 26.406 | 53.123 | 1.00 | 12.27 | 1RHP | 1306 |
| ATOM | 1197 | N | SER C | 26 | −12.597 | 23.357 | 54.952 | 1.00 | 16.41 | 1RHP | 1307 |
| ATOM | 1198 | CA | SER C | 26 | −12.303 | 22.021 | 54.430 | 1.00 | 15.23 | 1RHP | 1308 |
| ATOM | 1199 | C | SER C | 26 | −11.586 | 21.380 | 55.576 | 1.00 | 11.10 | 1RHP | 1309 |
| ATOM | 1200 | O | SER C | 26 | −11.220 | 22.050 | 56.541 | 1.00 | 13.68 | 1RHP | 1310 |
| ATOM | 1201 | CB | SER C | 26 | −11.263 | 21.913 | 53.286 | 1.00 | 17.29 | 1RHP | 1311 |
| ATOM | 1202 | OG | SER C | 26 | −11.381 | 22.673 | 52.082 | 1.00 | 22.75 | 1RHP | 1312 |
| ATOM | 1203 | N | LEU C | 27 | −11.375 | 20.097 | 55.440 | 1.00 | 8.00 | 1RHP | 1313 |
| ATOM | 1204 | CA | LEU C | 27 | −10.504 | 19.351 | 56.309 | 1.00 | 7.87 | 1RHP | 1314 |
| ATOM | 1205 | C | LEU C | 27 | −10.100 | 18.266 | 55.335 | 1.00 | 6.53 | 1RHP | 1315 |
| ATOM | 1206 | O | LEU C | 27 | −10.909 | 17.809 | 54.530 | 1.00 | 7.33 | 1RHP | 1316 |
| ATOM | 1207 | CB | LEU C | 27 | −11.289 | 18.855 | 57.493 | 1.00 | 8.86 | 1RHP | 1317 |
| ATOM | 1208 | CG | LEU C | 27 | −10.621 | 17.902 | 58.448 | 1.00 | 11.58 | 1RHP | 1318 |
| ATOM | 1209 | CD1 | LEU C | 27 | −10.876 | 18.345 | 59.867 | 1.00 | 12.04 | 1RHP | 1319 |
| ATOM | 1210 | CD2 | LEU C | 27 | −11.187 | 16.500 | 58.258 | 1.00 | 8.51 | 1RHP | 1320 |
| ATOM | 1211 | N | GLU C | 28 | −8.828 | 17.941 | 55.297 | 1.00 | 7.13 | 1RHP | 1321 |
| ATOM | 1212 | CA | GLU C | 28 | −8.302 | 16.962 | 54.385 | 1.00 | 6.39 | 1RHP | 1322 |
| ATOM | 1213 | C | GLU C | 28 | −7.628 | 15.948 | 55.297 | 1.00 | 7.75 | 1RHP | 1323 |
| ATOM | 1214 | O | GLU C | 28 | −6.964 | 16.343 | 56.262 | 1.00 | 7.35 | 1RHP | 1324 |
| ATOM | 1215 | CB | GLU C | 28 | −7.378 | 17.724 | 53.508 | 1.00 | 3.22 | 1RHP | 1325 |
| ATOM | 1216 | CG | GLU C | 28 | −7.239 | 17.076 | 52.168 | 1.00 | 2.88 | 1RHP | 1326 |
| ATOM | 1217 | CD | GLU C | 28 | −6.498 | 17.911 | 51.146 | 1.00 | 2.89 | 1RHP | 1327 |
| ATOM | 1218 | OE1 | GLU C | 28 | −6.447 | 19.143 | 51.188 | 1.00 | 2.38 | 1RHP | 1328 |
| ATOM | 1219 | OE2 | GLU C | 28 | −5.965 | 17.285 | 50.255 | 1.00 | 5.33 | 1RHP | 1329 |
| ATOM | 1220 | N | VAL C | 29 | −7.813 | 14.649 | 55.130 | 1.00 | 7.35 | 1RHP | 1330 |
| ATOM | 1221 | CA | VAL C | 29 | −7.185 | 13.674 | 56.000 | 1.00 | 7.77 | 1RHP | 1331 |
| ATOM | 1222 | C | VAL C | 29 | −6.281 | 12.894 | 55.064 | 1.00 | 9.16 | 1RHP | 1332 |
| ATOM | 1223 | O | VAL C | 29 | −6.783 | 12.368 | 54.071 | 1.00 | 13.88 | 1RHP | 1333 |
| ATOM | 1224 | CB | VAL C | 29 | −8.307 | 12.821 | 56.643 | 1.00 | 7.98 | 1RHP | 1334 |
| ATOM | 1225 | CG1 | VAL C | 29 | −7.808 | 11.485 | 57.116 | 1.00 | 8.51 | 1RHP | 1335 |
| ATOM | 1226 | CG2 | VAL C | 29 | −8.772 | 13.500 | 57.930 | 1.00 | 6.29 | 1RHP | 1336 |
| ATOM | 1227 | N | ILE C | 30 | −4.970 | 12.802 | 55.254 | 1.00 | 7.71 | 1RHP | 1337 |
| ATOM | 1228 | CA | ILE C | 30 | −4.140 | 12.115 | 54.290 | 1.00 | 8.40 | 1RHP | 1338 |
| ATOM | 1229 | C | ILE C | 30 | −3.575 | 10.897 | 54.962 | 1.00 | 8.05 | 1RHP | 1339 |
| ATOM | 1230 | O | ILE C | 30 | −3.133 | 10.981 | 56.100 | 1.00 | 9.35 | 1RHP | 1340 |
| ATOM | 1231 | CB | ILE C | 30 | −3.002 | 13.000 | 53.815 | 1.00 | 8.73 | 1RHP | 1341 |
| ATOM | 1232 | CG1 | ILE C | 30 | −3.458 | 14.424 | 53.547 | 1.00 | 11.24 | 1RHP | 1342 |
| ATOM | 1233 | CG2 | ILE C | 30 | −2.443 | 12.344 | 52.562 | 1.00 | 8.82 | 1RHP | 1343 |
| ATOM | 1234 | CD1 | ILE C | 30 | −2.626 | 15.198 | 52.519 | 1.00 | 8.46 | 1RHP | 1344 |
| ATOM | 1235 | N | LYS C | 31 | −3.580 | 9.776 | 54.281 | 1.00 | 10.02 | 1RHP | 1345 |
| ATOM | 1236 | CA | LYS C | 31 | −3.129 | 8.534 | 54.848 | 1.00 | 11.82 | 1RHP | 1346 |
| ATOM | 1237 | C | LYS C | 31 | −1.633 | 8.554 | 55.078 | 1.00 | 14.86 | 1RHP | 1347 |
| ATOM | 1238 | O | LYS C | 31 | −0.856 | 9.090 | 54.268 | 1.00 | 17.42 | 1RHP | 1348 |
| ATOM | 1239 | CB | LYS C | 31 | −3.486 | 7.430 | 53.901 | 1.00 | 13.31 | 1RHP | 1349 |
| ATOM | 1240 | CG | LYS C | 31 | −3.426 | 6.041 | 54.524 | 1.00 | 13.89 | 1RHP | 1350 |
| ATOM | 1241 | CD | LYS C | 31 | −3.538 | 4.985 | 53.415 | 1.00 | 13.46 | 1RHP | 1351 |
| ATOM | 1242 | CE | LYS C | 31 | −3.432 | 3.648 | 54.102 | 1.00 | 14.70 | 1RHP | 1352 |
| ATOM | 1243 | NZ | LYS C | 31 | −3.484 | 2.544 | 53.177 | 1.00 | 15.73 | 1RHP | 1353 |
| ATOM | 1244 | N | ALA C | 32 | −1.217 | 7.974 | 56.196 | 1.00 | 16.96 | 1RHP | 1354 |
| ATOM | 1245 | CA | ALA C | 32 | 0.203 | 7.889 | 56.489 | 1.00 | 18.59 | 1RHP | 1355 |
| ATOM | 1246 | C | ALA C | 32 | 0.848 | 7.053 | 55.408 | 1.00 | 19.63 | 1RHP | 1356 |
| ATOM | 1247 | O | ALA C | 32 | 0.242 | 6.038 | 55.052 | 1.00 | 20.59 | 1RHP | 1357 |
| ATOM | 1248 | CB | ALA C | 32 | 0.463 | 7.158 | 57.776 | 1.00 | 18.47 | 1RHP | 1358 |
| ATOM | 1249 | N | GLY C | 33 | 2.041 | 7.344 | 54.900 | 1.00 | 21.64 | 1RHP | 1359 |
| ATOM | 1250 | CA | GLY C | 33 | 2.659 | 6.503 | 53.883 | 1.00 | 18.91 | 1RHP | 1360 |
| ATOM | 1251 | C | GLY C | 33 | 4.122 | 6.855 | 53.743 | 1.00 | 17.85 | 1RHP | 1361 |
| ATOM | 1252 | O | GLY C | 33 | 4.614 | 7.723 | 54.463 | 1.00 | 16.79 | 1RHP | 1362 |
| ATOM | 1253 | N | PRO C | 34 | 4.878 | 6.248 | 52.840 | 1.00 | 15.38 | 1RHP | 1363 |
| ATOM | 1254 | CA | PRO C | 34 | 6.184 | 6.684 | 52.382 | 1.00 | 16.06 | 1RHP | 1364 |
| ATOM | 1255 | C | PRO C | 34 | 6.766 | 8.072 | 52.720 | 1.00 | 19.85 | 1RHP | 1365 |
| ATOM | 1256 | O | PRO C | 34 | 7.854 | 8.162 | 53.262 | 1.00 | 19.97 | 1RHP | 1366 |
| ATOM | 1257 | CB | PRO C | 34 | 6.026 | 6.402 | 50.923 | 1.00 | 14.50 | 1RHP | 1367 |
| ATOM | 1258 | CG | PRO C | 34 | 5.034 | 5.248 | 50.827 | 1.00 | 11.38 | 1RHP | 1368 |
| ATOM | 1259 | CD | PRO C | 34 | 4.583 | 4.969 | 52.249 | 1.00 | 13.74 | 1RHP | 1369 |
| ATOM | 1260 | N | HIS C | 35 | 6.060 | 9.184 | 52.467 | 1.00 | 22.43 | 1RHP | 1370 |
| ATOM | 1261 | CA | HIS C | 35 | 6.481 | 10.587 | 52.679 | 1.00 | 23.88 | 1RHP | 1371 |
| ATOM | 1262 | C | HIS C | 35 | 6.495 | 11.029 | 54.151 | 1.00 | 24.25 | 1RHP | 1372 |
| ATOM | 1263 | O | HIS C | 35 | 7.118 | 12.040 | 54.564 | 1.00 | 23.83 | 1RHP | 1373 |
| ATOM | 1264 | CB | HIS C | 35 | 5.519 | 11.586 | 51.944 | 1.00 | 25.95 | 1RHP | 1374 |
| ATOM | 1265 | CG | HIS C | 35 | 5.647 | 11.947 | 50.449 | 1.00 | 27.88 | 1RHP | 1375 |
| ATOM | 1266 | ND1 | HIS C | 35 | 5.175 | 13.076 | 49.893 | 1.00 | 28.98 | 1RHP | 1376 |
| ATOM | 1267 | CD2 | HIS C | 35 | 6.274 | 11.227 | 49.440 | 1.00 | 29.40 | 1RHP | 1377 |
| ATOM | 1268 | CE1 | HIS C | 35 | 5.490 | 13.065 | 48.611 | 1.00 | 28.39 | 1RHP | 1378 |
| ATOM | 1269 | NE2 | HIS C | 35 | 6.149 | 11.957 | 48.347 | 1.00 | 28.23 | 1RHP | 1379 |
| ATOM | 1270 | N | CYS C | 36 | 5.652 | 10.294 | 54.893 | 1.00 | 24.08 | 1RHP | 1380 |
| ATOM | 1271 | CA | CYS C | 36 | 5.346 | 10.655 | 56.244 | 1.00 | 22.94 | 1RHP | 1381 |
| ATOM | 1272 | C | CYS C | 36 | 4.821 | 9.488 | 57.031 | 1.00 | 22.41 | 1RHP | 1382 |
| ATOM | 1273 | O | CYS C | 36 | 3.809 | 8.924 | 56.621 | 1.00 | 23.35 | 1RHP | 1383 |
| ATOM | 1274 | CB | CYS C | 36 | 4.286 | 11.695 | 56.273 | 1.00 | 22.45 | 1RHP | 1384 |
| ATOM | 1275 | SG | CYS C | 36 | 4.582 | 12.432 | 57.874 | 1.00 | 24.01 | 1RHP | 1385 |
| ATOM | 1276 | N | PRO C | 37 | 5.351 | 9.079 | 58.179 | 1.00 | 22.38 | 1RHP | 1386 |
| ATOM | 1277 | CA | PRO C | 37 | 4.923 | 7.887 | 58.880 | 1.00 | 20.61 | 1RHP | 1387 |
| ATOM | 1278 | C | PRO C | 37 | 3.689 | 8.191 | 59.701 | 1.00 | 20.85 | 1RHP | 1388 |
| ATOM | 1279 | O | PRO C | 37 | 3.499 | 7.493 | 60.703 | 1.00 | 21.09 | 1RHP | 1389 |
| ATOM | 1280 | CB | PRO C | 37 | 6.123 | 7.541 | 59.687 | 1.00 | 19.35 | 1RHP | 1390 |
| ATOM | 1281 | CG | PRO C | 37 | 6.558 | 8.902 | 60.201 | 1.00 | 22.27 | 1RHP | 1391 |
| ATOM | 1282 | CD | PRO C | 37 | 6.369 | 9.786 | 58.959 | 1.00 | 22.54 | 1RHP | 1392 |
| ATOM | 1283 | N | THR C | 38 | 2.907 | 9.251 | 59.440 | 1.00 | 21.05 | 1RHP | 1393 |
| ATOM | 1284 | CA | THR C | 38 | 1.661 | 9.427 | 60.169 | 1.00 | 22.02 | 1RHP | 1394 |
| ATOM | 1285 | C | THR C | 38 | 0.669 | 10.136 | 59.282 | 1.00 | 21.59 | 1RHP | 1395 |
| ATOM | 1286 | O | THR C | 38 | 1.013 | 10.758 | 58.261 | 1.00 | 22.15 | 1RHP | 1396 |
| ATOM | 1287 | CB | THR C | 38 | 1.832 | 10.266 | 61.419 | 1.00 | 23.39 | 1RHP | 1397 |
| ATOM | 1288 | OG1 | THR C | 38 | 3.226 | 10.269 | 61.781 | 1.00 | 26.27 | 1RHP | 1398 |
| ATOM | 1289 | CG2 | THR C | 38 | 0.967 | 9.695 | 62.541 | 1.00 | 23.45 | 1RHP | 1399 |
| ATOM | 1290 | N | ALA C | 39 | −0.599 | 9.927 | 59.617 | 1.00 | 20.69 | 1RHP | 1400 |
| ATOM | 1291 | CA | ALA C | 39 | −1.675 | 10.598 | 58.912 | 1.00 | 16.27 | 1RHP | 1401 |
| ATOM | 1292 | C | ALA C | 39 | −1.566 | 12.080 | 59.245 | 1.00 | 14.47 | 1RHP | 1402 |
| ATOM | 1293 | O | ALA C | 39 | −1.052 | 12.489 | 60.297 | 1.00 | 14.04 | 1RHP | 1403 |
| ATOM | 1294 | CB | ALA C | 39 | −3.000 | 10.020 | 59.373 | 1.00 | 14.25 | 1RHP | 1404 |
| ATOM | 1295 | N | GLN C | 40 | −1.960 | 12.891 | 58.292 | 1.00 | 11.96 | 1RHP | 1405 |
| ATOM | 1296 | CA | GLN C | 40 | −1.808 | 14.315 | 58.442 | 1.00 | 13.66 | 1RHP | 1406 |
| ATOM | 1297 | C | GLN C | 40 | −3.192 | 14.898 | 58.287 | 1.00 | 14.83 | 1RHP | 1407 |
| ATOM | 1298 | O | GLN C | 40 | −3.867 | 14.507 | 57.335 | 1.00 | 16.68 | 1RHP | 1408 |
| ATOM | 1299 | CB | GLN C | 40 | −0.908 | 14.853 | 57.352 | 1.00 | 12.77 | 1RHP | 1409 |
| ATOM | 1300 | CG | GLN C | 40 | 0.417 | 14.114 | 57.177 | 1.00 | 13.21 | 1RHP | 1410 |
| ATOM | 1301 | CD | GLN C | 40 | 1.185 | 14.627 | 55.971 | 1.00 | 13.72 | 1RHP | 1411 |
| ATOM | 1302 | OE1 | GLN C | 40 | 1.754 | 15.726 | 55.972 | 1.00 | 13.29 | 1RHP | 1412 |
| ATOM | 1303 | NE2 | GLN C | 40 | 1.208 | 13.862 | 54.893 | 1.00 | 11.96 | 1RHP | 1413 |
| ATOM | 1304 | N | LEU C | 41 | −3.647 | 15.783 | 59.160 | 1.00 | 14.03 | 1RHP | 1414 |
| ATOM | 1305 | CA | LEU C | 41 | −4.945 | 16.395 | 59.079 | 1.00 | 13.36 | 1RHP | 1415 |
| ATOM | 1306 | C | LEU C | 41 | −4.708 | 17.839 | 58.707 | 1.00 | 13.45 | 1RHP | 1416 |
| ATOM | 1307 | O | LEU C | 41 | −4.384 | 18.636 | 59.585 | 1.00 | 15.22 | 1RHP | 1417 |
| ATOM | 1308 | CB | LEU C | 41 | −5.656 | 16.313 | 60.425 | 1.00 | 16.22 | 1RHP | 1418 |
| ATOM | 1309 | CG | LEU C | 41 | −6.624 | 15.143 | 60.634 | 1.00 | 18.80 | 1RHP | 1419 |
| ATOM | 1310 | CD1 | LEU C | 41 | −5.860 | 13.844 | 60.661 | 1.00 | 18.81 | 1RHP | 1420 |
| ATOM | 1311 | CD2 | LEU C | 41 | −7.310 | 15.234 | 61.968 | 1.00 | 18.09 | 1RHP | 1421 |
| ATOM | 1312 | N | ILE C | 42 | −4.850 | 18.212 | 57.444 | 1.00 | 10.23 | 1RHP | 1422 |
| ATOM | 1313 | CA | ILE C | 42 | −4.619 | 19.578 | 56.982 | 1.00 | 8.39 | 1RHP | 1423 |
| ATOM | 1314 | C | ILE C | 42 | −5.958 | 20.288 | 57.084 | 1.00 | 8.73 | 1RHP | 1424 |
| ATOM | 1315 | O | ILE C | 42 | −6.909 | 19.691 | 56.601 | 1.00 | 9.30 | 1RHP | 1425 |
| ATOM | 1316 | CB | ILE C | 42 | −4.147 | 19.561 | 55.509 | 1.00 | 8.07 | 1RHP | 1426 |
| ATOM | 1317 | CG1 | ILE C | 42 | −2.967 | 18.647 | 55.366 | 1.00 | 5.48 | 1RHP | 1427 |
| ATOM | 1318 | CG2 | ILE C | 42 | −3.754 | 20.951 | 55.054 | 1.00 | 5.60 | 1RHP | 1428 |
| ATOM | 1319 | CD1 | ILE C | 42 | −2.894 | 18.223 | 53.926 | 1.00 | 4.68 | 1RHP | 1429 |
| ATOM | 1320 | N | ALA C | 43 | −6.118 | 21.485 | 57.654 | 1.00 | 8.23 | 1RHP | 1430 |
| ATOM | 1321 | CA | ALA C | 43 | −7.399 | 22.199 | 57.737 | 1.00 | 6.73 | 1RHP | 1431 |
| ATOM | 1322 | C | ALA C | 43 | −7.334 | 23.609 | 57.183 | 1.00 | 6.32 | 1RHP | 1432 |
| ATOM | 1323 | O | ALA C | 43 | −6.610 | 24.479 | 57.644 | 1.00 | 6.95 | 1RHP | 1433 |
| ATOM | 1324 | CB | ALA C | 43 | −7.877 | 22.338 | 59.168 | 1.00 | 4.60 | 1RHP | 1434 |
| ATOM | 1325 | N | THR C | 44 | −8.060 | 23.863 | 56.139 | 1.00 | 7.46 | 1RHP | 1435 |
| ATOM | 1326 | CA | THR C | 44 | −8.084 | 25.125 | 55.456 | 1.00 | 8.80 | 1RHP | 1436 |
| ATOM | 1327 | C | THR C | 44 | −8.920 | 26.079 | 56.307 | 1.00 | 11.62 | 1RHP | 1437 |
| ATOM | 1328 | O | THR C | 44 | −10.119 | 25.822 | 56.485 | 1.00 | 17.79 | 1RHP | 1438 |
| ATOM | 1329 | CB | THR C | 44 | −8.724 | 24.835 | 54.110 | 1.00 | 9.69 | 1RHP | 1439 |
| ATOM | 1330 | OG1 | THR C | 44 | −8.237 | 23.581 | 53.651 | 1.00 | 10.89 | 1RHP | 1440 |
| ATOM | 1331 | CG2 | THR C | 44 | −8.358 | 25.840 | 53.068 | 1.00 | 13.08 | 1RHP | 1441 |
| ATOM | 1332 | N | LEU C | 45 | −8.405 | 27.142 | 56.917 | 1.00 | 13.82 | 1RHP | 1442 |
| ATOM | 1333 | CA | LEU C | 45 | −9.213 | 28.133 | 57.615 | 1.00 | 13.39 | 1RHP | 1443 |
| ATOM | 1334 | C | LEU C | 45 | −9.982 | 28.857 | 56.520 | 1.00 | 17.02 | 1RHP | 1444 |
| ATOM | 1335 | O | LEU C | 45 | −9.548 | 28.964 | 55.363 | 1.00 | 17.66 | 1RHP | 1445 |
| ATOM | 1336 | CB | LEU C | 45 | −8.349 | 29.160 | 58.326 | 1.00 | 12.01 | 1RHP | 1446 |
| ATOM | 1337 | CG | LEU C | 45 | −8.035 | 29.218 | 59.830 | 1.00 | 10.88 | 1RHP | 1447 |
| ATOM | 1338 | CD1 | LEU C | 45 | −8.864 | 30.342 | 60.459 | 1.00 | 12.78 | 1RHP | 1448 |
| ATOM | 1339 | CD2 | LEU C | 45 | −8.258 | 27.869 | 60.480 | 1.00 | 9.08 | 1RHP | 1449 |
| ATOM | 1340 | N | LYS C | 46 | −11.127 | 29.409 | 56.898 | 1.00 | 18.94 | 1RHP | 1450 |
| ATOM | 1341 | CA | LYS C | 46 | −11.975 | 30.129 | 55.975 | 1.00 | 19.52 | 1RHP | 1451 |
| ATOM | 1342 | C | LYS C | 46 | −11.210 | 31.263 | 55.308 | 1.00 | 19.26 | 1RHP | 1452 |
| ATOM | 1343 | O | LYS C | 46 | −11.162 | 31.280 | 54.088 | 1.00 | 19.19 | 1RHP | 1453 |
| ATOM | 1344 | CB | LYS C | 46 | −13.204 | 30.663 | 56.728 | 1.00 | 20.96 | 1RHP | 1454 |
| ATOM | 1345 | CG | LYS C | 46 | −14.446 | 30.853 | 55.800 | 1.00 | 23.85 | 1RHP | 1455 |
| ATOM | 1346 | CD | LYS C | 46 | −15.834 | 30.737 | 56.507 | 1.00 | 24.50 | 1RHP | 1456 |
| ATOM | 1347 | CE | LYS C | 46 | −16.107 | 29.393 | 57.233 | 1.00 | 24.31 | 1RHP | 1457 |
| ATOM | 1348 | NZ | LYS C | 46 | −17.406 | 29.400 | 57.882 | 1.00 | 22.25 | 1RHP | 1458 |
| ATOM | 1349 | N | ASN C | 47 | −10.504 | 32.184 | 55.931 | 1.00 | 19.46 | 1RHP | 1459 |
| ATOM | 1350 | CA | ASN C | 47 | −9.881 | 33.246 | 55.170 | 1.00 | 18.95 | 1RHP | 1460 |
| ATOM | 1351 | C | ASN C | 47 | −8.701 | 32.919 | 54.274 | 1.00 | 19.44 | 1RHP | 1461 |
| ATOM | 1352 | O | ASN C | 47 | −7.837 | 33.788 | 54.060 | 1.00 | 22.96 | 1RHP | 1462 |
| ATOM | 1353 | CB | ASN C | 47 | −9.481 | 34.309 | 56.134 | 1.00 | 19.64 | 1RHP | 1463 |
| ATOM | 1354 | CG | ASN C | 47 | −8.381 | 33.830 | 57.035 | 1.00 | 20.16 | 1RHP | 1464 |
| ATOM | 1355 | OD1 | ASN C | 47 | −8.696 | 33.322 | 58.115 | 1.00 | 20.16 | 1RHP | 1465 |
| ATOM | 1356 | ND2 | ASN C | 47 | −7.114 | 33.950 | 56.636 | 1.00 | 17.22 | 1RHP | 1466 |
| ATOM | 1357 | N | GLY C | 48 | −8.546 | 31.693 | 53.781 | 1.00 | 18.52 | 1RHP | 1467 |
| ATOM | 1358 | CA | GLY C | 48 | −7.441 | 31.367 | 52.875 | 1.00 | 16.75 | 1RHP | 1468 |
| ATOM | 1359 | C | GLY C | 48 | −6.495 | 30.292 | 53.395 | 1.00 | 15.78 | 1RHP | 1469 |
| ATOM | 1360 | O | GLY C | 48 | −6.439 | 29.146 | 52.930 | 1.00 | 12.27 | 1RHP | 1470 |
| ATOM | 1361 | N | ARG C | 49 | −5.752 | 30.760 | 54.383 | 1.00 | 15.95 | 1RHP | 1471 |
| ATOM | 1362 | CA | ARG C | 49 | −4.690 | 29.987 | 55.003 | 1.00 | 17.01 | 1RHP | 1472 |
| ATOM | 1363 | C | ARG C | 49 | −5.032 | 28.618 | 55.551 | 1.00 | 15.75 | 1RHP | 1473 |
| ATOM | 1364 | O | ARG C | 49 | −6.144 | 28.446 | 56.039 | 1.00 | 15.37 | 1RHP | 1474 |
| ATOM | 1365 | CB | ARG C | 49 | −4.065 | 30.662 | 56.194 | 1.00 | 20.82 | 1RHP | 1475 |
| ATOM | 1366 | CG | ARG C | 49 | −4.513 | 32.041 | 56.530 | 1.00 | 24.81 | 1RHP | 1476 |
| ATOM | 1367 | CD | ARG C | 49 | −3.812 | 33.257 | 55.888 | 1.00 | 28.50 | 1RHP | 1477 |
| ATOM | 1368 | NE | ARG C | 49 | −4.080 | 34.190 | 56.951 | 1.00 | 27.87 | 1RHP | 1478 |
| ATOM | 1369 | CZ | ARG C | 49 | −3.491 | 35.335 | 57.110 | 1.00 | 28.51 | 1RHP | 1479 |
| ATOM | 1370 | NH1 | ARG C | 49 | −2.679 | 35.834 | 56.195 | 1.00 | 27.90 | 1RHP | 1480 |
| ATOM | 1371 | NH2 | ARG C | 49 | −3.773 | 35.978 | 58.238 | 1.00 | 28.13 | 1RHP | 1481 |
| ATOM | 1372 | N | LYS C | 50 | −4.030 | 27.723 | 55.614 | 1.00 | 12.87 | 1RHP | 1482 |
| ATOM | 1373 | CA | LYS C | 50 | −4.176 | 26.362 | 56.132 | 1.00 | 10.89 | 1RHP | 1483 |
| ATOM | 1374 | C | LYS C | 50 | −3.375 | 26.151 | 57.433 | 1.00 | 8.52 | 1RHP | 1484 |
| ATOM | 1375 | O | LYS C | 50 | −2.550 | 27.004 | 57.763 | 1.00 | 7.32 | 1RHP | 1485 |
| ATOM | 1376 | CB | LYS C | 50 | −3.706 | 25.377 | 55.063 | 1.00 | 8.64 | 1RHP | 1486 |
| ATOM | 1377 | CG | LYS C | 50 | −4.263 | 25.740 | 53.730 | 1.00 | 9.78 | 1RHP | 1487 |
| ATOM | 1378 | CD | LYS C | 50 | −3.745 | 24.823 | 52.696 | 1.00 | 13.15 | 1RHP | 1488 |
| ATOM | 1379 | CE | LYS C | 50 | −4.506 | 23.531 | 52.847 | 1.00 | 17.10 | 1RHP | 1489 |
| ATOM | 1380 | NZ | LYS C | 50 | −5.789 | 23.593 | 52.160 | 1.00 | 20.25 | 1RHP | 1490 |
| ATOM | 1381 | N | ILE C | 51 | −3.575 | 25.083 | 58.205 | 1.00 | 7.65 | 1RHP | 1491 |
| ATOM | 1382 | CA | ILE C | 51 | −2.817 | 24.786 | 59.417 | 1.00 | 12.40 | 1RHP | 1492 |
| ATOM | 1383 | C | ILE C | 51 | −2.872 | 23.291 | 59.721 | 1.00 | 14.08 | 1RHP | 1493 |
| ATOM | 1384 | O | ILE C | 51 | −3.921 | 22.699 | 59.517 | 1.00 | 16.46 | 1RHP | 1494 |
| ATOM | 1385 | CB | ILE C | 51 | −3.363 | 25.458 | 60.682 | 1.00 | 10.95 | 1RHP | 1495 |
| ATOM | 1386 | CG1 | ILE C | 51 | −4.870 | 25.315 | 60.729 | 1.00 | 10.65 | 1RHP | 1496 |
| ATOM | 1387 | CG2 | ILE C | 51 | −2.931 | 26.892 | 60.719 | 1.00 | 11.31 | 1RHP | 1497 |
| ATOM | 1388 | CD1 | ILE C | 51 | −5.514 | 25.678 | 62.067 | 1.00 | 11.50 | 1RHP | 1498 |
| ATOM | 1389 | N | CYS C | 52 | −1.849 | 22.584 | 60.170 | 1.00 | 14.44 | 1RHP | 1499 |
| ATOM | 1390 | CA | CYS C | 52 | −2.017 | 21.178 | 60.474 | 1.00 | 15.58 | 1RHP | 1500 |
| ATOM | 1391 | C | CYS C | 52 | −2.657 | 21.015 | 61.828 | 1.00 | 15.49 | 1RHP | 1501 |
| ATOM | 1392 | O | CYS C | 52 | −2.286 | 21.738 | 62.754 | 1.00 | 17.27 | 1RHP | 1502 |
| ATOM | 1393 | CB | CYS C | 52 | −0.696 | 20.456 | 60.520 | 1.00 | 16.30 | 1RHP | 1503 |
| ATOM | 1394 | SG | CYS C | 52 | 0.148 | 20.507 | 58.940 | 1.00 | 14.00 | 1RHP | 1504 |
| ATOM | 1395 | N | LEU C | 53 | −3.527 | 20.026 | 61.979 | 1.00 | 16.67 | 1RHP | 1505 |
| ATOM | 1396 | CA | LEU C | 53 | −4.244 | 19.742 | 63.201 | 1.00 | 16.29 | 1RHP | 1506 |
| ATOM | 1397 | C | LEU C | 53 | −3.700 | 18.548 | 63.996 | 1.00 | 16.09 | 1RHP | 1507 |
| ATOM | 1398 | O | LEU C | 53 | −3.257 | 17.477 | 63.539 | 1.00 | 12.22 | 1RHP | 1508 |
| ATOM | 1399 | CB | LEU C | 53 | −5.706 | 19.445 | 62.907 | 1.00 | 15.90 | 1RHP | 1509 |
| ATOM | 1400 | CG | LEU C | 53 | −6.817 | 20.429 | 62.692 | 1.00 | 14.76 | 1RHP | 1510 |
| ATOM | 1401 | CD1 | LEU C | 53 | −8.048 | 19.596 | 62.424 | 1.00 | 11.02 | 1RHP | 1511 |
| ATOM | 1402 | CD2 | LEU C | 53 | −7.053 | 21.324 | 63.904 | 1.00 | 13.01 | 1RHP | 1512 |
| ATOM | 1403 | N | ASP C | 54 | −3.847 | 18.716 | 65.293 | 1.00 | 15.46 | 1RHP | 1513 |
| ATOM | 1404 | CA | ASP C | 54 | −3.336 | 17.717 | 66.175 | 1.00 | 16.44 | 1RHP | 1514 |
| ATOM | 1405 | C | ASP C | 54 | −4.232 | 16.496 | 66.131 | 1.00 | 17.89 | 1RHP | 1515 |
| ATOM | 1406 | O | ASP C | 54 | −5.413 | 16.498 | 66.449 | 1.00 | 18.29 | 1RHP | 1516 |
| ATOM | 1407 | CB | ASP C | 54 | −3.234 | 18.353 | 67.579 | 1.00 | 16.49 | 1RHP | 1517 |
| ATOM | 1408 | CG | ASP C | 54 | −2.045 | 17.926 | 68.442 | 1.00 | 13.82 | 1RHP | 1518 |
| ATOM | 1409 | OD1 | ASP C | 54 | −1.763 | 16.737 | 68.471 | 1.00 | 14.66 | 1RHP | 1519 |
| ATOM | 1410 | OD2 | ASP C | 54 | −1.397 | 18.771 | 69.071 | 1.00 | 14.80 | 1RHP | 1520 |
| ATOM | 1411 | N | LEU C | 55 | −3.569 | 15.448 | 65.686 | 1.00 | 19.38 | 1RHP | 1521 |
| ATOM | 1412 | CA | LEU C | 55 | −4.081 | 14.091 | 65.713 | 1.00 | 20.31 | 1RHP | 1522 |
| ATOM | 1413 | C | LEU C | 55 | −3.921 | 13.508 | 67.116 | 1.00 | 19.25 | 1RHP | 1523 |
| ATOM | 1414 | O | LEU C | 55 | −4.073 | 12.299 | 67.306 | 1.00 | 18.01 | 1RHP | 1524 |
| ATOM | 1415 | CB | LEU C | 55 | −3.311 | 13.215 | 64.717 | 1.00 | 20.95 | 1RHP | 1525 |
| ATOM | 1416 | CG | LEU C | 55 | −3.990 | 11.896 | 64.352 | 1.00 | 24.02 | 1RHP | 1526 |
| ATOM | 1417 | CD1 | LEU C | 55 | −5.269 | 12.267 | 63.589 | 1.00 | 22.26 | 1RHP | 1527 |
| ATOM | 1418 | CD2 | LEU C | 55 | −3.099 | 10.975 | 63.514 | 1.00 | 22.86 | 1RHP | 1528 |
| ATOM | 1419 | N | GLN C | 56 | −3.493 | 14.262 | 68.131 | 1.00 | 20.14 | 1RHP | 1529 |
| ATOM | 1420 | CA | GLN C | 56 | −3.516 | 13.739 | 69.515 | 1.00 | 24.66 | 1RHP | 1530 |
| ATOM | 1421 | C | GLN C | 56 | −4.542 | 14.605 | 70.258 | 1.00 | 24.72 | 1RHP | 1531 |
| ATOM | 1422 | O | GLN C | 56 | −4.864 | 14.428 | 71.450 | 1.00 | 24.83 | 1RHP | 1532 |
| ATOM | 1423 | CB | GLN C | 56 | −2.109 | 13.839 | 70.289 | 1.00 | 24.63 | 1RHP | 1533 |
| ATOM | 1424 | CG | GLN C | 56 | −1.686 | 15.132 | 71.056 | 1.00 | 28.71 | 1RHP | 1534 |
| ATOM | 1425 | CD | GLN C | 56 | −0.465 | 15.073 | 72.000 | 1.00 | 29.59 | 1RHP | 1535 |
| ATOM | 1426 | OE1 | GLN C | 56 | 0.108 | 14.012 | 72.319 | 1.00 | 31.55 | 1RHP | 1536 |
| ATOM | 1427 | NE2 | GLN C | 56 | −0.014 | 16.224 | 72.516 | 1.00 | 30.57 | 1RHP | 1537 |
| ATOM | 1428 | N | ALA C | 57 | −5.060 | 15.606 | 69.542 | 1.00 | 23.57 | 1RHP | 1538 |
| ATOM | 1429 | CA | ALA C | 57 | −5.960 | 16.507 | 70.200 | 1.00 | 23.86 | 1RHP | 1539 |
| ATOM | 1430 | C | ALA C | 57 | −7.318 | 15.825 | 70.073 | 1.00 | 23.95 | 1RHP | 1540 |
| ATOM | 1431 | O | ALA C | 57 | −7.645 | 15.152 | 69.089 | 1.00 | 22.98 | 1RHP | 1541 |
| ATOM | 1432 | CB | ALA C | 57 | −6.020 | 17.859 | 69.499 | 1.00 | 24.24 | 1RHP | 1542 |
| ATOM | 1433 | N | PRO C | 58 | −8.121 | 15.970 | 71.109 | 1.00 | 23.08 | 1RHP | 1543 |
| ATOM | 1434 | CA | PRO C | 58 | −9.555 | 15.808 | 71.036 | 1.00 | 23.38 | 1RHP | 1544 |
| ATOM | 1435 | C | PRO C | 58 | −10.164 | 16.580 | 69.883 | 1.00 | 21.52 | 1RHP | 1545 |
| ATOM | 1436 | O | PRO C | 58 | −10.829 | 15.999 | 69.024 | 1.00 | 21.64 | 1RHP | 1546 |
| ATOM | 1437 | CB | PRO C | 58 | −10.022 | 16.278 | 72.370 | 1.00 | 24.42 | 1RHP | 1547 |
| ATOM | 1438 | CG | PRO C | 58 | −8.882 | 17.214 | 72.780 | 1.00 | 23.19 | 1RHP | 1548 |
| ATOM | 1439 | CD | PRO C | 58 | −7.690 | 16.360 | 72.442 | 1.00 | 23.75 | 1RHP | 1549 |
| ATOM | 1440 | N | LEU C | 59 | −9.907 | 17.881 | 69.835 | 1.00 | 19.22 | 1RHP | 1550 |
| ATOM | 1441 | CA | LEU C | 59 | −10.478 | 18.785 | 68.871 | 1.00 | 18.29 | 1RHP | 1551 |
| ATOM | 1442 | C | LEU C | 59 | −10.862 | 18.369 | 67.460 | 1.00 | 18.98 | 1RHP | 1552 |
| ATOM | 1443 | O | LEU C | 59 | −11.760 | 19.034 | 66.968 | 1.00 | 20.69 | 1RHP | 1553 |
| ATOM | 1444 | CB | LEU C | 59 | −9.530 | 19.932 | 68.843 | 1.00 | 17.55 | 1RHP | 1554 |
| ATOM | 1445 | CG | LEU C | 59 | −9.777 | 21.251 | 68.191 | 1.00 | 16.59 | 1RHP | 1555 |
| ATOM | 1446 | CD1 | LEU C | 59 | −11.173 | 21.766 | 68.444 | 1.00 | 17.81 | 1RHP | 1556 |
| ATOM | 1447 | CD2 | LEU C | 59 | −8.741 | 22.202 | 68.778 | 1.00 | 16.36 | 1RHP | 1557 |
| ATOM | 1448 | N | TYR C | 60 | −10.391 | 17.370 | 66.704 | 1.00 | 19.60 | 1RHP | 1558 |
| ATOM | 1449 | CA | TYR C | 60 | −10.915 | 17.102 | 65.340 | 1.00 | 20.24 | 1RHP | 1559 |
| ATOM | 1450 | C | TYR C | 60 | −12.235 | 16.307 | 65.367 | 1.00 | 19.53 | 1RHP | 1560 |
| ATOM | 1451 | O | TYR C | 60 | −12.825 | 15.992 | 64.328 | 1.00 | 19.74 | 1RHP | 1561 |
| ATOM | 1452 | CB | TYR C | 60 | −9.885 | 16.323 | 64.483 | 1.00 | 20.83 | 1RHP | 1562 |
| ATOM | 1453 | CG | TYR C | 60 | −9.763 | 14.835 | 64.786 | 1.00 | 20.54 | 1RHP | 1563 |
| ATOM | 1454 | CD1 | TYR C | 60 | −9.064 | 14.400 | 65.891 | 1.00 | 18.22 | 1RHP | 1564 |
| ATOM | 1455 | CD2 | TYR C | 60 | −10.451 | 13.939 | 64.000 | 1.00 | 19.49 | 1RHP | 1565 |
| ATOM | 1456 | CE1 | TYR C | 60 | −9.049 | 13.060 | 66.216 | 1.00 | 18.66 | 1RHP | 1566 |
| ATOM | 1457 | CE2 | TYR C | 60 | −10.439 | 12.604 | 64.320 | 1.00 | 20.10 | 1RHP | 1567 |
| ATOM | 1458 | CZ | TYR C | 60 | −9.739 | 12.180 | 65.422 | 1.00 | 17.13 | 1RHP | 1568 |
| ATOM | 1459 | OH | TYR C | 60 | −9.761 | 10.847 | 65.738 | 1.00 | 21.91 | 1RHP | 1569 |
| ATOM | 1460 | N | LYS C | 61 | −12.651 | 15.898 | 66.567 | 1.00 | 18.84 | 1RHP | 1570 |
| ATOM | 1461 | CA | LYS C | 61 | −13.960 | 15.310 | 66.797 | 1.00 | 19.41 | 1RHP | 157I |
| ATOM | 1462 | C | LYS C | 61 | −14.961 | 16.380 | 66.447 | 1.00 | 18.76 | 1RHP | 1572 |
| ATOM | 1463 | O | LYS C | 61 | −15.631 | 16.292 | 65.426 | 1.00 | 17.04 | 1RHP | 1573 |
| ATOM | 1464 | CB | LYS C | 61 | −14.177 | 14.934 | 68.262 | 1.00 | 21.61 | 1RHP | 1574 |
| ATOM | 1465 | CG | LYS C | 61 | −13.217 | 13.803 | 68.581 | 1.00 | 26.45 | 1RHP | 1575 |
| ATOM | 1466 | CD | LYS C | 61 | −13.477 | 13.137 | 69.922 | 1.00 | 30.73 | 1RHP | 1576 |
| ATOM | 1467 | CE | LYS C | 61 | −12.340 | 12.127 | 70.148 | 1.00 | 30.54 | 1RHP | 1577 |
| ATOM | 1468 | NZ | LYS C | 61 | −11.057 | 12.844 | 70.252 | 1.00 | 34.82 | 1RHP | 1578 |
| ATOM | 1469 | N | LYS C | 62 | −14.930 | 17.452 | 67.250 | 1.00 | 19.13 | 1RHP | 1579 |
| ATOM | 1470 | CA | LYS C | 62 | −15.837 | 18.581 | 67.138 | 1.00 | 19.72 | 1RHP | 1580 |
| ATOM | 1471 | C | LYS C | 62 | −15.972 | 19.058 | 65.701 | 1.00 | 20.81 | 1RHP | 1581 |
| ATOM | 1472 | O | LYS C | 62 | −17.090 | 19.067 | 65.152 | 1.00 | 23.03 | 1RHP | 1582 |
| ATOM | 1473 | CB | LYS C | 62 | −15.349 | 19.771 | 67.974 | 1.00 | 22.32 | 1RHP | 1583 |
| ATOM | 1474 | CG | LYS C | 62 | −16.443 | 20.752 | 68.411 | 1.00 | 24.23 | 1RHP | 1584 |
| ATOM | 1475 | CD | LYS C | 62 | −17.130 | 19.967 | 69.511 | 1.00 | 27.07 | 1RHP | 1585 |
| ATOM | 1476 | CE | LYS C | 62 | −18.471 | 20.469 | 69.995 | 1.00 | 29.46 | 1RHP | 1586 |
| ATOM | 1477 | NZ | LYS C | 62 | −18.267 | 21.613 | 70.861 | 1.00 | 31.50 | 1RHP | 1587 |
| ATOM | 1478 | N | ILE C | 63 | −14.817 | 19.338 | 65.079 | 1.00 | 18.18 | 1RHP | 1588 |
| ATOM | 1479 | CA | ILE C | 63 | −14.764 | 19.883 | 63.744 | 1.00 | 14.14 | 1RHP | 1589 |
| ATOM | 1480 | C | ILE C | 63 | −15.480 | 18.964 | 62.774 | 1.00 | 14.44 | 1RHP | 1590 |
| ATOM | 1481 | O | ILE C | 63 | −16.417 | 19.442 | 62.132 | 1.00 | 14.17 | 1RHP | 1591 |
| ATOM | 1482 | CB | ILE C | 63 | −13.285 | 20.092 | 63.370 | 1.00 | 13.23 | 1RHP | 1592 |
| ATOM | 1483 | CG1 | ILE C | 63 | −12.624 | 21.065 | 64.352 | 1.00 | 12.65 | 1RHP | 1593 |
| ATOM | 1484 | CG2 | ILE C | 63 | −13.184 | 20.663 | 61.976 | 1.00 | 12.32 | 1RHP | 1594 |
| ATOM | 1485 | CD1 | ILE C | 63 | −11.104 | 21.227 | 64.208 | 1.00 | 9.86 | 1RHP | 1595 |
| ATOM | 1486 | N | ILE C | 64 | −15.254 | 17.655 | 62.668 | 1.00 | 15.03 | 1RHP | 1596 |
| ATOM | 1487 | CA | ILE C | 64 | −15.941 | 16.924 | 61.596 | 1.00 | 18.44 | 1RHP | 1597 |
| ATOM | 1488 | C | ILE C | 64 | −17.479 | 16.866 | 61.701 | 1.00 | 20.50 | 1RHP | 1598 |
| ATOM | 1489 | O | ILE C | 64 | −18.182 | 16.884 | 60.688 | 1.00 | 20.82 | 1RHP | 1599 |
| ATOM | 1490 | CB | ILE C | 64 | −15.278 | 15.523 | 61.520 | 1.00 | 16.61 | 1RHP | 1600 |
| ATOM | 1491 | CG1 | ILE C | 64 | −14.168 | 15.679 | 60.486 | 1.00 | 16.99 | 1RHP | 1601 |
| ATOM | 1492 | CG2 | ILE C | 64 | −16.204 | 14.397 | 61.089 | 1.00 | 17.11 | 1RHP | 1602 |
| ATOM | 1493 | CD1 | ILE C | 64 | −13.205 | 14.483 | 60.353 | 1.00 | 19.22 | 1RHP | 1603 |
| ATOM | 1494 | N | LYS C | 65 | −18.023 | 16.900 | 62.920 | 1.00 | 24.34 | 1RHP | 1604 |
| ATOM | 1495 | CA | LYS C | 65 | −19.457 | 16.939 | 63.182 | 1.00 | 25.15 | 1RHP | 1605 |
| ATOM | 1496 | C | LYS C | 65 | −19.907 | 18.238 | 62.532 | 1.00 | 26.01 | 1RHP | 1606 |
| ATOM | 1497 | O | LYS C | 65 | −20.609 | 18.194 | 61.501 | 1.00 | 25.76 | 1RHP | 1607 |
| ATOM | 1498 | CB | LYS C | 65 | −19.711 | 16.982 | 64.681 | 1.00 | 25.45 | 1RHP | 1608 |
| ATOM | 1499 | CG | LYS C | 65 | −21.077 | 17.427 | 65.188 | 1.00 | 25.85 | 1RHP | 1609 |
| ATOM | 1500 | CD | LYS C | 65 | −21.021 | 17.239 | 66.723 | 1.00 | 25.50 | 1RHP | 1610 |
| ATOM | 1501 | CE | LYS C | 65 | −21.919 | 18.181 | 67.555 | 1.00 | 24.36 | 1RHP | 1611 |
| ATOM | 1502 | NZ | LYS C | 65 | −21.492 | 18.213 | 68.956 | 1.00 | 24.69 | 1RHP | 1612 |
| ATOM | 1503 | N | LYS C | 66 | −19.429 | 19.409 | 62.972 | 1.00 | 23.88 | 1RHP | 1613 |
| ATOM | 1504 | CA | LYS C | 66 | −19.972 | 20.631 | 62.378 | 1.00 | 22.29 | 1RHP | 1614 |
| ATOM | 1505 | C | LYS C | 66 | −19.612 | 20.849 | 60.900 | 1.00 | 22.10 | 1RHP | 1615 |
| ATOM | 1506 | O | LYS C | 66 | −19.964 | 21.899 | 60.335 | 1.00 | 20.39 | 1RHP | 1616 |
| ATOM | 1507 | CB | LYS C | 66 | −19.547 | 21.867 | 63.186 | 1.00 | 19.61 | 1RHP | 1617 |
| ATOM | 1508 | CG | LYS C | 66 | −20.245 | 22.067 | 64.500 | 1.00 | 17.79 | 1RHP | 1618 |
| ATOM | 1509 | CD | LYS C | 66 | −19.559 | 21.271 | 65.564 | 1.00 | 20.40 | 1RHP | 1619 |
| ATOM | 1510 | CE | LYS C | 66 | −19.896 | 21.895 | 66.911 | 1.00 | 20.83 | 1RHP | 1620 |
| ATOM | 1511 | NZ | LYS C | 66 | −19.197 | 23.154 | 67.114 | 1.00 | 22.62 | 1RHP | 1621 |
| ATOM | 1512 | N | LEU C | 67 | −18.901 | 19.918 | 60.227 | 1.00 | 19.86 | 1RHP | 1622 |
| ATOM | 1513 | CA | LEU C | 67 | −18.724 | 20.053 | 58.777 | 1.00 | 16.77 | 1RHP | 1623 |
| ATOM | 1514 | C | LEU C | 67 | −19.669 | 19.122 | 58.057 | 1.00 | 15.19 | 1RHP | 1624 |
| ATOM | 1515 | O | LEU C | 67 | −20.100 | 19.376 | 56.940 | 1.00 | 14.99 | 1RHP | 1625 |
| ATOM | 1516 | CB | LEU C | 67 | −17.297 | 19.734 | 58.307 | 1.00 | 11.52 | 1RHP | 1626 |
| ATOM | 1517 | CG | LEU C | 67 | −16.122 | 20.645 | 58.683 | 1.00 | 8.95 | 1RHP | 1627 |
| ATOM | 1518 | CD1 | LEU C | 67 | −15.361 | 20.821 | 57.408 | 1.00 | 10.50 | 1RHP | 1628 |
| ATOM | 1519 | CD2 | LEU C | 67 | −16.495 | 22.030 | 59.178 | 1.00 | 7.43 | 1RHP | 1629 |
| ATOM | 1520 | N | LEU C | 68 | −20.040 | 18.039 | 58.703 | 1.00 | 16.44 | 1RHP | 1630 |
| ATOM | 1521 | CA | LEU C | 68 | −20.987 | 17.133 | 58.117 | 1.00 | 18.15 | 1RHP | 1631 |
| ATOM | 1522 | C | LEU C | 68 | −22.400 | 17.549 | 58.512 | 1.00 | 20.98 | 1RHP | 1632 |
| ATOM | 1523 | O | LEU C | 68 | −23.391 | 17.039 | 57.958 | 1.00 | 20.49 | 1RHP | 1633 |
| ATOM | 1524 | CB | LEU C | 68 | −20.630 | 15.738 | 58.591 | 1.00 | 16.29 | 1RHP | 1634 |
| ATOM | 1525 | CG | LEU C | 68 | −19.259 | 15.317 | 58.063 | 1.00 | 16.67 | 1RHP | 1635 |
| ATOM | 1526 | CD1 | LEU C | 68 | −18.728 | 14.099 | 58.795 | 1.00 | 16.28 | 1RHP | 1636 |
| ATOM | 1527 | CD2 | LEU C | 68 | −19.389 | 15.080 | 56.586 | 1.00 | 15.05 | 1RHP | 1637 |
| ATOM | 1528 | N | GLU C | 69 | −22.567 | 18.496 | 59.437 | 1.00 | 20.66 | 1RHP | 1638 |
| ATOM | 1529 | CA | GLU C | 69 | −23.915 | 18.922 | 59.749 | 1.00 | 22.32 | 1RHP | 1639 |
| ATOM | 1530 | C | GLU C | 69 | −24.537 | 19.892 | 58.721 | 1.00 | 21.03 | 1RHP | 1640 |
| ATOM | 1531 | O | GLU C | 69 | −24.695 | 21.104 | 58.943 | 1.00 | 18.46 | 1RHP | 1641 |
| ATOM | 1532 | CB | GLU C | 69 | −23.928 | 19.553 | 61.135 | 1.00 | 24.05 | 1RHP | 1642 |
| ATOM | 1533 | CG | GLU C | 69 | −23.318 | 18.749 | 62.255 | 1.00 | 27.39 | 1RHP | 1643 |
| ATOM | 1534 | CD | GLU C | 69 | −24.106 | 17.597 | 62.859 | 1.00 | 28.74 | 1RHP | 1644 |
| ATOM | 1535 | OE1 | GLU C | 69 | −24.273 | 16.546 | 62.206 | 1.00 | 30.01 | 1RHP | 1645 |
| ATOM | 1536 | OE2 | GLU C | 69 | −24.531 | 17.778 | 64.017 | 1.00 | 31.20 | 1RHP | 1646 |
| ATOM | 1537 | N | SER C | 70 | −24.902 | 19.318 | 57.567 | 1.00 | 23.43 | 1RHP | 1647 |
| ATOM | 1538 | CA | SER C | 70 | −25.652 | 19.973 | 56.485 | 1.00 | 26.03 | 1RHP | 1648 |
| ATOM | 1539 | C | SER C | 70 | −26.162 | 18.999 | 55.382 | 1.00 | 26.62 | 1RHP | 1649 |
| ATOM | 1540 | O | SER C | 70 | −26.707 | 17.912 | 55.657 | 1.00 | 29.15 | 1RHP | 1650 |
| ATOM | 1541 | CB | SER C | 70 | −24.822 | 21.044 | 55.755 | 1.00 | 27.68 | 1RHP | 1651 |
| ATOM | 1542 | OG | SER C | 70 | −24.148 | 21.997 | 56.581 | 1.00 | 25.67 | 1RHP | 1652 |
| TER | 1543 | | SER C | 70 | | | | | | 1RHP | 1653 |
| HETATM | 1544 | O | HOH C | 71 | 5.265 | 13.592 | 60.317 | 1.00 | 17.64 | 1RHP | 1654 |
| HETATM | 1545 | O | HOH C | 72 | −0.443 | 1.906 | 56.286 | 1.00 | 9.73 | 1RHP | 1655 |
| HETATM | 1546 | O | HOH C | 73 | −5.053 | 38.787 | 55.488 | 1.00 | 22.52 | 1RHP | 1656 |
| HETATM | 1547 | O | HOH C | 74 | 7.338 | 21.236 | 55.629 | 1.00 | 39.58 | 1RHP | 1657 |
| HETATM | 1548 | O | HOH C | 75 | −8.132 | 20.895 | 54.002 | 1.00 | 34.71 | 1RHP | 1658 |
| HETATM | 1549 | O | HOH C | 76 | −2.390 | 6.601 | 58.853 | 1.00 | 26.18 | 1RHP | 1659 |
| HETATM | 1550 | O | HOH C | 77 | 11.030 | 24.828 | 65.249 | 1.00 | 12.07 | 1RHP | 1660 |
| HETATM | 1551 | O | HOH C | 78 | −2.115 | 17.117 | 61.059 | 1.00 | 25.66 | 1RHP | 1661 |
| HETATM | 1552 | O | HOH C | 79 | −19.760 | 24.508 | 61.337 | 1.00 | 7.09 | 1RHP | 1662 |
| HETATM | 1553 | O | HOH C | 80 | −20.148 | 25.031 | 63.973 | 1.00 | 20.26 | 1RHP | 1663 |
| HETATM | 1554 | O | HOH C | 81 | 0.522 | 15.409 | 61.980 | 1.00 | 32.34 | 1RHP | 1664 |
| HETATM | 1555 | O | HOH C | 82 | −26.619 | 15.251 | 57.227 | 1.00 | 41.89 | 1RHP | 1665 |
| HETATM | 1556 | O | HOH C | 83 | −23.118 | 25.348 | 66.285 | 1.00 | 36.09 | 1RHP | 1666 |
| HETATM | 1557 | O | HOH C | 84 | 12.382 | 26.078 | 62.961 | 1.00 | 31.58 | 1RHP | 1667 |
| HETATM | 1558 | O | HOH C | 85 | −16.404 | 33.881 | 64.066 | 1.00 | 30.94 | 1RHP | 1668 |
| HETATM | 1559 | O | HOH C | 86 | −20.608 | 30.276 | 57.378 | 1.00 | 19.39 | 1RHP | 1669 |
| ATOM | 1560 | N | ASP D | 7 | −17.805 | 32.317 | 39.767 | 1.00 | 14.52 | 1RHP | 1670 |
| ATOM | 1561 | CA | ASP D | 7 | −18.716 | 31.584 | 40.626 | 1.00 | 18.32 | 1RHP | 1671 |
| ATOM | 1562 | C | ASP D | 7 | −18.497 | 30.089 | 40.453 | 1.00 | 17.38 | 1RHP | 1672 |
| ATOM | 1563 | O | ASP D | 7 | −18.200 | 29.613 | 39.345 | 1.00 | 16.68 | 1RHP | 1673 |
| ATOM | 1564 | CB | ASP D | 7 | −20.143 | 31.890 | 40.260 | 1.00 | 21.96 | 1RHP | 1674 |
| ATOM | 1565 | CG | ASP D | 7 | −21.035 | 32.355 | 41.403 | 1.00 | 25.68 | 1RHP | 1675 |
| ATOM | 1566 | OD1 | ASP D | 7 | −20.584 | 33.100 | 42.287 | 1.00 | 25.83 | 1RHP | 1676 |
| ATOM | 1567 | OD2 | ASP D | 7 | −22.203 | 31.966 | 41.402 | 1.00 | 31.15 | 1RHP | 1677 |
| ATOM | 1568 | N | LEU D | 8 | −18.703 | 29.340 | 41.526 | 1.00 | 14.97 | 1RHP | 1678 |
| ATOM | 1569 | CA | LEU D | 8 | −18.426 | 27.908 | 41.553 | 1.00 | 12.07 | 1RHP | 1679 |
| ATOM | 1570 | C | LEU D | 8 | −19.697 | 27.116 | 41.797 | 1.00 | 11.73 | 1RHP | 1680 |
| ATOM | 1571 | O | LEU D | 8 | −20.656 | 27.676 | 42.307 | 1.00 | 10.31 | 1RHP | 1681 |
| ATOM | 1572 | CB | LEU D | 8 | −17.421 | 27.549 | 42.680 | 1.00 | 8.34 | 1RHP | 1682 |
| ATOM | 1573 | CG | LEU D | 8 | −16.095 | 28.264 | 42.896 | 1.00 | 3.86 | 1RHP | 1683 |
| ATOM | 1574 | CD1 | LEU D | 8 | −16.381 | 29.569 | 43.563 | 1.00 | 4.23 | 1RHP | 1684 |
| ATOM | 1575 | CD2 | LEU D | 8 | −15.180 | 27.497 | 43.812 | 1.00 | 2.44 | 1RHP | 1685 |
| ATOM | 1576 | N | GLN D | 9 | −19.742 | 25.836 | 41.461 | 1.00 | 15.19 | 1RHP | 1686 |
| ATOM | 1577 | CA | GLN D | 9 | −20.889 | 24.971 | 41.688 | 1.00 | 15.25 | 1RHP | 1687 |
| ATOM | 1570 | C | GLN D | 9 | −20.475 | 23.831 | 42.598 | 1.00 | 16.80 | 1RHP | 1688 |
| ATOM | 1579 | O | GLN D | 9 | −19.403 | 23.887 | 43.209 | 1.00 | 19.16 | 1RHP | 1689 |
| ATOM | 1580 | CB | GLN D | 9 | −21.397 | 24.415 | 40.361 | 1.00 | 17.45 | 1RHP | 1690 |
| ATOM | 1581 | CG | GLN D | 9 | −20.486 | 23.610 | 39.417 | 1.00 | 18.07 | 1RHP | 1591 |
| ATOM | 1582 | CD | GLN D | 9 | −21.187 | 23.012 | 38.174 | 1.00 | 20.75 | 1RHP | 1692 |
| ATOM | 1583 | OE1 | GLN D | 9 | −20.587 | 22.605 | 37.161 | 1.00 | 22.22 | 1RHP | 1693 |
| ATOM | 1584 | NE2 | GLN D | 9 | −22.518 | 22.886 | 38.173 | 1.00 | 24.72 | 1RHP | 1694 |
| ATOM | 1585 | N | CYS D | 10 | −21.268 | 22.785 | 42.797 | 1.00 | 16.72 | 1RHP | 1695 |
| ATOM | 1586 | CA | CYS D | 10 | −20.839 | 21.657 | 43.608 | 1.00 | 15.62 | 1RHP | 1696 |
| ATOM | 1587 | C | CYS D | 10 | −19.805 | 20.907 | 42.844 | 1.00 | 15.91 | 1RHP | 1697 |
| ATOM | 1588 | O | CYS D | 10 | −20.055 | 20.652 | 41.671 | 1.00 | 17.43 | 1RHP | 1698 |
| ATOM | 1589 | CB | CYS D | 10 | −21.872 | 20.641 | 43.838 | 1.00 | 15.72 | 1RHP | 1699 |
| ATOM | 1590 | SG | CYS D | 10 | −23.189 | 21.476 | 44.662 | 1.00 | 15.05 | 1RHP | 1700 |
| ATOM | 1591 | N | LEU D | 11 | −18.718 | 20.484 | 43.478 | 1.00 | 15.87 | 1RHP | 1701 |
| ATOM | 1592 | CA | LEU D | 11 | −17.746 | 19.673 | 42.787 | 1.00 | 14.28 | 1RHP | 1702 |
| ATOM | 1593 | C | LEU D | 11 | −18.306 | 18.283 | 42.460 | 1.00 | 14.13 | 1RHP | 1703 |
| ATOM | 1594 | O | LEU D | 11 | −17.949 | 17.663 | 41.444 | 1.00 | 14.73 | 1RHP | 1704 |
| ATOM | 1595 | CB | LEU D | 11 | −16.537 | 19.575 | 43.673 | 1.00 | 16.16 | 1RHP | 1705 |
| ATOM | 1596 | CG | LEU D | 11 | −15.298 | 18.961 | 43.092 | 1.00 | 17.80 | 1RHP | 1706 |
| ATOM | 1597 | CD1 | LEU D | 11 | −14.834 | 19.802 | 41.918 | 1.00 | 19.43 | 1RHP | 1707 |
| ATOM | 1598 | CD2 | LEU D | 11 | −14.186 | 18.967 | 44.103 | 1.00 | 16.24 | 1RHP | 1708 |
| ATOM | 1599 | N | CYS D | 12 | −19.157 | 11.716 | 43.329 | 1.00 | 11.99 | 1RHP | 1709 |
| ATOM | 1600 | CA | CYS D | 12 | −19.722 | 16.374 | 43.197 | 1.00 | 9.52 | 1RHP | 1710 |
| ATOM | 1601 | C | CYS D | 12 | −21.015 | 16.389 | 42.415 | 1.00 | 10.12 | 1RHP | 1711 |
| ATOM | 1602 | O | CYS D | 12 | −21.858 | 17.255 | 42.663 | 1.00 | 12.74 | 1RHP | 1712 |
| ATOM | 1603 | CB | CYS D | 12 | −19.970 | 15.783 | 44.586 | 1.00 | 9.64 | 1RHP | 1713 |
| ATOM | 1604 | SG | CYS D | 12 | −18.441 | 15.632 | 45.545 | 1.00 | 12.15 | 1RHP | 1714 |
| ATOM | 1605 | N | VAL D | 13 | −21.209 | 15.399 | 41.529 | 1.00 | 10.06 | 1RHP | 1715 |
| ATOM | 1606 | CA | VAL D | 13 | −22.371 | 15.346 | 40.660 | 1.00 | 8.05 | 1RHP | 1716 |
| ATOM | 1607 | C | VAL D | 13 | −23.080 | 14.005 | 40.656 | 1.00 | 7.83 | 1RHP | 1717 |
| ATOM | 1608 | O | VAL D | 13 | −23.957 | 13.784 | 39.850 | 1.00 | 7.76 | 1RHP | 1718 |
| ATOM | 1609 | CB | VAL D | 13 | −21.884 | 15.723 | 39.265 | 1.00 | 7.65 | 1RHP | 1719 |
| ATOM | 1610 | CG1 | VAL D | 13 | −21.376 | 14.495 | 38.529 | 1.00 | 6.24 | 1RHP | 1720 |
| ATOM | 1611 | CG2 | VAL D | 13 | −22.997 | 16.458 | 38.552 | 1.00 | 8.76 | 1RHP | 1721 |
| ATOM | 1612 | N | LYS D | 14 | −22.710 | 13.077 | 41.506 | 1.00 | 9.96 | 1RHP | 1722 |
| ATOM | 1613 | CA | LYS D | 14 | −23.223 | 11.702 | 41.573 | 1.00 | 11.20 | 1RHP | 1723 |
| ATOM | 1614 | C | LYS D | 14 | −22.712 | 11.254 | 42.918 | 1.00 | 11.76 | 1RHP | 1724 |
| ATOM | 1615 | O | LYS D | 14 | −21.897 | 11.944 | 43.542 | 1.00 | 10.23 | 1RHP | 1725 |
| ATOM | 1616 | CB | LYS D | 14 | −22.570 | 10.656 | 40.701 | 1.00 | 12.03 | 1RHP | 1726 |
| ATOM | 1617 | CG | LYS D | 14 | −22.793 | 10.390 | 39.235 | 1.00 | 14.46 | 1RHP | 1727 |
| ATOM | 1618 | CD | LYS D | 14 | −21.716 | 9.409 | 38.733 | 1.00 | 16.61 | 1RHP | 1728 |
| ATOM | 1619 | CE | LYS D | 14 | −21.620 | 8.020 | 39.411 | 1.00 | 16.90 | 1RHP | 1729 |
| ATOM | 1620 | NZ | LYS D | 14 | −21.035 | 8.062 | 40.749 | 1.00 | 17.64 | 1RHP | 1730 |
| ATOM | 1621 | N | THR D | 15 | −23.087 | 10.088 | 43.387 | 1.00 | 12.10 | 1RHP | 1731 |
| ATOM | 1622 | CA | THR D | 15 | −22.490 | 9.606 | 44.591 | 1.00 | 12.35 | 1RHP | 1732 |
| ATOM | 1623 | C | THR D | 15 | −22.225 | 8.136 | 44.370 | 1.00 | 12.78 | 1RHP | 1733 |
| ATOM | 1624 | O | THR D | 15 | −22.701 | 7.501 | 43.422 | 1.00 | 14.19 | 1RHP | 1734 |
| ATOM | 1625 | CB | THR D | 15 | −23.434 | 9.914 | 45.743 | 1.00 | 13.04 | 1RHP | 1735 |
| ATOM | 1626 | OG1 | THR D | 15 | −23.441 | 11.331 | 45.842 | 1.00 | 14.19 | 1RHP | 1736 |
| ATOM | 1627 | CG2 | THR D | 15 | −22.976 | 9.396 | 47.085 | 1.00 | 13.63 | 1RHP | 1737 |
| ATOM | 1628 | N | THR D | 16 | −21.263 | 7.743 | 45.184 | 1.00 | 14.08 | 1RHP | 1738 |
| ATOM | 1629 | CA | THR D | 16 | −20.664 | 6.453 | 45.175 | 1.00 | 17.02 | 1RHP | 1739 |
| ATOM | 1630 | C | THR D | 16 | −21.055 | 5.770 | 46.456 | 1.00 | 20.54 | 1RHP | 1740 |
| ATOM | 1631 | O | THR D | 16 | −21.018 | 6.398 | 47.525 | 1.00 | 21.70 | 1RHP | 1741 |
| ATOM | 1632 | CB | THR D | 16 | −19.161 | 6.653 | 45.071 | 1.00 | 17.51 | 1RHP | 1742 |
| ATOM | 1633 | OG1 | THR D | 16 | −18.911 | 7.343 | 43.820 | 1.00 | 18.30 | 1RHP | 1743 |
| ATOM | 1634 | CG2 | THR D | 16 | −18.429 | 5.327 | 45.211 | 1.00 | 15.19 | 1RHP | 1744 |
| ATOM | 1635 | N | SER D | 17 | −21.554 | 4.544 | 46.236 | 1.00 | 24.15 | 1RHP | 1745 |
| ATOM | 1636 | CA | SER D | 17 | −21.846 | 3.611 | 47.304 | 1.00 | 24.54 | 1RHP | 1746 |
| ATOM | 1637 | C | SER D | 17 | −20.734 | 2.575 | 47.337 | 1.00 | 23.10 | 1RHP | 1747 |
| ATOM | 1638 | O | SER D | 17 | −20.228 | 2.248 | 48.412 | 1.00 | 20.89 | 1RHP | 1748 |
| ATOM | 1639 | CB | SER D | 17 | −23.239 | 2.932 | 47.081 | 1.00 | 27.08 | 1RHP | 1749 |
| ATOM | 1640 | OG | SER D | 17 | −23.753 | 2.871 | 45.737 | 1.00 | 28.11 | 1RHP | 1750 |
| ATOM | 1641 | N | GLN D | 18 | −20.267 | 2.012 | 46.235 | 1.00 | 21.48 | 1RHP | 1751 |
| ATOM | 1642 | CA | GLN D | 18 | −19.211 | 1.021 | 46.342 | 1.00 | 20.28 | 1RHP | 1752 |
| ATOM | 1643 | C | GLN D | 18 | −17.875 | 1.720 | 46.449 | 1.00 | 19.15 | 1RHP | 1753 |
| ATOM | 1644 | O | GLN D | 18 | −17.495 | 2.400 | 45.494 | 1.00 | 16.44 | 1RHP | 1754 |
| ATOM | 1645 | CB | GLN D | 18 | −19.158 | 0.104 | 45.115 | 1.00 | 23.26 | 1RHP | 1755 |
| ATOM | 1646 | CG | GLN D | 18 | −20.258 | −0.945 | 44.951 | 1.00 | 25.98 | 1RHP | 1756 |
| ATOM | 1647 | CD | GLN D | 18 | −19.854 | −2.096 | 44.027 | 1.00 | 28.98 | 1RHP | 1757 |
| ATOM | 1648 | OE1 | GLN D | 18 | −18.840 | −2.067 | 43.309 | 1.00 | 32.76 | 1RHP | 1758 |
| ATOM | 1649 | NE2 | GLN D | 18 | −20.626 | −3.179 | 44.018 | 1.00 | 31.27 | 1RHP | 1759 |
| ATOM | 1650 | N | VAL D | 19 | −17.203 | 1.689 | 47.594 | 1.00 | 18.77 | 1RHP | 1760 |
| ATOM | 1651 | CA | VAL D | 19 | −15.810 | 2.149 | 47.663 | 1.00 | 21.15 | 1RHP | 1761 |
| ATOM | 1652 | C | VAL D | 19 | −15.070 | 1.168 | 48.551 | 1.00 | 23.05 | 1RHP | 1762 |
| ATOM | 1653 | O | VAL D | 19 | −15.634 | 0.701 | 49.559 | 1.00 | 26.28 | 1RHP | 1763 |
| ATOM | 1654 | CB | VAL D | 19 | −15.611 | 3.603 | 48.263 | 1.00 | 19.09 | 1RHP | 1764 |
| ATOM | 1655 | CG1 | VAL D | 19 | −16.065 | 3.818 | 49.695 | 1.00 | 17.46 | 1RHP | 1765 |
| ATOM | 1656 | CG2 | VAL D | 19 | −14.113 | 3.843 | 48.204 | 1.00 | 21.28 | 1RHP | 1766 |
| ATOM | 1657 | N | ARG D | 20 | −13.833 | 0.785 | 48.179 | 1.00 | 25.39 | 1RHP | 1767 |
| ATOM | 1658 | CA | ARG D | 20 | −13.115 | −0.144 | 49.065 | 1.00 | 24.52 | 1RHP | 1768 |
| ATOM | 1659 | C | ARG D | 20 | −12.239 | 0.718 | 50.004 | 1.00 | 22.14 | 1RHP | 1769 |
| ATOM | 1660 | O | ARG D | 20 | −11.249 | 1.303 | 49.539 | 1.00 | 23.14 | 1RHP | 1770 |
| ATOM | 1661 | CB | ARG D | 20 | −12.245 | −1.155 | 48.253 | 1.00 | 25.68 | 1RHP | 1771 |
| ATOM | 1662 | CG | ARG D | 20 | −12.895 | −2.003 | 47.101 | 1.00 | 28.28 | 1RHP | 1772 |
| ATOM | 1683 | CD | ARG D | 20 | −13.190 | −1.217 | 45.782 | 1.00 | 30.29 | 1RHP | 1773 |
| ATOM | 1664 | NE | ARG D | 20 | −12.145 | −0.206 | 45.706 | 1.00 | 32.25 | 1RHP | 1774 |
| ATOM | 1665 | CZ | ARG D | 20 | −12.029 | 0.785 | 44.831 | 1.00 | 32.56 | 1RHP | 1775 |
| ATOM | 1666 | NH1 | ARG D | 20 | −12.693 | 0.877 | 43.674 | 1.00 | 33.01 | 1RHP | 1776 |
| ATOM | 1667 | NH2 | ARG D | 20 | −11.106 | 1.684 | 45.140 | 1.00 | 34.30 | 1RHP | 1777 |
| ATOM | 1668 | N | PRO D | 21 | −12.585 | 0.858 | 51.312 | 1.00 | 17.61 | 1RHP | 1778 |
| ATOM | 1669 | CA | PRO D | 21 | −12.144 | 1.942 | 52.191 | 1.00 | 15.55 | 1RHP | 1779 |
| ATOM | 1670 | C | PRO D | 21 | −10.633 | 2.082 | 52.226 | 1.00 | 15.10 | 1RHP | 1780 |
| ATOM | 1671 | O | PRO D | 21 | −10.049 | 3.167 | 52.198 | 1.00 | 13.23 | 1RHP | 1781 |
| ATOM | 1672 | CB | PRO D | 21 | −12.778 | 1.619 | 53.539 | 1.00 | 14.43 | 1RHP | 1782 |
| ATOM | 1673 | CG | PRO D | 21 | −13.025 | 0.136 | 53.498 | 1.00 | 14.96 | 1RHP | 1783 |
| ATOM | 1674 | CD | PRO D | 21 | −13.437 | −0.073 | 52.046 | 1.00 | 17.34 | 1RHP | 1784 |
| ATOM | 1675 | N | ARG D | 22 | −9.981 | 0.934 | 52.174 | 1.00 | 17.60 | 1RHP | 1785 |
| ATOM | 1676 | CA | ARG D | 22 | −8.549 | 0.932 | 52.070 | 1.00 | 20.85 | 1RHP | 1786 |
| ATOM | 1677 | C | ARG D | 22 | −8.470 | 0.900 | 50.556 | 1.00 | 21.60 | 1RHP | 1787 |
| ATOM | 1678 | O | ARG D | 22 | −8.713 | −0.136 | 49.929 | 1.00 | 24.81 | 1RHP | 1788 |
| ATOM | 1679 | CB | ARG D | 22 | −7.853 | −0.332 | 52.595 | 1.00 | 24.30 | 1RHP | 1789 |
| ATOM | 1680 | CG | ARG D | 22 | −6.317 | −0.162 | 52.660 | 1.00 | 29.12 | 1RHP | 1790 |
| ATOM | 1681 | CD | ARG D | 22 | −5.384 | 0.189 | 51.428 | 1.00 | 31.45 | 1RHP | 1791 |
| ATOM | 1682 | NE | ARG D | 22 | −4.002 | 0.111 | 51.948 | 1.00 | 34.32 | 1RHP | 1792 |
| ATOM | 1683 | CZ | ARG D | 22 | −2.905 | −0.376 | 51.311 | 1.00 | 35.95 | 1RHP | 1793 |
| ATOM | 1684 | NH1 | ARG D | 22 | −2.919 | −0.816 | 50.038 | 1.00 | 37.67 | 1RHP | 1794 |
| ATOM | 1685 | NH2 | ARG D | 22 | −1.740 | −0.411 | 51.980 | 1.00 | 35.52 | 1RHP | 1795 |
| ATOM | 1686 | N | HIS D | 23 | −8.156 | 2.067 | 50.016 | 1.00 | 18.71 | 1RHP | 1796 |
| ATOM | 1687 | CA | HIS D | 23 | −7.882 | 2.379 | 48.617 | 1.00 | 17.00 | 1RHP | 1797 |
| ATOM | 1688 | C | HIS D | 23 | −7.852 | 3.880 | 48.492 | 1.00 | 17.13 | 1RHP | 1798 |
| ATOM | 1689 | O | HIS D | 23 | −7.412 | 4.442 | 47.479 | 1.00 | 17.03 | 1RHP | 1799 |
| ATOM | 1690 | CB | HIS D | 23 | −8.929 | 1.874 | 47.605 | 1.00 | 15.74 | 1RHP | 1800 |
| ATOM | 1691 | CG | HIS D | 23 | −8.431 | 0.568 | 46.992 | 1.00 | 17.47 | 1RHP | 1801 |
| ATOM | 1692 | ND1 | HIS D | 23 | −8.648 | −0.688 | 47.378 | 1.00 | 16.93 | 1RHP | 1802 |
| ATOM | 1693 | CD2 | HIS D | 23 | −7.561 | 0.489 | 45.938 | 1.00 | 18.53 | 1RHP | 1803 |
| ATOM | 1694 | CE1 | HIS D | 23 | −7.965 | −1.509 | 46.631 | 1.00 | 15.03 | 1RHP | 1804 |
| ATOM | 1695 | NE2 | HIS D | 23 | −7.306 | −0.785 | 45.772 | 1.00 | 16.07 | 1RHP | 1805 |
| ATOM | 1696 | N | ILE D | 24 | −8.369 | 4.499 | 49.550 | 1.00 | 18.03 | 1RHP | 1806 |
| ATOM | 1697 | CA | ILE D | 24 | −8.432 | 5.934 | 49.687 | 1.00 | 18.95 | 1RHP | 1807 |
| ATOM | 1698 | C | ILE D | 24 | −7.064 | 6.409 | 50.192 | 1.00 | 20.11 | 1RHP | 1808 |
| ATOM | 1699 | O | ILE D | 24 | −6.382 | 5.658 | 50.907 | 1.00 | 20.85 | 1RHP | 1809 |
| ATOM | 1700 | CB | ILE D | 24 | −9.648 | 6.175 | 50.659 | 1.00 | 17.52 | 1RHP | 1810 |
| ATOM | 1701 | CG1 | ILE D | 24 | −10.891 | 5.704 | 49.899 | 1.00 | 16.34 | 1RHP | 1811 |
| ATOM | 1702 | CG2 | ILE D | 24 | −9.782 | 7.632 | 51.130 | 1.00 | 17.30 | 1RHP | 1812 |
| ATOM | 1703 | CD1 | ILE D | 24 | −12.131 | 5.690 | 50.760 | 1.00 | 13.45 | 1RHP | 1813 |
| ATOM | 1704 | N | THR D | 25 | −6.636 | 7.590 | 49.732 | 1.00 | 19.08 | 1RHP | 1814 |
| ATOM | 1705 | CA | THR D | 25 | −5.447 | 8.241 | 50.240 | 1.00 | 15.38 | 1RHP | 1815 |
| ATOM | 1706 | C | THR D | 25 | −5.832 | 9.524 | 50.960 | 1.00 | 16.64 | 1RHP | 1816 |
| ATOM | 1707 | O | THR D | 25 | −5.372 | 9.769 | 52.072 | 1.00 | 15.78 | 1RHP | 1817 |
| ATOM | 1708 | CB | THR D | 25 | −4.505 | 8.520 | 49.085 | 1.00 | 13.02 | 1RHP | 1818 |
| ATOM | 1709 | OG1 | THR D | 25 | −3.958 | 7.269 | 48.745 | 1.00 | 10.87 | 1RHP | 1819 |
| ATOM | 1710 | CG2 | THR D | 25 | −3.412 | 9.502 | 49.416 | 1.00 | 12.73 | 1RHP | 1820 |
| ATOM | 1711 | N | SER D | 26 | −6.676 | 10.345 | 50.367 | 1.00 | 16.68 | 1RHP | 1821 |
| ATOM | 1712 | CA | SER D | 26 | −7.075 | 11.586 | 50.991 | 1.00 | 17.91 | 1RHP | 1822 |
| ATOM | 1713 | C | SER D | 26 | −8.586 | 11.493 | 51.093 | 1.00 | 18.00 | 1RHP | 1823 |
| ATOM | 1714 | O | SER D | 26 | −9.203 | 10.628 | 50.455 | 1.00 | 19.53 | 1RHP | 1824 |
| ATOM | 1715 | CB | SER D | 26 | −6.685 | 12.745 | 50.101 | 1.00 | 18.18 | 1RHP | 1825 |
| ATOM | 1716 | OG | SER D | 26 | −6.792 | 14.036 | 50.687 | 1.00 | 23.68 | 1RHP | 1826 |
| ATOM | 1717 | N | LEU D | 27 | −9.217 | 12.320 | 51.882 | 1.00 | 14.21 | 1RHP | 1827 |
| ATOM | 1718 | CA | LEU D | 27 | −10.647 | 12.373 | 51.887 | 1.00 | 13.34 | 1RHP | 1828 |
| ATOM | 1719 | C | LEU D | 27 | −10.821 | 13.804 | 52.295 | 1.00 | 13.17 | 1RHP | 1829 |
| ATOM | 1720 | O | LEU D | 27 | −10.345 | 14.106 | 53.400 | 1.00 | 14.10 | 1RHP | 1830 |
| ATOM | 1721 | CB | LEU D | 27 | −11.293 | 11.502 | 52.964 | 1.00 | 13.24 | 1RHP | 1831 |
| ATOM | 1722 | CG | LEU D | 27 | −12.827 | 11.578 | 53.122 | 1.00 | 11.80 | 1RHP | 1832 |
| ATOM | 1723 | CD1 | LEU D | 27 | −13.441 | 10.447 | 52.349 | 1.00 | 12.67 | 1RHP | 1833 |
| ATOM | 1724 | CD2 | LEU D | 27 | −13.253 | 11.413 | 54.564 | 1.00 | 13.21 | 1RHP | 1834 |
| ATOM | 1725 | N | GLU D | 28 | −11.380 | 14.712 | 51.485 | 1.00 | 10.72 | 1RHP | 1835 |
| ATOM | 1726 | CA | GLU D | 28 | −11.626 | 16.022 | 52.055 | 1.00 | 11.24 | 1RHP | 1836 |
| ATOM | 1727 | C | GLU D | 28 | −13.097 | 16.215 | 52.302 | 1.00 | 12.43 | 1RHP | 1837 |
| ATOM | 1728 | O | GLU D | 28 | −13.936 | 15.641 | 51.612 | 1.00 | 13.85 | 1RHP | 1838 |
| ATOM | 1729 | CB | GLU D | 28 | −11.087 | 17.184 | 51.171 | 1.00 | 9.42 | 1RHP | 1839 |
| ATOM | 1730 | CG | GLU D | 28 | −11.613 | 17.656 | 49.840 | 1.00 | 6.58 | 1RHP | 1840 |
| ATOM | 1731 | CD | GLU D | 28 | −10.889 | 18.899 | 49.332 | 1.00 | 7.81 | 1RHP | 1841 |
| ATOM | 1732 | OE1 | GLU D | 28 | −10.561 | 19.776 | 50.137 | 1.00 | 11.12 | 1RHP | 1842 |
| ATOM | 1733 | OE2 | GLU D | 28 | −10.661 | 19.006 | 48.129 | 1.00 | 8.16 | 1RHP | 1843 |
| ATOM | 1734 | N | VAL D | 29 | −13.371 | 16.950 | 53.352 | 1.00 | 12.49 | 1RHP | 1844 |
| ATOM | 1735 | CA | VAL D | 29 | −14.704 | 17.227 | 53.792 | 1.00 | 15.01 | 1RHP | 1845 |
| ATOM | 1736 | C | VAL D | 29 | −14.803 | 18.732 | 53.597 | 1.00 | 16.20 | 1RHP | 1846 |
| ATOM | 1737 | O | VAL D | 29 | −13.949 | 19.470 | 54.112 | 1.00 | 15.11 | 1RHP | 1847 |
| ATOM | 1738 | CB | VAL D | 29 | −14.775 | 16.785 | 55.260 | 1.00 | 18.12 | 1RHP | 1848 |
| ATOM | 1739 | CG1 | VAL D | 29 | −16.103 | 17.242 | 55.850 | 1.00 | 17.46 | 1RHP | 1849 |
| ATOM | 1740 | CG2 | VAL D | 29 | −14.655 | 15.245 | 55.373 | 1.00 | 18.58 | 1RHP | 1850 |
| ATOM | 1741 | N | ILE D | 30 | −15.784 | 19.230 | 52.842 | 1.00 | 13.04 | 1RHP | 1851 |
| ATOM | 1742 | CA | ILE D | 30 | −15.895 | 20.672 | 52.556 | 1.00 | 10.04 | 1RHP | 1852 |
| ATOM | 1743 | C | ILE D | 30 | −17.252 | 21.176 | 53.111 | 1.00 | 12.59 | 1RHP | 1853 |
| ATOM | 1744 | O | ILE D | 30 | −18.275 | 20.610 | 52.682 | 1.00 | 17.79 | 1RHP | 1854 |
| ATOM | 1745 | CB | ILE D | 30 | −15.804 | 20.880 | 51.016 | 1.00 | 2.74 | 1RHP | 1855 |
| ATOM | 1746 | CG1 | ILE D | 30 | −14.807 | 19.946 | 50.374 | 1.00 | 2.57 | 1RHP | 1856 |
| ATOM | 1747 | CG2 | ILE D | 30 | −15.326 | 22.277 | 50.758 | 1.00 | 2.68 | 1RHP | 1857 |
| ATOM | 1748 | CD1 | ILE D | 30 | −14.731 | 19.871 | 48.856 | 1.00 | 2.71 | 1RHP | 1858 |
| ATOM | 1749 | N | LYS D | 31 | −17.390 | 22.173 | 54.027 | 1.00 | 11.90 | 1RHP | 1859 |
| ATOM | 1750 | CA | LYS D | 31 | −18.684 | 22.575 | 54.577 | 1.00 | 10.69 | 1RHP | 1860 |
| ATOM | 1751 | C | LYS D | 31 | −19.553 | 23.137 | 53.476 | 1.00 | 14.10 | 1RHP | 1861 |
| ATOM | 1752 | O | LYS D | 31 | −19.125 | 23.522 | 52.376 | 1.00 | 11.71 | 1RHP | 1862 |
| ATOM | 1753 | CB | LYS D | 31 | −18.588 | 23.669 | 55.662 | 1.00 | 12.98 | 1RHP | 1863 |
| ATOM | 1754 | CG | LYS D | 31 | −18.547 | 25.123 | 55.149 | 1.00 | 11.99 | 1RHP | 1864 |
| ATOM | 1755 | CD | LYS D | 31 | −18.463 | 26.265 | 56.193 | 1.00 | 15.56 | 1RHP | 1865 |
| ATOM | 1756 | CE | LYS D | 31 | −18.652 | 27.688 | 55.572 | 1.00 | 17.04 | 1RHP | 1866 |
| ATOM | 1757 | NZ | LYS D | 31 | −17.893 | 27.975 | 54.352 | 1.00 | 14.84 | 1RHP | 1867 |
| ATOM | 1758 | N | ALA D | 32 | −20.823 | 23.198 | 53.803 | 1.00 | 15.76 | 1RHP | 1868 |
| ATOM | 1759 | CA | ALA D | 32 | −21.836 | 23.727 | 52.922 | 1.00 | 15.43 | 1RHP | 1869 |
| ATOM | 1760 | C | ALA D | 32 | −21.710 | 25.219 | 52.915 | 1.00 | 14.22 | 1RHP | 1870 |
| ATOM | 1761 | O | ALA D | 32 | −21.466 | 25.845 | 53.940 | 1.00 | 14.48 | 1RHP | 1871 |
| ATOM | 1762 | CB | ALA D | 32 | −23.208 | 23.359 | 53.430 | 1.00 | 17.06 | 1RHP | 1872 |
| ATOM | 1763 | N | GLY D | 33 | −21.897 | 25.814 | 51.761 | 1.00 | 14.74 | 1RHP | 1873 |
| ATOM | 1764 | CA | GLY D | 33 | −21.833 | 27.256 | 51.671 | 1.00 | 13.58 | 1RHP | 1874 |
| ATOM | 1765 | C | GLY D | 33 | −22.236 | 27.626 | 50.270 | 1.00 | 12.95 | 1RHP | 1875 |
| ATOM | 1766 | O | GLY D | 33 | −22.691 | 26.771 | 49.515 | 1.00 | 10.21 | 1RHP | 1876 |
| ATOM | 1767 | N | PRO D | 34 | −21.995 | 28.835 | 49.815 | 1.00 | 13.16 | 1RHP | 1877 |
| ATOM | 1768 | CA | PRO D | 34 | −22.200 | 29.263 | 48.420 | 1.00 | 14.80 | 1RHP | 1878 |
| ATOM | 1769 | C | PRO D | 34 | −21.594 | 28.412 | 47.301 | 1.00 | 15.73 | 1RHP | 1879 |
| ATOM | 1770 | O | PRO D | 34 | −21.952 | 28.530 | 46.131 | 1.00 | 16.73 | 1RHP | 1880 |
| ATOM | 1771 | CB | PRO D | 34 | −21.658 | 30.655 | 48.375 | 1.00 | 16.22 | 1RHP | 1881 |
| ATOM | 1772 | CG | PRO D | 34 | −20.598 | 30.567 | 49.474 | 1.00 | 13.82 | 1RHP | 1882 |
| ATOM | 1773 | CD | PRO D | 34 | −21.329 | 29.850 | 50.592 | 1.00 | 13.38 | 1RHP | 1883 |
| ATOM | 1774 | N | HIS D | 35 | −20.591 | 27.585 | 47.598 | 1.00 | 15.17 | 1RHP | 1884 |
| ATOM | 1775 | CA | HIS D | 35 | −20.069 | 26.769 | 46.535 | 1.00 | 13.73 | 1RHP | 1885 |
| ATOM | 1776 | C | HIS D | 35 | −21.142 | 25.760 | 46.197 | 1.00 | 14.51 | 1RHP | 1886 |
| ATOM | 1777 | O | HIS D | 35 | −21.399 | 25.500 | 45.029 | 1.00 | 16.02 | 1RHP | 1887 |
| ATOM | 1778 | CB | HIS D | 35 | −18.743 | 26.059 | 46.931 | 1.00 | 10.05 | 1RHP | 1888 |
| ATOM | 1779 | CG | HIS D | 35 | −18.638 | 25.442 | 48.306 | 1.00 | 8.65 | 1RHP | 1889 |
| ATOM | 1780 | ND1 | HIS D | 35 | −18.259 | 26.085 | 49.387 | 1.00 | 9.12 | 1RHP | 1890 |
| ATOM | 1781 | CD2 | HIS D | 35 | −18.916 | 24.144 | 48.651 | 1.00 | 7.85 | 1RHP | 1891 |
| ATOM | 1782 | CE1 | HIS D | 35 | −18.301 | 25.233 | 50.372 | 1.00 | 7.93 | 1RHP | 1892 |
| ATOM | 1783 | NE2 | HIS D | 35 | −18.697 | 24.073 | 49.926 | 1.00 | 9.30 | 1RHP | 1893 |
| ATOM | 1784 | N | CYS D | 36 | −21.880 | 25.306 | 47.204 | 1.00 | 15.89 | 1RHP | 1894 |
| ATOM | 1785 | CA | CYS D | 36 | −22.815 | 24.195 | 47.056 | 1.00 | 16.88 | 1RHP | 1895 |
| ATOM | 1786 | C | CYS D | 36 | −23.610 | 24.094 | 48.377 | 1.00 | 17.77 | 1RHP | 1896 |
| ATOM | 1787 | O | CYS D | 36 | −22.965 | 24.112 | 49.439 | 1.00 | 18.78 | 1RHP | 1897 |
| ATOM | 1788 | CB | CYS D | 36 | −21.932 | 22.975 | 46.775 | 1.00 | 14.78 | 1RHP | 1898 |
| ATOM | 1789 | SG | CYS D | 36 | −22.637 | 21.338 | 46.581 | 1.00 | 13.26 | 1RHP | 1899 |
| ATOM | 1790 | N | PRO D | 37 | −24.952 | 24.021 | 48.467 | 1.00 | 16.82 | 1RHP | 1900 |
| ATOM | 1791 | CA | PRO D | 37 | −25.700 | 23.817 | 49.720 | 1.00 | 13.31 | 1RHP | 1901 |
| ATOM | 1792 | C | PRO D | 37 | −25.486 | 22.543 | 50.536 | 1.00 | 11.73 | 1RHP | 1902 |
| ATOM | 1793 | O | PRO D | 37 | −26.054 | 22.406 | 51.607 | 1.00 | 11.94 | 1RHP | 1903 |
| ATOM | 1794 | CB | PRO D | 37 | −27.109 | 23.978 | 49.261 | 1.00 | 13.25 | 1RHP | 1904 |
| ATOM | 1795 | CG | PRO D | 37 | −27.101 | 23.473 | 47.830 | 1.00 | 15.07 | 1RHP | 1905 |
| ATOM | 1796 | CD | PRO D | 37 | −25.876 | 24.245 | 47.353 | 1.00 | 16.67 | 1RHP | 1906 |
| ATOM | 1797 | N | THR D | 38 | −24.682 | 21.589 | 50.101 | 1.00 | 13.27 | 1RHP | 1907 |
| ATOM | 1798 | CA | THR D | 38 | −24.491 | 20.298 | 50.737 | 1.00 | 11.38 | 1RHP | 1908 |
| ATOM | 1799 | C | THR D | 38 | −23.041 | 20.142 | 51.174 | 1.00 | 13.71 | 1RHP | 1909 |
| ATOM | 1800 | O | THR D | 38 | −22.159 | 20.678 | 50.492 | 1.00 | 16.45 | 1RHP | 1910 |
| ATOM | 1801 | CB | THR D | 38 | −24.827 | 19.213 | 49.744 | 1.00 | 9.92 | 1RHP | 1911 |
| ATOM | 1802 | OG1 | THR D | 38 | −24.622 | 18.028 | 50.452 | 1.00 | 9.34 | 1RHP | 1912 |
| ATOM | 1803 | CG2 | THR D | 38 | −23.948 | 19.134 | 48.518 | 1.00 | 7.06 | 1RHP | 1913 |
| ATOM | 1804 | N | ALA D | 39 | −22.780 | 19.405 | 52.265 | 1.00 | 10.86 | 1RHP | 1914 |
| ATOM | 1805 | CA | ALA D | 39 | −21.424 | 19.061 | 52.674 | 1.00 | 7.71 | 1RHP | 1915 |
| ATOM | 1806 | C | ALA D | 39 | −20.839 | 18.218 | 51.547 | 1.00 | 7.61 | 1RHP | 1916 |
| ATOM | 1807 | O | ALA D | 39 | −21.628 | 17.761 | 50.719 | 1.00 | 10.19 | 1RHP | 1917 |
| ATOM | 1808 | CB | ALA D | 39 | −21.470 | 18.231 | 53.924 | 1.00 | 4.74 | 1RHP | 1918 |
| ATOM | 1809 | N | GLN D | 40 | −19.552 | 17.981 | 51.337 | 1.00 | 6.87 | 1RHP | 1919 |
| ATOM | 1810 | CA | GLN D | 40 | −19.091 | 17.089 | 50.260 | 1.00 | 8.38 | 1RHP | 1920 |
| ATOM | 1811 | C | GLN D | 40 | −17.955 | 16.238 | 50.792 | 1.00 | 10.17 | 1RHP | 1921 |
| ATOM | 1812 | O | GLN D | 40 | −17.213 | 16.748 | 51.639 | 1.00 | 15.35 | 1RHP | 1922 |
| ATOM | 1813 | CB | GLN D | 40 | −18.556 | 17.859 | 49.039 | 1.00 | 5.42 | 1RHP | 1923 |
| ATOM | 1814 | CG | GLN D | 40 | −19.572 | 18.551 | 48.148 | 1.00 | 2.36 | 1RHP | 1924 |
| ATOM | 1815 | CD | GLN D | 40 | −18.994 | 19.628 | 47.256 | 1.00 | 2.27 | 1RHP | 1925 |
| ATOM | 1816 | OE1 | GLN D | 40 | −18.462 | 19.386 | 46.193 | 1.00 | 3.32 | 1RHP | 1926 |
| ATOM | 1817 | NE2 | GLN D | 40 | −19.055 | 20.881 | 47.595 | 1.00 | 4.17 | 1RHP | 1927 |
| ATOM | 1818 | N | LEU D | 41 | −17.773 | 14.979 | 50.391 | 1.00 | 9.47 | 1RHP | 1928 |
| ATOM | 1819 | CA | LEU D | 41 | −16.705 | 14.135 | 50.893 | 1.00 | 7.65 | 1RHP | 1929 |
| ATOM | 1820 | C | LEU D | 41 | −16.061 | 13.608 | 49.635 | 1.00 | 8.17 | 1RHP | 1930 |
| ATOM | 1821 | O | LEU D | 41 | −16.790 | 12.938 | 48.901 | 1.00 | 8.28 | 1RHP | 1931 |
| ATOM | 1822 | CB | LEU D | 41 | −17.273 | 12.993 | 51.724 | 1.00 | 6.93 | 1RHP | 1932 |
| ATOM | 1823 | CG | LEU D | 41 | −17.650 | 13.257 | 53.194 | 1.00 | 6.51 | 1RHP | 1933 |
| ATOM | 1824 | CD1 | LEU D | 41 | −18.895 | 14.098 | 53.320 | 1.00 | 7.73 | 1RHP | 1934 |
| ATOM | 1825 | CD2 | LEU D | 41 | −17.982 | 11.947 | 53.861 | 1.00 | 6.45 | 1RHP | 1935 |
| ATOM | 1826 | N | ILE D | 42 | −14.786 | 13.910 | 49.311 | 1.00 | 7.03 | 1RHP | 1936 |
| ATOM | 1827 | CA | ILE D | 42 | −14.143 | 13.517 | 48.045 | 1.00 | 8.69 | 1RHP | 1937 |
| ATOM | 1828 | C | ILE D | 42 | −12.906 | 12.646 | 48.301 | 1.00 | 10.63 | 1RHP | 1938 |
| ATOM | 1829 | O | ILE D | 42 | −11.869 | 13.088 | 48.826 | 1.00 | 12.71 | 1RHP | 1939 |
| ATOM | 1830 | CB | ILE D | 42 | −13.757 | 14.807 | 47.227 | 1.00 | 5.48 | 1RHP | 1940 |
| ATOM | 1831 | CG1 | ILE D | 42 | −14.948 | 15.662 | 46.949 | 1.00 | 7.77 | 1RHP | 1941 |
| ATOM | 1832 | CG2 | ILE D | 42 | −13.331 | 14.468 | 45.828 | 1.00 | 3.68 | 1RHP | 1942 |
| ATOM | 1833 | CD1 | ILE D | 42 | −14.559 | 17.113 | 46.717 | 1.00 | 7.45 | 1RHP | 1943 |
| ATOM | 1834 | N | ALA D | 43 | −12.995 | 11.378 | 47.916 | 1.00 | 13.19 | 1RHP | 1944 |
| ATOM | 1835 | CA | ALA D | 43 | −11.946 | 10.400 | 48.190 | 1.00 | 14.18 | 1RHP | 1945 |
| ATOM | 1836 | C | ALA D | 43 | −11.135 | 10.107 | 46.954 | 1.00 | 13.61 | 1RHP | 1946 |
| ATOM | 1837 | O | ALA D | 43 | −11.704 | 9.711 | 45.924 | 1.00 | 12.69 | 1RHP | 1947 |
| ATOM | 1838 | CB | ALA D | 43 | −12.492 | 9.056 | 48.639 | 1.00 | 14.96 | 1RHP | 1948 |
| ATOM | 1839 | N | THR D | 44 | −9.841 | 10.421 | 47.099 | 1.00 | 12.34 | 1RHP | 1949 |
| ATOM | 1840 | CA | THR D | 44 | −8.836 | 10.184 | 46.087 | 1.00 | 8.88 | 1RHP | 1950 |
| ATOM | 1841 | C | THR D | 44 | −8.455 | 8.779 | 46.361 | 1.00 | 9.47 | 1RHP | 1951 |
| ATOM | 1842 | O | THR D | 44 | −8.077 | 8.357 | 47.449 | 1.00 | 9.36 | 1RHP | 1952 |
| ATOM | 1843 | CB | THR D | 44 | −7.576 | 11.009 | 46.235 | 1.00 | 8.71 | 1RHP | 1953 |
| ATOM | 1844 | OG1 | THR D | 44 | −7.915 | 12.396 | 46.225 | 1.00 | 9.42 | 1RHP | 1954 |
| ATOM | 1845 | CG2 | THR D | 44 | −6.593 | 10.641 | 45.142 | 1.00 | 9.32 | 1RHP | 1955 |
| ATOM | 1846 | N | LEU D | 45 | −8.644 | 8.052 | 45.326 | 1.00 | 12.98 | 1RHP | 1956 |
| ATOM | 1847 | CA | LEU D | 45 | −8.349 | 6.637 | 45.370 | 1.00 | 18.61 | 1RHP | 1957 |
| ATOM | 1848 | C | LEU D | 45 | −6.844 | 6.481 | 45.165 | 1.00 | 19.13 | 1RHP | 1958 |
| ATOM | 1849 | O | LEU D | 45 | −6.201 | 7.493 | 44.855 | 1.00 | 20.48 | 1RHP | 1959 |
| ATOM | 1850 | CB | LEU D | 45 | −9.131 | 5.947 | 44.234 | 1.00 | 18.47 | 1RHP | 1960 |
| ATOM | 1851 | CG | LEU D | 45 | −9.709 | 4.594 | 44.498 | 1.00 | 17.21 | 1RHP | 1961 |
| ATOM | 1852 | CD1 | LEU D | 45 | −10.935 | 4.848 | 45.358 | 1.00 | 21.32 | 1RHP | 1962 |
| ATOM | 1853 | CD2 | LEU D | 45 | −9.971 | 3.832 | 43.194 | 1.00 | 19.52 | 1RHP | 1963 |
| ATOM | 1854 | N | LYS D | 46 | −6.294 | 5.257 | 45.246 | 1.00 | 18.64 | 1RHP | 1964 |
| ATOM | 1855 | CA | LYS D | 46 | −4.897 | 5.030 | 44.895 | 1.00 | 16.74 | 1RHP | 1965 |
| ATOM | 1856 | C | LYS D | 46 | −4.631 | 5.440 | 43.446 | 1.00 | 17.50 | 1RHP | 1966 |
| ATOM | 1857 | O | LYS D | 46 | −3.608 | 6.051 | 43.145 | 1.00 | 17.84 | 1RHP | 1967 |
| ATOM | 1858 | CB | LYS D | 46 | −4.544 | 3.557 | 45.062 | 1.00 | 17.22 | 1RHP | 1968 |
| ATOM | 1859 | CG | LYS D | 46 | −3.928 | 3.306 | 46.423 | 1.00 | 18.95 | 1RHP | 1969 |
| ATOM | 1860 | CD | LYS D | 46 | −3.573 | 1.840 | 46.578 | 1.00 | 21.44 | 1RHP | 1970 |
| ATOM | 1861 | CE | LYS D | 46 | −2.727 | 1.637 | 47.831 | 1.00 | 24.05 | 1RHP | 1971 |
| ATOM | 1862 | NZ | LYS D | 46 | −3.394 | 2.150 | 49.021 | 1.00 | 26.12 | 1RHP | 1972 |
| ATOM | 1863 | N | ASN D | 47 | −5.575 | 5.189 | 42.516 | 1.00 | 16.43 | 1RHP | 1973 |
| ATOM | 1864 | CA | ASN D | 47 | −5.367 | 5.447 | 41.095 | 1.00 | 15.76 | 1RHP | 1974 |
| ATOM | 1865 | C | ASN D | 47 | −5.664 | 6.898 | 40.732 | 1.00 | 19.51 | 1RHP | 1975 |
| ATOM | 1866 | O | ASN D | 47 | −6.066 | 7.166 | 39.596 | 1.00 | 20.94 | 1RHP | 1976 |
| ATOM | 1867 | CB | ASN D | 47 | −6.262 | 4.587 | 40.172 | 1.00 | 12.81 | 1RHP | 1977 |
| ATOM | 1868 | CG | ASN D | 47 | −6.898 | 3.315 | 40.691 | 1.00 | 11.36 | 1RHP | 1978 |
| ATOM | 1869 | OD1 | ASN D | 47 | −7.040 | 3.077 | 41.891 | 1.00 | 11.50 | 1RHP | 1979 |
| ATOM | 1870 | ND2 | ASN D | 47 | −7.342 | 2.450 | 39.809 | 1.00 | 11.65 | 1RHP | 1980 |
| ATOM | 1871 | N | GLY D | 48 | −5.541 | 7.902 | 41.609 | 1.00 | 22.36 | 1RHP | 1981 |
| ATOM | 1872 | CA | GLY D | 48 | −5.848 | 9.292 | 41.231 | 1.00 | 24.24 | 1RHP | 1982 |
| ATOM | 1873 | C | GLY D | 48 | −7.298 | 9.577 | 40.820 | 1.00 | 25.06 | 1RHP | 1983 |
| ATOM | 1874 | O | GLY D | 48 | −7.620 | 10.667 | 40.356 | 1.00 | 24.23 | 1RHP | 1984 |
| ATOM | 1875 | N | ARG D | 49 | −8.191 | 8.595 | 40.964 | 1.00 | 23.39 | 1RHP | 1985 |
| ATOM | 1876 | CA | ARG D | 49 | −9.624 | 8.662 | 40.673 | 1.00 | 20.79 | 1RHP | 1986 |
| ATOM | 1877 | C | ARG D | 49 | −10.148 | 9.470 | 41.844 | 1.00 | 18.16 | 1RHP | 1987 |
| ATOM | 1878 | O | ARG D | 49 | −9.646 | 9.277 | 42.956 | 1.00 | 19.05 | 1RHP | 1988 |
| ATOM | 1879 | CB | ARG D | 49 | −10.262 | 7.250 | 40.725 | 1.00 | 22.21 | 1RHP | 1989 |
| ATOM | 1880 | CG | ARG D | 49 | −11.439 | 6.807 | 39.823 | 1.00 | 23.16 | 1RHP | 1990 |
| ATOM | 1881 | CD | ARG D | 49 | −11.042 | 6.460 | 38.360 | 1.00 | 26.45 | 1RHP | 1991 |
| ATOM | 1882 | NE | ARG D | 49 | −10.199 | 5.265 | 38.248 | 1.00 | 28.79 | 1RHP | 1992 |
| ATOM | 1883 | CZ | ARG D | 49 | −9.907 | 4.629 | 37.080 | 1.00 | 28.01 | 1RHP | 1993 |
| ATOM | 1884 | NH1 | ARG D | 49 | −10.314 | 5.003 | 35.852 | 1.00 | 26.05 | 1RHP | 1994 |
| ATOM | 1885 | NH2 | ARG D | 49 | −9.148 | 3.537 | 37.129 | 1.00 | 25.25 | 1RHP | 1995 |
| ATOM | 1886 | N | LYS D | 50 | −11.136 | 10.334 | 41.627 | 1.00 | 15.17 | 1RHP | 1996 |
| ATOM | 1887 | CA | LYS D | 50 | −11.798 | 11.097 | 42.682 | 1.00 | 11.03 | 1RHP | 1997 |
| ATOM | 1888 | C | LYS D | 50 | −13.204 | 10.565 | 42.721 | 1.00 | 9.74 | 1RHP | 1998 |
| ATOM | 1889 | O | LYS D | 50 | −13.815 | 10.510 | 41.664 | 1.00 | 10.47 | 1RHP | 1999 |
| ATOM | 1890 | CB | LYS D | 50 | −11.971 | 12.546 | 42.365 | 1.00 | 8.93 | 1RHP | 2000 |
| ATOM | 1891 | CG | LYS D | 50 | −10.708 | 13.303 | 42.174 | 1.00 | 7.28 | 1RHP | 2001 |
| ATOM | 1892 | CD | LYS D | 50 | −10.103 | 13.566 | 43.499 | 1.00 | 3.80 | 1RHP | 2002 |
| ATOM | 1893 | CE | LYS D | 50 | −9.380 | 14.807 | 43.157 | 1.00 | 4.95 | 1RHP | 2003 |
| ATOM | 1894 | NZ | LYS D | 50 | −9.737 | 15.780 | 44.150 | 1.00 | 9.23 | 1RHP | 2004 |
| ATOM | 1895 | N | ILE D | 51 | −13.760 | 10.162 | 43.839 | 1.00 | 9.86 | 1RHP | 2005 |
| ATOM | 1896 | CA | ILE D | 51 | −15.164 | 9.781 | 43.929 | 1.00 | 8.48 | 1RHP | 2006 |
| ATOM | 1897 | C | ILE D | 51 | −15.732 | 10.697 | 45.007 | 1.00 | 12.32 | 1RHP | 2007 |
| ATOM | 1898 | O | ILE D | 51 | −14.958 | 11.337 | 45.748 | 1.00 | 16.76 | 1RHP | 2008 |
| ATOM | 1899 | CB | ILE D | 51 | −15.332 | 8.346 | 44.378 | 1.00 | 5.63 | 1RHP | 2009 |
| ATOM | 1900 | CG1 | ILE D | 51 | −14.603 | 8.069 | 45.671 | 1.00 | 4.49 | 1RHP | 2010 |
| ATOM | 1901 | CG2 | ILE D | 51 | −14.840 | 7.465 | 43.266 | 1.00 | 4.95 | 1RHP | 2011 |
| ATOM | 1902 | CD1 | ILE D | 51 | −14.665 | 6.598 | 46.093 | 1.00 | 6.76 | 1RHP | 2012 |
| ATOM | 1903 | N | CYS D | 52 | −17.044 | 10.846 | 45.149 | 1.00 | 11.92 | 1RHP | 2013 |
| ATOM | 1904 | CA | CYS D | 52 | −17.556 | 11.649 | 46.256 | 1.00 | 12.01 | 1RHP | 2014 |
| ATOM | 1905 | C | CYS D | 52 | −18.356 | 10.654 | 47.074 | 1.00 | 12.93 | 1RHP | 2015 |
| ATOM | 1906 | O | CYS D | 52 | −18.693 | 9.596 | 46.535 | 1.00 | 14.85 | 1RHP | 2016 |
| ATOM | 1907 | CB | CYS D | 52 | −18.439 | 12.756 | 45.749 | 1.00 | 11.15 | 1RHP | 2017 |
| ATOM | 1908 | SG | CYS D | 52 | −17.648 | 14.013 | 44.695 | 1.00 | 9.14 | 1RHP | 2010 |
| ATOM | 1909 | N | LEU D | 53 | −18.674 | 10.808 | 48.348 | 1.00 | 13.33 | 1RHP | 2019 |
| ATOM | 1910 | CA | LEU D | 53 | −19.412 | 9.767 | 49.060 | 1.00 | 12.89 | 1RHP | 2020 |
| ATOM | 1911 | C | LEU D | 53 | −20.682 | 10.369 | 49.674 | 1.00 | 13.93 | 1RHP | 2021 |
| ATOM | 1912 | O | LEU D | 53 | −20.765 | 11.601 | 49.828 | 1.00 | 14.10 | 1RHP | 2022 |
| ATOM | 1913 | CB | LEU D | 53 | −18.551 | 9.184 | 50.167 | 1.00 | 9.53 | 1RHP | 2023 |
| ATOM | 1914 | CG | LEU D | 53 | −17.108 | 8.806 | 49.952 | 1.00 | 5.90 | 1RHP | 2024 |
| ATOM | 1915 | CD1 | LEU D | 53 | −16.479 | 8.468 | 51.288 | 1.00 | 4.94 | 1RHP | 2025 |
| ATOM | 1916 | CD2 | LEU D | 53 | −17.035 | 7.688 | 48.965 | 1.00 | 7.43 | 1RHP | 2026 |
| ATOM | 1917 | N | ASP D | 54 | −21.698 | 9.584 | 50.054 | 1.00 | 14.18 | 1RHP | 2027 |
| ATOM | 1918 | CA | ASP D | 54 | −22.917 | 10.204 | 50.566 | 1.00 | 18.28 | 1RHP | 2028 |
| ATOM | 1919 | C | ASP D | 54 | −22.789 | 10.634 | 52.005 | 1.00 | 18.78 | 1RHP | 2029 |
| ATOM | 1920 | O | ASP D | 54 | −22.518 | 9.809 | 52.874 | 1.00 | 16.53 | 1RHP | 2030 |
| ATOM | 1921 | CB | ASP D | 54 | −24.151 | 9.266 | 50.469 | 1.00 | 21.16 | 1RHP | 2031 |
| ATOM | 1922 | CG | ASP D | 54 | −25.415 | 9.850 | 51.111 | 1.00 | 22.28 | 1RHP | 2032 |
| ATOM | 1923 | OD1 | ASP D | 54 | −25.803 | 10.950 | 50.738 | 1.00 | 25.28 | 1RHP | 2033 |
| ATOM | 1924 | OD2 | ASP D | 54 | −25.976 | 9.235 | 52.024 | 1.00 | 22.53 | 1RHP | 2034 |
| ATOM | 1925 | N | LEU D | 55 | −23.139 | 11.895 | 52.228 | 1.00 | 18.08 | 1RHP | 2035 |
| ATOM | 1926 | CA | LEU D | 55 | −23.042 | 12.546 | 53.513 | 1.00 | 19.91 | 1RHP | 2036 |
| ATOM | 1927 | C | LEU D | 55 | −23.631 | 11.797 | 54.710 | 1.00 | 23.01 | 1RHP | 2037 |
| ATOM | 1928 | O | LEU D | 55 | −23.383 | 12.129 | 55.872 | 1.00 | 23.55 | 1RHP | 2038 |
| ATOM | 1929 | CB | LEU D | 55 | −23.662 | 13.907 | 53.265 | 1.00 | 18.17 | 1RHP | 2039 |
| ATOM | 1930 | CG | LEU D | 55 | −24.361 | 14.797 | 54.294 | 1.00 | 18.62 | 1RHP | 2040 |
| ATOM | 1931 | CD1 | LEU D | 55 | −23.435 | 15.247 | 55.396 | 1.00 | 18.28 | 1RHP | 2041 |
| ATOM | 1932 | CD2 | LEU D | 55 | −24.913 | 16.013 | 53.544 | 1.00 | 19.09 | 1RHP | 2042 |
| ATOM | 1933 | N | GLN D | 56 | −24.431 | 10.761 | 54.543 | 1.00 | 27.77 | 1RHP | 2043 |
| ATOM | 1934 | CA | GLN D | 56 | −24.948 | 10.061 | 55.720 | 1.00 | 30.72 | 1RHP | 2044 |
| ATOM | 1935 | C | GLN D | 56 | −24.556 | 8.578 | 55.603 | 1.00 | 31.55 | 1RHP | 2045 |
| ATOM | 1936 | O | GLN D | 56 | −25.171 | 7.698 | 56.243 | 1.00 | 34.65 | 1RHP | 2046 |
| ATOM | 1937 | CB | GLN D | 56 | −26.501 | 10.241 | 55.769 | 1.00 | 30.02 | 1RHP | 2047 |
| ATOM | 1938 | CG | GLN D | 56 | −27.076 | 11.692 | 55.798 | 1.00 | 29.97 | 1RHP | 2048 |
| ATOM | 1939 | CD | GLN D | 56 | −27.142 | 12.439 | 54.452 | 1.00 | 28.27 | 1RHP | 2049 |
| ATOM | 1940 | OE1 | GLN D | 56 | −26.974 | 11.869 | 53.360 | 1.00 | 26.71 | 1RHP | 2050 |
| ATOM | 1941 | NE2 | GLN D | 56 | −27.358 | 13.755 | 54.502 | 1.00 | 26.59 | 1RHP | 2051 |
| ATOM | 1942 | N | ALA D | 57 | −23.546 | 8.224 | 54.783 | 1.00 | 28.28 | 1RHP | 2052 |
| ATOM | 1943 | CA | ALA D | 57 | −23.280 | 6.814 | 54.575 | 1.00 | 24.44 | 1RHP | 2053 |
| ATOM | 1944 | C | ALA D | 57 | −22.567 | 6.238 | 55.784 | 1.00 | 25.24 | 1RHP | 2054 |
| ATOM | 1945 | O | ALA D | 57 | −21.932 | 6.984 | 56.539 | 1.00 | 25.01 | 1RHP | 2055 |
| ATOM | 1946 | CB | ALA D | 57 | −22.411 | 6.631 | 53.367 | 1.00 | 22.94 | 1RHP | 2056 |
| ATOM | 1947 | N | PRO D | 58 | −22.608 | 4.916 | 56.019 | 1.00 | 26.00 | 1RHP | 2057 |
| ATOM | 1948 | CA | PRO D | 58 | −21.625 | 4.245 | 56.900 | 1.00 | 26.49 | 1RHP | 2058 |
| ATOM | 1949 | C | PRO D | 58 | −20.152 | 4.497 | 56.505 | 1.00 | 23.58 | 1RHP | 2059 |
| ATOM | 1950 | O | PRO D | 58 | −19.323 | 4.900 | 57.325 | 1.00 | 23.38 | 1RHP | 2060 |
| ATOM | 1951 | CB | PRO D | 58 | −22.076 | 2.764 | 56.862 | 1.00 | 26.11 | 1RHP | 2061 |
| ATOM | 1952 | CG | PRO D | 58 | −23.058 | 2.641 | 55.694 | 1.00 | 27.60 | 1RHP | 2062 |
| ATOM | 1953 | CD | PRO D | 58 | −23.722 | 4.029 | 55.656 | 1.00 | 26.28 | 1RHP | 2063 |
| ATOM | 1954 | N | LEU D | 59 | −19.843 | 4.326 | 55.214 | 1.00 | 22.75 | 1RHP | 2064 |
| ATOM | 1955 | CA | LEU D | 59 | −18.540 | 4.544 | 54.593 | 1.00 | 21.40 | 1RHP | 2065 |
| ATOM | 1956 | C | LEU D | 59 | −17.496 | 5.416 | 55.269 | 1.00 | 22.52 | 1RHP | 2066 |
| ATOM | 1957 | O | LEU D | 59 | −16.397 | 4.939 | 55.541 | 1.00 | 24.31 | 1RHP | 2067 |
| ATOM | 1958 | CB | LEU D | 59 | −18.713 | 5.133 | 53.222 | 1.00 | 19.55 | 1RHP | 2068 |
| ATOM | 1959 | CG | LEU D | 59 | −19.472 | 4.337 | 52.225 | 1.00 | 19.62 | 1RHP | 2069 |
| ATOM | 1960 | CD1 | LEU D | 59 | −19.721 | 5.209 | 50.999 | 1.00 | 18.42 | 1RHP | 2070 |
| ATOM | 1961 | CD2 | LEU D | 59 | −18.710 | 3.034 | 51.967 | 1.00 | 19.57 | 1RHP | 2071 |
| ATOM | 1962 | N | TYR D | 60 | −17.783 | 6.670 | 55.608 | 1.00 | 22.05 | 1RHP | 2072 |
| ATOM | 1963 | CA | TYR D | 60 | −16.737 | 7.536 | 56.108 | 1.00 | 22.16 | 1RHP | 2073 |
| ATOM | 1964 | C | TYR D | 60 | −16.451 | 7.308 | 57.573 | 1.00 | 23.88 | 1RHP | 2074 |
| ATOM | 1965 | O | TYR D | 60 | −15.463 | 7.799 | 58.133 | 1.00 | 26.76 | 1RHP | 2075 |
| ATOM | 1966 | CB | TYR D | 60 | −17.113 | 8.988 | 55.891 | 1.00 | 23.16 | 1RHP | 2076 |
| ATOM | 1967 | CG | TYR D | 60 | −18.163 | 9.546 | 56.829 | 1.00 | 23.33 | 1RHP | 2077 |
| ATOM | 1968 | CD1 | TYR D | 60 | −19.489 | 9.279 | 56.604 | 1.00 | 25.03 | 1RHP | 2078 |
| ATOM | 1969 | CD2 | TYR D | 60 | −17.758 | 10.331 | 57.883 | 1.00 | 23.74 | 1RHP | 2079 |
| ATOM | 1970 | CE1 | TYR D | 60 | −20.436 | 9.796 | 57.451 | 1.00 | 27.72 | 1RHP | 2080 |
| ATOM | 1971 | CE2 | TYR D | 60 | −18.698 | 10.846 | 58.738 | 1.00 | 26.83 | 1RHP | 2081 |
| ATOM | 1972 | CZ | TYR D | 60 | −20.042 | 10.582 | 58.516 | 1.00 | 28.65 | 1RHP | 2082 |
| ATOM | 1973 | OH | TYR D | 60 | −21.001 | 11.124 | 59.370 | 1.00 | 30.10 | 1RHP | 2083 |
| ATOM | 1974 | N | LYS D | 61 | −17.346 | 6.607 | 58.241 | 1.00 | 24.74 | 1RHP | 2084 |
| ATOM | 1975 | CA | LYS D | 61 | −17.075 | 6.284 | 59.626 | 1.00 | 25.38 | 1RHP | 2085 |
| ATOM | 1976 | C | LYS D | 61 | −15.881 | 5.317 | 59.620 | 1.00 | 23.26 | 1RHP | 2086 |
| ATOM | 1977 | O | LYS D | 61 | −14.920 | 5.534 | 60.361 | 1.00 | 22.10 | 1RHP | 2087 |
| ATOM | 1978 | CB | LYS D | 61 | −18.353 | 5.695 | 60.223 | 1.00 | 26.71 | 1RHP | 2088 |
| ATOM | 1979 | CG | LYS D | 61 | −19.422 | 6.814 | 60.307 | 1.00 | 26.47 | 1RHP | 2089 |
| ATOM | 1980 | CD | LYS D | 61 | −20.616 | 6.690 | 59.286 | 1.00 | 23.38 | 1RHP | 2090 |
| ATOM | 1981 | CE | LYS D | 61 | −21.839 | 7.553 | 59.600 | 1.00 | 21.99 | 1RHP | 2091 |
| ATOM | 1982 | NZ | LYS D | 61 | −22.205 | 7.387 | 60.998 | 1.00 | 18.61 | 1RHP | 2092 |
| ATOM | 1983 | N | LYS D | 62 | −15.848 | 4.323 | 58.717 | 1.00 | 20.56 | 1RHP | 2093 |
| ATOM | 1984 | CA | LYS D | 62 | −14.671 | 3.472 | 58.642 | 1.00 | 19.69 | 1RHP | 2094 |
| ATOM | 1985 | C | LYS D | 62 | −13.576 | 4.345 | 58.069 | 1.00 | 19.69 | 1RHP | 2095 |
| ATOM | 1986 | O | LYS D | 62 | −12.590 | 4.598 | 58.760 | 1.00 | 20.23 | 1RHP | 2096 |
| ATOM | 1987 | CB | LYS D | 62 | −14.838 | 2.244 | 57.715 | 1.00 | 21.51 | 1RHP | 2097 |
| ATOM | 1988 | CG | LYS D | 62 | −15.325 | 0.992 | 58.474 | 1.00 | 22.74 | 1RHP | 2098 |
| ATOM | 1989 | CD | LYS D | 62 | −15.881 | −0.172 | 57.627 | 1.00 | 23.56 | 1RHP | 2099 |
| ATOM | 1990 | CE | LYS D | 62 | −14.791 | −0.933 | 56.839 | 1.00 | 26.97 | 1RHP | 2100 |
| ATOM | 1991 | NZ | LYS D | 62 | −15.291 | −2.204 | 56.325 | 1.00 | 26.58 | 1RHP | 2101 |
| ATOM | 1992 | N | ILE D | 63 | −13.788 | 4.887 | 56.870 | 1.00 | 17.99 | 1RHP | 2102 |
| ATOM | 1993 | CA | ILE D | 63 | −12.800 | 5.677 | 56.165 | 1.00 | 17.65 | 1RHP | 2103 |
| ATOM | 1994 | C | ILE D | 63 | −12.032 | 6.621 | 57.058 | 1.00 | 19.02 | 1RHP | 2104 |
| ATOM | 1995 | O | ILE D | 63 | −10.816 | 6.479 | 57.052 | 1.00 | 19.06 | 1RHP | 2105 |
| ATOM | 1996 | CB | ILE D | 63 | −13.517 | 6.423 | 55.024 | 1.00 | 17.89 | 1RHP | 2106 |
| ATOM | 1997 | CG1 | ILE D | 63 | −13.783 | 5.397 | 53.934 | 1.00 | 19.31 | 1RHP | 2107 |
| ATOM | 1998 | CG2 | ILE D | 63 | −12.712 | 7.580 | 54.462 | 1.00 | 16.42 | 1RHP | 2108 |
| ATOM | 1999 | CD1 | ILE D | 63 | −14.860 | 5.870 | 52.941 | 1.00 | 19.55 | 1RHP | 2109 |
| ATOM | 2000 | N | ILE D | 64 | −12.568 | 7.462 | 57.933 | 1.00 | 19.14 | 1RHP | 2110 |
| ATOM | 2001 | CA | ILE D | 64 | −11.678 | 8.363 | 58.623 | 1.00 | 18.43 | 1RHP | 2111 |
| ATOM | 2002 | C | ILE D | 64 | −10.891 | 7.583 | 59.653 | 1.00 | 21.19 | 1RHP | 2112 |
| ATOM | 2003 | O | ILE D | 64 | −9.752 | 7.983 | 59.907 | 1.00 | 24.24 | 1RHP | 2113 |
| ATOM | 2004 | CB | ILE D | 64 | −12.474 | 9.491 | 59.259 | 1.00 | 15.95 | 1RHP | 2114 |
| ATOM | 2005 | CG1 | ILE D | 64 | −13.266 | 10.226 | 58.178 | 1.00 | 14.71 | 1RHP | 2115 |
| ATOM | 2006 | CG2 | ILE D | 64 | −11.521 | 10.464 | 59.936 | 1.00 | 15.17 | 1RHP | 2116 |
| ATOM | 2007 | CD1 | ILE D | 64 | −14.040 | 11.471 | 58.632 | 1.00 | 14.68 | 1RHP | 2117 |
| ATOM | 2008 | N | LYS D | 65 | −11.376 | 6.454 | 60.206 | 1.00 | 21.90 | 1RHP | 2118 |
| ATOM | 2009 | CA | LYS D | 65 | −10.604 | 5.642 | 61.166 | 1.00 | 21.89 | 1RHP | 2119 |
| ATOM | 2010 | C | LYS D | 65 | −9.392 | 4.981 | 60.485 | 1.00 | 20.01 | 1RHP | 2120 |
| ATOM | 2011 | O | LYS D | 65 | −8.233 | 5.177 | 60.856 | 1.00 | 18.28 | 1RHP | 2121 |
| ATOM | 2012 | CB | LYS D | 65 | −11.474 | 4.533 | 61.771 | 1.00 | 23.01 | 1RHP | 2122 |
| ATOM | 2013 | CG | LYS D | 65 | −12.628 | 5.075 | 62.601 | 1.00 | 23.16 | 1RHP | 2123 |
| ATOM | 2014 | CD | LYS D | 65 | −13.655 | 3.966 | 62.928 | 1.00 | 23.33 | 1RHP | 2124 |
| ATOM | 2015 | CE | LYS D | 65 | −14.907 | 4.624 | 63.547 | 1.00 | 20.65 | 1RHP | 2125 |
| ATOM | 2016 | NZ | LYS D | 65 | −15.738 | 3.636 | 64.207 | 1.00 | 19.75 | 1RHP | 2126 |
| ATOM | 2017 | N | LYS D | 66 | −9.633 | 4.283 | 59.386 | 1.00 | 17.38 | 1RHP | 2127 |
| ATOM | 2018 | CA | LYS D | 66 | −8.581 | 3.565 | 58.691 | 1.00 | 16.82 | 1RHP | 2128 |
| ATOM | 2019 | C | LYS D | 66 | −7.553 | 4.553 | 58.138 | 1.00 | 19.37 | 1RHP | 2129 |
| ATOM | 2020 | O | LYS D | 66 | −6.385 | 4.195 | 57.906 | 1.00 | 22.16 | 1RHP | 2130 |
| ATOM | 2021 | CB | LYS D | 66 | −9.162 | 2.746 | 57.545 | 1.00 | 16.19 | 1RHP | 2131 |
| ATOM | 2022 | CG | LYS D | 66 | −10.495 | 2.063 | 57.845 | 1.00 | 16.53 | 1RHP | 2132 |
| ATOM | 2023 | CD | LYS D | 66 | −10.758 | 0.912 | 56.884 | 1.00 | 16.55 | 1RHP | 2133 |
| ATOM | 2024 | CE | LYS D | 66 | −10.129 | −0.375 | 57.431 | 1.00 | 16.55 | 1RHP | 2134 |
| ATOM | 2025 | NZ | LYS D | 66 | −10.078 | −1.415 | 56.424 | 1.00 | 16.54 | 1RHP | 2135 |
| ATOM | 2026 | N | LEU D | 67 | −7.976 | 5.817 | 57.915 | 1.00 | 17.16 | 1RHP | 2136 |
| ATOM | 2027 | CA | LEU D | 67 | −7.072 | 6.835 | 57.411 | 1.00 | 12.69 | 1RHP | 2137 |
| ATOM | 2028 | C | LEU D | 67 | −6.407 | 7.472 | 58.593 | 1.00 | 12.91 | 1RHP | 2138 |
| ATOM | 2029 | O | LEU D | 67 | −5.391 | 8.120 | 58.441 | 1.00 | 12.92 | 1RHP | 2139 |
| ATOM | 2030 | CB | LEU D | 67 | −7.788 | 7.923 | 56.649 | 1.00 | 10.85 | 1RHP | 2140 |
| ATOM | 2031 | CG | LEU D | 67 | −8.389 | 7.646 | 55.296 | 1.00 | 8.81 | 1RHP | 2141 |
| ATOM | 2032 | CD1 | LEU D | 67 | −9.375 | 8.725 | 55.005 | 1.00 | 6.81 | 1RHP | 2142 |
| ATOM | 2033 | CD2 | LEU D | 67 | −7.337 | 7.603 | 54.226 | 1.00 | 10.59 | 1RHP | 2143 |
| ATOM | 2034 | N | LEU D | 68 | −6.910 | 7.392 | 59.793 | 1.00 | 14.40 | 1RHP | 2144 |
| ATOM | 2035 | CA | LEU D | 68 | −6.173 | 7.974 | 60.886 | 1.00 | 18.35 | 1RHP | 2145 |
| ATOM | 2036 | C | LEU D | 68 | −5.102 | 7.018 | 61.446 | 1.00 | 21.78 | 1RHP | 2146 |
| ATOM | 2037 | O | LEU D | 68 | −3.961 | 7.432 | 61.767 | 1.00 | 24.70 | 1RHP | 2147 |
| ATOM | 2038 | CB | LEU D | 68 | −7.212 | 8.385 | 61.908 | 1.00 | 15.58 | 1RHP | 2148 |
| ATOM | 2039 | CG | LEU D | 68 | −7.607 | 9.831 | 62.025 | 1.00 | 15.30 | 1RHP | 2149 |
| ATOM | 2040 | CD1 | LEU D | 68 | −7.470 | 10.611 | 60.741 | 1.00 | 13.31 | 1RHP | 2150 |
| ATOM | 2041 | CD2 | LEU D | 68 | −9.018 | 9.809 | 62.477 | 1.00 | 12.44 | 1RHP | 2151 |
| ATOM | 2042 | N | GLU D | 69 | −5.448 | 5.716 | 61.560 | 1.00 | 21.92 | 1RHP | 2152 |
| ATOM | 2043 | CA | GLU D | 69 | −4.530 | 4.739 | 62.118 | 1.00 | 22.44 | 1RHP | 2153 |
| ATOM | 2044 | C | GLU D | 69 | −3.549 | 4.133 | 61.117 | 1.00 | 24.23 | 1RHP | 2154 |
| ATOM | 2045 | O | GLU D | 69 | −3.892 | 3.590 | 60.055 | 1.00 | 19.20 | 1RHP | 2155 |
| ATOM | 2046 | CB | GLU D | 69 | −5.304 | 3.618 | 62.789 | 1.00 | 23.09 | 1RHP | 2156 |
| ATOM | 2047 | CG | GLU D | 69 | −6.117 | 4.100 | 63.991 | 1.00 | 21.18 | 1RHP | 2157 |
| ATOM | 2048 | CD | GLU D | 69 | −7.498 | 4.553 | 63.546 | 1.00 | 24.14 | 1RHP | 2158 |
| ATOM | 2049 | OE1 | GLU D | 69 | −8.351 | 3.701 | 63.295 | 1.00 | 24.62 | 1RHP | 2159 |
| ATOM | 2050 | OE2 | GLU D | 69 | −7.724 | 5.748 | 63.388 | 1.00 | 20.63 | 1RHP | 2160 |
| ATOM | 2051 | N | SER D | 70 | −2.315 | 4.440 | 61.536 | 1.00 | 26.05 | 1RHP | 2161 |
| ATOM | 2052 | CA | SER D | 70 | −1.032 | 4.058 | 60.962 | 1.00 | 28.78 | 1RHP | 2162 |
| ATOM | 2053 | C | SER D | 70 | −0.163 | 5.334 | 60.961 | 1.00 | 31.93 | 1RHP | 2163 |
| ATOM | 2054 | O | SER D | 70 | −0.642 | 6.465 | 61.239 | 1.00 | 34.97 | 1RHP | 2164 |
| ATOM | 2055 | CB | SER D | 70 | −1.101 | 3.571 | 59.506 | 1.00 | 26.86 | 1RHP | 2165 |
| ATOM | 2056 | OG | SER D | 70 | −0.003 | 2.730 | 59.169 | 1.00 | 27.94 | 1RHP | 2166 |
| TER | 2057 | | SER D | 70 | | | | | | 1RHP | 2167 |
| HETATM | 2058 | O | HOH D | 71 | −23.973 | 16.779 | 44.740 | 1.00 | 13.95 | 1RHP | 2168 |
| HETATM | 2059 | O | HOH D | 72 | −17.180 | 28.365 | 48.480 | 1.00 | 23.72 | 1RHP | 2169 |
| HETATM | 2060 | O | HOH D | 73 | −9.171 | 13.988 | 48.326 | 1.00 | 31.83 | 1RHP | 2170 |
| HETATM | 2061 | O | HOH D | 74 | −22.795 | 1.635 | 43.626 | 1.00 | 26.68 | 1RHP | 2171 |
| HETATM | 2062 | O | HOH D | 75 | −10.471 | 19.184 | 45.434 | 1.00 | 22.67 | 1RHP | 2172 |
| HETATM | 2063 | O | HOH D | 76 | −18.191 | 22.704 | 45.485 | 1.00 | 21.44 | 1RHP | 2173 |
| HETATM | 2064 | O | HOH D | 77 | −20.572 | 30.070 | 43.583 | 1.00 | 27.27 | 1RHP | 2174 |
| HETATM | 2065 | O | HOH D | 76 | −21.589 | 12.323 | 61.827 | 1.00 | 17.42 | 1RHP | 2175 |
| HETATM | 2066 | O | HOH D | 79 | −21.689 | 21.199 | 55.389 | 1.00 | 18.48 | 1RHP | 2116 |
| HETATM | 2067 | O | HOH D | 80 | −24.007 | 5.555 | 44.718 | 1.00 | 24.26 | 1RHP | 2177 |
| HETATM | 2068 | O | HOH D | 81 | −8.147 | −0.132 | 40.099 | 1.00 | 31.46 | 1RHP | 2178 |
| HETATM | 2069 | O | HOH D | 82 | −18.108 | 30.796 | 47.146 | 1.00 | 21.51 | 1RHP | 2179 |
| HETATM | 2070 | O | HOH D | 83 | 0.640 | 0.298 | 58.176 | 1.00 | 28.28 | 1RHP | 2180 |
| HETATM | 2071 | O | HOH D | 84 | −9.364 | 0.859 | 62.217 | 1.00 | 31.94 | 1RHP | 2181 |
| HETATM | 2072 | O | HOH D | 85 | −21.347 | 1.353 | 50.812 | 1.00 | 15.69 | 1RHP | 2182 |
| HETATM | 2073 | O | HOH D | 86 | −22.976 | −4.191 | 45.150 | 1.00 | 39.39 | 1RHP | 2183 |
| HETATM | 2074 | O | HOH D | 87 | −26.514 | 7.814 | 46.052 | 1.00 | 21.45 | 1RHP | 2184 |
| HETATM | 2075 | O | HOH D | 88 | −4.948 | −0.311 | 44.657 | 1.00 | 44.64 | 1RHP | 2185 |
| HETATM | 2076 | O | HOH D | 89 | −27.810 | 10.314 | 46.717 | 1.00 | 5.64 | 1RHP | 2186 |
| HETATM | 2077 | O | HOH D | 90 | −17.325 | −3.329 | 54.771 | 1.00 | 35.52 | 1RHP | 2187 |
| HETATM | 2078 | O | HOH D | 91 | −22.204 | 15.952 | 48.642 | 1.00 | 26.24 | 1RHP | 2188 |
| HETATM | 2079 | O | HOH D | 92 | −8.632 | 2.408 | 34.626 | 1.00 | 18.65 | 1RHP | 2189 |
| HETATM | 2080 | O | HOH D | 93 | −10.155 | −1.758 | 40.779 | 1.00 | 31.61 | 1RHP | 2190 |
| HETATM | 2081 | O | HOH D | 94 | −27.841 | 11.796 | 49.192 | 1.00 | 33.30 | 1RHP | 2191 |
| HETATM | 2082 | O | HOH D | 95 | −9.773 | −4.065 | 58.208 | 1.00 | 19.84 | 1RHP | 2192 |
| HETATM | 2083 | O | HOH D | 96 | −7.550 | 1.318 | 37.396 | 1.00 | 28.59 | 1RHP | 2193 |

| | | | | | |
|---|---|---|---|---|---|
| CONECT | 31 | 30 | 230 | 1RHP | 2194 |
| CONECT | 45 | 44 | 349 | 1RHP | 2195 |
| CONECT | 230 | 31 | 229 | 1RHP | 2196 |
| CONECT | 349 | 45 | 348 | 1RHP | 2197 |
| CONECT | 554 | 553 | 753 | 1RHP | 2198 |
| CONECT | 568 | 567 | 872 | 1RHP | 2199 |
| CONECT | 753 | 554 | 752 | 1RHP | 2200 |
| CONECT | 872 | 568 | 871 | 1RHP | 2201 |
| CONECT | 1076 | 1075 | 1275 | 1RHP | 2202 |
| CONECT | 1090 | 1089 | 1394 | 1RHP | 2203 |
| CONECT | 1275 | 1076 | 1274 | 1RHP | 2204 |
| CONECT | 1394 | 1090 | 1393 | 1RHP | 2205 |
| CONECT | 1590 | 1589 | 1789 | 1RHP | 2206 |
| CONECT | 1604 | 1603 | 1908 | 1RHP | 2207 |
| CONECT | 1789 | 1590 | 1788 | 1RHP | 2208 |
| CONECT | 1908 | 1604 | 1907 | 1RHP | 2209 |

| | | | |
|---|---|---|---|
| MASTER | 42 0 0 4 12 0 0 6 2079 4 16 24 | 1RHP | 2210 |

| | | |
|---|---|---|
| END | 1RHP | 2211 |

1-12. (canceled)

13. A compound that modulates PF4 activity comprising functional groups I, II, III, IV, VIII, IX and X wherein the distance between the functional groups in three-dimensions is about:
2.25±0.05 Å between groups I and II;
6.03±1.37 Å between groups I and III;
6.92±1.60 Å between groups I and IV;
8.57±2.60 Å between groups I and VIII;
14.20±1.53 Å between groups I and IX;
12.54±1.51 Å between groups I and X;
6.00±2.43 Å between groups II and III;
7.01±1.84 Å between groups II and IV;
9.09±1.22 Å between groups II and VIII;
14.45±0.24 Å between groups II and IX;
13.28±0.37 Å between groups II and X;
2.31±0.07 Å between groups III and IV;
9.19±1.40 Å between groups III and VIII;
10.91±1.74 Å between groups III and IX;
7.06±2.49 Å between groups III and X;
9.02±0.63 Å between groups IV and VIII;
10.46±0.46 Å between groups IV and IX;
6.52±1.26 Å between groups IV and X;
6.87±0.96 Å between groups VIII and IX
9.84±1.05 Å between groups VIII and X; and
7.25±0.49 Å between groups 1× and X
wherein functional group I corresponds to the OD1 atom of the amino acid side chain Asp7, functional group II corresponds to the OD2 atom of the amino acid side chain Asp7, functional group III corresponds to the NE2 atom of the amino acid side chain Gln9, functional group IV corresponds to the OE1 atom of the amino acid side chain Gln9, functional group VIII corresponds to the CG atom of the amino acid side chain Leu8, and functional group X corresponds to the CG atom of the amino acid side chain Leu11 in the PF4 sequence set forth in FIG. 1C (SEQ ID NO:1), and
wherein said compound is not PF4, IL-8, a PF4 Mutant or a peptide having the amino acid sequence selected from the group consisting of SEQ ID NOS:34-154.

14. The compound of claim 13 further comprising functional groups V, VI, and VII wherein the distance between the functional groups in three-dimensions is about
30.27±2.92 Å between groups I and V;
29.94±2.49 Å between groups I and VI;
30.41±4.31 Å between groups I and VII;
30.83±1.99 Å between groups II and V;
30.33±1.97 Å between groups II and VI;
31.24±4.03 Å between groups II and VII;
26.35±2.76 Å between groups III and V;
26.57±2.02 Å between groups III and VI;
26.31±3.05 Å between groups III and VII;
25.58±1.40 Å between groups IV and V;
25.80±1.31 Å between groups IV and VI;
25.34±2.81 Å between groups IV and VII;
3.85±1.54 Å between groups V and VI;
10.21±2.21 Å between groups V and VII;
23.10±2.21 Å between groups V and VIII;
17.29±1.68 Å between groups V and IX;
19.25±2.12 Å between groups V and X;
14.07±0.94 Å between groups VI and VII;
21.84±2.74 Å between groups VI and VIII;
16.42±2.03 Å between groups VI and IX;
19.95±2.02 Å between groups VI and X;
25.38±4.39 Å between groups VII and VIII;
20.60±3.57 Å between groups VII and IX; and
18.76±3.72 Å between groups VII and X
wherein functional group V corresponds to the OE1 atom of the amino acid side chain Gln18, functional group VI corresponds to the NE2 atom of the amino acid side chain Gln18, functional group VII corresponds to the NE2 atom of the amino acid side chain His23, and functional group IX corresponds to the CB atom of the amino acid side chain Val13 in the PF4 sequence set forth in FIG. 1C (SEQ ID NO:1).

15-24. (canceled)

25. The compound of claim 13 wherein the root-mean-squared deviation of the functional group distances is less than 1.0 angstroms.

26-27. (canceled)

28. The compound of claim 13 that is a PF4 antagonist or a PF4 antagonist.

29. (canceled)

30. The compound of claim 28 further comprising a detectable label.

31. The compound of claim 13, wherein said compound is a polypeptide having an amino acid sequence selected from the group consisting of SEQ ID NO:157 and SEQ ID NO:159.

32. A method for identifying a compound that modulates PF4 activity, which method comprises comparing:
(a) a three-dimensional structure of a candidate compound, to
(b) a three-dimensional structure of a PF4 pharmacophore,
wherein said PF4 pharmacophore comprises functional groups I, II, III, IV, VIII, IX and X
wherein the distance between the functional groups in three-dimensions is about:
2.25±0.05 Å between groups I and II;
6.03±1.37 Å between groups I and III;
6.92±1.60 Å between groups I and IV;
8.57±2.60 Å between groups I and VIII;
14.20±1.53 Å between groups I and IX;
12.54±1.51 Å between groups I and X;
6.00±2.43 Å between groups II and III;
7.01±1.84 Å between groups II and IV;
9.09±1.22 Å between groups II and VIII
14.45±0.24 Å between groups II and IX;
13.28±0.37 Å between groups II and X;
2.31±0.07 Å between groups III and IV;
9.19±1.40 Å between groups III and VIII
10.91±1.74 Å between groups III and IX;
7.06±2.49 Å between groups III and X;
9.02±0.63 Å between groups IV and VIII;
10.46±0.46 Å between groups IV and IX;
6.52±1.26 Å between groups IV and X;
6.87±0.96 Å between groups VIII and IX
9.84±1.05 Å between groups VIII and X; and
7.25±0.49 Å between groups 1× and X
wherein functional group I corresponds to the OD1 atom of the amino acid side chain Asp7, functional group II corresponds to the OD2 atom of the amino acid side chain Asp7, functional group III corresponds to the NE2 atom of the amino acid side chain Gln9, functional group IV corresponds to the OE1 atom of the amino acid side chain Gln9, functional group VIII corresponds to the CG atom of the amino acid side chain Leu8, and functional group X corresponds to the CG atom of the amino acid side chain Leu11 in the PF4 sequence set forth in FIG. 1C (SEQ ID NO:1),
wherein said candidate compound is not PF4, IL-8, a PF4 Mutant or a peptide having the amino acid sequence selected from the group consisting of SEQ ID NOS:34-156; and
wherein similarity between the three-dimensional structures of the candidate compound and the PF4 pharmacophore is indicative of the candidate compound's ability to modulate PF4 activity.

33. The method according to claim 32 wherein the root-mean square deviation (RMSD) between the three-dimensional structures of the candidate compound and the PF4 pharmacophore is not greater than about 1.0 angstrom.

34. The method according to claim 32 wherein the candidate compound is a peptidomimetic, a PF4 agonist or a PF4 antagonist.

35-45. (canceled)

46. The method according to claim 32 wherein the PF4 pharmacophore further comprising functional groups V, VI, and VII wherein the distance between the functional groups in three-dimensions is about
30.27±2.92 Å between groups I and V;
29.94±2.49 Å between groups I and VI;
30.41±4.31 Å between groups I and VII;
30.83±1.99 Å between groups II and V;
30.33±1.97 Å between groups II and VI;
31.24±4.03 Å between groups II and VII;
26.35±2.76 Å between groups III and V;
26.57±2.02 Å between groups III and VI;
26.31±3.05 Å between groups III and VII;
25.58±1.40 Å between groups IV and V;
25.80±1.31 Å between groups IV and VI;
25.34±2.81 Å between groups IV and VII;
3.85±1.54 Å between groups V and VI;
10.21±2.21 Å between groups V and VII;
23.10±2.21 Å between groups V and VIII;
17.29±1.68 Å between groups V and IX;
19.25±2.12 Å between groups V and X;
14.07±0.94 Å between groups VI and VII;
21.84±2.74 Å between groups VI and VIII;
16.42±2.03 Å between groups VI and IX;
19.95±2.02 Å between groups VI and X;
25.38±4.39 Å between groups VII and VIII;
20.60±3.57 Å between groups VII and IX; and
18.76±3.72 Å between groups VII and X
wherein functional group V corresponds to the OE1 atom of the amino acid side chain Gln18, functional group VI corresponds to the NE2 atom of the amino acid side chain Gln18, functional group VII corresponds to the NE2 atom of the amino acid side chain His23, and functional group IX corresponds to the CB atom of the amino acid side chain Val13 in the PF4 sequence set forth in FIG. 1C (SEQ ID NO:1).

47-56. (canceled)

57. A PF4 polypeptide having the amino acid sequence set forth in FIG. 1C (SEQ ID NO:1) and comprising at least one amino acid substitution that modulates interaction of the PF4 with heparan sulfate.

58. The PF4 polypeptide according to claim 57, wherein said mutation is selected from the group consisting of: Lys61→Gln, Lys62→Glu, Lys65→Gln and Lys66→Glu.

59. A PF4 polypeptide having the amino acid sequence set forth in FIG. 1C (SEQ ID NO:1) and comprising at least one amino acid substitution selected from the group consisting of Gln9→Arg, Gln9→Ala and Asp7→Ala.

60. A PF4 polypeptide having the amino acid sequence set forth in FIG. 1C (SEQ ID NO:1) and comprising at least one amino acid substitution selected from the group consisting of: Leu11→Ser, Val13→Gln, and Thr16→Ala.

61. A PF4 polypeptide having the amino acid sequence set forth in FIG. 1C (SEQ ID NO:1) and comprising at least one amino acid substitution selected from the group consisting of: Gln18→Ala, Val19→Ser, and His23→Ala.

62. A mutant PF4 polypeptide having the amino acid sequence selected from the group consisting of SEQ ID NOS:2-30.

63. An isolated polypeptide comprising an amino acid sequence selected from the group consisting of SEQ ID NO:157-160.

64. A compound according to claim 13, selected from the group consisting of the compound of Formula (I): 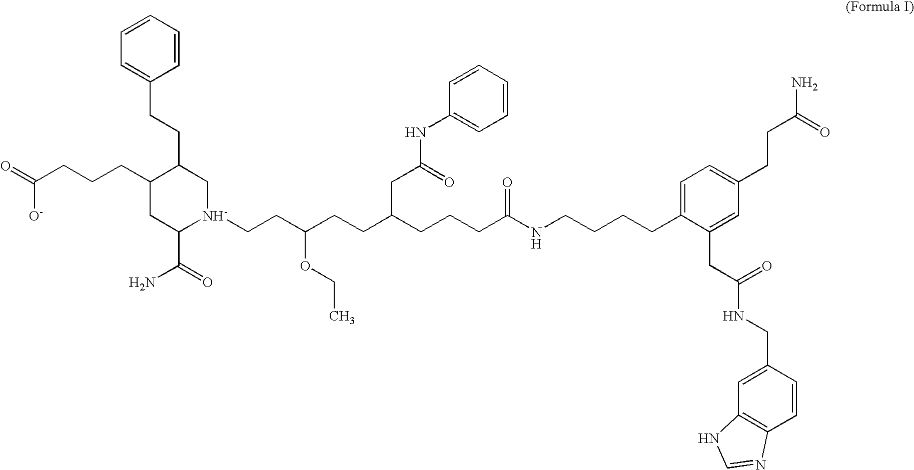
the compound of Formula II (FIG. 8), the compound of Formula III (FIG. 8), the compound of Formula IV (FIG. 8), the compound of Formula V (FIG. 8), the compound of Formula VI (FIG. 8), the compound of Formula VII (FIG. 8), and the compound of Formula VIII (FIG. 9A).

65-67. (canceled)

68. The compound according to claim 30, wherein the PF4 antagonist has a chemical structure as provided by Formula VII (FIG. 9A).

69. The compound according to claim 68, which has a chemical structure as provided by Formula VIII (FIG. 9B).

70-71. (canceled)

72. The compound according to claim 30, wherein the PF4 antagonist comprises a polypeptide having an amino acid sequence selected from the group consisting of SEQ ID NOS:34-156 and SEQ ID NO:159.

73. (canceled)

74. The compound according to claim 30, wherein the detectable label is selected from the group consisting of: a metal, a radioactive isotope, a radioopaque agent, a radiolucent agent, a contrast agent, a dye, and an enzyme that catalyzes a calorimetric or fluorometric reaction.

75. A method for detecting PF4 binding sites in an individual, which method comprises:
(c) administering, to the individual, a detectable marker according to claim 30; and
(d) detecting the presence of said detectable marker in the individual.

76. A method for detecting sites of angiogenesis in an individual, which method comprises:
(e) administering, to the individual, a detectable marker according to claim 30; and
(f) detecting the presence of said detectable marker in the individual.

77. A method for detecting an infection in an individual, which method comprises:
(g) administering, to the individual, a detectable marker according to claim 30; and
(h) detecting the presence of said detectable marker in the individual.

78-81. (canceled)
